(19) United States
(12) Patent Application Publication
(10) Pub. No.: US 2024/0132993 A1
(43) Pub. Date: Apr. 25, 2024

(54) SYSTEMS AND METHODS FOR EXTRACTING RARE EARTH ELEMENTS WITH ENGINEERED MICROORGANISMS

(71) Applicant: Cornell University, Ithaca, NY (US)

(72) Inventors: Buz BARSTOW, Ithaca, NY (US); Alexa SCHMITZ, Ithaca, NY (US); Brooke PIAN, Ithaca, NY (US); Sean MEDIN, Ithaca, NY (US)

(21) Appl. No.: 18/547,434

(22) PCT Filed: Feb. 18, 2022
(86) PCT No.: PCT/US2022/017101

§ 371 Date: Aug. 21, 2023

Related U.S. Application Data
(60) Provisional application No. 63/220,475, filed on Jul. 10, 2021.
(60) Provisional application No. 63/152,798, filed on Feb. 23, 2021.

(51) Int. Cl. C22B 3/18; C12N 1/20; C12N 9/02; C12N 9/06; C12N 15/74; C22B 59/00

(52) U.S. Cl. CPC C22B 3/18; C12N 1/205; C12N 9/001; C12N 9/0026; C12N 15/74; C12Y 103/03011; C12Y 105/08004; C22B 59/00; C12R 2001/01

(57) ABSTRACT

Provided are modified bacteria for use in bioleaching rare earth elements (REEs). The modified bacteria contain at least one engineered genetic change that is correlated with improved bioleaching of the REEs, relative to REE bioleaching by unmodified bacteria of the same species as the modified bacteria. Also provided is a method for extracting REEs by contacting a composition containing REEs with biolixiviant produced by the modified bacteria. Kits that include containers that hold the modified bacteria are also provided.

### CROSS REFERENCE TO RELATED APPLICATIONS

This application claims priority to U.S. provisional application No. 63/220,475, filed Jul. 10, 2021, and to U.S. provisional application No. 63/152,798, filed Feb. 23, 2021, the disclosures of each of which are incorporated herein by reference.

### SEQUENCE LISTING

The instant application contains a Sequence Listing which has been submitted electronically in ASCII format and is hereby incorporated by reference in its entirety. Said ASCII copy, created on Feb. 17, 2022, is named 018617_01341_SL.txt and is 979,969 bytes in size.

### BACKGROUND

Rare earth elements (REE) are essential for the manufacturing of modern electronics, sustainable energy technologies including electric motors and wind turbine generators; solid state lighting; battery anodes; high-temperature superconductors; and high-strength lightweight alloys. All of these applications place increasing demands on the global REE supply chain. As the world demand for sustainable energy grows, finding a reliable and sustainable source of REE is critical.

Current methods for refining REE often involve harsh chemicals, high temperatures, high pressures and generate a considerable amount of toxic waste. These processes give sustainable energy technologies reliant on REE a high environmental and carbon footprint. As a consequence, due to its high environmental standards, the United States has no capacity to produce purified REE.

There is growing interest in biological methods to supplement, if not completely replace traditional REE extraction and purification methods. Bioleaching is used to extract 5% of the world's gold, and ≈15% of the world's copper supply, and biomining in Chile alone accounts for 10% of the world's Cu supply.

The performance of REE-bioleaching lags behind thermochemical processes. For example, while thermochemical methods have 89-98% REE extraction efficiency from monazite ore, Aspergillus species can only achieve≈3-5%. The acid-producing microbe Gluconobacter oxydans B58 can recover≈50% of REE from FCC catalysts. However, techno-economic analysis indicates that even this extraction efficiency is still not high enough for commercial viability.

Recent efforts to improve bioleaching have focused exclusively on process optimization. It is believed that no previous genetic approaches have yet been taken for any bioleaching microbe. With recent advances in tools for reading and writing genomes, genetic engineering is an attractive solution for enhancing bioleaching. However, applying these tools to non-model microorganisms like G. oxydans can be a significant challenge. While there have been some advances for editing the genome of G. oxydans it has remained unknown where the genome can be edited to improve bioleaching results. Thus, there is an ongoing and unmet need for improved compositions, engineered organisms, and methods for separating REEs from compositions that contain them. The present disclosure is pertinent to this need.

### BRIEF SUMMARY

The present disclosure provides a description of a whole genome knockout collection for Gluconobacter oxydans B58, and use of it to comprehensively characterize the genomics of rare earth elements (REEs) bioleaching. In total, 304 genes that notably alter production of G. oxydans' acidic bio-lixiviant, including 165 that make statistically significant changes, were identified. Based in part on this analysis, the present disclosure provides modified bacteria for use in bioleaching REEs. The modified bacteria comprise at least one engineered genetic change that is correlated with improved bioleaching of the REEs, relative to REE bioleaching by unmodified bacteria of the same species as the modified bacteria. The at least one genetic change results in decreased expression, or increased expression, of at least one gene. In non-limiting embodiments, at least one gene for which expression is modified encodes a protein that participates in phosphate-specific transport system signaling, or encodes a protein that participates in pyrroloquinoline quinone (PQQ) synthesis. In non-limiting examples, expression of a gene that encodes a protein that participates in the phosphate-specific transport system signaling is suppressed. In certain embodiments, the suppressed gene is pstS, pstB or pstC. In certain embodiments, a gene that encodes a protein that participates in the PQQ synthesis is increased. In non-limiting embodiments, the expression of at least one of the genes pqqA, pqqB, pqqC, pqqD, pqqE, tldD and tldE, is increased. In addition to these and other genetic modifications described herein, the modified bacteria exhibit increase expression of mgdh relative to expression of mgdh by unmodified bacteria. In certain embodiments, expression of pstS, pstB, pstC, or a combination thereof is reduced, or expression of pqqA, pqqB, pqqC, pqqD, pqqE, tldD, tldE, or a combination thereof is increased. In these contexts expression of mgdh may also be increased.

In another aspect, the disclosure provides for contacting a composition comprising the REEs with a composition produced by the described modified bacteria. The composition produced by the bacteria may be considered a lixiviant, or a biolixiviant because it is produced by the described bacteria. The disclosure provides separating REEs from the composition after contacting the composition with the biolixiviant. The separated REEs are suitable for use in a wide range of applications that will be apparent to those skilled in the art.

In another aspect, the disclosure provides kits that contain one or more sealable containers in which the described modified bacteria are held. The kits may further comprise printed material, such as instructions for use of the modified bacteria to form a biolixiviant, and/or to extract REEs from a composition where they are present.

### BRIEF DESCRIPTION OF THE FIGURES

FIG. 1. Knockout Sudoku was used to curate a saturating coverage transposon insertion mutant collection for Gluconobacter oxydans B58. (A) The G. oxydans B58 genome contains 3,283 genes. 2,570 genes were fully annotated with a BLAST hit, Interpro ID, and gene ontology (GO) group. An additional 163 genes have an annotation and GO group, but lack an Interpro ID, 399 only retrieved a BLAST hit, but no GO group, and 150 were unable to be assigned any annotation. (B) A Monte Carlo (MC) estimate of the number of genes represented by at least one mutant as a function of the number of mutants collected demonstrated that picking 25,000 mutants would yield at least one disruption for 95% of genes, while picking 50,000 mutants would yield at least one disruption for 99% of genes. In total, we picked 49,256 single-gene disruption mutants and located at least one disruption for 2,733 genes. A Monte Carlo simulation of picking with random drawing from the sequenced progenitor collection (PC) without replacements demonstrates that the genome coverage was truly saturated. The center of each curve is the mean value of the unique gene disruption count from 1,000 simulations while the upper and lower part of each curve represent two standard deviations around this mean. (C) A Fisher's Exact Test for gene ontology enrichment among the non-disrupted (putatively essential) genes revealed significant enrichment (p<0.05, yellow line) of genes involved in translation and other ribosome-related functions. (D) The curated condensed collection (CC) contains 17,706 isolated colonies across 185 plates. High-throughput sequencing of the CC confirmed the location for 4,419 unique disruption strains, representing disruptions in 2,556 genes. 177 genes located in the PC were not located in the CC. No disruption mutant was detected in 550 genes.

FIG. 2. throughput pH screens of the G. oxydans whole genome knockout collection were used to identify genes that control REE bioleaching. (A) Thymol blue (TB) was used to measure the endpoint acidity of biolixiviant produced by each well of the condensed collection. The ratio of TB absorbance (A) at 435 and 545 nm is linearly related to pH between 2 and 3.4. CC plate 65 contains biolixiviant produced by δpstB strain in wells F7 and G7 (arrowhead), whose absorbance at 435 nm and 545 nm is shown, along with the average absorbance of all wells on the plate. The dashed line represents a typical absorbance spectrum for WT-produced biolixiviant. The A435/A545 ratio for these two wells compared with the average ratio of the plate is well below the lower bound (LB) for the plate, indicating that δpstB produces a much more acidic biolixiviant than the average strain. (B) Bromophenol blue (BPB) was used to measure rate of change in pH at the onset of glucose conversion to organic acids. Rate was measured over a six minute period within five minutes of adding bacteria to a glucose and BPB solution. Condensed collection (CC) plate 162 contains the δtldE strain in wells F11-C12 (arrowheads), whose changes in absorbance over time are graphed along with the average for that plate. A comparison of the normalized rate over OD for each well versus the plate average shows how V/OD for these wells was below the lower bound for CC plate 162. (C) All 185 plates of the CC were screened for acidification using the TB and BPB assays. Hits from both screens were verified in comparison with proxy WT strains. In total, 176 disruption strains were shown to significantly contribute to acidification by t-test with a Bonferroni-corrected alpha (ζ=0.05/#of comparisons). (D) The 25 largest reductions in biolixiviant pH, and 50 largest increases in biolixiviant pH. (E) All significant changes in acidification rate.

FIG. 3. Genes involved in phosphate signaling, carbohydrate metabolism and PQQ synthesis were significantly overrepresented in the significant hits from high-throughput screens of acidification by G. oxydans. Fisher's Exact Test was used to test for gene ontology enrichment (p<0.05, yellow dashed line). Numbers at base of bars are how many genes from the significant hits are from that gene ontology (GO), out of the total in the genome (in parentheses). Genes selected for further analysis of endpoint pH and bioleaching (FIG. 4) that contribute to an enriched GO are listed above the bars. (A and B) Enriched GO among genes that decrease and increase end point pH. (C and D) Enriched GO among genes that increase and decrease initial acidification rate. Abbreviations: FBP: fructose-bisphosphate; GDP-Man:DolP: dolichyl-phosphate beta-D-mannosyltransferase; GGT: glutathione hydrolase; G6P: glucose 6-phosphate; HTA: homoserine O-acetyltransferase; DD-transepeptidase: D-Ala-D-Ala carboxypeptidase; HAG: hydroxyacylglutathione; Membr: membraneMoco: Mo-molybdopterin cofactor; MS: monosaccharide; MT: mannosyltransferase; M6P: mannose-6-phosphate; Pᵢ: inorganic phosphate; PLP: pyridoxal phosphate; PQQ: pyrroloquinoline quinone; PSK: phosphorelay sensor kinase; Q: queuosine; RNase H: DNA-RNA hybrid ribonuclease; SAM: S-adenosyl-L-methionine; TPP: thiamine pyrophosphate; TOP1: topoisomerase type 1; HK: histidine kinase; UDP-G: uracil-diphosphate glucose; 6-PGL: 6-phosphogluconolactonase.

FIG. 4. Increased acidification strains of G. oxydans B58 are able to increase rare earth extraction from retorted phosphor powder (RPP). (A and B) A subset of 20 disruption strains were tested for acidification with direct pH measurement. pH measurements significantly different from pWT (black circle) are labeled with asterisks: *, p<0.05; **, p<0.01; ***, p<0.001 (n=5, df=18). Error bars represent standard deviation. (C and D) Ten disruption strains with the lowest final biolixiviant pH and four with the highest were tested for RPP bioleaching capabilities. Outer gray bars represent total REE extracted. Inner multi-colored bars represent fractional contributions of each REE and are Y, La, Ce, Eu, GD, Tb from bottom to top in each bar. Error bars represent standard error for total REE extracted. Percentages are total REE extraction efficiency (based on previously published REE amounts in the RPP). (C) Using a two-tailed t-test between each mutant and pWT demonstrated eight strains were significantly better or worse at bioleaching total REE (+, p<0.05; n=5, df=18). With a Bonferonni correction, only one was significantly better (**, p<0.01/12), but two of the higher pH biolixiviants that extracted detectable REE were significantly attenuated in bioleaching capability (***, p<0.001/12). (D) Disruption mutants for mgdh and pqqC are only able to extract less than 1% of the REE that wild-type G. oxydans can, but still extract significantly more REE than glucose alone when measured at a lesser dilution (***, p<0.001/2). (E) Total REE extraction linearly correlates with pH. Error bars represent standard deviation for pH and standard error for total REE extracted.

FIG. 5. Clean insertion and deletion mutations targeting genes of interest confer improvements in REE extraction relative to unmodified (WT) bacteria. Biolixiviants produced using modified strains that have increased expression of mgdh driven by an introduced tufB promoter, or clean deletions of pstS or pstB improved REE extraction by 12% and 34% over wild type, respectively. Biolixiviant produced by a clean deletion of mgdh with almost no REE extraction capabilities is included as a control.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the effective filing date of this application or patent.

The disclosure includes modified microorganisms having any modified single gene, and modifications of all combinations of genes described herein in the text, figures, figure legends, and tables of this disclosure.

Any gene and any combination of the genes that are described herein may be excluded from the claims of this disclosure. In embodiments, a modified microorganism of the disclosure may comprise or consist of only one modification of a single gene. In embodiments, a modified microorganism of the disclosure may comprise or consist of any combination of gene modifications described herein. In embodiments, only one or only a combination of genes that influence bioleaching of REEs are modified.

In non-limiting embodiments, the disclosure provides modified bacteria in which the expression of at least one of the genes pqqA, pqqB, pqqC, pqqD, pqqE, tldD and tldE, is increased. In certain embodiments, expression of pstS, pstB, pstC, or a combination thereof is reduced, or expression of pqqA, pqqB, pqqC, pqqD, pqqE, tldD, tldE, or a combination thereof is increased. In certain embodiments, the modified bacteria exhibit increased expression of mgdh relative to expression of mgdh by unmodified bacteria, wherein the increased expression of mgdh is in the context of at least one other described genetic modification.

In embodiments, the modified bacteria comprises or consist of mutations that are selected from mutations in all of the genes listed in Table A, and including all numbers and ranges of numbers of genes between 1 gene and the total genes in Table A.

The disclosure includes modifications that disrupt one or a combination of genes, modifications that increase expression of one or a combination of genes, or a combination of modifications that decrease expression of one or more genes and modifications that increase expression of one or more genes. Thus, the modifications involve altering the expression of one or more genes. Increasing, e.g., overexpressing a gene, can be achieved using various techniques that will be apparent to those skilled in the art when given the benefit of the present disclosure. In embodiments, increasing expression of a gene is achieved by substituting an endogenous promoter with a promoter that increases expression of the gene, relative to expression of the gene that is produced by the endogenous promoter. By “substituting” a promoter it is meant that the endogenous promoter (e.g., the promoter that is ordinarily operatively linked to the gene of interest without genetic engineering) has been changed so that is does not drive expression of the gene in the modified bacteria, and therefore the substituted promoter drives gene expression. By making this change, more mRNA is transcribed, thus facilitating production of more protein encoded by the pertinent gene that is operatively linked to the promoter. Substituting a promoter can include inserting a new promoter, while leaving the endogenous promoter in place, or inserting the new promoter in place of the endogenous promoter. The promoter that is inserted so that it is operably linked to and therefore drives expression of the described gene(s) can be heterologous to the bacteria, meaning it is taken or derived from a different organism, or it may be endogenous to the organism but has been introduced into a new location such that it can drive expression of the described gene(s). Various prokaryotic promoters that are suitable for this purpose are known in the art and include, for example, tufa and tufB. The substituted promoter (e.g., the promoter that is introduced into the bacteria) may be a constitutive or inducible promoter. The substituted promoter may be a core promoter, a proximal promoter, or a distal promoter.

Representative and non-limiting embodiments of promoters that can be used to increase expression of one or more genes as described herein include:
P_{tufB} which has the sequence:

(SEQ ID NO: 1)
Ccgaaggcatcgtttacggtgctctcgaagtcatgcgccg
tcgcggcggcacgactgcagatccggtggccatgttccac
tcggctctggataacgtgaagcctgcggttgaagtccgct
cgcgtcgcgtcggtggcgcaacctatcaggttccggttga
agtccgcgccgagcgccgtcaggctctcgcgatccgctgg
ctgatcgatgcctcccgcaagcgtggcgagaacacgatgc
aggagcgtctgtccaacgaactgatggatgcggtcaacaa
ccgtggctccgctgtcaagaagcgcgaagacacgcaccgc
atggctgaagccaacaaggcattcagccactatcgctggt
aatcagaaccggttaaagggcttccggcagtgtgtcgatc
tcaggatcgtgcactgactggtttgaactttcagtcttac
tcccgttccaggtggagcggggttttggagaaagacg;

P₁₁₂, which has the sequence:

(SEQ ID NO: 2)
Ggaactgactcctgatttcgttctgttttcatgggatcaa
tgaacggtcaggcgaaaatgttgcatcggggtgcaggaaa
tttcccgaaaaaaggaaaagacaggctggagcccgcggaa
atcaggcaaaaatcaggtgatatttttttcggattccgtt
tccggaggttcggtatttttcgtcgcaagctcggcgagct
gggctcgggcacgggaaacacgggccctcatcgttccggg
ggcacatcgcagacgcgggcggcatcttcataggacagtt
cctgtgctcccacgagaatcagggcctcccgcaacagatc
gggcagcttccacagcagttctcccaggtcctgaacggca
tcgcgggcattctggcgtgacggtgcagccgtggaaggct
gcatgtcctgctcgatgtcgagcatgatttcctgctcgcg
gctgcgacggcgcgtctgctcgtagaaggcatttctctgt
attgtgaacagccaggctttgaggaccgttccctgctgga
actg

P₁₁₄, which has the sequence:

(SEQ ID NO: 3)
Gtggcagatgttgagaaatatccgcagttccttccctggt
gtgtgaaggcaagcatccggacccagacggaacaggagct
tgtggcggatctgacgatcgggttcggcccgttccgcgag
accttcaccagccgcgtgacgctggagcggccttcgcgta
tccgggtgcgctacgagaaagggcctttccgttacctgaa
taatgtctggacgttcacgccggatccgcggggctgtctg
gtcgatttcttcgtggatttcgagttccgttcgcgccttc
tgcagaatgcgatgggtgtcgtgttcaatgagggcgtgcg
cctgatggtctccgccttcatcaagcgggcacgggacatt
tacggcacgcaggcgacgaaagcggtccccccggcaccgg
gcctttcccaaaggacataaaatttcgtattatttccaca
accattcggtgcgtgggccgttacaggtctcaagcaccgt
catggtgacatgaaaaggattacgta

As an alternative or in addition to promoter modification, the disclosure includes addition of and/or repositioning of enhancer elements to increase expression of the described gene(s).

As an alternative or in addition to changing promoters, the disclosure includes increasing copy number of the gene that is to be overexpressed. In embodiments, one or more copies of the gene can be inserted into a bacterial chromosome, or can be introduced into bacteria using a plasmid. A list of genes for which overexpression is encompassed by the disclosure is provided on Table A. The additional copies of the gene may be in tandem, such as in a polycistronic configuration, or may be separated by segments of the bacterial chromosome or plasmid. In embodiments, a composition comprising the described bacteria are modified by transformation using one or more plasmids, which may be configured to be replicated and transferred to other bacteria in a bacterial population, such as by horizontal transfer.

In another embodiment, the disclosure comprises decreasing expression of genes. Decreasing expression can be achieved using any suitable approach. In embodiments, decreasing expression comprises disrupting the gene such that the protein encoded by the gene is not produced, or a protein produced by the gene does not function in the same way as if it had not been modified. In embodiments, a protein that is encoded by a modified gene of this disclosure is produced but does not function to impede bioleaching of REEs from a composition comprising them. In embodiments, a modification of a gene comprises a knock-out of some or all of the gene. Modifications of the genes can be achieved using any suitable genetic engineering techniques. In non-limiting embodiments, the modification comprises an insertion, a deletion, or a combination thereof. The disclosure includes insertion within, or a deletion of any segment of a gene, including but not limited to a insertion or deletion of a single nucleotide, such that the encoded protein is not produced or its function is eliminated or reduced. In embodiments, an insertion replaces some or all of the described gene(s). In a non-limiting embodiment, the described gene(s) is modified by insertion of a transposable element. In non-limiting embodiments, the genes are modified using compositions and methods described in U.S. Pat. No. 11,053,493, from which the entire description is incorporated herein by reference. In embodiments, a modification of a gene comprises an insertion as described in Anzai, Isao A., et al. “Rapid curation of gene disruption collections using Knockout Sudoku.” Nature Protocols 12.10: 2110-2137 (2017), from which the entire disclosure is incorporated herein by reference. In alternative approaches, site specific nuclease, such as Cas nucleases, can be used to modify any of the described genes. In embodiments, a type I, type II or type III CRISPR system can be used. Thus, in embodiments, a guide-RNA directed nuclease can make any of the described modifications. In embodiments, recombination of a chromosome or plasmid can be used, such as by introducing a recombination template comprising additional copies of a gene, and/or a promoter, to facilitate recombination of the recombination template into a desired location. In an embodiment, homologous recombination is used, and as such, the recombination template includes left and right homology arms to specify the location of recombination. In embodiments, a transposon system can be used to interrupt a gene sequence, such as the Sleeping Beauty transposon system.

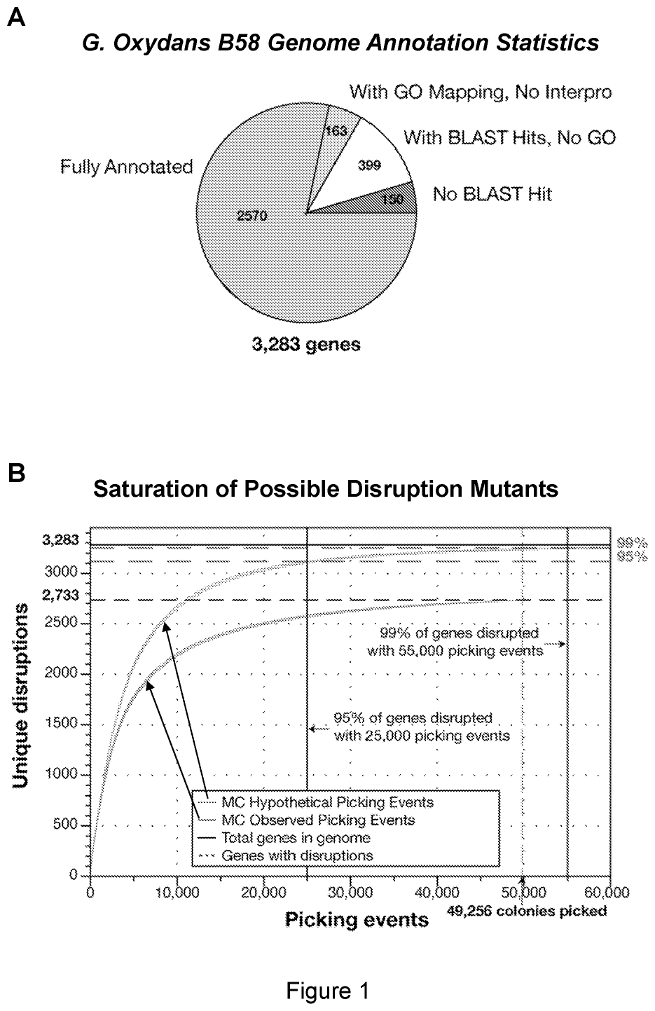

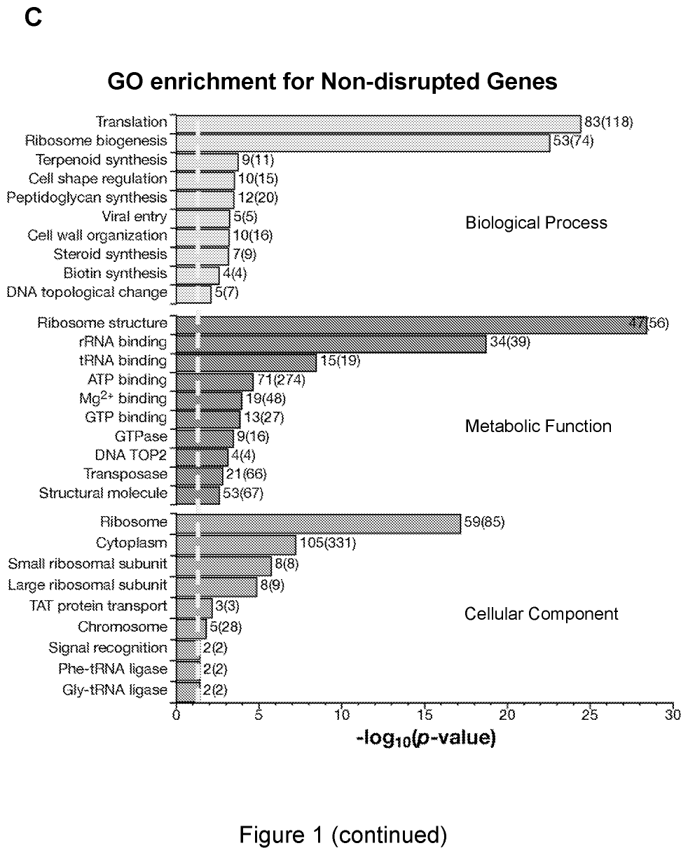

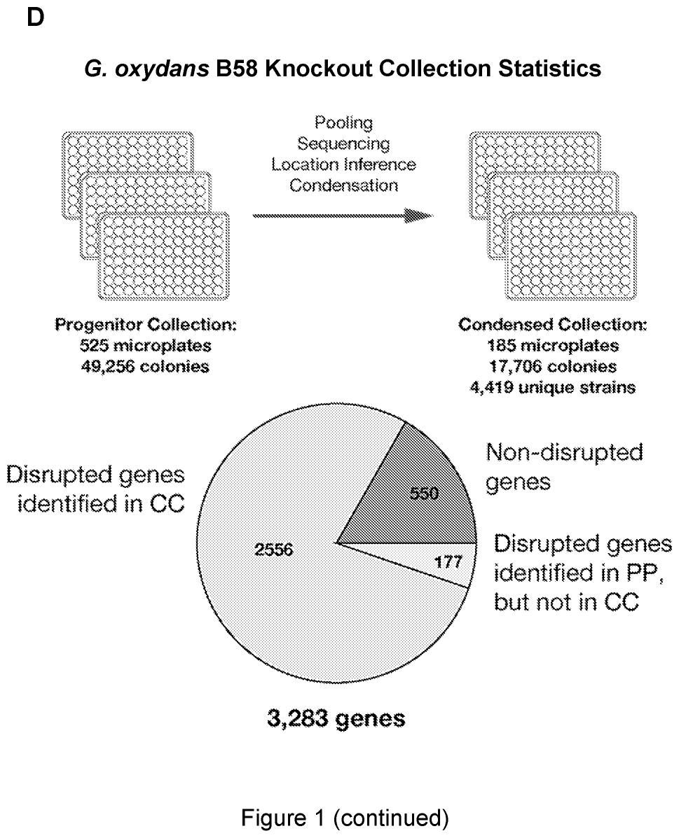

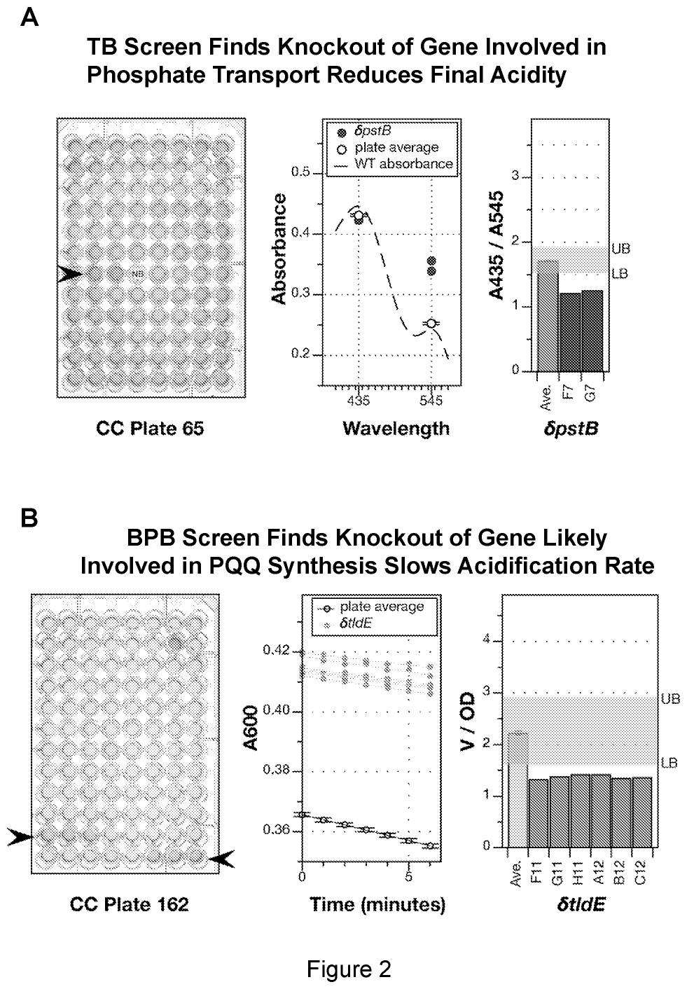

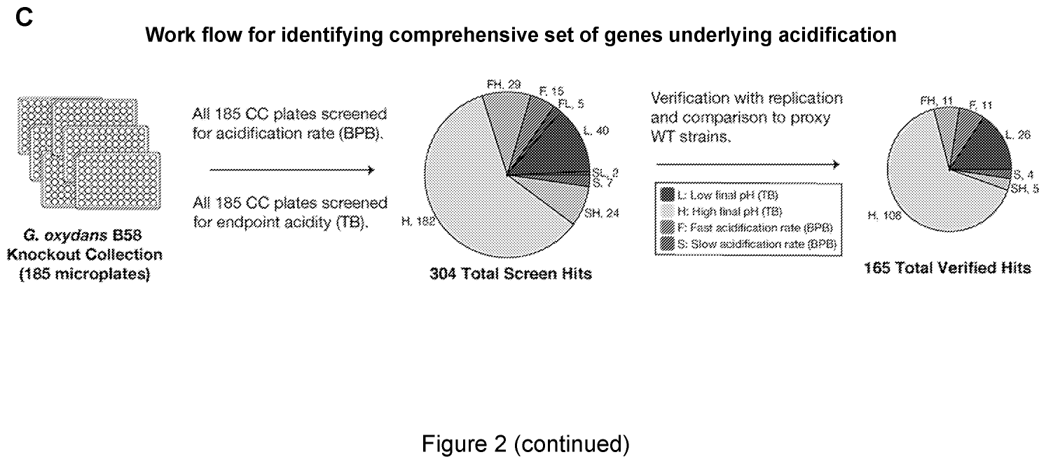

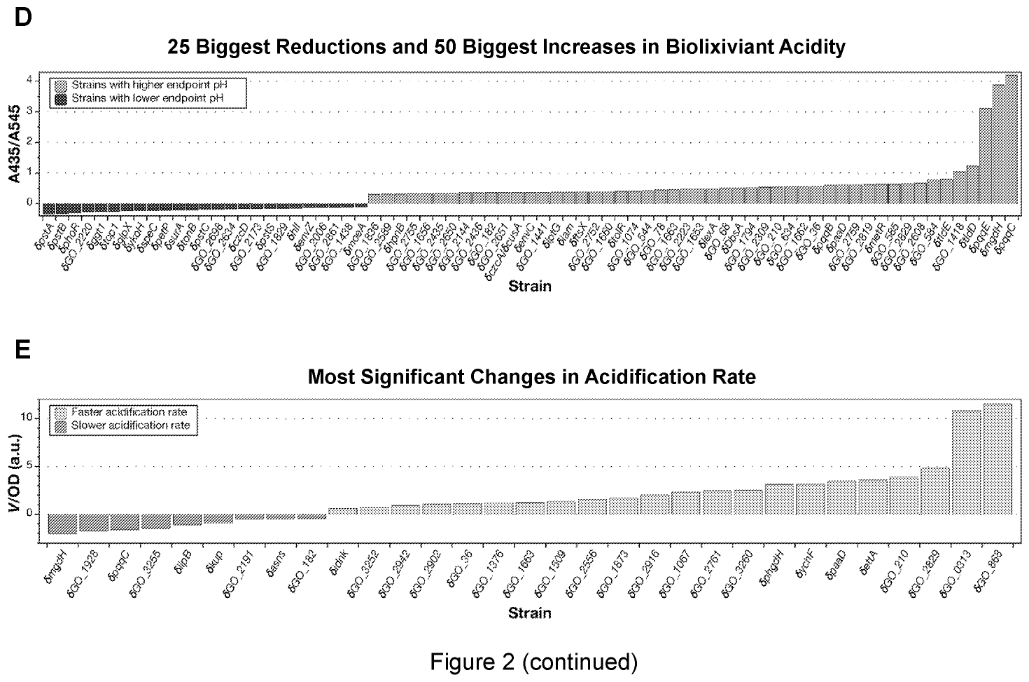

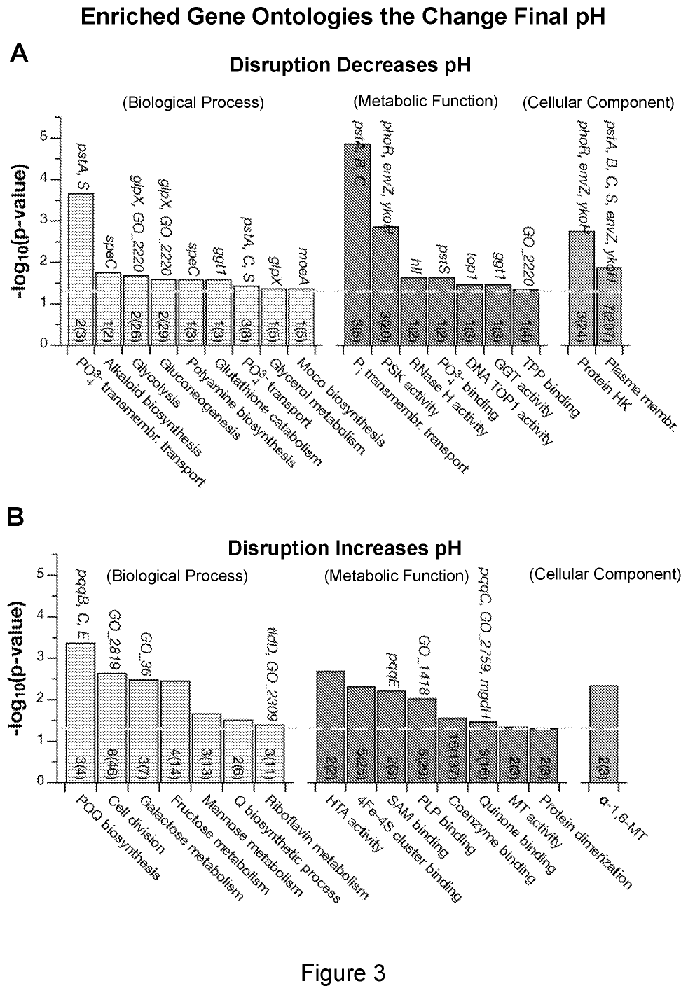

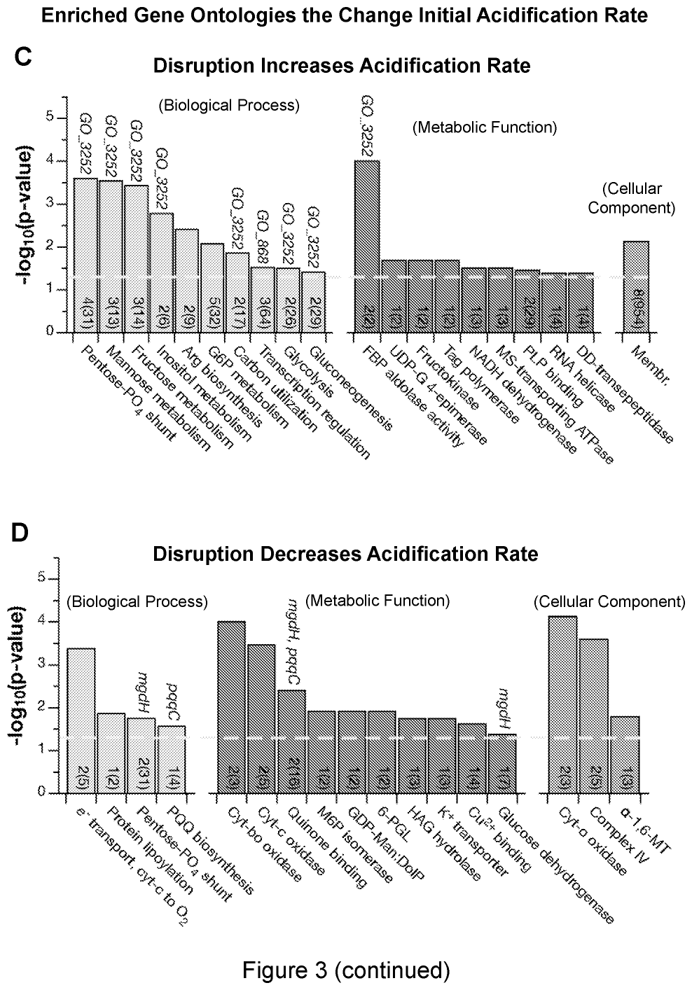

In embodiments, the modified bacteria comprise a modification of at least one gene described in FIG. 1, FIG. 2, or FIG. 3. In embodiments, the modified bacteria comprise a modification of at least one gene as in Table A. Table A includes gene names and additional information regarding the type of analysis that were used in determining the effects of each gene in the assays that are further described below. In Table A, H=high acidity; L=low acidity; F=fast acidification; S=slow acidification. Table A includes the amino acid sequences of the proteins encoded by the listed genes. The disclosure includes all amino acid sequences that are 80-99% identical to the described amino acid sequences, and all polynucleotide sequences encoding said amino acid sequences. Polynucleotides that encode the described amino acids constitute the coding regions of the described genes.

In embodiments, the disclosure comprises increasing expression of at least one gene described in Table A. In embodiments, the disclosure comprises decreasing expression of at least one gene described in Table A. In embodiments, the disclosure comprises increasing expression of at least one gene and decreasing expression of at least one gene described in Table A. In non-limiting embodiments, modified bacteria of this disclosure are modified such that they exhibit decreased expression of at least one of the following genes: GO_1415, pstA, pstB, pstC, pstS, ggtl, surA, petP, ykoH, speC, and tonB. In non-limiting embodiments, modified bacteria of this disclosure are modified such that they exhibit increased expression of at least mgdh, and/or genes involved in PQQ synthesis (e.g., pqqA, pqqB, pqqC, pqqD, pqqE, and tldD, also referred to as pqqABCDE as an operon, and tldE), or a combination thereof. In embodiments, any one or any combination of proteins expressed by the pqqA, pqqB, pqqC, pqqD, pqqE, and tldD genes can be modified to increase their activity, such as by modifying amino acids in an active site, or amino acids that improve structural stability, and the like.

Combinations of modifications that increase and decrease expression of genes are included in the disclosure. The disclosure also includes mixed populations of bacteria, wherein some of the members of the population have different genetic modifications than other members of the population.

TABLE A

| | | | | BPB | | | |
|---|---|---|---|---|---|---|---|
| | | | TB | Acidifi- | | | |
| | | | Final | cation | | | |
| | | | pH | Rate | | | |
| | | | Screen | Screen | Up or | | SEQ |
| Locus | Gene | Features | Pheno- | Pheno- | Down | Amino Acid | ID |
| Tag | Name | Disrupted | type | type | Regulate | Sequence | NO: |
| GO_ | ettA | Energy- | H | F | Both | MAAYQYVYVMKDLTKSYPGGREVFK | 4 |
| 3277 | | dependent | | | | GITLSFLPGVKIGVLGVNGAGKSTL | |
| | | translational | | | | LKIMAGIEKEYGGEAWAAEGARIGY | |
| | | throttle | | | | LEQEPKLDESLTVGENVAQGFGELK | |
| | | proteinEttA | | | | KAVDRFNEISMKFAEPMSDDEMTAL | |
| | | | | | | LAEQADLQEKIDAGDGWELDRKLEI | |
| | | | | | | ALDALRCPSADSPVTNLSGGEKRRV | |
| | | | | | | ALCRLLLEKPDILLLDEPTNHLDAE | |
| | | | | | | SVSWLEKTLRDYAGTVMVITHDRYF | |
| | | | | | | LDNVTNWILEIERGRGYPFEGNYSS | |
| | | | | | | WLTQKRKRLAQEEKEESSRQRALAA | |
| | | | | | | EQEWISSSPKARQAKSKARITKYEE | |
| | | | | | | MLAANAEKAGGTADIVITPGPRLGG | |
| | | | | | | TVIEAENLTKGFGDRLLIDNLSFKL | |
| | | | | | | PPGGIVGVIGPNGAGKSTLFKMITG | |
| | | | | | | DDQPDSGSLKIGETVKLGYVDQSRN | |
| | | | | | | TLDDSKTVWEEISGGTDVIQLGKRT | |
| | | | | | | VPSRAYVGAFNFKGSDQQKRVGVLS | |
| | | | | | | GGERNRVHLAKMLKQDSNVILLDEP | |
| | | | | | | TNDLDVDTLRALEDALAEFAGCAVI | |
| | | | | | | ITHDRWFLDRLATHILAFEGDSHVE | |
| | | | | | | WFEGNFQDYEADKRRRLGPDATEPG | |
| | | | | | | RIKYRPLAR | |
| GO_ | glpX | Fructose-1,6- | H | F | Both | MIDPQNVLPYRVTDRNLALELVRVT | 5 |
| 1534 | | bisphosphatase, | | | | EAAAIASAHWTGRGQKNEADGAAVQ | |
| | | GlpX type | | | | AMRAAFDTVAIDGVVTIGEGEMDEA | |
| | | (EC 3.1.3.11) | | | | PMLYIGEKVGSGGPAMDIAVDPLEG | |
| | | | | | | TNLCAKSLPNALTVVALAERGKFLH | |
| | | | | | | APDIYMEKIVVGPDLPEGVVDLDAS | |
| | | | | | | IGNNLRSLAKAKKRDVSDMVLCALD | |
| | | | | | | RERHEELIAHAREAGARVMLLSDGD | |
| | | | | | | VAAAIATCVDGGGVDLYAGSGGAPE | |
| | | | | | | GVLAAAAIRCMEGQMQGRLLFEDDT | |
| | | | | | | QRERAREMANGLDPARKLFLHDMAS | |
| | | | | | | GHVLFSATGVTSGPLLKGVERPSPS | |
| | | | | | | RARTHSIVMRSKSGTVRYIESHHTF | |
| | | | | | | KPKVKAAS | |
| GO_ | GO_ | hypothetical | H | F | Both | MDLCWTTSCRSTTSRGRAATSACRD | 6 |
| 289 | 0289 | protein | | | | YTASHHDHSPPTIGATLRNGALATP | |
| | | | | | | RLCLGAGPLVLDSARLHLAKRLVAA | |
| | | | | | | LVNSFV | |
| GO_ | GO_ | D-alany1- | H | F | Both | MQVALEPETLPATIFRSEAVRQMRF | 7 |
| 1067 | 1067 | D-alanine | | | | PFQKAFLLCAMTAMGLGSAKAQYAG | |
| | | carboxypeptidase | | | | HISSYVMDARTGAVLSATDAELQRY | |
| | | (EC 3.4.16.4) | | | | PASLTKLMTLYLTFRALEAHQITLD | |
| | | | | | | EQVPVSIHASIQAPSKLGLVPGTRL | |
| | | | | | | TVEQGILGLVTKSANDAACALGEFL | |
| | | | | | | GGGDEVRFAQLMTQQARALGMTNTT | |
| | | | | | | FRNASGLPDPDQVTTAHDLALLSQH | |
| | | | | | | LISDFPQYYHYFNVPSFYFHRRMVP | |
| | | | | | | NHDPMLKIYAGADGLKTGYTDLAGH | |
| | | | | | | NLITSAQRGNVRLIGVVMGAPNNTR | |
| | | | | | | RSMEMVSLLDKGFADEGVAPQPLLH | |
| | | | | | | PVAPSGVLMASARRRGHFRHSVLLA | |
| | | | | | | SSRPMEVADAPTSPRRYGRISHHGA | |
| | | | | | | AVRMVSARHVVAHKKRSRHS | |
| GO_ | GO_ | ABC-type | H | F | Both | MICRFSPKVTLSLLSACLLT | 8 |
| 1076 | 1076 | transport | | | | GPVAMTALSSVALAQTAGAV | |
| | | system involved in | | | | SPADAASAVTPISALYDALK | |
| | | resistance | | | | AAQKTGKTAQQRATMIAPAV | |
| | | to organic | | | | DRAFDLEAILRRSVGVRYNS | |
| | | solvents, | | | | LSPSDRTRLLGSFRQFTIAR | |
| | | auxiliary | | | | YASSFKPEAPAAFTVSPQTR | |
| | | component | | | | PNPTGGLIVDTTIGGTDGGD | |
| | | | | | | VTPIDYIMTNGTSGWRITDV | |
| | | | | | | LLNAHISQVAAQRADFGGAL | |
| | | | | | | SSGGASGLADHLDSKTAHFL | |
| | | | | | | HD“ | |
| GO_ | GO_ | Sphingosine | H | F | Both | MRIALIHNARSRKNRRNGSS | 01 |
| 1658 | 1658 | kinase | | | | FAVEAQALLGKDFVPSDTRE | |
| | | and enzymes | | | | GTTEHVRQLYERGIDTILID | |
| | | related to | | | | GGDGTVSTALTAIARAYPAD | |
| | | eukaryotic | | | | RLPDIVVLASGNTNLIAGDV | |
| | | diacylglycerol | | | | GFGLRGMEAIQRLRQGDLRS | |
| | | kinase | | | | SVRTPIRLSWPGTDRMPVLG | |
| | | | | | | LFGGCAGYARAVRIAHSPTV | |
| | | | | | | LRFAPHDLAVGITLLSTFVS | |
| | | | | | | LMFRKSREEWLRGDPLRIET | |
| | | | | | | GGHVYDGQSFMFLTTGLSHL | |
| | | | | | | SRGIWPFWDAEPNVEGLRYL | |
| | | | | | | DVSAWPDSLLRATAALLCGR | |
| | | | | | | APRWLRRHPDYVSGRTDDMT | |
| | | | | | | LVTESDFVLDGEVFSAAPGG | |
| | | | | | | VLRLERASRFRFLHA | |
| GO_ | GO_ | Putative | H | F | Both | MAGGLLVGMGILAAGTTLAA | 10 |
| 1865 | 1865 | membrane- | | | | ARHIGDGSLAFSLLRAGSRA | |
| | | spanning | | | | GVVGGLADWFAVTALFRHPL | |
| | | protein | | | | GLPIPHTAILPRQKERLGQA | |
| | | | | | | LGRFISGQFFTEDDVRRALS | |
| | | | | | | KIDLSGLIADMLNDPANRQT | |
| | | | | | | LVTSMRSAIPLMFDRMEDGR | |
| | | | | | | AKTAISRALPVLLNGEEMAP | |
| | | | | | | LVSRGMRAMVDSEMHQEVLS | |
| | | | | | | FLLERIKTTVTSRESDLRHF | |
| | | | | | | VEERVREQGGRFLGWAIGGS | |
| | | | | | | VASRVLMALHAEFERVDPMD | |
| | | | | | | SELRHGFTTWVRGEIDRIEN | |
| | | | | | | DPARRKDMADTITSVLTHTS | |
| | | | | | | LKGWSSELWDRLRRMVEEDS | |
| | | | | | | GREDGWSATVIDAAIVQLAS | |
| | | | | | | ALRSNESLRLKVNQAVESTV | |
| | | | | | | QRILPGLREKLSGFIAAVMA | |
| | | | | | | GWDGNDLAARLESRVGRDLA | |
| | | | | | | YIRINGTVVGFLAGAALDGV | |
| | | | | | | SRLFFGV | |
| GO_ | GO_ | ATP-dependent | H | F | Both | MYAVKESLESHLSAKTTSGP | 11 |
| 1873 | 1873 | RNA | | | | SKPRTTATPRRGRPSASRTA | |
| | | helicase | | | | AAATSPTVADKGETSPVTET | |
| | | Atu1833 | | | | PASKAPRTRRTKTAATATEK | |
| | | | | | | TAEVKTPARRTRKAATPAAE | |
| | | | | | | SATESEAPAPKTTGRRKKAV | |
| | | | | | | TEPEAVTELAEAAVAAPAPR | |
| | | | | | | RRRSTKAPEAVTEEAEAAPK | |
| | | | | | | RRGRPRKTPVVEQPAAEVIT | |
| | | | | | | EETVKAPAAPRRRGRPRKVD | |
| | | | | | | VAVAETPVEAPVEKKARQSR | |
| | | | | | | RKASAPVMEAPEPAAEETPA | |
| | | | | | | VQTESTKGEKPARRRRTKAA | |
| | | | | | | AATSAVVEKTVEAPAVVETI | |
| | | | | | | VVAEDVSDRPRFADLGLGEP | |
| | | | | | | IMRAIEELGYEHPTPIQAQA | |
| | | | | | | IPEVLKGHDVLGVAQTGTGK | |
| | | | | | | TASFTLPMLQKLAGSRARAR | |
| | | | | | | MPRSLILEPTRELALQVAEN | |
| | | | | | | FKLYGKYLRLTHALLIGGES | |
| | | | | | | MAEQRDVLNRGVDVLIATPG | |
| | | | | | | RLLDLFGRGGLLLTQTSTLV | |
| | | | | | | IDEADRMLDMGFIPDIEKIV | |
| | | | | | | ALLPAHRQTLFFSATMAPEI | |
| | | | | | | RRLADAFLRHPVEITVSRQS | |
| | | | | | | SVATTIEEALVIVPEDEKRR | |
| | | | | | | TLKKLLRRENVQSAIVFCNR | |
| | | | | | | KRDVDMIQQYLTKHGIEAGH | |
| | | | | | | LHGDLAQSLRFSTLERFRSG | |
| | | | | | | DLKFLVCSDVAARGIDIGGL | |
| | | | | | | SHVFNYDLPFNAEDYVHRIG | |
| | | | | | | RTGRAGNEGHAFSLATPRDR | |
| | | | | | | RLLEAIETLTGKVIARPVLE | |
| | | | | | | GITTVDWAPEDGERRPAETA | |
| | | | | | | TPAPQDVAEEGEQRLRKRRR | |
| | | | | | | GGRKRNRGDRDENETVQEVA | |
| | | | | | | PTAVASVAVIEAPVSHRSVE | |
| | | | | | | LAPAFENDGPKTGFGGDTPA | |
| | | | | | | FMLVPRRRKVTVSGSTDPAA | |
| | | | | | | PVQHDGRHYGNE | |
| GO_ | yqg | Putative | H | F | Both | MPLFNPHDLRNLLQSGQRVL | 12 |
| 2019 | | pre-16S | | | | GLDPGSKTIGVALTDVSLML | |
| | | rRNA | | | | ASPLIGLKRRKLGENAQELA | |
| | | nuclease Yqg | | | | KIVRAQDVGALVVGLPLSLD | |
| | | | | | | GSFGPAARAASDWTQALSEK | |
| | | | | | | LGIPAGLWDERLSSSAVNRF | |
| | | | | | | LIKDADMTRGRRAEVVDKMA | |
| | | | | | | AAYMLQGWLDASRPESPEIF | |
| GO_ | GO_ | Phosphogluconate | H | F | Both | MSLNSVVESVTARIIERSKI | 13 |
| 210 | 210 | dehydratase (EC | | | | SRRRYLALMERNRAKGVLRP | |
| | | 4.2.1.12) | | | | KLACGNLAHAIAASSPDKPD | |
| | | | | | | LMRPTGTNIGVITTYNDMLS | |
| | | | | | | AHQPYGRYPEQIKLFAREVG | |
| | | | | | | ATAQVAGGAPAMCDGVTQGQ | |
| | | | | | | EGMELSLFSRDVIAMSTAVG | |
| | | | | | | LSHGMFEGVALLGICDKIVP | |
| | | | | | | GLLMGALRFGHLPAMLIPAG | |
| | | | | | | PMPSGLPNKEKQRIRQLYVQ | |
| | | | | | | GKVGQDELMEAENASYHSPG | |
| | | | | | | TCTFYGTANTNQMMVEIMGL | |
| | | | | | | MMPDSAFINPNTKLRQAMTR | |
| | | | | | | SGIHRLAEIGLNGEDVRPLA | |
| | | | | | | HCVDEKAIVNAAVGLLATGG | |
| | | | | | | STNHSIHLPAIARAAGILID | |
| | | | | | | WEDISRLSSAVPLITRVYPS | |
| | | | | | | GSEDVNAFNRVGGMPTVIAE | |
| | | | | | | LTRAGMLHKDILTVSRGGFA | |
| | | | | | | DYARRASLEGDELVYSHAKP | |
| | | | | | | STDTDILRDVAAPFRPDGGM | |
| | | | | | | RLMTGNLGRAIYKSSAIAAE | |
| | | | | | | HLTVEAPARVFQDQHDVITA | |
| | | | | | | YQNGELERDVVVVVRFQGPE | |
| | | | | | | ANGMPELHKLTPTLGVLQDR | |
| | | | | | | GFKVALLTDGRMSGASGKVP | |
| | | | | | | AAIHVGPEAQVGGPIARVRD | |
| | | | | | | GDMIRVCAVTGQIEVLVDAA | |
| | | | | | | EWESRRPVPPPLPALGTGRE | |
| | | | | | | LFALMRSVHDPAEAGGSAML | |
| | | | | | | AQMDRVIEAVGDDIH | |
| GO_ | GO_ | Phytoene | H | F | Both | MVFSRMRASSGLPCAQADLD | 14 |
| 2105 | 2105 | synthase | | | | HVERIVTASGTSFARGMSIL | |
| | | (EC 2.5.1.32) | | | | PPDRRQAMFAVYAFCRQVDD | |
| | | | | | | IADGDAGVADPMAALQEWHR | |
| | | | | | | RIDQLYEGVATDALDRILIV | |
| | | | | | | AIHRYQLQAKDFHDVIDGMA | |
| | | | | | | MDCGAPIVAPDEATLDLYCD | |
| | | | | | | RVASAVGRLSVCVFGDSSDN | |
| | | | | | | ARRVAYHLGRALQLTNILRD | |
| | | | | | | IAEDAGRGRLYLPAELLTRF | |
| | | | | | | DVPKDPQEALYAHGLDQVAR | |
| | | | | | | ILAERAKDHFREARNAMRLC | |
| | | | | | | DSTAMRPARMMAASYAPILS | |
| | | | | | | ALEKRGWKTPDIAPKVCKPW | |
| | | | | | | RQLRTLAAYVK | |
| GO_ | GO_ | Adenosyl | H | F | Both | MGSKGHFMTTQDYKVRDITL | 15 |
| 2223 | 2223 | homocysteinase | | | | ADWGRKEISIAEGEMPGLMA | |
| | | (EC 3.3.1.1) | | | | LREEYKDSQPLKGARIAGCL | |
| | | | | | | HMTIQTAVLIETLIALGATV | |
| | | | | | | RWSSCNIFSTQDHAAAAIAA | |
| | | | | | | AGIPVFAWKGLTEEEFNWCI | |
| | | | | | | EQTIHGPDGWTPNMILDDGG | |
| | | | | | | DLTIMMHDKYPEMLKDVRGI | |
| | | | | | | SEETTTGVHRLWEMSKKGTL | |
| | | | | | | KVPAINVNDSVTKSKFDNLY | |
| | | | | | | GCRESLVDAIRRGTDVMMAG | |
| | | | | | | KVAVVAGYGDVGKGSAASLR | |
| | | | | | | NAGCRVLVTEVDPICALQAA | |
| | | | | | | MEGYEVVTMENAAPRGDIFV | |
| | | | | | | TCTGNVDIITIDHMREMKDR | |
| | | | | | | AIVCNIGHFDSEIQVEALRN | |
| | | | | | | YRWNNIKPQVDEIELAPNRR | |
| | | | | | | IILLSEGRLVNLGNATGHPS | |
| | | | | | | FVMSASFTNQTLAQIELWTA | |
| | | | | | | KPGQYEVKVYTLPKALDEKV | |
| | | | | | | AALHLAKVGAELSKMSQKQA | |
| | | | | | | DYIDVPVNGPFKHEEYRY | |
| GO_ | GO_ | Riboflavin | H | F | Both | MFSGIIERLGTVRSACVRDR | 16 |
| 2309 | 2309 | synthase | | | | AMDLTVETGFPDLELGESVA | |
| | | eubacterial/ | | | | VNGVCLTVETFDAAGVATFH | |
| | | eukaryotic | | | | LSGETLSRTPLDQLKTGSRV | |
| | | (EC 2.5.1.9)\ | | | | NLERAVAASTRLSGHIVQGH | |
| | | Diamino | | | | VDGVATLASVEKAGDSYALR | |
| | | hydroxyphosphoribosyl | | | | VFVPQALRQYVVEKGSITFD | |
| | | aminopyrimidine | | | | GISLTVNELHDDITRGNQAG | |
| | | deaminase (EC | | | | FEVGLTIIPHTWEHTNLSTL | |
| | | 3.5.4.26) | | | | SVGDRMDVEVDVLSKYVENL | |
| | | | | | | LRFSPSLGKVAS | |
| GO_ | GO_ | hypothetical | H | F | Both | MRRCVAAIRKAALFGLVFAG | 17 |
| 2456 | 2456 | protein | | | | IAGAGSASAAEAAWTASKCG | |
| | | | | | | AEPQAPAVKAATVAQYNESV | |
| | | | | | | DRVTAYEKAARVYNACVSAQ | |
| | | | | | | ANREETAISQEASARISHVH | |
| | | | | | | AGSAAVQSHIAASFQTLSSN | |
| | | | | | | LAAASRKLGHH | |
| GO_ | GO_ | TonB-dependent | H | F | Both | MRSRYCLCATTALALSVSAA | 18 |
| 2571 | 2571 | receptor | | | | FAQSDVAPKSRSHRPTQTHK | |
| | | | | | | SAATKEDSPSMMGSTSPTDE | |
| | | | | | | ARSETVRVQGSRRSSPGAGL | |
| | | | | | | MVHEDAAKSRSTVTQEFISK | |
| | | | | | | QTPGVNPMQLIAMLPGVNTT | |
| | | | | | | SVDPLGLNGGNMTMRGLNAS | |
| | | | | | | QIGFTLEGFPINDIGNYAVY | |
| | | | | | | PQEIVDSENLRSITVEQGSA | |
| | | | | | | DLDSPHISASGGAVNMYLLD | |
| | | | | | | PKDHFGGHLDGTYGSYNSRR | |
| | | | | | | IFGRMDTGKIGNTNLKGYVS | |
| | | | | | | FSAAHEDSWRGPGSQRKLHG | |
| | | | | | | ETKWVNEWGQGNRISLAIVG | |
| | | | | | | NQSNSTLFPSMSLANWNKYG | |
| | | | | | | VNYTYTGKWNPSSPSTNYYK | |
| | | | | | | LHQNPFTNIYASAPSTFTLT | |
| | | | | | | DHLTLTETPYFWYGNGNGGG | |
| | | | | | | AYNESLSSQQYGAQTLSASI | |
| | | | | | | GQYNSSNTKSLLVYNPSNTQ | |
| | | | | | | TYRPGAVTKLALHTGANRLM | |
| | | | | | | IGYWFEYSKQIQTSPYSLVN | |
| | | | | | | MATGTPLDVYGGGTNMVLSN | |
| | | | | | | GVTAEYRDTLTQTRIHTLFI | |
| | | | | | | DDSLSLLNNHLTLEAGLKYA | |
| | | | | | | MVARQGHNFLPDTSTGPYIN | |
| | | | | | | GSWNEPLPAASIRYKLNNEI | |
| | | | | | | QLFASGTTNFRIPMNTALYD | |
| | | | | | | SGTYTSGSGYSTKANTNMKP | |
| | | | | | | EISISEEAGIRYQGALFNGT | |
| | | | | | | LSYFHYNFTNRLYSETVVQS | |
| | | | | | | NGAYYSTSVNGGSSHADGVD | |
| | | | | | | FEIGTRPILYHLRPYISAEY | |
| | | | | | | IDARTDSNVAAGGSTNDYVH | |
| | | | | | | SKGKFAPQTAKVQVAFNLDY | |
| | | | | | | DDGAFFSGFGLKYVGKQYAT | |
| | | | | | | FNNDTSIPGYVTMDVHAGYR | |
| | | | | | | FRNYGVLKNPVLKLNIQNIT | |
| | | | | | | NNHYLGFVNGTASNGKATTG | |
| | | | | | | VFGSAIAAGSTTYYVASPFF | |
| | | | | | | VGGSASVDF | |
| GO_ | GO_ | hypothetical | H | F | Both | MKKCHNPARLRGICRLAQDD | 19 |
| 2699 | 2699 | protein | | | | RSQYIGIMRLLPTLPRLLLV | |
| | | | | | | AALAMTPTILVPWHHAQAQD | |
| | | | | | | ADDAEAEQEAEAAQKKAEAR | |
| | | | | | | KAAQRAAPPSALPGAEASDD | |
| | | | | | | DAGHARSDVNPTTALFDAIN | |
| | | | | | | RGSLNAAKEALNRGADMGGH | |
| | | | | | | NVLDQTPLDMAIDLNRKDIM | |
| | | | | | | FLLLSMRTYNPDGKIENSVS | |
| | | | | | | DEGVEMKNGSGHLTIGGKSV | |
| | | | | | | TPKRSLVAASPHFDTSGGKP | |
| | | | | | | DPSVGFLGFAGH | |
| GO_ | GO_ | ROK family | H | F | Both | MADYRLGIDLGGTKIEIAVL | 20 |
| 2761 | 2761 | sugar | | | | NRSGDLVLRERIPNPGIYNE | |
| | | kinase or | | | | AVLAIRDLVTDVDRRLGAVP | |
| | | transcriptional | | | | SHRVSAGQHTSTLGIGIPGS | |
| | | regulator | | | | ISPETGLIKNANATWLNNQP | |
| | | | | | | FGQDLESALARPVRTENDAN | |
| | | | | | | CAADGAAKGMLTVFGVIIGT | |
| | | | | | | GMGAAIVNNGRVLEGRHHIA | |
| | | | | | | GEWGHLPLPWPTEEDMPARD | |
| | | | | | | CFCGNKGCMERYLCGPALAA | |
| | | | | | | DWKGPGHRNTAGIEDAAANG | |
| | | | | | | DQAAIAALGRYTERFARACA | |
| | | | | | | MVINFLDPDVIVLGGGVSNL | |
| | | | | | | HTLYERVPPLLAKHVITPVC | |
| | | | | | | TTPIVRNKHGDSSGVRGAAW | |
| | | | | | | LWDVTE | |
| GO_ | GO_ | hypothetical | H | F | Both | MQKIFSLSEIGASDVSHDLV | 21 |
| 2829 | 2829 | protein | | | | SFDIFDTLVYRRYLEVNEVH | |
| | | | | | | DLASAYALSLLGQFGKENPG | |
| | | | | | | ALTLTRYDITNVMKAAAHER | |
| | | | | | | IEEPTLEAVWSRLFTARIGH | |
| | | | | | | TEKALTLGRKVAAFEYEIDR | |
| | | | | | | QNLYAVEGAAEMLAALKAQG | |
| | | | | | | KTVIAISDMYFSQTEIEGIL | |
| | | | | | | LQTGLARHIDRVFVSSQENL | |
| | | | | | | TKHSGNLFTHVWKQFDIAPA | |
| | | | | | | KTLHLGDNTHSDVAMPTSLG | |
| | | | | | | GNAIHVAHAPLLRIKRPDYG | |
| | | | | | | RRPDIHMEIGDLSKLFLTQL | |
| | | | | | | LLCAQSDGSDRLFFLSRDGC | |
| | | | | | | LLHKVLEKWNSPLFRNFFTP | |
| | | | | | | IHSEDLFLSRAVTCWLNVNF | |
| | | | | | | QDKWLLQSIGHAFWLHHGKA | |
| | | | | | | TPRQISGMLGIDATPAGLDA | |
| | | | | | | DKVYHSSMDTFTVLEAYEAS | |
| | | | | | | GL VEEIRTALLHKRAMAAS | |
| | | | | | | YLKDAGVLNHQSVHVCDVGY | |
| | | | | | | SGTVVRDLNTFFLQEGPQGL | |
| | | | | | | GGSIPQVYFHCIASNANYSG | |
| | | | | | | NARTALPHVIFQKDVILNDG | |
| | | | | | | LLPGELKDSFAWLEMFFKHP | |
| | | | | | | LYGPLLGYRKDGDRTVPSYD | |
| | | | | | | VAAQEDPHHPCHLILNTIKS | |
| | | | | | | DPSDIVLLWMSAVGFWSQFT | |
| | | | | | | SPLIERFLNPDESTIEQMLS | |
| | | | | | | DVYEVDAVSGKTRSVVLVAP | |
| | | | | | | ELSDDEIRHRAQREDYWIPG | |
| | | | | | | SLVASRLARTRSAFETDAEQ | |
| | | | | | | KSLVNKLRALANGGTAGKGS | |
| | | | | | | KQAEKNQDAFDPAFYRRFYR | |
| | | | | | | DLSALPNDRALEDHYFTHGK | |
| | | | | | | LVGRYGSEAMMKREQAALEQ | |
| | | | | | | RLPRDFDAGAYFMANPDLPV | |
| | | | | | | TQPVSWTATRHYLNIGAVQN | |
| | | | | | | RPYHYHFPGLDEAFEALLAT | |
| | | | | | | NEISLSDAERKDYQDGVPAR | |
| | | | | | | ILLLRRLGAISAPWFNMLDL | |
| | | | | | | QEFSALNFEWCGKPASAAEA | |
| | | | | | | ILTFLKDGIERIAPLSVSVA | |
| | | | | | | FDPDFYRRQYTDTADLSDVD | |
| | | | | | | AYRHWLEAGSLMHRAPSEEW | |
| | | | | | | ALQHMIGQRTYPPAFHWDRF | |
| | | | | | | QATDRARFARSTRLDLLDAF | |
| | | | | | | LSTAVPPRDDLVGGPGAGD | |
| | | | | | | LWTWRAARAQGAGDQHLSTE | |
| | | | | | | CLREAARVEPHRGVFWHTLG | |
| | | | | | | DRLQSQGDLHGALEAYTRCL | |
| | | | | | | KTDTPNRWSHINCIRLSADL | |
| | | | | | | GFYKKGLEHLRAAQAAWKEM | |
| | | | | | | QPWREARTHLFMRWFDHAAH | |
| | | | | | | HSYDRITQDDIRARTHPDAR | |
| | | | | | | FLAFMNTFVPAVASIGIPAP | |
| | | | | | | LTLARTDGPILILSGSGLSE | |
| | | | | | | RTRWEASLRIAPEDARQVIV | |
| | | | | | | FSREQVALFAESLPGASVAL | |
| | | | | | | YHEVETDGLIVDTLLRAKAL | |
| | | | | | | GVRNIYWAGPLGRDSTGEGP | |
| | | | | | | DLSDLAWSDFLLSRDSRETG | |
| | | | | | | RTLRSIHAATMCDDVVLTMP | |
| | | | | | | SLITRFHDLGLRPRLGVSAL | |
| | | | | | | MEALADMPRTTETAPGKIRI | |
| | | | | | | FCSLTGRPVRVSTSDEDAYA | |
| | | | | | | PRIGQKALLKALDTLLETYP | |
| | | | | | | DVSLLVEGVDPALPLSIRNS | |
| | | | | | | TRIERLGSELTSDQRLAALS | |
| | | | | | | RSSIALDLRHVPRDQRSLAD | |
| | | | | | | EATWSGIPCLVLSDNPALGS | |
| | | | | | | ASTPGYAKWAQISEILKAWI | |
| | | | | | | GQPQTLEDVTLAARTHFEEA | |
| | | | | | | VSPTPVVWSPVRAVPTTKAR | |
| | | | | | | PRILFANLFAPPQTTGGATR | |
| | | | | | | VLSDNAGYMLAHAGDDYDFA | |
| | | | | | | ILASDDENSHRGVTRVDSWK | |
| | | | | | | GAPVFRIATPQEIDMDWRTY | |
| | | | | | | NSEVDAQTRRIITLFKPDLV | |
| GO_ | GO_ | Acetylornithine | H | F | Both | MIPALMPNYKRADLAFEQGE | 22 |
| 2902 | 2902 | aminotransferase | | | | GVWLTATNGRRYLDFGAGIA | |
| | | (EC | | | | VTSLGHAHPKLVKTIAEQAA | |
| | | 2.6.1.11) | | | | KVMHVSNLYRIPQAEKLAEL | |
| | | | | | | LVQNTFADSVLFCNSGAEAN | |
| | | | | | | EGMVKMIRRAQFENGHPERT | |
| | | | | | | NILCFNGAFHGRTLAMISAT | |
| | | | | | | GNPAYLKGFGPVVEGFDHAP | |
| | | | | | | FNNTNTLRDAITPHTAGIVV | |
| | | | | | | EPVQGESGIKPATREFMEGL | |
| | | | | | | RAVCDEYGLYLGFDEVQTGV | |
| | | | | | | GRTGKLFAHEWYGVRPDVIS | |
| | | | | | | VAKGIGGGFPLGAVLATEEL | |
| | | | | | | ARHLTPGSHGTTFGGNPLAC | |
| | | | | | | AAGVTVLEEILSPGFLEHVR | |
| | | | | | | SVGDAFGRMLEDVVSRSEGV | |
| | | | | | | FDNVRGIGLMRGLHCVPPVA | |
| | | | | | | DVMQAVLNQELLTVSAGDNV | |
| | | | | | | LRLVPPLIVTETECREACER | |
| | | | | | | LVKAADSLKVPATQENAS | |
| GO_ | GO_ | Transcriptional | H | F | Both | MSQETNAPELHQLTAQIVTA | 23 |
| 3260 | 3260 | regulator | | | | YVSNNDIPADALPALIRSVH | |
| | | | | | | DSLATVNVPEEAPVEKPVPA | |
| | | | | | | VSPRKSVFPDYIICLEDGKK | |
| | | | | | | LKLLRRHLKTAYNMTPQEYR | |
| | | | | | | ERWGLPPEYPMVAPNYANHR | |
| | | | | | | SSLARKIGLGRRRED | |
| GO_ | GO_ | UDP-glucose 4- | H | F | Both | MRYLVTGGAGFVGSHVVLAL | 24 |
| 36 | 36 | epimerase (EC | | | | RDAGHDVVVLDNLSTGYREA | |
| | | 5.1.3.2) | | | | VPAGVPFHKVDLLDYAATSA | |
| | | | | | | VVAQGNWDGVLHFAALSLVG | |
| | | | | | | DSMRDPFHYLRQNYLTALNL | |
| | | | | | | VQICAGHGVRKIVFSSTAAL | |
| | | | | | | FGGPERLDPIPETAPVQPGS | |
| | | | | | | PYGESKFMIERVLHWADAIY | |
| | | | | | | GLRSACLRYFNAAGADPQGR | |
| | | | | | | AGEDHRPETHLIPLTIDAAL | |
| | | | | | | GRRPALKLFGTDYPTRDGSC | |
| | | | | | | VRDYIHVTDLADAHIRALAQ | |
| | | | | | | IDQRSVTYNIGNGHGYSNLE | |
| | | | | | | IIQSVERVSGRKVPWEPAPR | |
| | | | | | | REGDPALLVADSTTLRNDTE | |
| | | | | | | WAPRFGDIDSIVETALRWRE | |
| | | | | | | SHPHGYGG | |
| GO_ | GO_ | hypothetical | H | F | Both | MSPENDQDRNRPDPGDYARA | 25 |
| 406 | 406 | protein | | | | PKQPAGPASGARPQARFDRE | |
| | | | | | | RLSNDYDDEPPPRRRPAAAG | |
| | | | | | | GASGAGRLTSVLGNDPATRK | |
| | | | | | | LVGGAVGIGVVLLLAVGGWS | |
| | | | | | | LMGSHHGGIPIIGPPPGPVK | |
| | | | | | | DRPADPGGMQIMGGDDGDTD | |
| | | | | | | MTGNGEAHLAPGPEQPDTKA | |
| | | | | | | LARQYGVPPGTPAPETPKAD | |
| | | | | | | APKADGAAPDNAPAAQTPAI | |
| | | | | | | PPEAAAPADTGQMSPGTALP | |
| | | | | | | ATVPEKPQDQAAAPAEAPKA | |
| | | | | | | APPKEAPKKEEAPVHHAARP | |
| | | | | | | AEKPLPAPVPEPENVGPPKA | |
| | | | | | | AAPAAETSGGTHEVQLGALD | |
| | | | | | | SEAAARKEWDSLRHQAPALF | |
| | | | | | | AGHTPLFEKTIRGDHTFVRL | |
| | | | | | | RIGGFADLKSARAYCVKLHA | |
| | | | | | | QSVACTPAQF | |
| GO_ | GO_ | Transcriptional | H | F | Both | MKKAVTLNSVAVEAGVSRAT | 26 |
| 868 | 868 | regulator, LacI | | | | ASLVLRDSPLVSLETRDRVI | |
| | | family | | | | GAMDKLGYIYNRGAANLRGQ | |
| | | | | | | KTGTIGLVLCDIGSPFYSQL | |
| | | | | | | MLGVDEVIVDANIVAILVNS | |
| | | | | | | AENPDRQLRQIRRLREHGVD | |
| | | | | | | GLILCPAAGSSDALLSEIER | |
| | | | | | | LHLPCVQVLRHVSQRNGNYV | |
| | | | | | | GPDYADGTSLAVTHLVRHGR | |
| | | | | | | RKIAFLGGKPVHSAARERLD | |
| | | | | | | GFRKTLKKYKLEHDLIIPTQ | |
| | | | | | | LENLSDLGSFPELLASSTPP | |
| | | | | | | DAVVCYNDMLAQSVMGYLLA | |
| | | | | | | RGKMPGRDFAVIGADDLPQS | |
| | | | | | | AVSFPTLTTIVTDPVGVGRN | |
| | | | | | | AARLLLDRIDNPATSSTRIL | |
| | | | | | | VSGQLMIRQSCGGQLS | |
| GO_ | hemK | Peptide chain | H | F | Both | MMMAKDDLLREASQALEQAG | 27 |
| 1937 | | release | | | | IEDARREARLLLCWATGRDL | |
| | | factor N(5)- | | | | GGLLSLDGVEPAQKSRFAEA | |
| | | glutaminemethyl | | | | LKRRLEREPLAFITGETGFW | |
| | | transferase (EC | | | | TLDLETGRDTLIPRADSEAL | |
| | | 2.1.1.297) | | | | IEALLDVCPDRNAPLSILDL | |
| | | | | | | GTGTGCLLLAALSEYPQATG | |
| | | | | | | VGVDLSPQAVALAQRNSVRT | |
| | | | | | | GLEKRTAFLAGSWADALNAR | |
| | | | | | | FDVVLSNPPYIETGDLAGLM | |
| | | | | | | PEVLQYEPARALDGGTGGLD | |
| | | | | | | AYRILCAALPALLVPGGYAI | |
| | | | | | | LEMGIGQIDAVSALGVASGL | |
| | | | | | | RDVAHKADLGGIERALVLQS | |
| | | | | | | DG | |
| GO_ | ntrX | Nitrogen | H | F | Both | MEHEILIVDDEPDIRFLIEG | 28 |
| 174 | | regulation | | | | ILNDEGYKTRTAANSDQALE | |
| | | protein NtrX | | | | LFRAHCPSLAILDIWLQGSR | |
| | | | | | | LDGIELLKIFQTEEPGLPTL | |
| | | | | | | MISGHGTIETAVSSLKLGAY | |
| | | | | | | DFIEKPFQSDRLLVVVRRAL | |
| | | | | | | EAARLRRENAELRLRAGPET | |
| | | | | | | TLSGDGAVISAVRAQIERVA | |
| | | | | | | PTNSRVLISGPAGSGKEVAA | |
| | | | | | | RMIHARSRRAEGPFIALNCA | |
| | | | | | | TLAPNRFEEELFGLEGEDGQ | |
| | | | | | | PMRRGVLERAHRGTLLLDEV | |
| | | | | | | ADMPPETQGKIVRALQDQTF | |
| | | | | | | ERLGGNTRVKVDIRVIATTN | |
| | | | | | | RDLQSEIAAHRFREDLYYRL | |
| | | | | | | AVVPLRIPSLRERREDIPGL | |
| | | | | | | ARHFLERCAQSSGLPVRELS | |
| | | | | | | VDALAALQSYEWPGNVRELR | |
| | | | | | | NLMERLLIMMPGTGNDPIRA | |
| | | | | | | DMLPATISQGAPSMTRLNSG | |
| | | | | | | ADVMSLPLREARDLFETQYL | |
| | | | | | | QVQLMRFGGNISRTASFVCM | |
| | | | | | | ERSALHRKLKQLGVTTNEER | |
| | | | | | | NTAPSTPVSG | |
| GO_ | paaD | PaaD-like | H | F | Both | MSEAMTDITPSEDTATAPAP | 29 |
| 1871 | | protein | | | | GTAPDQEAVIAAIATVYDPE | |
| | | (DUF59) | | | | IPVNIYELGLIYAIDLHDDG | |
| | | involved in | | | | RVHIEMTLTAPNCPSAQELP | |
| | | Fe-Scluster | | | | EMVRDAVSHVPGVSQATVEI | |
| | | assembly | | | | VWDPPWDMSRMSDDARLALN | |
| | | | | | | MF | |
| GO_ | tps1 | Trehalose-6- | H | F | Both | MIQVPFPPSRAALLLDFDGT | 30 |
| 2181 | | phosphate | | | | LVDIAPTPESVRVPQGLAAD | |
| | | phosphatase (EC | | | | LLRLRDMLDGALAIITGRPI | |
| | | 3.1.3.12) | | | | AQIDHFLPDIPHAVAGEHGV | |
| | | | | | | MMRHAPGQALRERKLPVVPG | |
| | | | | | | EWIQAVEKAAADHPGASVEH | |
| | | | | | | KKAGMVLHYRRAPEAESVFR | |
| | | | | | | ELASVWPVENRGFHLQDAQM | |
| | | | | | | AIELRPLGIDKGKALRELMA | |
| | | | | | | EPPFAGRLPVFAGDDATDRD | |
| | | | | | | GVRAARQMGGAGWLIPDDFP | |
| | | | | | | DAATFRRWLHDLSEGHGWGA | |
| GO_ | mur | Manganese | H | F | Both | MVADTKIAERRAGPGNRKAP | 31 |
| 3261 | | uptake | | | | SSPVPDDSHIARLCVESGLK | |
| | | regulation | | | | MTGQRRVIAHVLSVADDHPD | |
| | | protein | | | | VEELYRRASEIDSRISVATV | |
| | | MUR | | | | YRTVRLLEEKGILERRDFGG | |
| | | | | | | GRARYEASDSGNHYHLIDVD | |
| | | | | | | SGRVIEFEDEEPVRLLAQLA | |
| | | | | | | QRLGFDLVSHRIELFGRRAE | |
| | | | | | | PDDRKKSPSENRNKSGS | |
| GO_ | GO_ | Shikimate 5- | H | F | Both | MIDGHTKLAGVMGWPVEHSR | 32 |
| 2463 | 2463 | dehydrogenase I | | | | SPLMHNHWCRVNGVNGAYVP | |
| | | alpha (EC 1.1.1.25) | | | | LPTRPEGFDQALRGLAAAGF | |
| | | | | | | QGVNVTIPHKEAAMLACDEL | |
| | | | | | | TPTAKRAGAVNTICFVAGRI | |
| | | | | | | IGDCTDGTGFCDNLSAHDVE | |
| | | | | | | ISGRAMVLGAGGAARAVAAA | |
| | | | | | | LLDRGCEVVIANRTLERAEA | |
| | | | | | | LVEALGGGEAVAWYEWPSLL | |
| | | | | | | SGCSLLVNATSMGMGGKAGL | |
| | | | | | | DWDAVLREAAPGLCVTDIVY | |
| | | | | | | TPRETPLLLAAQARGLRTVD | |
| | | | | | | GLGMLVHQARAGFRAWFGVD | |
| | | | | | | PQADQTTFDLLAASLRTDA | |
| GO_ | cyoA | Cytochrome O | H | S | Up | MMKAGPMKKLWRYLPALPAL | 33 |
| 2506 | | ubiquinol | | | | MLSGCTVDLLQPRGPVAEMN | |
| | | oxidase | | | | RDVMVAEFVIMMLVVVPTCA | |
| | | subunit II | | | | ATLYFAWKYRASNKEAEYLP | |
| | | (EC 1.10.3.—) | | | | TWDHSTAIEYVIWGVPAILI | |
| | | | | | | ALLGAISWWSTHAYDPYRPL | |
| | | | | | | QTADNVKPLNVQVVSLDWKW | |
| | | | | | | LFIYPDLGIATINQLDVPTN | |
| | | | | | | TPLNFQITSDTVMTSFFIPR | |
| | | | | | | LGSMIYSMPGEQTQLHLLAS | |
| | | | | | | ESGDYLGEASQFSGRGFSDM | |
| | | | | | | KFRTLAMDPAQFNDWVEKVK | |
| | | | | | | SGSENLDDTTYPKYAAPQEA | |
| | | | | | | APVQYFAHVQPDLFDGIVAK | |
| | | | | | | YNNGMMVDKKTGKVMHMQSA | |
| | | | | | | SNTAPSDTGMKE | |
| GO_ | cyoD | Cytochrome O | H | S | Up | MTQAPTTTMTGDSHGSYPSY | 34 |
| 2503 | | ubiquinol | | | | LIGFVLAVILTVASFAAVMS | |
| | | oxidase | | | | HALSPGMALAALTVLAVVQI | |
| | | subunit IV | | | | VVHLVFFLHMNTSTEQSWNL | |
| | | (EC 1.10.3.—) | | | | MCFIFAAASVIVIIGGTIFI | |
| | | | | | | MHDTAINMMSR | |
| GO_ | lam | Lysine 2,3- | H | S | Up | MDDMVKTAPRHSTKRHTLRT | 35 |
| 2812 | | aminomutase | | | | PSDLIDAGLAPEADRATLEA | |
| | | (EC | | | | VGERFTMAIPPAFQDLITHP | |
| | | 5.4.3.2) | | | | DDPIARQVIPDARELITLPH | |
| | | | | | | EDADPIGDDALSPVPGIVHR | |
| | | | | | | YADRALLKPLLVCPLYCRFC | |
| | | | | | | FRREHVGPGGGLLSDAQLEA | |
| | | | | | | ALDWVRQHPDIREIILTGGD | |
| | | | | | | PLMLAPRRLKHIVQSLSDIP | |
| | | | | | | HIETIRIHSRVPVADPGRMT | |
| | | | | | | EELLDAMETDRSMWLVVHAN | |
| | | | | | | HANELTPQAIKGIRAVLSRA | |
| | | | | | | IPVLSQSVLLRGVNDTVESL | |
| | | | | | | EALLRAFLKARVKPYYLHHL | |
| | | | | | | DAAAGTGHFHVPVAEGQALL | |
| | | | | | | RQLRGRVTGLAWPTYVLDIP | |
| | | | | | | GGRGKVPIGPEYLDPASPGT | |
| | | | | | | VSTPDGEACSFT | |
| GO_ | GO_ | NADH | H | S | Up | MASRSEILIVGGGVAGLSLA | 36 |
| 923 | 0923 | dehydrogenase | | | | TRLGKSMGKSGKARITLIDK | |
| | | (EC | | | | SFSHVWKPMLHCFASGTLSN | |
| | | 1.6.99.3) | | | | ENDKVNFISQASGHHFEFWP | |
| | | | | | | GEVASIDRENREVVLSPLLE | |
| | | | | | | ADGTVILESRRMKYDTIVIA | |
| | | | | | | IGSCANDFGTPGVKEHCMSI | |
| | | | | | | DNLVEANAFNEKFRMELLRA | |
| | | | | | | FGNNSELDIAIVGGGATGVQ | |
| | | | | | | LAAELHKALEIVGPYNLHAF | |
| | | | | | | GKAPPKLHVTLLQSGARILP | |
| | | | | | | AFPESVSAAAQQELEHIGVT | |
| | | | | | | VRTNARVAAADDHGFTLKDG | |
| | | | | | | SYVPAKLRVWAAGVKAPEVT | |
| | | | | | | TAYGGLTINRTGQILVNPNL | |
| | | | | | | SSIDDEHIFALGDCSFIQDD | |
| | | | | | | PLPATAQVARQQAKHLARYL | |
| | | | | | | PAWIEHGQKVPSCIFHNKGA | |
| | | | | | | IVALGKYNGWAALPGGTVWG | |
| | | | | | | GGISHGFSARMAHLMLYRQH | |
| | | | | | | QIELFGYYRGLMSFYSDWVE | |
| | | | | | | TFVRPSVRLD | |
| GO_ | GO_ | hypothetical | H | S | Up | MSGCSDPEGIFAPDSAAVRA | 37 |
| 1060 | 1060 | protein | | | | FRARLDSQPSATAALQARCS | |
| | | | | | | TPIRVIRLSVDRPVTEDILT | |
| | | | | | | LLQVRETHQVMTRHVRLLCG | |
| | | | | | | ETVLSDAWNWYVPERLSPAM | |
| | | | | | | NTLLEQTDTPFGRVVRQTAF | |
| | | | | | | RRQRLETRFPGRASGIVLEN | |
| | | | | | | RALLLRGADNAPISLVVEDY | |
| | | | | | | LPAAIRP | |
| GO_ | GO_ | FIG00687856: | H | S | Up | MPDVLEQRLIGELTTPVDPG | 38 |
| 1659 | 1659 | Predicted | | | | VVAFADALARACPVLPLGVL | |
| | | nucleotidyl | | | | FYGSLLRKADPDGILDFYII | |
| | | transferases | | | | TENAAGFAGGLVARTGNLVL | |
| | | | | | | PPNVRYSEFRHGGRVLRAKI | |
| | | | | | | AVLSRAQFEARTGLGALDTT | |
| | | | | | | IWARFCQPVRLVWVRDPQSA | |
| | | | | | | DVILSLIAGCVTTATCWAAL | |
| | | | | | | LGDVSMTALEFWQTLFAHTY | |
| | | | | | | ASELRVEKKGRGNSILEGQE | |
| | | | | | | ARYAALLTLGWARGRLQFSA | |
| | | | | | | HGDRLEPVIDAALRRKAARR | |
| | | | | | | WALIQISGRPRNVSRLLKAA | |
| | | | | | | FTFENGASYLAWKIQRHTGF | |
| | | | | | | DMQLSPFESRHPLVMLPRLL | |
| | | | | | | WRARGLLARSKA | |
| GO_ | GO_ | AMP-binding | H | S | Up | MSGNPNGASPGLTEANQNPT | 39 |
| 1662 | 1662 | enzyme, | | | | ANPVPTPSRSGLERRYGDFP | |
| | | associated | | | | SFAAALDYAAQGESGFNIYS | |
| | | with | | | | GRGQLLEALPYRLLREQARS | |
| | | serinepalmitoyl | | | | MACRLLGLGLVPGDRVAIVA | |
| | | transferase | | | | ESDGDFARIFFGCQYAGLVP | |
| | | | | | | APLPLPVAFGGREGYVTTLR | |
| | | | | | | GMIQSAAARAVVVPDVIGSW | |
| | | | | | | TADIVDGLDLVFGGSPADLY | |
| | | | | | | RHAEARVELPEISPTALSYL | |
| | | | | | | QFSSGSTRFPMGVSVTQAAG | |
| | | | | | | MANARAIARDGLHVYPAEDP | |
| | | | | | | RDDRCVSWLPLYHDMGLVGF | |
| | | | | | | FLTPLTCQLSVDLLPTREFA | |
| | | | | | | RRPHVWLDLISRNRGTIAYS | |
| | | | | | | PSFGYELCARRSGQADLDLS | |
| | | | | | | CWRIAGIGGDMIRHHILEGF | |
| | | | | | | AERFASNGFRATSFVASYGM | |
| | | | | | | AEATLAISFAPLDTGIQTDT | |
| | | | | | | IDLRRLEKDGIAEPSNDPSH | |
| | | | | | | PLRTFVLCGEALPDHQIEVR | |
| | | | | | | DAAGHDLADRQVGTVYVRGP | |
| | | | | | | SLMCGYFRRPDETEAVLDAD | |
| | | | | | | GWLNTGDLGYHLNGQIVVTG | |
| | | | | | | RAKDLIIINGRNIWPQDLEW | |
| | | | | | | SAESEVPSLRSRDVAVFSVD | |
| | | | | | | GDEGEKIVALVQCRATEDES | |
| | | | | | | RNQLRDEVTSLFRRQHGVDV | |
| | | | | | | DVILVPPRTLPQTSSGKLTR | |
| | | | | | | AKAKTMLLSGQFEQQPETTS | |
| | | | | | | SVA | |
| GO_ | GO_ | hypothetical | H | S | Up | MKVAFPLIGQRHQTLHALPI | 40 |
| 1663 | 1663 | protein | | | | ALEVSARHPDVAVHVSCLTV | |
| | | | | | | SHLELARSLATLYPEARVQF | |
| | | | | | | DLLPISPKLRRRIELHGLRV | |
| | | | | | | VDRLIGLFASRHYFRTFDAI | |
| | | | | | | IVPEATSLQLRRMGVGRPKM | |
| | | | | | | IWTGHGAGDRAIGFARHLGK | |
| | | | | | | FDFLLVPGRKVEQRMLEKSI | |
| | | | | | | IRPGAYHRGTYAKFDLVRRM | |
| | | | | | | DAKRPKLFNNNRPTILYNPH | |
| | | | | | | FLRRLSSWPEMGHQVLQFFA | |
| | | | | | | TQDRYNLVFAPHFRLFDNHR | |
| | | | | | | EEGEALRRQYGHLPHMLIDP | |
| | | | | | | GSHRSIDMTYTMGADLYLGD | |
| | | | | | | VSSQVAEFMIRPRPCLFLNA | |
| | | | | | | HHVKWHGNPDYQFWTLGPVT | |
| | | | | | | ENVSDLGSKIENAFETHPRF | |
| | | | | | | LEAQRQYVLETFETLGDEPT | |
| | | | | | | APAAADAIVDFLKRAA | |
| GO_ | GO_ | Mannose-1- | H | S | Up | MSQKIVPVILSGGSGSRLWP | 41 |
| 182 | 182 | phosphate | | | | VSRSSYPKQFWPLLSKYSLI | |
| | | guanylyltransferase | | | | QETALRGARAGLADPIVICN | |
| | | (EC 2.7.7.13)/ | | | | AEHRFIVAEQLRDVGVENAR | |
| | | Mannose-6- | | | | IVLEPVGRNSAPAIAAAAFL | |
| | | phosphate isomerase | | | | VAETDPDAVLWIMAADAAIT | |
| | | (EC 5.3.1.8) | | | | DEAALYSALDHAVAAAGQGR | |
| | | | | | | IVTFGMKPTRVETGYGYIES | |
| | | | | | | GAPLSGLEGVCEVSRFVEKP | |
| | | | | | | DAATAEAFFRDGRYLWNSGM | |
| | | | | | | FVTQAGVFLSEIQTFEPALY | |
| | | | | | | EHVGQAVRTRQSDLDFDRLD | |
| | | | | | | DASFRQAPDISVDYAVAERT | |
| | | | | | | KRAAVVPGTFGWSDIGSWDA | |
| | | | | | | LWELTSKDEAGNATFGDVFL | |
| | | | | | | DDARNCYVRSDGIVATVAGV | |
| | | | | | | EDLIVVVTQDAVMVSHRDRA | |
| | | | | | | QDVKHMVSRLKKAGRKEATA | |
| | | | | | | HNRMYRPWGFYESLIQADRF | |
| | | | | | | QVKRIVVEPGQKLSLQKHFH | |
| | | | | | | RAEHWVVVGGTAVVTRDADQ | |
| | | | | | | IMVRENESVYLPLGCVHRLE | |
| | | | | | | NPGRIPLTLIEVQSGPYLGE | |
| | | | | | | DDIVRIEDVYSRN | |
| GO_ | GO_ | Sensory | H | S | Up | MTVTRSGSPDLNPRRWRRLW | 42 |
| 1993 | 1993 | transduction | | | | YRRDALRSIRMFDTVGARVM | |
| | | histidine kinase | | | | TLIVATTLPLAIIASLLAWH | |
| | | | | | | SYQQNVGNSAMRTERDTQLA | |
| | | | | | | ISEITTDLDQTHTLLDMLAD | |
| | | | | | | GDISSGNALREFALVQTVSQ | |
| | | | | | | HHYCMLMLTDVSGRPSVVLP | |
| | | | | | | PPSTQDAAICSSPELAAPAT | |
| | | | | | | NSPTARTPVVGVDVLKGDRG | |
| | | | | | | PLLKFVVPILSNNSVSGYII | |
| | | | | | | AVRTLGWQRSHLPKGDSRLL | |
| | | | | | | LGTDNNSRHFLAMPDGTLYS | |
| | | | | | | LFPDRPVTAELPARAFARLK | |
| | | | | | | RDISSLSLHDVFTSQGITYA | |
| | | | | | | FQNAYGPVSLIVATERTAEE | |
| | | | | | | SHALNIFLIRVSLIVGLLVL | |
| | | | | | | ELMAVALGARLFLVDPLEKL | |
| | | | | | | ALAVADWRKGAAFAPRISHS | |
| | | | | | | IPLEIRHLERAFLRATRRLS | |
| | | | | | | KHEQDLEQSARNQDMLIREI | |
| | | | | | | HHRVKNNLQVVASLLNLQAS | |
| | | | | | | RIRSHEAREEFRLVRDRVRA | |
| | | | | | | LATLHRYLYSESGLSALDVQ | |
| | | | | | | SFLEELCSILLSANGMNAQT | |
| | | | | | | RIRLQLDIEHVLISPDQAVP | |
| | | | | | | IALIVTEVVSNALRYAFPEN | |
| | | | | | | RAGHIVINLHKVVSTDAEKE | |
| | | | | | | GLVELKLGDDGIGINAGQAT | |
| | | | | | | ESRTRREGIGMQLIRGFARQ | |
| | | | | | | INADMTVSNENGTWYTLRFI | |
| | | | | | | PERPSLTALAMARKAISYGE | |
| | | | | | | DSGL | |
| GO_ | GO_ | Dolichol- | H | S | Up | MAVLNEAENILPVCQELADT | 43 |
| 2191 | 2191 | phosphate | | | | FGADPSAEILAIDDGSTDAT | |
| | | mannosyltransferase | | | | VKKLLEARQTLLPRLRIISH | |
| | | | | | | PKRLGKSAALRTGITAAKGQ | |
| | | | | | | WIATIDGDGQDDPSAILKML | |
| | | | | | | DQATSASGAAPLVVGVRRKR | |
| | | | | | | NDRLSRRIATRFANGLRRRL | |
| | | | | | | LNDGCPDTGAPLKLFPRDLF | |
| | | | | | | LKIPQFEGVHRFLPALLGHY | |
| | | | | | | GAPLICIEVQHRARLHGSSK | |
| | | | | | | YTNFNRALVGIRDLLGVMWL | |
| | | | | | | QNRTHLPDHLTEH | |
| GO_ | GO_ | Diaminopimelate | H | S | Up | MSAPLPSDPATDPSFSDMLE | 44 |
| 2479 | 2479 | decarboxylase | | | | TRPSLKMDARDGLMFEGVPL | |
| | | (EC | | | | HVIAAAVGTPCWITGAETLR | |
| | | 4.1.1.20) | | | | RRAKALRTAFEARNLPVNMH | |
| | | | | | | FAMKSQDHQATLTILRQCGY | |
| | | | | | | GVDIVSGGEMQRALHAGIQP | |
| | | | | | | SGIVFSGVGKSDAELRAAVE | |
| | | | | | | HDIAQVNVESVEELYRLDDI | |
| | | | | | | ARACGRVARAALRVNPDVDA | |
| | | | | | | DTHDKISTGRAGDKFGIEHR | |
| | | | | | | RAVALYGEAASLKNVRLVGL | |
| | | | | | | AVHLGSQMLTATPFREGYAR | |
| | | | | | | LADMVREIRAAGHTVESVDC | |
| | | | | | | GGGLGIRYRDEIAPSPDMLA | |
| | | | | | | GVIAETLGDLDVRLSIEPGR | |
| | | | | | | WLSAPTGILLTRVIETKAGN | |
| | | | | | | PDFVVIDAAMNDLARPSLYE | |
| | | | | | | SWHGIMPVAPSGLTSPTKLW | |
| | | | | | | DIVGPVCESSDIFARDRALP | |
| | | | | | | AETKRGDLIALLDTGAYGSV | |
| | | | | | | MSSTYNTRPLAAQVLIDNGK | |
| | | | | | | WEIIRQRQSVAELIAAETVP | |
| | | | | | | EWLTAKDDHG | |
| GO_ | GO_ | Mitochondrial | H | S | Up | MPDTIEVTRLDNGLTIITER | 45 |
| 2557 | 2557 | processing | | | | MDRVETVSFGAYVSIGTRDE | |
| | | peptidase- | | | | TADNNGVSHFLEHMAFKGTE | |
| | | like protein | | | | RRSASRIAEEIENVGGYINA | |
| | | (EC 3.4.24.64) | | | | YTARETTAYYVKLLKNDLAL | |
| | | | | | | GVDIIGDILTHSTFLDAEIE | |
| | | | | | | RERGVILQEIGQANDTPDDI | |
| | | | | | | IFDQFQERAFPEQPMGRPTL | |
| | | | | | | GSEERVSTMTRDTLMSYMRE | |
| | | | | | | HYTTHNITIAAAGNLHHQQV | |
| | | | | | | VDLVKEHFRDLPTHQTPRPR | |
| | | | | | | AASYEGGELRTPRELDQAHL | |
| | | | | | | VMGFPSVSYMHPDHYAVMIL | |
| | | | | | | STLLGGGMSSRLFQEIRERR | |
| | | | | | | GLVYSVYSFASPFSDSGLFG | |
| | | | | | | LYAGTGEEQTAELVPVMIDE | |
| | | | | | | LKRLQDGLSAEELSRARAQL | |
| | | | | | | KSSLLMSLESTGSRCEQLAR | |
| | | | | | | QIQVHNRPVPTAETVGKIDA | |
| | | | | | | VTEDDILRVARTIFSGTPTF | |
| | | | | | | TAIGPIDNMPSLEDITARLA | |
| | | | | | | A | |
| GO_ | GO_ | NifU protein | H | S | Up | MSGRLERKDMTTMFIETEDT | 46 |
| 3255 | 3255 | | | | | PNPATLKFLPGRSVTGDARP | |
| | | | | | | VDFGDADVAAGRSELATALF | |
| | | | | | | DQPNVRRVFLGGDFVSVTKS | |
| | | | | | | DDISWGDLKPVVLGTITTFF | |
| | | | | | | ESGRPVLSGTQAAPEHDVSP | |
| | | | | | | EDAEVVSRIQDLLDTRVRPA | |
| | | | | | | VAGDGGDIAFRGYKDGVVYL | |
| | | | | | | AMQGACSGCPSSRATLKHGV | |
| | | | | | | ENMLRHYVPEVASVEQVED | |
| GO_ | kdtA | 3-deoxy-D-manno- | H | S | Up | MTLFPRLLRLWLGTCLRTTR | 47 |
| 2438 | | octulosonic acid | | | | WQVSGSPRALETLTTPAQGT | |
| | | transferase | | | | VVAFWHRSLTLSPALWFWAR | |
| | | (EC 2.4.99.12)(EC | | | | TLEPRLELRVLISRNPDGML | |
| | | 2.4.99.13) | | | | IADVVRPWGIIGIHGSSSKK | |
| | | | | | | GKNKGGAAVLRTALKELEAG | |
| | | | | | | SIVAITPDGPRGPAELVQPG | |
| | | | | | | AVALSRLARCAVVPVGMAST | |
| | | | | | | SLRLPSWDGLVFPLPFGRGA | |
| | | | | | | LIMGEPLFQPDAALLQNALN | |
| | | | | | | DVSLRAESVVRHRQSNLADR | |
| | | | | | | LWRVAGTLMAPALTVMLRIR | |
| | | | | | | LHRGRELPGRLRERMGLERT | |
| | | | | | | GPRRGHRPPGQLLWIHAASV | |
| | | | | | | GETLCALPLAEALLEALPEM | |
| | | | | | | RILFTTATVTGSEIVARHPL | |
| | | | | | | YGQRIIHRFIPHDVPRWLRR | |
| | | | | | | FLNLWQPEGAIFVESELWPG | |
| | | | | | | IIAACSRRDIPVMLVNGRLS | |
| | | | | | | DRSARLWTRLGDPARRMMKR | |
| | | | | | | LSWVAARGPEDAARFRALGA | |
| | | | | | | LPVYEDGDLKQDAPPLAYDE | |
| | | | | | | TEYARLESLIGERPVFVAAS | |
| | | | | | | THPGEEELVLQAAERARRLQ | |
| | | | | | | PDLLTIIVPRHPARGAELAA | |
| | | | | | | RFDLPRRAAGQDPTPQTQIW | |
| | | | | | | LADTLGELGLLYRLADRCFL | |
| | | | | | | GNSLAGKGGGHNPFEPLRLG | |
| | | | | | | IPTATGPKMENWREAIATVS | |
| | | | | | | DTIHIVNDVECLTRWLESPL | |
| | | | | | | PPVRTTGLQRSVVSVLRDRI | |
| | | | | | | LKTVER | |
| GO_ | kup | Kup system | H | S | Up | MPEHDGDHASNPPHGVGIPN | 48 |
| 1459 | | potassium | | | | DSGEIVQTIEQARSEGHTHE | |
| | | uptake | | | | IGGEEDGSSHHRPAGMGALL | |
| | | protein | | | | AVLGVVYGDIGTSPLYALQS | |
| | | | | | | SVSIVSSPKAPAQPWEIMGL | |
| | | | | | | ASLTFWALMLIVTIKYVILI | |
| | | | | | | MRADHDGEGGIIALMSLAQR | |
| | | | | | | VCKSQHFRWLFGLVGIAGTC | |
| | | | | | | LFFGDSIITPAISVLSAVEG | |
| | | | | | | IETSVPSASHIIIPLAMVVL | |
| | | | | | | VALFSVQVLGTGKIGKAFGP | |
| | | | | | | IMVCWFSVLAILGIKGIFLY | |
| | | | | | | PHILLALSPTFALEFIIMHG | |
| | | | | | | YLSFIALGSVVLSVTGAEAL | |
| | | | | | | YADMGHFGRAPIRKAWLFFV | |
| | | | | | | LPSLTLNYFGQAALLIRDPH | |
| | | | | | | ALSNPFYLLVPHWAQIPMLI | |
| | | | | | | LATFATVIASQAGISGSFSL | |
| | | | | | | CRQLIQLGYLPRTRIMHTNA | |
| | | | | | | SEEAQIYLPSLNWILAFGAL | |
| | | | | | | VLVLAFRSSSALAAAYGIAV | |
| | | | | | | TGTFLCTCVLAMVVFRRVFK | |
| | | | | | | WKSATVGIVFGFFFIVDSIF | |
| | | | | | | FSANVLKIPDGGWVPLAIGI | |
| | | | | | | ISTIIMTTWKRGRSLIAARQ | |
| | | | | | | QADSMPMGSFLARLPQSRTI | |
| | | | | | | RVPGLAVFLTANPDIVPNSL | |
| | | | | | | LHNLKHNKVLHDHILFVTVE | |
| | | | | | | NLDKPEAERGHRAIVQELAP | |
| | | | | | | NIHRVIVRYGFMEMPNLPRA | |
| | | | | | | LLELNALGVAFDAIQASYFT | |
| | | | | | | SHELVVRSRVPKMQLWRMWI | |
| | | | | | | FLLLLRNAASTTEFLRIPPD | |
| | | | | | | RVVEFGVRIAI | |
| GO_ | mgdH | Glucose | H | S | Up | MSTTSRPGLWALITAAAFAL | 49 |
| 2781 | | dehydrogenase, | | | | CGAILTVGGAWVAAIGGPLY | |
| | | PQQ- | | | | YVILGLALLATAFLSFRRNP | |
| | | dependent | | | | AALYLFAVVVFGTVIWELTV | |
| | | (EC 1.1.5.2) | | | | VGLDIWALIPRSDIVIILGI | |
| | | | | | | WLLLPFVSRQIGGTRTTVLP | |
| | | | | | | LAGAVGVAVLALFASLFTDP | |
| | | | | | | HDISGELPTQIANASPADPD | |
| | | | | | | NVPASEWHAYGRTQAGDRWS | |
| | | | | | | PLNQINASNVSNLKVAWHIH | |
| | | | | | | TKDMMNSNDPGEATNEATPI | |
| | | | | | | EFNNTLYMCSLHQKLFAVDG | |
| | | | | | | ATGNVKWVYDPKLQINPGFQ | |
| | | | | | | HLTCRGVSFHETPANATDSD | |
| | | | | | | GNPAPTDCAKRIILPVNDGR | |
| | | | | | | LVEVDADTGKACSGFGTNGE | |
| | | | | | | IDLRVPNQPYTTPGQYEPTS | |
| | | | | | | PPVITDKLIIANSAITDNGS | |
| | | | | | | VKQASGATQAFDVYTGKRVW | |
| | | | | | | VFDASNPDPNQLPDDSHPVF | |
| | | | | | | HPNSPNSWIVSSYDRNLNLV | |
| | | | | | | YIPMGVGTPDQWGGDRTKDS | |
| | | | | | | ERFAPGIVALNADTGKLAWF | |
| | | | | | | YQTVHHDLWDMDVPSQPSLV | |
| | | | | | | DVTQKDGTLVPAIYAPTKTG | |
| | | | | | | DIFVLDRRTGKEIVPAPETP | |
| | | | | | | VPQGAAPGDHTSPTQPMSQL | |
| | | | | | | TLRPKNPLNDSDIWGGTIFD | |
| | | | | | | QMFCSIYFHTLRYEGPFTPP | |
| | | | | | | SLKGSLIFPGDLGMFEWGGL | |
| | | | | | | AVDPQRQVAFANPISLPFVS | |
| | | | | | | QLVPRGPGNPLWPEKDAKGT | |
| | | | | | | GGETGLQHNYGIPYAVNLHP | |
| | | | | | | FLDPVLLPLGIKMPCRTPPW | |
| | | | | | | GYVAGIDLKTNKVVWQHRNG | |
| | | | | | | TLRDSMYGSSLPIPLPPIKI | |
| | | | | | | GVPSLGGPLSTAGNLGFLTA | |
| | | | | | | SMDYYIRAYNLTTGKVLWQD | |
| | | | | | | RLPAGAQATPITYAINGKQY | |
| | | | | | | IVTYAGGHNSFPTRMGDDII | |
| | | | | | | AYALPDQK | |
| GO_ | mreC | Rod shape- | H | S | Up | MLSIHARQVLAKAVLPILIL | 50 |
| 388 | | determining | | | | LAVGLVLLGLVRRPAVDGVR | |
| | | protein | | | | LMATDFMAPAYHGLVWPQER | |
| | | MreC | | | | VKVWLTDLRGATDLAKENAR | |
| | | | | | | LRDENRALRHWYDVAVALAA | |
| | | | | | | ENGRLKKSLHWIPETVPQYV | |
| | | | | | | TGRVTRDDGGPYSRAVLLDV | |
| | | | | | | GSGHDVRIGDVALDAAGLLG | |
| | | | | | | RVTEVGPHTVRVLMINDDAS | |
| | | | | | | RIPVTLGSSHGDAIMAGDDT | |
| | | | | | | ASPRLIFYPQDHHPVEGERV | |
| | | | | | | ETRGQSTMPAGLPVGTVHYS | |
| | | | | | | APNRPVVVPDADLDRLDIVR | |
| | | | | | | VFDYGDDDSQAPDAPGRVRV | |
| | | | | | | KKLPQNPLTGPLPFSWLPNL | |
| | | | | | | PDMPGRGGQ | |
| GO_ | mreD | Rod shape- | H | S | Up | MVAENSTPHLHSAVEPKQTF | 51 |
| 389 | | determining | | | | RRALDMAARAAMPSLFIVFS | |
| | | protein | | | | AILLSAPFGIPGQAQLQFGI | |
| | | MreD | | | | AMCTVWFWAYSRPRSMPALA | |
| | | | | | | VFLCGLVVEIFSFGPPGTVL | |
| | | | | | | LSLLVIYGVAHHWRYGLSRL | |
| | | | | | | GFIAGWLIFSVFAALASFFQ | |
| | | | | | | WALVCLHAVALLSPAPGLFQ | |
| | | | | | | AALTIGIYPSLTALFVWGRR | |
| | | | | | | TFANPDQA | |
| GO_ | pqqC | Coenzyme PQQ | H | S | Up | MTLLTPDQLEAQLRQIGAER | 52 |
| 2303 | | synthesis | | | | YHNRHPFHRKLHDGKLDKAQ | |
| | | protein C | | | | VQAWALNRYYYQARIPAKDA | |
| | | | | | | TLLARLPTAELRREWRRRIE | |
| | | | | | | DHDGTEPGTGGVARWLMLTD | |
| | | | | | | GLGLDRDYVESLEGLLPATR | |
| | | | | | | FSVDAYVNFVRDQSILAAIA | |
| | | | | | | SSLTELFSPTIISERVSGML | |
| | | | | | | RHYDFVSEKTLAYFTPRLTQ | |
| | | | | | | APRDSDFALAYVRENARTPE | |
| | | | | | | QQKEVLGALEFKCSVLWTML | |
| | | | | | | DALDYAYVEGHIPPGAFVP | |
| GO_ | pqqE | Coenzyme PQQ | H | S | Up | MTLPSPPMSLLAELTHRCPL | 53 |
| 2305 | | synthesis | | | | SCPYCSNPLELERKAAELDT | |
| | | protein E | | | | ATWTAVLEQAAELGVLQVHF | |
| | | | | | | SGGEPMARPDLVELVSVAQK | |
| | | | | | | LNLYSNLITSGVLLDEPKLE | |
| | | | | | | ALDRAGLDHIQLSFQDVTEA | |
| | | | | | | GAERIGGLKGAQARKIAAAR | |
| | | | | | | LIRASGIPMTLNFVVHRENV | |
| | | | | | | ARIPEMFALARELGAGRVEI | |
| | | | | | | AHTQYYGWGLKNRDALLPSR | |
| | | | | | | DQLEESTRAVEAERAKGGLS | |
| | | | | | | IDYVTPDYHADRPKPCMGGW | |
| | | | | | | GQRFVNVTPSGRVLPCHAAE | |
| | | | | | | IIPDVAFPNVKDVTLSEIWN | |
| | | | | | | LSPLFNMFRGTDWMPEPCRS | |
| | | | | | | CERKERDWGGCRCQALALTG | |
| | | | | | | NAANTDPVCSLSPFHNLVEK | |
| | | | | | | AATGVPEKPELLYRRF | |
| GO_ | tldD | TldD protein, | H | S | Up | MSVAADALGGLATTDALFFG | 54 |
| 2196 | | part of | | | | RSDSKLTRDDARALVNRGLD | |
| | | TldE/TldD | | | | GVDDGELFLEYRENESISLD | |
| | | proteolyticcomplex | | | | DGTIRSASFNTSSGFGLRAV | |
| | | | | | | LGTETAFAHADDISRDALER | |
| | | | | | | AVSTVGAVRQGRSGIMAPGP | |
| | | | | | | QATNQRLYGDSRPLEGTDFA | |
| | | | | | | ARAAVLSEIDAYARGLDSRV | |
| | | | | | | VQVSAVLSSEWQAVQIMRRA | |
| | | | | | | DSGGDVADLRPLVRMNVSVV | |
| | | | | | | VEKDGQRESGSCGLGGRYEL | |
| | | | | | | DRLLAPETWRDAVDKALKQA | |
| | | | | | | LITLEATPAPAGEMDVVLGP | |
| | | | | | | GWPGILLHEAVGHGLEGDFN | |
| | | | | | | RKGTSSFSGMIGKRVASPGV | |
| | | | | | | TVVDDGTLPERRGSLSVDDE | |
| | | | | | | GTPTSRTVLIEDGILTGYLQ | |
| | | | | | | DRLNARLMGTKSTGNGRRES | |
| | | | | | | YAHAPMPRMTNTLMLEGSAT | |
| | | | | | | TDEMIRSMKRGLYAVNFGGG | |
| | | | | | | QVDITSGKFVFAASEAYLVE | |
| | | | | | | EGKIVRPVKGATLIGNGADA | |
| | | | | | | MNQISMIGSDVALDPGIGTC | |
| | | | | | | GKAGQGVPVGVGQPTLKISG | |
| | | | | | | LTVGGTA | |
| GO_ | tldE | TldE protein, | H | S | Up | MTTTPVEALLAAARRHGADH | 55 |
| 2558 | | part of | | | | ADAILVRDESESALVRKGVP | |
| | | TldE/TldD | | | | EGIERSESVALGLRVFRGKR | |
| | | proteolyticcomplex | | | | AATVSTSVLNEAEFDRLAEQ | |
| | | | | | | ACAMALVVPEDQYAGLAEAA | |
| | | | | | | LQGRFDAVGLDLECSSAPSM | |
| | | | | | | DDLLARAREAEDTALSFEGI | |
| | | | | | | TNTNGASAGHGRTSVALGTS | |
| | | | | | | AGFFGAYSRTGHSTSASVLA | |
| | | | | | | GEGATMQRDYAYRSAVHLED | |
| | | | | | | LESPAVIGREAAERVLARIN | |
| | | | | | | PGRPRTGTYSVIYDPRVSST | |
| | | | | | | LLGHLVGAINGAAIARGTSF | |
| | | | | | | LKDSLGKQILSAGLTVHDDP | |
| | | | | | | RRIRGAASRPFDAEGCAALP | |
| | | | | | | LDLIADGVLQTWLLDSRSGR | |
| | | | | | | QLNMPTNGRASRGVASPPSP | |
| | | | | | | SVTNLHLAPGTLSSVALRSD | |
| | | | | | | ISEGILITELMGSSVNMLTG | |
| | | | | | | DYSRGASGFMIRNGEIAEPV | |
| | | | | | | AELTVAGNLKDMFARMIPGS | |
| | | | | | | DLMFRQSVNAPSIRIDGMNI | |
| | | | | | | AGL | |
| GO_ | GO_ | Tryptophan | H | S | Up | MTNTPSPLSSPLANSLRSGP | 56 |
| 2863 | 2863 | synthase | | | | DDRGRFGIFGGRFVAETLMP | |
| | | beta chain | | | | LLLELDEAYRAAQADPEFRR | |
| | | (EC | | | | ELDYYLKDYVGRPSPLWFAQ | |
| | | 4.2.1.20) | | | | HLTEELGGAKVYFKREELNH | |
| | | | | | | TGSHKLNNVMGQILVARRMG | |
| | | | | | | KTRIVAETGAGQHGVATATV | |
| | | | | | | CALFGLKCTIYMGATDVERQ | |
| | | | | | | KPNVFRMHLLGAEVKPVTAG | |
| | | | | | | AGTLKDAMNEAMRDWVANVA | |
| | | | | | | DTYFLVGTVAGPHPYPEMVR | |
| | | | | | | DFQSVIGVEVKEQITQAEGR | |
| | | | | | | LPDVIVAAIGGGSNAMGIFH | |
| | | | | | | PFLDDASVRLIGVEAAGHGL | |
| | | | | | | DSGKTAASISRGRPGVLHGN | |
| | | | | | | RTYLLQDKHGQIEEAHSISA | |
| | | | | | | GLDYPGIGPEHSWLNDIGRA | |
| | | | | | | EYVGVTDEEALEAFQVCTRT | |
| | | | | | | EGIIPALECAHGLAHVMKIA | |
| | | | | | | PAMAKDQIIVLNVSGRGDKD | |
| | | | | | | IFTVAHHLGVKL | |
| GO_ | atu4171 | ATP-dependent | H | | Up | MPFPDTHPALKRALEARGYE | 57 |
| 673 | | RNA | | | | QLTPVQEAVLQPGLDERDLL | |
| | | helicase | | | | VSAQTGSGKTVAFGLAIAPT | |
| | | Atu4171 | | | | LLGDADRLPPSPQPMALVIA | |
| | | | | | | PTRELALQVQSELKWLYAET | |
| | | | | | | GARIASCIGGTDARSEAREL | |
| | | | | | | NRGVHIVVGTPGRLCDHLSR | |
| | | | | | | GSLDLSALRCVILDEADEML | |
| | | | | | | DMGFRDELEKLLDAAPTERR | |
| | | | | | | TLLFSATIAREIASLARRYQ | |
| | | | | | | KNAERIDTVSGAKQHSDITY | |
| | | | | | | RCVITQPQEIERSLVNVLRF | |
| | | | | | | YESPTAMVFCNTRMMVNQVQ | |
| | | | | | | ATLLERGFASVAISGEMGQN | |
| | | | | | | ERSRAIESLRSGQARVCVAT | |
| | | | | | | DVAARGIDVPALNLVIHASI | |
| | | | | | | PTAAETLLHRSGRTGRAGRK | |
| | | | | | | GTSVLMVPLNQRRRAERLLQ | |
| | | | | | | MAKIQAEWEAVPTADAIAEQ | |
| | | | | | | DKTRLMHDPILTNAVDDMSD | |
| | | | | | | ELVNQLVETYDATKLAAALV | |
| | | | | | | GLYRARLPKVEQIRPMSVEA | |
| | | | | | | PRRTERGERAPREEHTMSGE | |
| | | | | | | WFKMGVGRTERADPKWLIPL | |
| | | | | | | ICRLGGVQKREIGSIRIDQE | |
| | | | | | | QTYFQIADESVARFKSCLAG | |
| | | | | | | AEADEVTIEPSEAPAGGMGP | |
| | | | | | | RGRNPGEGKRFGGGGRSGGG | |
| | | | | | | FKGGPRGGAGGGYKGRGGSG | |
| | | | | | | YGRPKPAAGDGPRGDGSSRK | |
| | | | | | | RRS | |
| GO_ | ccmA | ABC transporter | H | | Up | MTSPSDFPPPPVPGRLLDVE | 58 |
| 1481 | | involved in | | | | DVTVFRGDRLVLDGLSLTLD | |
| | | cytochrome | | | | AGDAMILTGPNGAGKSTLLR | |
| | | cbiogenesis, | | | | TISGLRRPDSGEVIRYGDLA | |
| | | ATPase | | | | WLGHQDALKPGLTLAQNLAL | |
| | | component CcmA | | | | AEKLGTNSLPDALEALDLTH | |
| | | | | | | LTDLPARLLSSGQKRRAAFA | |
| | | | | | | RVMLSGAPLWLLDEPTVGLD | |
| | | | | | | VASIERLGAVMAAHRAKGGA | |
| | | | | | | MIVTTHVPLPLDNTRSHELP | |
| | | | | | | SLAHVESFWLS | |
| GO_ | clpX | ATP-dependent Clp | H | | Up | MSNKSGDSKNTLYCSFCGKS | 59 |
| 1879 | | protease ATP- | | | | QHEVRKLIAGPTVFICDECV | |
| | | binding subunit | | | | ELCMDIIREEHKTHLVKSRD | |
| | | ClpX | | | | GVPTPKEICKVLDDYVIGQF | |
| | | | | | | EAKRALSVAVHNHYKRLAHA | |
| | | | | | | AKSSDIEIAKSNILLIGPTG | |
| | | | | | | SGKTLLAQTLARILDVPFTM | |
| | | | | | | ADATTLTEAGYVGEDVENII | |
| | | | | | | LKLLQSADYNVDRAQRGIVY | |
| | | | | | | IDEIDKISRKSDNPSITRDV | |
| | | | | | | SGEGVQQALLKLMEGTVASV | |
| | | | | | | PPQGGRKHPQQEFLQVDTTN | |
| | | | | | | MLFICGGAFAGLDKIISARG | |
| | | | | | | KGSGIGFGADVRSDDERRLG | |
| | | | | | | AILQSVEPEDLLKFGLIPEF | |
| | | | | | | IGRLPVIAALNDLDESALIQ | |
| | | | | | | ILSKPKNALIKQYGRLFEME | |
| | | | | | | GVKLTFTEDALAAIAKRAIE | |
| | | | | | | RKTGARGLRSILENILLGTM | |
| | | | | | | FDLPGLEGVEEVVINREVAE | |
| | | | | | | SKAQPVYVYGKGKSEPAEQS | |
| | | | | | | A | |
| GO_ | cysG | Precorrin-2 oxidase | H | | Up | MNTQPHHSSPDSPQDGGWFP | 60 |
| 1671 | | (EC 1.3.1.76) | | | | ISIRLSGARVLLVGGGEIAV | |
| | | Sirohydrochlorin | | | | NKGRLLLDHGAWIDVLAEKL | |
| | | ferrochelatase | | | | HPVVQGWVESGRVCHVGERA | |
| | | activity of CysG | | | | DDEVLRRLLPGCRLVYAATD | |
| | | (EC 4.99.1.4)/ | | | | SRDTNRQVAALADELNIPVC | |
| | | Uroporphyrinogen-III | | | | AVDDPGPSSFITPAQVRRGM | |
| | | methyltransferase | | | | VRVAVSTEGAAPVLARRLRE | |
| | | (EC 2.1.1.107) | | | | QIETLLPEGTGRLAAYMQSR | |
| | | | | | | RVLVSGRYPNVQDRKRIWED | |
| | | | | | | FLDGPAAEAARSGDESRADA | |
| | | | | | | RLEALLNGDRKPGEVWLVGA | |
| | | | | | | GPGDPDLLTLKALHLMQNAD | |
| | | | | | | SVLYDNLVSPALLDMVRRDA | |
| | | | | | | ELVFVGKQRDRHALPQDEIN | |
| | | | | | | REMVRRAQAGERVLRLKGGD | |
| | | | | | | PFIFGRGGEEIEALVAAGVA | |
| | | | | | | FRLVPGISAANGCAAYSGIP | |
| | | | | | | LTHRDCAQACLFVTGHAKAD | |
| | | | | | | GVLDLPWDDMADRRQTVVIY | |
| | | | | | | MGISTLPQLAAGLLGKGLPA | |
| | | | | | | DWPVAIVERGTQPGQRVFTG | |
| | | | | | | TLATIAQQAAEAQVKSPALV | |
| | | | | | | IVGQVVRHRVVSP | |
| GO_ | DbsA | Periplasmicthiol: | H | | Up | MTRLSLSRRFFVSAAPALAV | 61 |
| 1605 | | disulfide | | | | AGTAAGTARAAGTGSTDARL | |
| | | interchange | | | | SPRIIGNPNAKVLVQEWFSL | |
| | | protein | | | | TCTHCAHFATEEFPKIKEQL | |
| | | DsbA | | | | IDTGKIRYQFHDFCGDRVGL | |
| | | | | | | TAAMVARSLPEERYVPFLEA | |
| | | | | | | LFSSQMQWAFAAGGDPMQRL | |
| | | | | | | QQMSALAGVSAAQFDAISKD | |
| | | | | | | NVFAEALFDQVKKDSDTYNI | |
| | | | | | | QGTPYFRFNNTHYDQDPETY | |
| | | | | | | EKFADLVAKAS | |
| GO_ | dusB | RNA- | H | | Up | MTASAPSAAPDAPAAAPPNR | 62 |
| 171 | | dihydrouridine | | | | VLKPIDLGQGVVIEDPVILA | |
| | | synthase | | | | PLSGVTDLPFRQLARDLGAG | |
| | | DusB | | | | LVVSEMIASWAMIRENENTM | |
| | | | | | | RMARMAERGPNAVQLAGCDP | |
| | | | | | | EAMAQAAKISVDGGANLIDI | |
| | | | | | | NFGCPVKKVAIGQMAGSALM | |
| | | | | | | RDEVLAGKLLEATARAVNVP | |
| | | | | | | VTLKMRMGWDHNSLNAPRLA | |
| | | | | | | KIAEESGIRLVTVHGRTRQM | |
| | | | | | | FYNGTADWRFVKTVKDAVSL | |
| | | | | | | PVIVNGDINTIRDAREALHQ | |
| | | | | | | SQADGVMIGRGCYGRPWFTA | |
| | | | | | | QVAQSLRTGEDVLDPDLATE | |
| | | | | | | KEIALRHYRMMLDHFGERPG | |
| | | | | | | LRLARKHVSWYSAGLPGSAT | |
| | | | | | | FRSTINGVESAAEAIALLTA | |
| | | | | | | FYDRHIEAGVVRNREAGPTG | |
| | | | | | | SLSRDGTREAA | |
| GO_ | envC | Murein hydrolase | H | | Up | MKAPDPRPFLPLVLLLSPGW | 63 |
| 2710 | | activator EnvC | | | | AAAHHASHHHARHTEKPAVA | |
| | | | | | | AASEGQKALARAQAARRTLE | |
| | | | | | | KRQADEAAVLKAKQIASAQA | |
| | | | | | | EAKARQDNARTLAFTAQTHT | |
| | | | | | | AQSAVDTTQSRILALKASIA | |
| | | | | | | ELMDKRTAVEADIRQQNAAL | |
| | | | | | | QPLLPVAARLSIAPDAALLA | |
| | | | | | | SPETASESVTALSVLGGFSR | |
| | | | | | | LTQQRAQALQSREDELHAIG | |
| | | | | | | IDLDSRQKELAELLAQQTRE | |
| | | | | | | RNAAAARTRIAARQEAVADQ | |
| | | | | | | GAQKARKAVADAMQAAADLS | |
| | | | | | | AEIDALVRQEAQARAELEKE | |
| | | | | | | AAALTRQHQLERARHARSQA | |
| | | | | | | QALSSSGQGVSSGSGHAPVS | |
| | | | | | | GRVAVRWGQTTEAGPATGIT | |
| | | | | | | YAALSSTPVQAPCTGRVEFA | |
| | | | | | | GPFRSFGQMLILDCGRNYRF | |
| | | | | | | VLSGLGQLNVSGGQSIRKAA | |
| | | | | | | TLGQMPAADGMLFVQLRHGT | |
| | | | | | | QVVSPAPFL | |
| GO_ | ftsE | Cell-division- | H | | Up | MIRLLNVSMMPPGVGQPVLR | 64 |
| 2773 | | associated, ABC- | | | | NLTLTVAQGEFRWLLGPSGA | |
| | | transporter- | | | | GKSSLLRLLTLETRPSAGQM | |
| | | like | | | | DLLGMSVSQASRATLRNLRR | |
| | | signaling | | | | RIGFVPQDYRLIGEWTVFDN | |
| | | protein | | | | VALPLRLRGASERDTRREAF | |
| | | FtsE | | | | AVLEWLDVAHLADKRPGTLS | |
| | | | | | | GGEQQRAAIARALIGRPEIL | |
| | | | | | | LADEPTNALEDAQARRLLAT | |
| | | | | | | FQELVDMGTTVIVATHNEAL | |
| | | | | | | VREAPAASIVLQDGTLADAD | |
| | | | | | | TQDGIAARRSDRA | |
| | GO_ | 3-phosphoshikimate | H | | Up | MQVSRPLTVSASPKGLSGRT | 65 |
| | 1058GO_ | 1- | | | | RVPGDKSISHRSLMFAALAS | |
| | 1058 | carboxyvinyl | | | | GRTYVTGLLEGEDVLRTADA | |
| | | transferase | | | | MRALGATITREGADWVIEGR | |
| | | (EC 2.5.1.19) | | | | GVGALTEPADVLDMGNSGTA | |
| | | | | | | ARLLSGILSSHGFNSIMTGD | |
| | | | | | | ASLRSRPMRRVTVPLAANGS | |
| | | | | | | EFLTREGGRLPMAVRGTGEA | |
| | | | | | | KPIEYRLPVASAQVKSAILL | |
| | | | | | | AGLNAHGTTVVEEPVATRDH | |
| | | | | | | TENMLRHFGVEVDVSRIDAG | |
| | | | | | | GRRIALTGPVRMTARDVTVP | |
| | | | | | | GDPSSAAFPIVAALLVPGSD | |
| | | | | | | IWIEGVGLNPLRTGLFTTLI | |
| | | | | | | EMGASLSIENERVEGGEPVG | |
| | | | | | | DLHVRYSQLKGVDVPPERAP | |
| | | | | | | SMIDEYPVLAVACAFAEGPS | |
| | | | | | | RLRGLEELRVKESDRLASTV | |
| | | | | | | ALLNVNGAETEVIGDDLIVK | |
| | | | | | | GHHGPLGGGTVQTHMDHRLA | |
| | | | | | | MSAVVLGLAAQKPVNVDDTA | |
| | | | | | | FIETSFPGFVDLMNALGAGL | |
| | | | | | | TP | |
| GO_ | GO_ | Fructokinase (EC | H | | Up | MSAPQHDLLCIGNAIVDVLA | 66 |
| 1072 | 1072 | 2.7.1.4) | | | | PVGQDLIDGLGAAAGSMTLI | |
| | | | | | | DAPTAHAIESRVDIENVTGG | |
| | | | | | | GSGANTAVVAARMGAKVAYL | |
| | | | | | | GKVTADEAGDHFTRDIREQG | |
| | | | | | | ITFPSEPLPAADGTPTARCI | |
| | | | | | | VLVTPEGQRTMFTYLGACTE | |
| | | | | | | FTPEDVHESVVADAAITYLE | |
| | | | | | | GYLYDKPHAQEAFEHAARLA | |
| | | | | | | RKAGRQVALTLSDTFCVERH | |
| | | | | | | RAAFHELVAGHVDILFANEA | |
| | | | | | | ELLALYEVTDFEEAITQVST | |
| | | | | | | ETKLAVITRGEKGAVVIGDG | |
| | | | | | | ERHDVPTTEVKVVDTTGAGD | |
| | | | | | | AFAAGFLAGLSKKHDLVTCA | |
| | | | | | | KLGNQAAGEIITRYGARPTE | |
| | | | | | | TFTLTA | |
| GO_ | GO_ | Fe-S oxidoreductase | H | | Up | MMRTLFLQPPSFDGFDGGAG | 67 |
| 1074 | 1074 | | | | | SRYQAKREIKSFWYPTWLAQ | |
| | | | | | | PAALVPGSRLIDAPPAKMGM | |
| | | | | | | DPILEDVKNRDLVVMHTSTP | |
| | | | | | | SFASDVRVAQMLKDANPRLM | |
| | | | | | | IGMVGAKVAVQPMESMEKGG | |
| | | | | | | PIDFVARNEFDFTIKEIAEG | |
| | | | | | | KPLAEVDGITWRNEKGEIIA | |
| | | | | | | NKDRAMIEDMDSLPFVTEVY | |
| | | | | | | KRDLNINDYFIGYLKHPYIS | |
| | | | | | | IYTGRGCKSRCTFCLWPQTV | |
| | | | | | | GGHRYRTRSPEHVAAEVRLA | |
| | | | | | | KQYFPEVQEFMFDDDTFTDD | |
| | | | | | | LPRAEAIAREMGKLGVTWSC | |
| | | | | | | NAKANVPYETLKVLKENGLR | |
| | | | | | | LLLVGYESGNQQILHNIKKG | |
| | | | | | | MRVETAKEFTRNCHKLGIKI | |
| | | | | | | HGTFIVGLPGETKETIQETI | |
| | | | | | | EFAKEINPHTLQVSLAAPYP | |
| | | | | | | GTFLHKQATENGWLNEAEAE | |
| | | | | | | LIDESGVQIAPLHYPHLSHT | |
| | | | | | | EIFESVEEFYRKFYFRGSKI | |
| | | | | | | ASIVNEMVRSPQMMKRRLRE | |
| | | | | | | GVEFFQFLKDRHAA | |
| GO_ | GO_ | Ceramide | H | | Up | MPLPLTIAAGFCALVSAAGN | 68 |
| 1075 | 1075 | glucosyltransferase | | | | LQALAGATLLARFRRTERKA | |
| | | (EC 2.4.1.80) | | | | DDALRLSDRIWPSVTVLKPL | |
| | | | | | | HGNEPLLEDALESVFTQDYP | |
| | | | | | | DFQIVFGVQDREDTALAVIE | |
| | | | | | | RLRARHPRIPVSVVIDPQEH | |
| | | | | | | GPNRKVGNLMNMYGEVRHDI | |
| | | | | | | IVISDSDIHASPNYLRHVVT | |
| | | | | | | SLEEQGTGLVTTLYAGRPAA | |
| | | | | | | GTLVQQLGACQINHNFLPGV | |
| | | | | | | MMSRFLGRQDCLGATMALRR | |
| | | | | | | QTLEEIGGLEALVDHVADDA | |
| | | | | | | ELGQLIRVRGENITIAPTLT | |
| | | | | | | HTTVGEHSISDLLAHELRWG | |
| | | | | | | RTVKNVAPVGYGLSAIQLPL | |
| | | | | | | FWAVTAVLFRPNAWWTWFML | |
| | | | | | | LLTWLVRAIGSRIMDRATEC | |
| | | | | | | PLPAAIPLLVVRDWLSAAIM | |
| | | | | | | VGSARGSRVAWRGRTVHIAR | |
| | | | | | | RKRNSASCAPSLQAGATHRS | |
| | | | | | | VRS | |
| GO_ | GO_ | hypothetical | H | | Up | MDGPWLSSLSRLRKVGKHEG | 69 |
| 1077 | 1077 | protein | | | | PSSIRLTAYRPIVVFASMSG | |
| | | | | | | CIIRIRAVANRAQMDLWMPE | |
| | | | | | | LVTGNLIQCVMNLISTHGTV | |
| | | | | | | YRTKARS | |
| GO_ | GO_ | NADPH- | H | | Up | MPDHQPTGPSAPGTDALSQL | 70 |
| 1097 | 1097 | dependent 7- | | | | GRATTTPQSPEEAVLERVPS | |
| | | cyano-7- | | | | PHQGRQYVVRFTAPEFTSLC | |
| | | deazaguanine | | | | PVTGQPDFAHIVIDYIPGEW | |
| | | reductase | | | | IVESKSLKLFLTSFRNHGAF | |
| | | (EC 1.7.1.13) | | | | HEDCSIAIAERLVALLDPQW | |
| | | | | | | LRIGAYWYPRGGIPIDVFWQ | |
| | | | | | | TGEPPKGVWIPAQDVPGYRG | |
| | | | | | | RG | |
| GO_ | GO_ | Histidine | H | | Up | MMDLVEGTEEASGALMLPPA | 71 |
| 1106 | 1106 | kinase/response | | | | PAVGRASILCVHLLALAAAE | |
| | | regulator | | | | GGGEAALLLRDADGVRVLGG | |
| | | hybridprotein | | | | EGSAIAAEGAACLMDGQSLD | |
| | | | | | | ACTVRLLPVRSGAVEVWLCV | |
| | | | | | | RRNAPALEHVLALCALQVDD | |
| | | | | | | LLRERAAAPQSGEHELVERM | |
| | | | | | | QLRMQRMADTADVAFYRCDF | |
| | | | | | | ATRVVTGDARFASLWGLPPE | |
| | | | | | | RLAVGVPIDELLMFLHPDDR | |
| | | | | | | LVYDGSLEDELRDEGCYELR | |
| | | | | | | FRILVSSPSMPGSGQAQSRS | |
| | | | | | | PRSSLLRHVLLRGWREDESR | |
| | | | | | | PDSRRSVGLAMDVSSASMTA | |
| | | | | | | EALRSSEAFTRLLLSSLPDC | |
| | | | | | | IHILDCEGCIRFVNEGGIRS | |
| | | | | | | MEMDSPIIMHGLPWVDLWRG | |
| | | | | | | QPRRRAALAVQTALAGETAR | |
| | | | | | | FQGYAMTMRGQRRFWDVAVT | |
| | | | | | | PVFGEDGDVRRLLAIGRDLT | |
| | | | | | | EANQSAERLQLALEAGAIAG | |
| | | | | | | TWMWDDSTSRMTGDARLAQT | |
| | | | | | | LGLDPARMREGVMPNVIYDS | |
| | | | | | | VDPRDRFAVVQAVTAATRRG | |
| | | | | | | GKCRFEFRVDTPEGQRWFEG | |
| | | | | | | NGRCDLGDEGRVARFPGIVF | |
| | | | | | | DIDRSKRQALRQAALVELGD | |
| | | | | | | QLRALDDTSMMEEVAARIIC | |
| | | | | | | RELDASGAGYGVVNDDWTGM | |
| | | | | | | TVGGAPEVTRPLVGMRMFAD | |
| | | | | | | YGEFRPILGRGEPVAIADVH | |
| | | | | | | TDALTAGYDARYDAEGIVAI | |
| | | | | | | LCVPIFKFGRFVGLMFVCHD | |
| | | | | | | APHIWTDEEIVFTRAVADRT | |
| | | | | | | HASMRQARTQQQIRDLNVML | |
| | | | | | | EERILQRTRERDRLWNIARD | |
| | | | | | | LFIIIDRRGYYVAVSPSWEE | |
| | | | | | | TQGYRVDELAGLRLDALCHP | |
| | | | | | | DDRRMVLDTFERLLAGTPWP | |
| | | | | | | TAGLDVRMRRSDGTWRTYNW | |
| | | | | | | NCNDEGDAIYAVGRDLTERN | |
| | | | | | | ELEEQLRQAQKMEAVGQLTG | |
| | | | | | | GLAHDFNNLLAGIGGGLELI | |
| | | | | | | GLRLAQGRTDGLGRYIAASQ | |
| | | | | | | DAVRRAASLTHRLLAFSRRQ | |
| | | | | | | TLDPTPTDMNALVRGLESLL | |
| | | | | | | RGTVGPGIELLFDLQPGLWL | |
| | | | | | | TRVDANQLENAILNLCINAR | |
| | | | | | | DAMHDRGTMLRLESANRVLT | |
| | | | | | | ADVATDMSIRAGDYVVLTVQ | |
| | | | | | | DEGCGMPPEIVQRAFDPFFT | |
| | | | | | | TKPLGEGTGLGLSMIYGFTQ | |
| | | | | | | QSGGQVEIHSTPGQGTVVSL | |
| | | | | | | WLPRYQGQETIRPEPPLLPV | |
| | | | | | | ASQPRLLEGQRVLVVDDEEA | |
| | | | | | | VRMIVSDMVTDLGGIVLTAS | |
| | | | | | | DGPSAEALAAEGVPPAVLIS | |
| | | | | | | DIGMPGGMNGRELGEQMLKR | |
| | | | | | | WPGLKVLFITGYAEQSVLGD | |
| | | | | | | QALVPGCALLVKPFTVAAFS | |
| | | | | | | RKLAVLLKGD | |
| GO_ | GO_ | DNA protection | H | | Up | MVSRTDRHVTQNNTADNTKN | 72 |
| 1168 | 1168 | during starvation | | | | VSIETLNARLSDLIDLALIT | |
| | | protein | | | | KQAHWNLKGPQFIGVHEMLD | |
| | | | | | | GFRSSIDGFSDTVAERAVQL | |
| | | | | | | GGTALGTVQDVSKNSACKPY | |
| | | | | | | PNNIYRVADHLAALIDRYAT | |
| | | | | | | VANNMRESIKVTDEAGDDDT | |
| | | | | | | ADVFTEVSRGLDKHLWFLEA | |
| | | | | | | HVQEPTGQMRDGDHKGSRS | |
| GO_ | GO_ | hypothetical | H | | Up | MSFKRRLSALLSSRGKLEYA | 73 |
| 1174 | 1174 | protein | | | | IHLTETGQAVQGFALLSRLA | |
| | | | | | | ATGDAEAAFRVGRAYLDGLG | |
| | | | | | | VPPSLEDGARWIYQAAEAGH | |
| | | | | | | IEAAFVLATLYTVGLPEGFE | |
| | | | | | | IRTAGEGLDLSHVPQVGPRH | |
| | | | | | | PDFHLGLRWAKIAADAGSPD | |
| | | | | | | AQALLGYILTNGPEDLRDLT | |
| | | | | | | QARSWYDRSAAAGCSQGHLG | |
| | | | | | | VALSILHEAHSDEDLSAAAR | |
| | | | | | | HLIEATKGGLGTAFDILGRM | |
| | | | | | | YESGSGVPRDLGKAASYFHQ | |
| | | | | | | AAERNIVTAQARYGLMLLEG | |
| | | | | | | TGTPRHYGRAETWLKRAAAN | |
| | | | | | | GDTQSAALLGDLCANGGDLP | |
| | | | | | | PNLMEASKWYRLAAEQKHAG | |
| | | | | | | AARALGLLYLTGNGVHQDPD | |
| | | | | | | VAAHWFRVASEAGDAHADAD | |
| | | | | | | FGNLILAGASATPDEKQALH | |
| | | | | | | ARFEKAAEKGDLVGAYNLGV | |
| | | | | | | CFAEGVTGTKDGREAARWMQ | |
| | | | | | | KAADGVVNAQYWYGRMLLEG | |
| | | | | | | RGVQPDPTQALYWMEKAANA | |
| | | | | | | GMAEAQVTVAGLLVDGSING | |
| | | | | | | RQDHEKALTLYRKAAESGNV | |
| | | | | | | DAMFSLAAMYGGGHDVPENR | |
| | | | | | | PQAQLWFRKAAQRGNGLAQM | |
| | | | | | | MLGRYLVRGLAGVTDPVEGR | |
| | | | | | | IWLERAKAQNIRDAEVELAL | |
| | | | | | | LDEAQPDDDD | |
| GO_ | GO_ | Bacteriocin/ | H | | Up | MEHTQSLSGPDGRVSPTVIR | 74 |
| 1176 | 1176 | lantibiotic | | | | LYAVLAAARYHGLELDIRDF | |
| | | efflux ABC | | | | AAEPGEDSPSPATLARWLNE | |
| | | transporter, | | | | QGAVAKGMRLRWRYLVKIRN | |
| | | permease/ | | | | SPPVVLMFKDGSAGLMVRAD | |
| | | ATP-binding | | | | AEKSVVWLRDPMGGEGDTPV | |
| | | protein | | | | PVDELRLMQVWTGDVLLVKR | |
| | | | | | | RRDESEADAKFDLLWFAKMV | |
| | | | | | | LREKKVMRDIAFASLILSIL | |
| | | | | | | QIFPALIVMQVVDRVVNYHS | |
| | | | | | | MATLVSLSGFVIILSFYEIL | |
| | | | | | | LTYARRELSLILSTRLDARI | |
| | | | | | | SLHAFNRLLALPLEFYEREQ | |
| | | | | | | TGEILGRFMAVFKVRDFLTG | |
| | | | | | | QLMSTLLDLFTLIVVLPVLF | |
| | | | | | | VMSPTLAWMTLAAAGCIGLI | |
| | | | | | | VVVFLPPVTRVIGRQVLAEM | |
| | | | | | | KRGSILYETVAGIRTVKTLA | |
| | | | | | | LETTRRELWDERTADVVRWK | |
| | | | | | | LAAGRMASWPQTLVMPFEIF | |
| | | | | | | INRGIILVGAYLILTNASSM | |
| | | | | | | QAGALMGFMMLGGRVASPLV | |
| | | | | | | NLAKLMEAFNEVSVSLSEAG | |
| | | | | | | MVLNQPTETKALTTGMRPVV | |
| | | | | | | KGALSFNHVDFSYPGSTTKA | |
| | | | | | | LNDVTFDIPAGTMLGLVGRS | |
| | | | | | | GSGKSTITRLLQGVSRNYTG | |
| | | | | | | YLRLDGVDLREINLTHLRRS | |
| | | | | | | FGVVLQDNFLFRGTIRDNIT | |
| | | | | | | AGRPGLTIDDAIRAARLAGA | |
| | | | | | | EEFIERMPAGYDTWIEEGST | |
| | | | | | | NISGGQRQRLAIARAVISDP | |
| | | | | | | KLMILDEATSALDPESEALV | |
| | | | | | | NANLQRIGKGRTMVIVSHRL | |
| | | | | | | SSLVNCHQIAVMDQGKLVDI | |
| | | | | | | APHRILLERCEIYRMLWLQQ | |
| | | | | | | NRHMTDNDVPGSAGQLTEGE | |
| GO_ | GO_ | O- | H | | Up | MSSENWRTATRLLHEAPNRT | 75 |
| 1380 | 1380 | succinylhomoserine | | | | EFGETSEALFLTSGFVYDSA | |
| | | sulfhydrylase | | | | EQAAATFTGDVQHFQYSRFG | |
| | | | | | | NPTVDTLQERLALLEGAEAC | |
| | | | | | | VATATGMGAVSSAILSTVKA | |
| | | | | | | GDRVVASRAIFGSCYWIVTN | |
| | | | | | | LLPRYGIETELVDGTDYDAW | |
| | | | | | | ERALSRPTAAVLIESPSNPM | |
| | | | | | | LDVLDIARVAELTHKAGGLL | |
| | | | | | | IVDNVFATPLGQSPLRLGAD | |
| | | | | | | VIVYSCTKHIDGQGRVLGGA | |
| | | | | | | VLGSSKWINETLQPFTRNTG | |
| | | | | | | NALSPFNAWVLLKGLETLQL | |
| | | | | | | RTDAMARNAAAVADALAELP | |
| | | | | | | GIVQVRYPGRADHPQHELAK | |
| | | | | | | AQMSNGGSMVAFVVDKDREG | |
| | | | | | | AFAFMNAFKVIAISNNLGDA | |
| | | | | | | RSLATHPATTTHMRVSEEER | |
| | | | | | | ARLGITDGAIRLSVGLEDPA | |
| | | | | | | DLIDDLKRGAAAVAALA | |
| GO_ | GO_ | Large exoproteins | H | | Up | MAVPDFPRPPRRRFRPFCPG | 76 |
| 1389 | 1389 | involved in heme | | | | PHFPRHLHIARWAALACVTP | |
| | | utilization | | | | IALFAAAGSVFLWELAHGPV | |
| | | oradhesion | | | | DITRVSHLVEPVSIAAGKRP | |
| | | | | | | GHPAGRLSWDTLRIQWQPAA | |
| | | | | | | GGVPAGLVLMARGLKVTRFD | |
| | | | | | | NLIAERADEADAVLSLSALF | |
| | | | | | | EGVIAPRTLRLENATLALRR | |
| | | | | | | LPDGDVDMDLPNQKRGGRGV | |
| | | | | | | PTRLDRLRAIDVHNVSITLA | |
| | | | | | | GLPQDRTAVIGPVEMQARRI | |
| | | | | | | RVAPHSPDFVWTGTARTMVM | |
| | | | | | | LDGLRTTLTAQARQVGNTGR | |
| | | | | | | LHLDSTPFEPAELGVFSPLA | |
| | | | | | | ADWHVPVSVGADAVFVPHGM | |
| | | | | | | DEQPSELTLNLTLGDGQVFQ | |
| | | | | | | KTAEPIHLHAGQASVHLRMD | |
| | | | | | | RPGLDGGATVSVPSAGLDVA | |
| | | | | | | DNAGALTHVHASASLRLDSL | |
| | | | | | | RQPRVMDGDAEAGLDGVRFA | |
| | | | | | | TLGSIWPASIIKGGRRWISR | |
| | | | | | | NITDGTGRDLEVRAHLHGDH | |
| | | | | | | GPDSILPVSVQGQLTGRGLT | |
| | | | | | | VNWLRPVPPATGLDASLHFD | |
| | | | | | | GPETLVIDLSRGVQPTGVKE | |
| | | | | | | NVLLPDGEIRIGDLYAKDQT | |
| | | | | | | GDISTHLTGPLAGFMSALAH | |
| | | | | | | PRLHLLARHPLPFTHPEGMV | |
| | | | | | | DAHVRLTLPLVAHIPDGALH | |
| | | | | | | VWTQAMFSGVHLGNVLMGQP | |
| | | | | | | VDGASGKMSATEDGLDLTGD | |
| | | | | | | GRLAGIPTHVVLHENFQGGA | |
| | | | | | | PSRVLQTIEARSVLDPQSTA | |
| | | | | | | KARIAPGGLFDGHAVLNAHF | |
| | | | | | | VQQANEMSDLKLSLDMAQAA | |
| | | | | | | LTVPIWAKPMGEPATAMVHI | |
| | | | | | | GFQKGRMSVLDGLQAHGTGL | |
| | | | | | | SVAGRGVTRAGKLTGIVLEG | |
| | | | | | | FRIGRTEGDARIALPQAVDQ | |
| | | | | | | PIGVTVDADPLDLAPMFAPH | |
| | | | | | | PPTAAAPVSQGSSGAKGASM | |
| | | | | | | GDNWSISLNAPHVYYGPKAQ | |
| | | | | | | VGGVVSQIELRNGHLTSGRF | |
| | | | | | | ALDAPTRVRAVLADTGREHP | |
| | | | | | | FVLDIDNLGTLLEGLGLYDR | |
| | | | | | | IRGGQTHLDGVFTPDETTVR | |
| | | | | | | KVPEGRNTKSAGLWGGLPPF | |
| | | | | | | RGHVEMGPSQFLRPPLTLTA | |
| | | | | | | VSDLSPLHWLTNHLDRFEIS | |
| | | | | | | HLATRLSLAGNLLVLHDGVI | |
| | | | | | | GNQALGATMEGPIDLTTSTL | |
| | | | | | | NLNGTIVPLFGLNALPGRLP | |
| | | | | | | VLGHLLSPEKGGGLLAATFD | |
| | | | | | | LHGTVEKPDLSVNPLSMLLP | |
| | | | | | | GVMRRILH | |
| GO_ | GO_ | 5-aminolevulinate | H | | Up | MNYDAMFQSALDGLHADGSY | 77 |
| 1418 | 1418 | synthase (EC | | | | RYFADLERRAGNFPKAFHHG | |
| | | 2.3.1.37) | | | | LGRDVTVWCSNDYLGMGQHP | |
| | | | | | | EVLTAMHKALDETGAGAGGT | |
| | | | | | | RNISGTNHYHVELEKELASL | |
| | | | | | | HGKESALLFNSGYLSNWVTL | |
| | | | | | | GTIAGRLRNCVVLSDELNHA | |
| | | | | | | SMIEGIRHSRAEKQIFRHND | |
| | | | | | | IEDLERRLKELPADVPKIIA | |
| | | | | | | FESVYSMDGDIAPIEAFCDL | |
| | | | | | | ADKYGAMTYLDEVHAVGMYG | |
| | | | | | | DHGAGVAEKLGLSHRLTVIE | |
| | | | | | | GTLGKGYGVVGGYIAASAAL | |
| | | | | | | CDFVRSFGSGFIFSTALPPM | |
| | | | | | | IAAGALASVRYLRSSSAERE | |
| | | | | | | GQQRAVAYLRQALDKAGIPH | |
| | | | | | | VMNPSHIVPVMVGEAELCRS | |
| | | | | | | LSDELLNRFGNYIQPINFPT | |
| | | | | | | VPRGTERLRITPTPLHTNEM | |
| | | | | | | IDELVEALATLWQERQLKTS | |
| | | | | | | KSAAA | |
| GO_ | GO_ | hypothetical | H | | Up | MRFSPSVLTRIGRWAAVVLP | 78 |
| 1436 | 1436 | protein | | | | LALTHVRVAGEADLDLLAVL | |
| | | | | | | LLLHSGLTGRRQGGWDWFRE | |
| | | | | | | PWVVATFCWWGWQMLCTLWV | |
| | | | | | | SPGHGALVQSLLAIRFPLAA | |
| | | | | | | AALGCWLLKDALWRRRVLWL | |
| | | | | | | ACACGVYIAFQMLIQAVFGR | |
| | | | | | | NLFGIPRFHDGTLTGPYEHP | |
| | | | | | | RAAAPLSRLILPLLMVGCAA | |
| | | | | | | VEGARSRLVRTLGLCTATVV | |
| | | | | | | AVGIMVLAGQRMPLALSLLG | |
| | | | | | | IGVCALLYRPMRPAALAAAA | |
| | | | | | | MLPVLVLVARVFSPGSFFHL | |
| | | | | | | VTLARQQLTHFGQSPYGEIY | |
| | | | | | | THAIVMAQAHPWIGQGYDAY | |
| | | | | | | RHFCSDPSTFHGISGLSEAV | |
| | | | | | | PERGWLDLCVQHPHNHYLQA | |
| | | | | | | LVNAGVPGLILFVLMIATWL | |
| | | | | | | KAIWPGRNGAAISIGLFAAV | |
| | | | | | | FIQEWPIASSSDFLNLPLSG | |
| | | | | | | WGFLLLGLALAYRTFRGVDG | |
| | | | | | | FQAGRDRPIS | |
| GO_ | GO_ | GDP-mannose 4,6- | H | | Up | MPTALITGITGQDGAYLSQL | 79 |
| 1441 | 1441 | dehydratase (EC | | | | LLGKGYRVVGLLRRSASADV | |
| | | 4.2.1.47) | | | | IGERLRWLGILDDVELLDGN | |
| | | | | | | MTDLSSLIRIVETVKPDEIY | |
| | | | | | | NLAAQSFVAASWQQPLLTGN | |
| | | | | | | VTGMGAVNMLEAARIVKSDA | |
| | | | | | | RFYQASSSEMYGLIQEPVQN | |
| | | | | | | EKTPFYPRSPYAAAKLYAHW | |
| | | | | | | MTVNYRESFGMHASSGILFN | |
| | | | | | | HESPLRGIEFVTRKVTDGVA | |
| | | | | | | RIKLGLAKELALGNLDATRD | |
| | | | | | | WGHARDYVRAMYLMLQQEVP | |
| | | | | | | DDYVIATGRTTSIRDLCRIA | |
| | | | | | | FSSVGLNYEDHVVTNPAFLR | |
| | | | | | | PAEVEVLLGDASKAKKTLAW | |
| | | | | | | EPETTLEEMITEMVEADLAR | |
| | | | | | | HSKRNGL | |
| GO_ | GO_ | GDP-mannose 4,6- | H | | Up | MRLLITGLRGFVGQHLQHQV | 80 |
| 1442 | 1442 | dehydratase (EC | | | | RKRFPGSEIMAGIPDIRDAQ | |
| | | 4.2.1.47) | | | | AVEKVIAAEKPDHCVHLAAV | |
| | | | | | | STIGAARKSPDHAWDVNLRG | |
| | | | | | | TLNVARAMLRHVPHSTFLFA | |
| | | | | | | STAEAYGTTFQLGTALTEDA | |
| | | | | | | PLAPGNTYAATKAAADLALS | |
| | | | | | | AMAREGLRVVRMRPFNHTGP | |
| | | | | | | GQSPDFVVPAFASQIARIAK | |
| | | | | | | GLQKPEISVGNLDAQRDFLD | |
| | | | | | | VRDVCDAYLDVLTAKKPLTP | |
| | | | | | | GTILNVCSGETRSIRSILDD | |
| | | | | | | LLAISGINAEIVTDPDRLRP | |
| | | | | | | SDIPVARGDATLITSTLGWR | |
| | | | | | | RQIAWEDTLRNVYEDCMRKT | |
| | | | | | | TA | |
| GO_ | GO_ | Lipopolysaccharide | H | | Up | MTKEYTIWIDVEDIFRYFEN | 81 |
| 1446 | 1446 | N-acetylglucosaminyl | | | | NTRPSGIQRLVFEILSVIRH | |
| | | transferase I | | | | QAAAKPDIGRIVLTRRNTGA | |
| | | | | | | RAETGPLLSPVSFDALNTLF | |
| | | | | | | STHTEETTPTQAGERSAAHA | |
| | | | | | | PSHSLMRRLRHAVIRRIESL | |
| | | | | | | PPELARPLLNLAVNQLRALQ | |
| | | | | | | LLRRYARAKFQRATPSRNTV | |
| | | | | | | QAPLSPTAPAATSVDVPKPG | |
| | | | | | | DIFLILGAAWSEPDFGERLG | |
| | | | | | | RMRRAYGIQPVLLLYDLIPA | |
| | | | | | | VRPEWCAISLIRDFRHWLDT | |
| | | | | | | TLPQCGRLLAISHATAETVE | |
| | | | | | | DYARKQRLKLLAPVQTIPIG | |
| | | | | | | SGFGPPHKVGNERPKGLPTK | |
| | | | | | | GSYVLFVSTLEARKNHLLAF | |
| | | | | | | RIWRRLVTELPRDQVPTLVF | |
| | | | | | | AGRVGWLVSDLMQQLENTEW | |
| | | | | | | LRGKIRLLRDPSDEELAHLY | |
| | | | | | | DGCMFTIFPSLYEGWGLPVT | |
| | | | | | | ESLVNGRPCIASNTTSIPEA | |
| | | | | | | GGPLTRYFNPEDLDDAYRVV | |
| | | | | | | RETIEDRAGLKKWQDEVREQ | |
| | | | | | | FQPVPWERSADAILDACHSA | |
| | | | | | | HLTGRMNGQTS | |
| GO_ | GO_ | glycosyl transferase, | H | | Up | MTLWIDIDDLLHHLLHHSRP | 82 |
| 1447 | 1447 | group 1 | | | | SGIQRVVFEIGSALRNLAGH | |
| | | | | | | NVQFVRRGPGATDARDFRTV | |
| | | | | | | DWTMVETVFREATNSSIKPG | |
| | | | | | | SSSVNAPPLTVQALEEVAPE | |
| | | | | | | DTLSAFLRTESRILKGLAGL | |
| | | | | | | PRTFARLGLKALQTRLEARR | |
| | | | | | | ARPELPTFSEGTQLADVARP | |
| | | | | | | GDVFLTLGSPWHHASYSQTV | |
| | | | | | | RWLRDDLRLSYALLMYDLVP | |
| | | | | | | IRCPEWCNRGIITTFRAWHQ | |
| | | | | | | DILPLADTLFAISHATAKDV | |
| | | | | | | RAYLAEQDIGTDIPVHPIPL | |
| | | | | | | GTGFGLSDGGMSAEALVREP | |
| | | | | | | YVLFVSTIEARKNHALLFRV | |
| | | | | | | WRRMLEEMPAERVPTLVFAG | |
| | | | | | | REGWLVSDFMQQLENADWLK | |
| | | | | | | GKIRFIRNPTDEDLRRLYAD | |
| | | | | | | CSFTVFPSFFEGWGLPVTES | |
| | | | | | | LSMGRPCVVSNTTSIPEAGG | |
| | | | | | | PLGRYFSPYSLDEAYTVIRK | |
| | | | | | | TIEDPEGLAAWTAKVRAEFR | |
| | | | | | | PVPWEDSARAILDRVL | |
| GO_ | GO_ | Radical SAM | H | | Up | MTTAPPPGPLSLYIHWPFCL | 83 |
| 1460 | 1460 | family | | | | AKCPYCDFNSHVREVIPQQR | |
| | | enzyme, | | | | FAAALRRELEHDAARLTRDG | |
| | | similar | | | | VKRPLRSIFFGGGTPSLMAP | |
| | | tocoproporphyrinogen | | | | ETVAALIEDAHRLFDAEDDL | |
| | | III oxidase, | | | | EITLEANPTSVEAGKFAAFR | |
| | | oxygen- | | | | QAGVNRVSLGVQSLRDDALH | |
| | | independent, | | | | KLGREHSATQAIRALETART | |
| | | clustered | | | | LFPRISFDLIYARPGQSDAD | |
| | | with nucleoside- | | | | WTDELTTALDLVADHLSLYQ | |
| | | triphosphatase RdgB | | | | LTIEPGTKFEAMHRRGELTL | |
| | | | | | | PDEDTAARLYDLTGEIAARH | |
| | | | | | | GLLPYEVSNYARPGAESRHN | |
| | | | | | | LTYWRYADYIGIGPGAHGRL | |
| | | | | | | TLDGELYATRRHRAPEPWAE | |
| | | | | | | RVEKTGSGSTEETLLTPQEK | |
| | | | | | | GREALLMGLRLSEGIDEARF | |
| | | | | | | AARTDRTLMECVDPALLEAC | |
| | | | | | | IEENYLERANGILRATGEGR | |
| | | | | | | LRLEAILARLVT | |
| GO_ | GO_ | 7-carboxy-7- | H | | Up | MSYAVKEMFVTLQGEGAQTG | 84 |
| 1506 | 1506 | deazaguanine | | | | RASVFCRFAGCNLWSGREQD | |
| | | synthase (EC | | | | RATAACTFCDTDFIGTDGEG | |
| | | 4.3.99.3) | | | | GGRFETAEALASTIEACWTD | |
| | | | | | | TADDSGRRYVVFTGGEPLLQ | |
| | | | | | | LDDALIAAVKAHGFEIAVET | |
| | | | | | | NGTIVAPAGIDWVCVSPKPG | |
| | | | | | | GALVQTEGAELKLVYPQPEL | |
| | | | | | | SPELFEQLSFRHFWLQPMDG | |
| | | | | | | PDRIANTQAAVAYCLHHPRW | |
| | | | | | | RLSLQTHKLIGIP | |
| GO_ | GO_ | Outer membrane | H | | Up | MAFLVSGQALAAPATSFTPA | 85 |
| 1516 | 1516 | protein | | | | QRAEIVGIMRDALQNDPSIL | |
| | | | | | | TDAIRAIREKAEEQKQDSTL | |
| | | | | | | AAVKAHQSELQSAPDFAIRG | |
| | | | | | | NPHGRITVVEFYDPRCSYCR | |
| | | | | | | SMMGEVDSFLSRHPDVRLVE | |
| | | | | | | KVVPVLGNNSVLDTRAIFAA | |
| | | | | | | SAQGKYEAMRRALMADTTKP | |
| | | | | | | SMERIVELAQANGIDTKKLT | |
| | | | | | | ADMSSPQTVALINTNLDQGR | |
| | | | | | | AVGLDGTPTFIFGTAAVAPG | |
| | | | | | | ALEADQMDAFLERARKA | |
| GO_ | GO_ | ATP-dependent Clp | H | | Up | MKMHAGSGNDMDITRMTPTR | 86 |
| 1530 | 1530 | protease | | | | LDDEPDAPEPETREDDNKTL | |
| | | proteolytic | | | | NSPISELEGRLFDQRKVLIF | |
| | | subunit(EC | | | | GGINDKIARDVTGRLLALAG | |
| | | 3.4.21.92) | | | | TSDKPIDVYVNSPGGHVESG | |
| | | | | | | DTIHDMIRFVDSIAPINMIG | |
| | | | | | | TGWVASAGALIYAAGRPERR | |
| | | | | | | VCLPNTRFLLHQPMGGVRGP | |
| | | | | | | ATDIDIEAREIIKMRERLNR | |
| | | | | | | IFAKETGQTYEKVAKDTDRN | |
| | | | | | | YWMSANEAIAYGLVNRIVHS | |
| | | | | | | ATELK | |
| GO_ | GO_ | Phosphoribosyl-ATP | H | | Up | MGKPATKPAPKPSKQQDDKK | 87 |
| 156 | 156 | pyrophosphatase (EC | | | | SDLQQELVLQRLYDTVQSRR | |
| | | 3.6.1.31) | | | | GTDPSLSHSARLMARGRNKI | |
| | | | | | | AQKFGEEAVECLIEAVNGNR | |
| | | | | | | KELIGESADVLYHLIVMWVD | |
| | | | | | | AGVSPEDVWTELKRREGTSG | |
| | | | | | | IAEKAARPKEKLG | |
| GO_ | GO_ | N-acetyltransferase | H | | Up | MTITCERVVSPLPPEDLDAL | 88 |
| 158 | 158 | /Phosphoribosyl | | | | IEATSAGILDGGGFGWLQPP | |
| | | formimino-5- | | | | GHQALARYFEGLLLVPERSF | |
| | | aminoimidazole | | | | YVVRENGVICGAGQLVRPPA | |
| | | carboxamideribotide | | | | SYEAHAATANLTGFFVAPYA | |
| | | isomerase (EC | | | | RGRGLGRALLEAMLKGAKAI | |
| | | 5.3.1.16) | | | | GCKVVNCDIRETHVAAIGLF | |
| | | | | | | RSFEFEHWGTHPYYARIGGQ | |
| | | | | | | TVRGLFLSKLLANENEAARW | |
| | | | | | | QSSIAMPDTSSAASDTMTDT | |
| | | | | | | PTPAHDLTLYPAIDLKDGAC | |
| | | | | | | VRLRRGEMEDATHYSDDPGA | |
| | | | | | | QAKLFAEAGCRHLHVVDLNG | |
| | | | | | | AFAGRSTNIPAIESIVKATN | |
| | | | | | | LPVQLGGGIRDMAAIERWLE | |
| | | | | | | AGVSRVILGSVAVKDPELVR | |
| | | | | | | QAARAFPGRIVAGIDARQGR | |
| | | | | | | VATEGWAEVSELEANDLALR | |
| | | | | | | MEDAGVAAVIFTEITRDGML | |
| | | | | | | AGLDLEQTADMARRLSIPVI | |
| | | | | | | ASGGVGSLEHLKALRDVARD | |
| | | | | | | VPGISGAIVGRALYDGRIAL | |
| | | | | | | KDALDVLGSC | |
| GO_ | GO_ | Citrate | H | | Up | MSENRSVTFGLDGLSRQFPL | 89 |
| 1600 | 1600 | synthase (si) | | | | LEGTIGPDVVDMRALSQKTG | |
| | | (EC 2.3.3.1) | | | | VFSFDPGLGSTATCTSAISY | |
| | | | | | | IDGDKGVLLHRGYPIEDLAL | |
| | | | | | | NASFTETAYLLLYGELPTAA | |
| | | | | | | QYQAFRTDMNTHRLLNEQIR | |
| | | | | | | NFFNGFRRDAHPMAILCGTV | |
| | | | | | | GALSAFYHDGLDISEPKARD | |
| | | | | | | LSARRLIAKVPTIAAWAYKY | |
| | | | | | | SIGEPFIYPDEEMSFSENFL | |
| | | | | | | HMLFARPGNHYRVNPVLARA | |
| | | | | | | MDRILLLHADHEQNASTTTV | |
| | | | | | | RLVGSTGANPYACIAAGIAA | |
| | | | | | | LWGPAHGGANEAALGMLETI | |
| | | | | | | GTREGIPAFLNEVKNRDSGV | |
| | | | | | | RLMGFGHRVYKNFDPRAKIL | |
| | | | | | | QATCHEVIEELGLKRDPLLD | |
| | | | | | | LAMELERVATEDDYFVSRRL | |
| | | | | | | YPNVDFYSGLILKALGIPKS | |
| | | | | | | MFTVLFAVARTVGWVSQWKE | |
| | | | | | | MIEEPSVRISRPRQLYIGAA | |
| | | | | | | ERSFVPMSERR | |
| GO_ | GO_ | Putative phosphatase | H | | p | MTRIREHGIRVVGDVHGDFN | 90 |
| 1603 | 1603 | | | | | AFRHATATDRFVIQLGDLVD | |
| | | | | | | HGPDSAGVMELMLELLEQQR | |
| | | | | | | GLFILGNHDRKLGRALEGRR | |
| | | | | | | LRRDPPLEETLRQISLPEYE | |
| | | | | | | GLPERAYRAIDQAPTWLRIG | |
| | | | | | | RSLFVHGGFHTAMLSHSPVP | |
| | | | | | | GLGEMSAPLSRALFGETTGR | |
| | | | | | | MQPDGYPERRLTWINRIPEG | |
| | | | | | | MTVYVGHDRRSTDGRPWRRT | |
| | | | | | | GRLGGTAVFTDLGAGKGGHL | |
| | | | | | | AWIDLNEP | |
| GO_ | GO_ | Acyl-CoA: | H | | Up | MSHRDAKTPNGRRGHNRPVL | 91 |
| 1612 | 1612 | 1-acyl-sn- | | | | EGKPDFPASRPTTSTTLYSR | |
| | | glycerol-3- | | | | LRCAGRLSLVLIWAFWACSM | |
| | | phosphate | | | | QAILVRLPGRLKIMMPRIFW | |
| | | acyltransferase | | | | KGVCRILGIRIRVIGHSAGG | |
| | | (EC 2.3.1.51) | | | | VRTARDVREGKRPVVFVANH | |
| | | | | | | CSWLDIAIIGSTLPVVFVAK | |
| | | | | | | GEVGKWPLIGTASRLGRTIF | |
| | | | | | | VSRNRRETGRELHDMAARLW | |
| | | | | | | DGDDIVLFPEGTSSDGSRVL | |
| | | | | | | PFLSSFFAVAKPGRLEQVGM | |
| | | | | | | PKAPPVLIQPVSVVYDSLEG | |
| | | | | | | LPVGRSRRNVFSWYGDMDLA | |
| | | | | | | PHLWSFGQWRSMGASLMLHD | |
| | | | | | | PITPDDFRSRKDLSRATFEA | |
| | | | | | | VNNGAAELRRGQPKVTGP | |
| GO_ | GO_ | Response regulator | H | | Up | MAPSLTVLLIEDDFLIRTCL | 92 |
| 1616 | 1616 | receiver | | | | AEFLLDSGLTVREADNCAEA | |
| | | | | | | RAIIGAEPKLDALVADMTLP | |
| | | | | | | DGDGMTLVQPARERWPDLPV | |
| | | | | | | IYISGHGDLRRDETSGDPAR | |
| | | | | | | DRFISKPYTLASILEALLDM | |
| | | | | | | TSAASD | |
| GO_ | GO_ | hypothetical | H | | Up | MKRLHFSNMDQAVVFFASRT | 93 |
| 1618 | 1618 | protein | | | | GSLALATQETLSVMCFQSRF | |
| | | | | | | QTCIHKER | |
| GO_ | GO_ | FIG00688344: | H | | Up | MTLPVLVLAGSRDGENDVLA | 94 |
| 1653 | 1653 | hypothetical | | | | KLGQVSHKALLPVAGQPMLA | |
| | | protein | | | | RVLDTIARTPGLGPVTISIE | |
| | | | | | | NPDCIRDLAGDATILRSAPS | |
| | | | | | | PSESVAEAIARIGTPCLVTT | |
| | | | | | | ADHALLRPEWIQEFLAKAQG | |
| | | | | | | CDLAAAVALRATVERDVPGT | |
| | | | | | | KRTYIHLSDMSFSGCNLFLI | |
| | | | | | | GTPKGRNVIELWKRLQQNRK | |
| | | | | | | RPLRMALTLGIGTLLRAVTR | |
| | | | | | | TLDPTALYRRIRTLTGANVR | |
| | | | | | | LVTLSDGRAAVDVDKPSDLT | |
| | | | | | | LAEKILAQTTP | |
| GO_ | GO_ | Sphingolipid (S)- | H | | Up | MSNFKAPWRNMSAIRTGRSF | 95 |
| 1654 | 1654 | alpha-hydroxylase | | | | DLGKMNLQQLWFAYLTYPTI | |
| | | (no EC) | | | | LLYFALIAVSTWAALHFSTA | |
| | | | | | | LWATLIPVPVVIVVYPLAWY | |
| | | | | | | AIHRFILHGRWLYRNHWTAS | |
| | | | | | | LWKRIHFDHHQDPHLLDVLF | |
| | | | | | | GSPLNTVPTMAIITMPIGGL | |
| | | | | | | IAGWSGAFCALSTALVMTCI | |
| | | | | | | YEFFHCIQHLAYKPRWKWVA | |
| | | | | | | DIKQLHVLHHFHDEDGNYGI | |
| | | | | | | TNYVPDRLFSSFYREARDRP | |
| | | | | | | RSKHVFNLGYDIEEAHRYPW | |
| | | | | | | VMDLTGSPPRDRPDGARPAS | |
| | | | | | | ARAAADRNRAA | |
| GO_ | GO_ | Lipopolysaccharide | H | | Up | MKQRAHILDRYLLTQMAPPF | 96 |
| 1656 | 1656 | export system | | | | AIALLAMLVALLLERLLSLF | |
| | | permease | | | | DYLASAGSSLGTCIALLTDL | |
| | | proteinLptF | | | | LPHYFGIALPAALCIAVFLT | |
| | | | | | | IRSMSDNNEIDALQAGRVSL | |
| | | | | | | MRISRPFMIVGLLLGAASVF | |
| | | | | | | LYGYIQPVARYDYRAGFYFA | |
| | | | | | | EHTGWAPHLQAGMFASTSSK | |
| | | | | | | AVMTADSVSHAGTRLRRVFI | |
| | | | | | | REVNANGVAHIITARTGALT | |
| | | | | | | ISEKTRSTRLDLWNGEIVDD | |
| | | | | | | PFQATKPHKPTVTHFEHVVR | |
| | | | | | | VIDRPNKETSFRSRGADERE | |
| | | | | | | LTLFELAHDLRYGLPGIEYR | |
| | | | | | | TLRAEMDFRMARAIAIPFIP | |
| | | | | | | PLAVALAISARRRKSVWGLI | |
| | | | | | | AVAVILIGFDQTLMFGHSLA | |
| | | | | | | STGRLPIWLAIWVPEVVFCV | |
| | | | | | | GCLAALLRRSRGSWRRRKFT | |
| | | | | | | RAPA | |
| GO_ | | Serine | H | | Up | MTDIFAKHHGLREAYEGLTA | 97 |
| 1660GO_ | | palmitoyltransferase | | | | ASPRNPFEVVIERPISASVG | |
| 1660 | | (EC 2.3.1.50) | | | | IIEGRETLLFGTNNYLGLSQ | |
| | | | | | | SKKAIGAAVETAETMGVGTT | |
| | | | | | | GSRIANGTFGLHRKLEAKLA | |
| | | | | | | EFFRRKHCMVFSTGYQANLG | |
| | | | | | | TISALVNKDDVLLLDADSHA | |
| | | | | | | SIYDGAKLSGAQVIRFRHND | |
| | | | | | | PVDLEKRLARLKDHPGAKLI | |
| | | | | | | VAEGIYSMTGNVAPLDKFVD | |
| | | | | | | IKTRHGAYLMADEAHSFGVL | |
| | | | | | | GAHGRGVAEMQGCEDGIDFV | |
| | | | | | | VGTFSKSLGTVGGYCVTNHD | |
| | | | | | | GVDLMRLCSRPYMFTASLPP | |
| | | | | | | EIIAATMAALEDMQARPELR | |
| | | | | | | TKLQENAARLHAGLQKVGLK | |
| | | | | | | TGEHVSPVVAVTLETVDQAV | |
| | | | | | | GFWNALLENGVYVNLSLPPA | |
| | | | | | | TPDNRPLLRCSVMAAHSPEE | |
| | | | | | | IDRAVAVFGEVARHFGL | |
| GO_ | GO_ | Homoserine O- | H | | Up | MDISASPSIADGPVYTHQTV | 98 |
| 1749 | 1749 | acetyltransferase | | | | RLDSGLDLECGVHLAPLEVA | |
| | | (EC | | | | YCTYGTLSPARDNAILVCHA | |
| | | 2.3.1.31) | | | | LTGDQYLAERNPLTGKPGWW | |
| | | | | | | SRMVGPGLPIDTDRYFVVCS | |
| | | | | | | NVLGGCMGTTGPRSICAETG | |
| | | | | | | KAWDSEFPPITMHDIVAAQA | |
| | | | | | | KLIDHLGVDRLFAVIGGSMG | |
| | | | | | | GMQALTWAADFPDRVFAAMP | |
| | | | | | | IATSPFHSAQNIAFNEVSRQ | |
| | | | | | | AIFADPDWHDGHYRDFGAIP | |
| | | | | | | ARGLGVARMMAHITYLSEEA | |
| | | | | | | LSRKFGRRVRHDAATAVPAS | |
| | | | | | | SSPSLFGEMFEVESYLRHQG | |
| | | | | | | SSFVRRFDANSYLTITRAMD | |
| | | | | | | YFDLAAEHDGDLANPFRKSQ | |
| | | | | | | TRFCVVSFSSDWLFPTSQSR | |
| | | | | | | LLVRALNRAGANVSFVEIES | |
| | | | | | | DRGHDAFLLEEPDFDRTIRG | |
| | | | | | | FIAGAAEHAALKVGER | |
| GO_ | GO_ | UDP-N- | H | | Up | MSGFPSHLLAGERYAVCGLG | 99 |
| 1794 | 1794 | acetylmuramoyl | | | | RNGTAVVQALLRMGAEVQAW | |
| | | alanine--D- | | | | DDRNANLPAQPNLTVAPLTD | |
| | | glutamate | | | | LSGMTALILSPGIPHLLPKA | |
| | | ligase(EC 6.3.2.9) | | | | HPVADLARAQNVQILSDAEI | |
| | | | | | | LYRAARKSGSKAAFVAVTGT | |
| | | | | | | NGKSTTTALIAHLFTTAGRP | |
| | | | | | | CAAGGNLGTASLALPLLPDD | |
| | | | | | | GVYVIEMSSYMLERLDRFHA | |
| | | | | | | NAACLLNLTPDHLDRHGDMA | |
| | | | | | | GYAAAKAHIFDNMGPDDLAV | |
| | | | | | | IGTDDDWCRSIASQVASRGV | |
| | | | | | | QVAELDADTLPPYDGPALPG | |
| | | | | | | RHNAQNVGAALAIARHLGLD | |
| | | | | | | DAVIRTGLRSFPGLEHRLQK | |
| | | | | | | VAECDGVSFINDSKATNAEA | |
| | | | | | | VSKALAAYDNVMWIAGGVAK | |
| | | | | | | AGGIESLAPFFAHIAQAFLI | |
| | | | | | | GQDADVLAATLETHGVPFQQ | |
| | | | | | | CGTLEKAVPAAFEAARNENI | |
| | | | | | | PVVLLSPACASFDQFRSFED | |
| | | | | | | RGSHFLQICDNIVKSGHSGT | |
| | | | | | | NPMQKQED | |
| GO_ | GO_ | FIG00688361: | H | | Up | MDAESKSEGAATGGIAADRL | 100 |
| 1806 | 1806 | hypothetical | | | | RSIIERVERLEEERKALAGD | |
| | | protein | | | | IKDIFSEAKSAGFDVKVIKQ | |
| | | | | | | IIRLRKQEPAEIEEQETLLD | |
| | | | | | | IYRRALGM | |
| GO_ | GO_ | Transcriptional | H | | Up | MGKKDEDRRIGERGENQMDW | 101 |
| 1836 | 1836 | regulator, LysR | | | | DKLRIFHAVAEAGSFTHAGD | |
| | | family | | | | RLGLSQSAVSRQISALEDVL | |
| | | | | | | RVPLFHRHARGLILTEQGDV | |
| | | | | | | LNRTVREVFSKLALTQAFLS | |
| | | | | | | ESKERAAGKIKITTTTGFGL | |
| | | | | | | SWLSPRLNRFLELHPDIEVT | |
| | | | | | | LLLEDADLDLGMREADVAIR | |
| | | | | | | LHPPTQPDLVQRHLANFPMP | |
| | | | | | | IYASAEYLERNGTPHSLEDL | |
| | | | | | | ANHQIISFAGWHLPLPNVNW | |
| | | | | | | LLDLLRQEGVARQGSRRLAI | |
| | | | | | | NNISAVANAIAAGTGIGSLP | |
| | | | | | | LYAATGYPELVRILPNQQVP | |
| | | | | | | LVEAYFVYPEELRTSKRIAV | |
| | | | | | | FRDFLLSEISNLKGHE | |
| GO_ | GO_ | Integration | H | | Up | MSTVTRANLVEHLYSRVGLS | 102 |
| 1854 | 1854 | host | | | | RHDSSMILESLLGVISDRLE | |
| | | factor alpha | | | | AGESVKLSGFGTFSVRQKGE | |
| | | subunit | | | | RIGRNPKTGVEVPILPRAVL | |
| | | | | | | VFRPSQLLRDRMNGSENGAA | |
| | | | | | | DEASSHDR | |
| GO_ | GO_ | Uncharacterized | H | | Up | MTDFSPPPSPDTITVEASAS | 103 |
| 1856 | 1856 | UPF0118 membrane | | | | TGTIQRRARGFLALFFVAIG | |
| | | protein | | | | LYTLKGFLPALLWGCVFAIS | |
| | | | | | | IWPLYRRAELRFGRSDWLPM | |
| | | | | | | VFTLAVALIFLVPVSLVGVK | |
| | | | | | | VADEARSALEWIDDVRNNGI | |
| | | | | | | PMPEWVPHLPFLSAQATNWW | |
| | | | | | | QNHMTSPQRLSHLLHSVDVG | |
| | | | | | | HGMQMTKQVGSQLARRGTLF | |
| | | | | | | AFSLLTLFFLLKDGDSVIRK | |
| | | | | | | CLLGSQRLFGEQGESLAKQM | |
| | | | | | | ISSVHGTLAGLVLVGLGEGA | |
| | | | | | | IMGIVYMATGAPQPLLFAMV | |
| | | | | | | TAVAAMIPFLAWPTVGLVAL | |
| | | | | | | LLLAKSSMIGAIVVLAIGSV | |
| | | | | | | VIFIADHFIRPALIGGSTKM | |
| | | | | | | PFLWVLLGILGGAETWGLLG | |
| | | | | | | LFLGPAIMAALHLLWTLWTE | |
| | | | | | | VNPRKGVYAEDKGS | |
| GO_ | GO_ | Putative outer | H | | Up | MRSRLTFSIGATAVLALSST | 104 |
| 1909 | 1909 | membrane protein | | | | AMATPLQDPYGTWTVQGEND | |
| | | | | | | AISTLKGTSDQYYTSGLRIN | |
| | | | | | | WTSGTDNLPRPIAKLNHILM | |
| | | | | | | GDGVQRISIGLQQIIDTPRD | |
| | | | | | | TQADNPPQGDRPYAGLLLGT | |
| | | | | | | VNLINDTDLSRTVMGIQFGM | |
| | | | | | | LGPSGLGRQVQNGFHKAISD | |
| | | | | | | TPSTGWSHQLANQPIFQVQA | |
| | | | | | | GRIWRVPVLNVYGIHADVLP | |
| | | | | | | AISGAAGDYRTYADVATTFR | |
| | | | | | | IGQGLDSDFGNATIGPGLDG | |
| | | | | | | TDAFRATRPFAWYFYGGVEG | |
| | | | | | | QAVGYDVTLQGSTVRPNAPH | |
| | | | | | | VEKVWDVGEIHAGVAVMWHG | |
| | | | | | | VRLSYSQNWQTAQFETQKAG | |
| | | | | | | LFNYGSLKLSVKF | |
| GO_ | GO_ | UTP--glucose-1- | H | | Up | MIKPLKKAVLPVAGLGTRFL | 105 |
| 1922 | 1922 | phosphate | | | | PATKAMPKEMLPVVDKPLIQ | |
| | | uridylyl | | | | YAIDEAREAGIEEFCLVTGR | |
| | | transferase | | | | GKDSLIDYFDIAYELEATLK | |
| | | (EC 2.7.7.9) | | | | ERGKKSALEALAPSSVQAGS | |
| | | | | | | LVAVRQQEPLGLGHAIWCAR | |
| | | | | | | SFIGDDPFAILLPDDIVKGR | |
| | | | | | | SCIGQLVEAYNQTGGNVVAV | |
| | | | | | | TEVPPEHTNRYGILDVGSDD | |
| | | | | | | GKLVEVKGLVEKPAPEDAPS | |
| | | | | | | NLSIIGRYVLTPDVMKYLAK | |
| | | | | | | LERGAGNEVQLTDAMAKTIG | |
| | | | | | | EVPFHGLRYEGTRYDCGDKA | |
| | | | | | | GFLEAQIAFSIDREDLGASV | |
| | | | | | | KAFLKKYKQYVDAP | |
| GO_ | GO_ | UTP--glucose-1- | H | | Up | MPFRHDFDPTSLREYDIRGI | 106 |
| 1923 | 1923 | phosphate | | | | VGKTLHPADAFAIGRTFASM | |
| | | uridylyltransferase | | | | VIRAGGKRIVVGYDGRLSSP | |
| | | (EC 2.7.7.9)/ | | | | ALAEALVRGAVESGAEVTRI | |
| | | Phosphomannomutase | | | | GCGPTPMLYFASVADGADGA | |
| | | (EC 5.4.2.8) | | | | VMVTGSHNPPDYNGFKMMMG | |
| | | | | | | GKPFYGDQIRELGRLSASGD | |
| | | | | | | VLPATNGTAARIDISGRYID | |
| | | | | | | RLVQDYDGIRPLRVVWDNGN | |
| | | | | | | SAAGAVLSRLVERLPGEHTV | |
| | | | | | | LFGEIDGHFPNHHPDPTVEK | |
| | | | | | | NLQDLIRVVDEKQADLGIAF | |
| | | | | | | DGDADRIGIVDNRGQIFWGD | |
| | | | | | | QMLVLLAQDVLSRHPGATII | |
| | | | | | | ADVKASQILFDEIAKAGGQP | |
| | | | | | | LMWKTGHSLIKTKMAETGSP | |
| | | | | | | LAGEMSGHIFFADKWYGFDD | |
| | | | | | | ALYAAVRVLGIVSRLPGPLS | |
| | | | | | | DFRDSLPVTVTTPELRFNCD | |
| | | | | | | DKRKFEVITEVAERLRKEGS | |
| | | | | | | DVSEIDGVRVNTADGWWLLR | |
| | | | | | | ASNTQAVLVARAEARDEAGL | |
| | | | | | | DRLKAALAAQLEASGLDAPD | |
| | | | | | | FSGENAGH | |
| GO_ | GO_ | hypothetical | H | | Up | MSPPSSNRPFSQPSDQPSVG | 107 |
| 1932 | 1932 | protein | | | | TVLRARREELGWRLEDVAEW | |
| | | | | | | LRIRPKLLAALEADDLSKLP | |
| | | | | | | GVAYAVGFLRTYAHAMQLDA | |
| | | | | | | DALVERFRRDTRGAVTRKPE | |
| | | | | | | LVFPQPEGDRGLPVGVLVGA | |
| | | | | | | GLVVVVAAYVGWYRFTEHDN | |
| | | | | | | PAQRQVPAVAELMPGAATPA | |
| | | | | | | MTSPQVASVMPGRAPTPEPH | |
| | | | | | | APVTASSVPATPAPVPTQNA | |
| | | | | | | PAPELPAAPAGAQSTPPSAA | |
| | | | | | | PSVAPPASDDDSETTPPAGE | |
| | | | | | | AAPTQQTDTQQTGAIGRPAT | |
| | | | | | | DLPPAVPEGAVVLRALAPVW | |
| | | | | | | TQVRDRDGHVLMSRVMQPGE | |
| | | | | | | SWQGDPAGAPFRMSFGNAGG | |
| | | | | | | IVLTTAGATSAPLGKEGQVR | |
| | | | | | | RNVEVTADAIRSGAFGSGVA | |
| | | | | | | LPVQPNAPVSVPGAASAPLA | |
| | | | | | | VAPAPVPPRAPSVSVPRKAP | |
| | | | | | | TAPEVSADDLNARQLEHQPL | |
| | | | | | | EQSSPPH | |
| GO_ | GO_ | hypothetical | H | | Up | MIAIGPAMASQPAWLKAATI | 108 |
| 1945 | 1945 | protein | | | | IVGTFILEDVATVLSAIAAR | |
| | | | | | | AGEVSIPLALGALYFGVAVG | |
| | | | | | | DMGLYGLGAAGARWPYLKRF | |
| | | | | | | LTLPKRERTQDWFSTNVIRV | |
| | | | | | | VAISRFVPGARLPLYTACGF | |
| | | | | | | FRAPFLPFAMTAVLATLVWT | |
| | | | | | | TCLFLLAMRVGGWLLAHQGG | |
| | | | | | | WRWAGLAGFVLCIVVVGRLI | |
| | | | | | | ARLQTVSQ | |
| GO_ | GO_ | Hypothetical | H | | Up | MTLPDRTDIPAPHDDLVVRP | 109 |
| 1984 | 1984 | protein | | | | VTTRADLKLFMTLPRRIYAG | |
| | | associated | | | | MAGFVPAFDMEQDDLLNPKK | |
| | | with | | | | APIFRHASIRYFLAWRGNTA | |
| | | Serinepalmitoyl | | | | VGRIAAIVDHRAIEHWGMKI | |
| | | transferase | | | | GCFGALDAVPEGSVVSALLE | |
| | | | | | | MARNWLRLQGMQTMRGPVTL | |
| | | | | | | SGNGESGLMVEGQDQPLMVA | |
| | | | | | | MPWHPRLLGKLVEDAGYKPV | |
| | | | | | | EDLLSYKLDLDDQTESRFKV | |
| | | | | | | PGDLKIGEGRLGAIAIRRLS | |
| | | | | | | KKQIAQQGEILRQLYNDAWS | |
| | | | | | | DKFNFVPLQDYEMKAMIKQL | |
| | | | | | | GPVLRPEHYVQIDQNGEPVA | |
| | | | | | | MALVVPNIYDIAGDLGGAPS | |
| | | | | | | PLGWVKLVARLTTHRFHSAR | |
| | | | | | | VILLGVTQRLRGTVLGALLP | |
| | | | | | | SLAIAELMRRRKSLPYSWVE | |
| | | | | | | LGWIQASDSNMRNLAESIVP | |
| | | | | | | EPYKRYRLYERPIDDPA | |
| GO_ | GO_ | Oxidoreductase, | H | | Up | MNTAQQLRVGIVGAGHFGRF | 110 |
| 1999 | 1999 | Gfo/Idh/MocA | | | | HALKSAANPAEQLVALYDPD | |
| | | family | | | | PARAAIVAREARCGIATSYE | |
| | | | | | | NLLEQVDAVIIAAPAEYHFR | |
| | | | | | | LTSQALRAGRHALVEKPIAA | |
| | | | | | | TLDEAHALADLARETGKVLQ | |
| | | | | | | VGHLLRYSAEHQAITERIKA | |
| | | | | | | PLYIEATRIAPYKPRGTDVS | |
| | | | | | | VILDLMIHDLDLVLAIVDSP | |
| | | | | | | IAEIDALGAAVSSAHEDIAN | |
| | | | | | | ARVRFENGCVATITASRISL | |
| | | | | | | KTERRMRLFSQDGYLSADFM | |
| | | | | | | ERKLSFIGRERGMPLPGTGG | |
| | | | | | | FRREAISWKDHDNLAVEHEA | |
| | | | | | | FAASCLHGTPVLVDAQAGIR | |
| | | | | | | ALDAAIRVTDSIRKSRQIME | |
| | | | | | | LSGLIPASDKN | |
| GO_ | GO_ | LSU ribosomal | H | | Up | MKSGIHPDYHEITVIMTDGT | 111 |
| 2004 | 2004 | protein | | | | EYKTHSCYGEPGATLRLDVD | |
| | | L31pprotein | | | | PKSHPAWTGVQRMMDTGGQV | |
| | | L31p, zinc- | | | | AKFNKRFAGIGTRTK | |
| | | independent | | | | | |
| GO_ | GO_ | hypothetical | H | | Up | MTLPLMPKATAVWLIEKTGL | 112 |
| 2006 | 2006 | protein | | | | TFTQIAEFCGMHPLEVQAIA | |
| | | | | | | DGEVAAGINGYDPIKNHQLA | |
| | | | | | | EAEIKRCEADTNARLKIMPT | |
| | | | | | | SSPVKRRAKGARYTPVAKRN | |
| | | | | | | DRPDAIAFVLRQFPQLSEAQ | |
| | | | | | | IVKLLGTTKDTITKVRDRQH | |
| | | | | | | WNSANIKPRDPVILGLCTQT | |
| | | | | | | DLNAAVTAANDRLAREGHAL | |
| | | | | | | PVVEAYVPDSDSHA | |
| GO_ | GO_ | Phosphoribosylamin | H | | Up | MARRRQLYEGKAKVLFEGPE | 113 |
| 2062 | 2062 | oimidazole- | | | | PGTLVQYFKDDATAGNGAKS | |
| | | succinocarboxamides | | | | GIITGKGVLNNRISEYLMLK | |
| | | ynthase | | | | LHEINIPTHFIRRLNMREQL | |
| | | (EC 6.3.2.6) | | | | IREVEIIPLEVVVRNVAAGS | |
| | | | | | | LSKRLGIPEGTRLPRTIIEY | |
| | | | | | | YYKNDALGDPMVSEEHIAAF | |
| | | | | | | NWAAPQDMDDMNQLALRIND | |
| | | | | | | FLMGMFTAVGITLVDFKLEF | |
| | | | | | | GRIWEGEEMRILLADEISPD | |
| | | | | | | NCRLWDSKTNEKMDKDRFRR | |
| | | | | | | DMGRVEEAYQEVAKRLGILP | |
| | | | | | | ESGNGDLKGPEAVQ | |
| GO_ | GO_ | Anthranilate | H | | Up | MTVSADFTSLLHKAALGRHL | 114 |
| 2075 | 2075 | phosphoribosyl | | | | DSHEAETAFHAIMAGEVDPI | |
| | | transferase | | | | QLAAFLTALKLRGETFAELT | |
| | | (EC 2.4.2.18) | | | | GAVQAVRHHMTVLPDVPAGA | |
| | | | | | | IDVCGTGGDGLKTLNVSTAV | |
| | | | | | | AFVLAGLGVPVAKHGNRALS | |
| | | | | | | SATGATDVLEVLGIPPTDDL | |
| | | | | | | ALQGRRLREDGLVFLAAPQH | |
| | | | | | | HPAMRHAAPVRKALGFRTLF | |
| | | | | | | NLLGPLCNPAQVRHQLIGVF | |
| | | | | | | DGRWCEPVARALGALGSLSV | |
| | | | | | | WVVHGSTEEGGSDELTLAGP | |
| | | | | | | SQVSAWQDDTLFSFGIEPDM | |
| | | | | | | AGLAAAPISAIRGGDAQTNA | |
| | | | | | | AALLALLDGAGGAYRDTVLL | |
| | | | | | | NAAAALHVAGRGDIVKAGAI | |
| | | | | | | DVPAFRRNVGMAADSIDRGL | |
| | | | | | | ARAALEAARMSAHSIAPKDA | |
| | | | | | | GRS | |
| GO_ | GO_ | LSU ribosomal | H | | Up | MGKSNVIQIRLVSSAETGYF | 115 |
| 2086 | 2086 | protein | | | | YVTKKNARSATGKMEVRKYD | |
| | | L33pprotein | | | | PVARKHVVFREAKIK | |
| | | L33p, zinc- | | | | | |
| | | independent | | | | | |
| GO_ | hpnB | Hopene-associated | H | | Up | MLFGTALTSLGAWIYLSLFH | 116 |
| 2109 | | glycosyltransferase | | | | GKFWQKGPILAQKPTPVCAP | |
| | | HpnB | | | | DVAVVVPARDEADSIRECLT | |
| | | | | | | SLLEQDYDGKLSVILVDDES | |
| | | | | | | ADGTGDIARALPDPHHRLTV | |
| | | | | | | ISGQKRPAGWSGKLWAVHQG | |
| | | | | | | EQEALTRIGPYGYILLTDAD | |
| | | | | | | IMHAPGHLASLVAKAREDDL | |
| | | | | | | DLVSEMVALNCESTAERFLV | |
| | | | | | | PAFVYFFAMLYPFSRIASEH | |
| | | | | | | SRIAGAAGGTILLRRRALER | |
| | | | | | | IGGISALRGALIDDCTLAAH | |
| | | | | | | VKRSGGRLYLGHSALAWSVR | |
| | | | | | | PYRGMKDVWHMIARTAYVQL | |
| | | | | | | RYSPVLLIATIIGMATIWLL | |
| | | | | | | PVALALFGKGRERRVGLLTY | |
| | | | | | | LLSCLTFVPTLRRFGLPLWR | |
| | | | | | | AIPLPLVAAFYMAATIGSAF | |
| | | | | | | DHHRGVGVRWKNRSYTDETS | |
| GO_ | GO_ | Predicted integral | H | | Up | MSKRYVTKTLKKTLPVLLSL | 117 |
| 2111 | 2111 | membrane protein | | | | LGVALFTFIAAKAGIHPVME | |
| | | | | | | ALSKVGVGGFLLLAACQLLI | |
| | | | | | | DMGLGVAWHAAVPLLSVRRL | |
| | | | | | | MGARLVRDSAGACLPFSQLG | |
| | | | | | | GMVIGVRATLAGVDPRTVKG | |
| | | | | | | EELHWPEGVAANLVDITTEV | |
| | | | | | | LGQIAFVLIALLCLIGHHGA | |
| | | | | | | SRFVWPLIGGMVLLSLGIAG | |
| | | | | | | FIWTQQRGGVMVRKAAAFLG | |
| | | | | | | KHIAAEWRDSLIGNTETFQL | |
| | | | | | | RLESLWSRPDRISLGAFCHL | |
| | | | | | | LCWMGSAAMTWLALQLLGAH | |
| | | | | | | VGFFSSVAIEGVVCGIMSAG | |
| | | | | | | FLVPGALGVQEAAYVALGMI | |
| | | | | | | FGIDAEISLSLSLLRRGRDI | |
| | | | | | | LIGIPVLLAWQIVEMRRLRH | |
| | | | | | | APPSEASKSTPAPSPASARK | |
| | | | | | | TVIPPAVTALAGNIKKEATS | |
| | | | | | | RDGKKTEDTPFATAPRVL | |
| GO_ | GO_ | UDP-N- | H | | Up | MKVLVTGVAGFIGFHVAHAL | 118 |
| 2144 | 2144 | acetylglucosamine 4- | | | | LKQGMEVVGVDTLNAYYDPA | |
| | | epimerase | | | | LKAARLEQLEPYPGFSFLKV | |
| | | | | | | DVASPAAMQDLVARHPDLEG | |
| | | | | | | VIHLAAQAGVRHSMVDPYSY | |
| | | | | | | VTSNVMGQVALLEACRHLKK | |
| | | | | | | LTHVVYASSSSVYGRNQSVP | |
| | | | | | | FRETDRVERPSSVYAVTKRA | |
| | | | | | | AELMSESYAYLHGIPQTGLR | |
| | | | | | | FFTVYGPWGRPDMAYYGFAK | |
| | | | | | | AISEGRPVTLYEGKHLSRDF | |
| | | | | | | TYIDDIVRGVQRVLGRPPEA | |
| | | | | | | GMSRVLNLGGDKPERVTRMI | |
| | | | | | | ELLEQNLGKKAFVERRPRPV | |
| | | | | | | ADMESTWASLENVREFCGWK | |
| | | | | | | PVVSFEEGMKEFCLWFRKFH | |
| | | | | | | GI | |
| GO_ | GO_ | hypothetical | H | | Up | MRVLCCALVAALGMSAGVAK | 119 |
| 2145 | 2145 | protein | | | | AEDPITQAQARLPTAPLTIT | |
| | | | | | | TRDGQKHEFTVELAKTYRQQ | |
| | | | | | | EVGEMFRKHLPENEGMLFMW | |
| | | | | | | ATPQVSDMWMRNTLVPLDIV | |
| | | | | | | FIDSTNHIHAIAENAVPLSE | |
| | | | | | | AILRSDGVVANTLELAGGVT | |
| | | | | | | AKLGIRVGDAVTSSALKH | |
| GO_ | GO_ | hypothetical | H | | Up | MKHKSCRKTFFSALALSAIA | 120 |
| 2148 | 2148 | protein | | | | FFSGQAQARHSSGHHGRTVF | |
| | | | | | | HSHRSSSHRHSYAHVIQCVA | |
| | | | | | | YAKTASEVVLHGNARDWWYN | |
| | | | | | | AAGVYARGSAPQAGSVLNFR | |
| | | | | | | AIRRMPLGHVAVVRSVEDSR | |
| | | | | | | TIYIDQSHWASNGIAHNVRV | |
| | | | | | | VDVSPNNDWSAVRVALNDRS | |
| | | | | | | GRLGSIYPTYGFIYPHSDNG | |
| | | | | | | DRNPAPHVVMARASTVSGFR | |
| | | | | | | HRAVLNGSDALSHPMNSTEV | |
| | | | | | | AEAPDDAFTSDAPDRSIR | |
| GO_ | GO_ | Ribonucleotide | H | | Up | MTDPDDSALRSVTLPAAWDD | 121 |
| 2185 | 2185 | reductase of | | | | EAAQALAQITLNGGPVRLAA | |
| | | class II | | | | EAARWVDTIDACPPLPGTPA | |
| | | (coenzymeB12- | | | | NTPSPGRSLSYLLLMQQMAP | |
| | | dependent) (EC | | | | NTALWQCQPDQTPGFTIRLS | |
| | | 1.17.4.1) | | | | SFVQEAGFAAEHFVACLRLA | |
| | | | | | | CDALRRLHAATRIERTGELP | |
| | | | | | | LFDLPAQPEDEAAGLILLTD | |
| | | | | | | LDACLAALGLDYDSDDARTA | |
| | | | | | | ACAMAALATTVARAGTKLSP | |
| | | | | | | PSIPESPLPGLRTIASSVCA | |
| | | | | | | TEEGRHFCPIETGFSSPAAT | |
| | | | | | | EGLLGVETCGLAPAFSPLRE | |
| | | | | | | DGHLRASTLARLACRGLTPE | |
| | | | | | | SALALALAGETPLPPVRPQA | |
| | | | | | | QAAMHAAVKNVVDFLPAVPE | |
| | | | | | | PDLADLQARLARGVRRPLPM | |
| | | | | | | RQTGFTQRAAVGGHSLFMRT | |
| | | | | | | SEFEDGTLGEISLTPPRESP | |
| | | | | | | MARGLMDCLGHAVSIGLQYG | |
| | | | | | | APLEAFVERFAYTRFGPAGT | |
| | | | | | | VEGDPSTAYATSMLDYAFRT | |
| | | | | | | LSEAYLGEHMPDAPRVEPSS | |
| | | | | | | EDPAPMLPFGRGSGESPEWK | |
| | | | | | | DRGRRLKLVS | |
| GO_ | GO_ | Uncharacterized | H | | Up | MTLNLRHYALLGLLAFLLAL | 122 |
| 2192 | 2192 | integral | | | | PGRMTLPPLDRDEPRYMEAS | |
| | | membrane, | | | | EQMLLSHNFIDVRFQDKPRY | |
| | | glycosyltransferase | | | | LQPAGIYWLEAASTAAAEKI | |
| | | | | | | FGPSVLRKTWPYRIPSLLAA | |
| | | | | | | TIIVPLTAWIGATLFGGATG | |
| | | | | | | LMAAGLLMVSTLFVAESHMA | |
| | | | | | | TIDTVLLLDILCIEAALLCA | |
| | | | | | | LTDRQKSRPTHLRVAVAYWL | |
| | | | | | | ALGVGLMLKGPVVLIPGFGT | |
| | | | | | | PLALWFLEKDRSWWPRLRPR | |
| | | | | | | WGWMLMIAVVVPWCVAIEVI | |
| | | | | | | SGGDFFARAVGRNFLGKVTH | |
| | | | | | | GQEAHGLPPGFHLLVFGLAF | |
| | | | | | | WPGSLFAALAIPSVWKNRKL | |
| | | | | | | PQVRFLLSWIVPHWLVFELI | |
| | | | | | | ATKLPHYVLPTYPAIAILTA | |
| | | | | | | ASLMAWRPLTLSRWAKALLG | |
| | | | | | | VYGVLWAVIGIAFCLAGSIA | |
| | | | | | | LYKLEHTFSLSALIALGGSL | |
| | | | | | | PLMLGAIMMLLKQQRRQAAF | |
| | | | | | | CAMGAAVIAHAGLFLSVIPN | |
| | | | | | | LQTIRLSPRIADLFEDVRPC | |
| | | | | | | NDSVLISSSYSEPSLVFLVG | |
| | | | | | | PNTQLIGPEAAAAYLHDHPQ | |
| | | | | | | CSLALIDIKDKNIFMSTLKK | |
| | | | | | | FGINVVEYNKIEGLNYSNGH | |
| | | | | | | HLNLELFAPL | |
| GO_ | GO_ | S- | H | | Up | MNALAQLGMVSELPRDIQNG | 123 |
| 2210 | 2210 | adenosylmethionine | | | | AAHLVEEERKDYFIERDGER | |
| | | decarboxylase | | | | YAGNHLLIDFWDARNLDDPM | |
| | | proenzyme | | | | RIDETLCEAAVAAGATILHS | |
| | | (EC 4.1.1.50), | | | | HFHHFTPNGGVSGVIVLAES | |
| | | prokaryotic | | | | HISIHTWPERNYAAVDVFMC | |
| | | class 1B | | | | GACDPNLSIPVMQRLFQAGR | |
| | | | | | | IEVDAVRRGRVQDKAVKAA | |
| GO_ | GO_ | tRNA | H | | Up | MNAPKTTEAKTAIIVAGPTC | 124 |
| 2214 | 2214 | dimethylallyl | | | | SGKSALALDLARTFGGTVIN | |
| | | transferase | | | | ADSMQVYRDLRILTARPDAA | |
| | | (EC 2.5.1.75) | | | | DEAAVPHRLYGVLDAAVPGS | |
| | | | | | | VAWWRAEALREMDAAWAEGR | |
| | | | | | | MPVLCGGTGMYLRALTDGLV | |
| | | | | | | EVPDPGDAARTEARGLAEEI | |
| | | | | | | GPEGLHARLMQVDPETASGL | |
| | | | | | | RPNDTQRISRAWEVWTGTGR | |
| | | | | | | GLAWWRSQPGLPPAPCRFVS | |
| | | | | | | VRLDPERDGLRRAIDSRFAQ | |
| | | | | | | MLDAGAIEEVSHLLERGLDP | |
| | | | | | | VLPAMRAHGVPELASVLRGD | |
| | | | | | | VTLEEARKSAVLAIGRYTRR | |
| | | | | | | QATWFRHHALGEAEDSMVSL | |
| | | | | | | RRYTHSAQESESHYEKIENF | |
| | | | | | | ISERVDAAALSS | |
| GO_ | GO_ | hypothetical | H | | Up | MLVHYGYGVVGIIVMFESMG | 125 |
| 2262 | 2262 | protein | | | | LPLPAESVIIAASLYAGSTH | |
| | | | | | | HLEIRWIALAAVLGAIMGDN | |
| | | | | | | IGYLIGHHFGYGILKKHGYK | |
| | | | | | | VGMTEERLMLGRYLFRKHGG | |
| | | | | | | IVVFLGRFIAVLRVFVALLA | |
| | | | | | | GANRMPWHSFLFFNAMGGIC | |
| | | | | | | WAGGYAFVTYELGKQIEKIS | |
| | | | | | | GPVGVVMAILGVSCLIGALV | |
| | | | | | | FLKKNEKRLTEEALREAEAD | |
| | | | | | | EKRDEARADTKPS | |
| GO_ | GO_ | Histidinol | H | | Up | MKRLDTSAAGFSEDFAKLLA | 126 |
| 2297 | 2297 | dehydrogenase | | | | ARGSDERSVAEPVRAILADV | |
| | | (EC 1.1.1.23) | | | | RSRGDEALCDYTARFDRLTL | |
| | | | | | | PAEKLRISTEEIASEAARVP | |
| | | | | | | ADLMDALRTAARRIETFHAA | |
| | | | | | | QMPKDLDFTDEDGIRLGMRW | |
| | | | | | | TPLDAVGLYVPGGKAAYPSS | |
| | | | | | | VLMNALPARVAGVKRLAMCV | |
| | | | | | | PSPGGVLNPLVLAAAQLCGV | |
| | | | | | | EEIYRIGGAQAVGAMAFGTD | |
| | | | | | | LIALVDRIVGPGNAYVAEAK | |
| | | | | | | RQVFGHVGIDSIAGPSEVVV | |
| | | | | | | VADGQNDPRLVALDLLAQAE | |
| | | | | | | HDEQAQAILITTDAAFADRA | |
| | | | | | | AEAVRKELETLPRTTIASKS | |
| | | | | | | WDDHGAIIVVRSLEEAAEIV | |
| | | | | | | NALAPEHLEVMLDAPRDFSA | |
| | | | | | | MIRHAGAIFMGRYCPEAVGD | |
| | | | | | | YVGGPNHVLPTSRTARFASG | |
| | | | | | | LSVFDFIKRTTTIETDEAGL | |
| | | | | | | RRIGPAGVALATAEGLDAHA | |
| | | | | | | LSLSVRLEKN | |
| GO_ | GO_ | hypothetical | H | | Up | MPRHHSYRNRSLLALMVLEI | 127 |
| 2348 | 2348 | protein | | | | CVPRMAVAASPASAATPVTQ | |
| | | | | | | APLIQASVTQAPVTQTEEWI | |
| | | | | | | HVPSTERPPIQNYPHAGVVA | |
| | | | | | | QPRRVVASQNHAPQGRQGQW | |
| | | | | | | GAFSYGNGESAGFGPVGRYG | |
| | | | | | | VAPWAEDWSFLRDKSRRDDP | |
| | | | | | | FDPLKFIALNDAKTIWLSFS | |
| | | | | | | GETRLRNWYEETPFLGKKGG | |
| | | | | | | SNSGRFGVRNLYGADLHLGE | |
| | | | | | | HVRLFGQLINADAAGWKGFG | |
| | | | | | | YNTTYRKRLDLQQAFIEFKG | |
| | | | | | | KLAGAQTGFMFGRQQFLDAP | |
| | | | | | | SYVLYNRETPNVPLSWNGGR | |
| | | | | | | IYAIWPNIRVDAFDFVQTKT | |
| | | | | | | DATLMFHDTEDYGTRLYGGD | |
| | | | | | | ITALVPQFSIGGETVHSFLD | |
| | | | | | | VFYYGYRYGGSLSVVPLASG | |
| | | | | | | SLKGTSSRGNVGFRWYGTAA | |
| | | | | | | SFEYSFGGLYQDGTFQKSGS | |
| | | | | | | EHKSGVQAYSINTIVGYRHT | |
| | | | | | | PSPLHPFIGVQADLYSGGAN | |
| | | | | | | GTNGPVRTYMAPFNPQTNYL | |
| | | | | | | DTTTYIQPSNLVSLSPVLSV | |
| | | | | | | TPWKGFASIQFKVPFMWREN | |
| | | | | | | ADGAIWNSSGPYTFSKTYHG | |
| | | | | | | GYIGVVPQASLKLQLNRHLT | |
| | | | | | | WQIYGARFMASNGLHAAGGK | |
| | | | | | | SGSYAQSNVVFRF | |
| GO_ | GO_ | hypothetical | H | | Up | MRQPLPARLALIALLGCGLA | 128 |
| 2355 | 2355 | protein | | | | ALPDSARAEPAIMPPPPPAP | |
| | | | | | | PAPPSLQAPLTASGTLVIPA | |
| | | | | | | TCLDRLSIVGGENAHIVSGS | |
| | | | | | | GSLEKHGDRLTFTTSPQDCA | |
| | | | | | | TQDSAVVMVPALTSIDIQLP | |
| | | | | | | HSRLTTYRITGVNGDVTAIS | |
| | | | | | | GRGNIDIDQASGLTLTMQST | |
| | | | | | | GNVSVGHVTGRLSVQNLSSG | |
| | | | | | | DLHIGDLAASSASITAMSSG | |
| | | | | | | DIRVEQGTVDVLTVRDYGSS | |
| | | | | | | TITLNAEARDADITLLGSGD | |
| | | | | | | ITLRKVSEALKKRPIGSGTL | |
| | | | | | | SIGDATSSRITSVNTPDGAA | |
| | | | | | | ILSDATRKILDRLDIQLDSP | |
| | | | | | | DHVSRTTDHNRHHSGGYGVI | |
| | | | | | | GVLLKIALIVWAVRLFRRYR | |
| | | | | | | RTGVLPFRKTLDKAAAGYAE | |
| | | | | | | GVQSWSRHRAAWRDTPGASA | |
| | | | | | | TAVVRDTMAGFRRQQPDPAE | |
| | | | | | | DFSRSVAPGHPLARLQDRLV | |
| | | | | | | RMERRLGLMEQFVTSPDFSL | |
| | | | | | | ERQFRDLERADGRRA | |
| GO_ | GO_ | Creatinine | H | | Up | MLFLRRLSCRVALLCVGMGL | 129 |
| 2373 | 2373 | amidohydrolase | | | | SVPAVRAQGVLEAPPHCSGL | |
| | | (EC 3.5.2.10) | | | | ALQAEFACRSWTEVAQDVKA | |
| | | | | | | GTDTVIIPVGGTEQSGPYMA | |
| | | | | | | VGKHNVRAQVLADAIAVQAG | |
| | | | | | | HTLVAPVVAYVPEGSTSPRT | |
| | | | | | | SHMKFPGTISIPPAVFEGLL | |
| | | | | | | KGAAESFRVQGFRRIVLLGD | |
| | | | | | | HGGYQSFMAQVAQELNRAWK | |
| | | | | | | GQAAVLYLRDYYEVVPHQYA | |
| | | | | | | EALRAQGHAAEVGLHAELSD | |
| | | | | | | TSLMLAVDPSLVRQDALRAA | |
| | | | | | | PKPGVAEGVYGGDPRRASAG | |
| | | | | | | LGRIGTEMQIRTAVAAIQSF | |
| | | | | | | QRSHP | |
| GO_ | GO_ | hypothetical | H | | Up | MTFKTPLLVAMTLLGAAAAH | 130 |
| 2374 | 2374 | protein | | | | AQEYSPSAPMVPVPADASAP | |
| | | | | | | VAAPVAPSGIQTIPGMPPVI | |
| | | | | | | DPKNIYSETTPSHISPAIAH | |
| | | | | | | DPARVYVPNLRGDSVSVIDP | |
| | | | | | | ASFQVVDTFRVGHSPQHVVP | |
| | | | | | | SWDLRMLWVINNSEGRPDGS | |
| | | | | | | LTPINPATAKPGPSIAVDDP | |
| | | | | | | YNMYFTPDGKYAITVAEAHK | |
| | | | | | | RLDFRDPHTMELKGSVETPE | |
| | | | | | | CKGVNHADFSIDGKYAIFTC | |
| | | | | | | EFGGYVAKVDTVNLKMIGML | |
| | | | | | | KLSKGGMPQDILTAPDGHKF | |
| | | | | | | YVADMMADGVFVVDGDSFTE | |
| | | | | | | TGFIPTGIGTHGLYPSRDGK | |
| | | | | | | LMYVANRGSHRIHGPKHGPG | |
| | | | | | | GVSIIDFATDKVIKTWMIPG | |
| | | | | | | GGSPDMGNVSADGKLLWLSG | |
| | | | | | | RFDDVVYAIDTDTGAMRKIP | |
| | | | | | | VGAEPHGLTVWPQPGRYSVG | |
| | | | | | | HTGILR | |
| GO_ | GO_ | 5-hydroxyisourate | H | | Up | MSSLSTHVLDTVSGKPAAGV | 131 |
| 2388 | 2388 | hydrolase | | | | SLRLLQGDRVLFEGQTNTDG | |
| | | (EC 3.5.2.17) | | | | RCPELRDVAVSKGIYCLEFQ | |
| | | | | | | IGDYFRKGGQVLSDPPFLDV | |
| | | | | | | VPIVFGLAAEAHAHVPLLAA | |
| | | | | | | PFGYSTYRGS | |
| GO_ | GO_ | Pyridoxamine | H | | Up | MSDIPLIDLKADPFALFAAW | 132 |
| 2435 | 2435 | 5′-phosphate | | | | MSDAEKSEPNDPNAMAVATA | |
| | | oxidase | | | | TPDGRPSVRMLLLKGVDERG | |
| | | (EC 1.4.3.5) | | | | FVFYTNLESRKGRELLSNPH | |
| | | | | | | VALLFHWKSLRRQIRIEGPV | |
| | | | | | | EAVSTTEADAYFASRSRMSR | |
| | | | | | | LGAIASDQSRPLDDRSTFEE | |
| | | | | | | RLKAVDGKYGDGPIPRPANW | |
| | | | | | | SGFRVLPEAIEFWQDRPYRL | |
| | | | | | | HDRAVWTRDGNGWNVTRLYP | |
| GO_ | GO_ | hypothetical | H | | Up | MLIDIKNILLPLNGSGDLEA | 133 |
| 2436 | 2436 | protein | | | | VMSVALDFARRFDAHLSAVV | |
| | | | | | | VGSDPSEVATLAGEGISAGM | |
| | | | | | | VNEMIDTATTEAQRRAISIR | |
| | | | | | | KAFDAFIHEHGIRRVEPSKI | |
| | | | | | | GSSAGDGVSASLDVLNGTEH | |
| | | | | | | DSLTWRSRLADMTLVPNLAK | |
| | | | | | | DGDPRASETLHAILFDSGRP | |
| | | | | | | LVIAPPAPPKTVGKRIAIAW | |
| | | | | | | NGTPEASLALRCILPWAHKA | |
| | | | | | | EGVQVLTCQDYQRRGPGADE | |
| | | | | | | VVTYLRMHGISATSREFEAV | |
| | | | | | | NRDIGAGLLKAATEFNADML | |
| | | | | | | GMGAYSHSRLRQMILGGVTR | |
| | | | | | | HILEQAQLTVLMSR | |
| GO_ | GO_ | Polyphosphate | H | | Up | MRSVFLCVPEDPIVTTEDSR | 134 |
| 2485 | 2485 | kinase | | | | PAPRRRSPRKPRNAVTPAQN | |
| | | (EC 2.7.4.1) | | | | RGRRRQTAAARAEDYAHMLT | |
| | | | | | | SPERFLNRELSWLDFNQRVI | |
| | | | | | | DEAENPRNPLLERVRFLSIS | |
| | | | | | | SSNLDEFYSVRVAGLVGQVR | |
| | | | | | | EGTVVRSPDGLTPAQQLVQV | |
| | | | | | | RQKARHLLAEQQRVWHLLEG | |
| | | | | | | ELKEAGIVILPTDSLDETDL | |
| | | | | | | AQLSTLFDERVFPVLTPMAV | |
| | | | | | | DPSHPLPFIPNMGLALHMRL | |
| | | | | | | KDAASSAHVMDGLILLPAQV | |
| | | | | | | PRFLRLPARLKEGQETPDQI | |
| | | | | | | RFVLLEDLITLFAGRLFPGL | |
| | | | | | | VIGAAGVLRVIRDTDVEFED | |
| | | | | | | EAEDLVRSYETALKQRRRGV | |
| | | | | | | CIHLALDRKLPDTLGREMGE | |
| | | | | | | ELGVGEEDVVVLPSFVGVTD | |
| | | | | | | LKQLIVDDRPDLVFPPYTPR | |
| | | | | | | FPERVLDYDGDCFAAIRAKD | |
| | | | | | | MLVHHPFESFDVVVQFLRQA | |
| | | | | | | ALDPNVLAIKQTLYRTSRDS | |
| | | | | | | PIVHALIEAAEAGKSVTAMV | |
| | | | | | | ELRARFDEEANIRLSRALEA | |
| | | | | | | AGVQVVFGFAHLKTHAKLSL | |
| | | | | | | VVRRENGSLRSYAHFGTGNY | |
| | | | | | | HPITARIYTDLSFFTCDPKL | |
| | | | | | | ASDSARLFNYMTGYAIPAKM | |
| | | | | | | DALAFSPITIRSTLEQLIQD | |
| | | | | | | EIDHAKAGRPGRIWLKMNSL | |
| | | | | | | VDAELIDRLYKASQAGVKII | |
| | | | | | | GIIRGICCLRPGVPGLSDNI | |
| | | | | | | EIKSIVGRFLEHARVFAFGN | |
| | | | | | | GHRMPSHKAKVFISSADWMV | |
| | | | | | | RNMDWRVEAMVPITNPTVHA | |
| | | | | | | QILGQIMTMNIKDNLQSWTL | |
| | | | | | | TRDGYWHRVSPGAHPFSAHE | |
| | | | | | | YFMNNPSLSGRGSAAREKVL | |
| | | | | | | PEAHRPRERPDRILED | |
| GO_ | GO_ | UDP-N- | H | | Up | MPARRIALNVVLDGVVSAAA | 135 |
| 2496 | 2496 | acetylglucosamine | | | | APVARWLADPAGGWLHPLWF | |
| | | 4,6-dehydratase | | | | IAGGGITLLVGGLPFRIPQQ | |
| | | (EC | | | | YWRFSGVADLFNIACASVLS | |
| | | 4.2.1.135) | | | | ALLFAELLHVAGYPLPTPTF | |
| | | | | | | PIIHALVLLVFLGAIRMMWR | |
| | | | | | | LAARRRSLALDGERILLLGA | |
| | | | | | | DHEADLFIRAMERDSGNNRR | |
| | | | | | | VVGLLTGGAQQAGRRIHDCP | |
| | | | | | | ILGTISETPAILERLFAAGK | |
| | | | | | | LPDALVITAGEIKGRELAQI | |
| | | | | | | LEAARVYDIDVQRTPSLTAL | |
| | | | | | | QPADRVELRPIAIEDLLNRP | |
| | | | | | | PVALDSQGMARLICGRVIAV | |
| | | | | | | TGAGGSIGSELARQIASFQP | |
| | | | | | | AMLLLIESSEYALWQINLDI | |
| | | | | | | SERFADVKRQQIIADVRDRR | |
| | | | | | | RIAEVFGMYRPHLVFHAAAL | |
| | | | | | | KHVPIVEDNPVEGVLTNVIG | |
| | | | | | | TRIVADEAERAGAQAMVMIS | |
| | | | | | | TDKAVNPSSLMGASKRCAEV | |
| | | | | | | YGQALDMRARAGQGGMRCVT | |
| | | | | | | VRFGNVLGSTGSVVPLFRRQ | |
| | | | | | | LEHGGPLTVTHPDMTRYFMT | |
| | | | | | | VPEAVGLVLQAAVRGTHERA | |
| | | | | | | QNDATDLRLRQGGIFVLDMG | |
| | | | | | | DPVRIMDLAFQMIRLAGLRP | |
| | | | | | | ETDIEIRFTGLRPGEKLFEE | |
| | | | | | | LFHGREAPVPTDAPGLRMAS | |
| | | | | | | PRTVDFKVAAAAMDELETAC | |
| | | | | | | HASDLGAIMSILYRLVPEFL | |
| | | | | | | HNRDGGMPVAMSHPDPEMIA | |
| | | | | | | P | |
| GO_ | GO_ | Putative | H | | Up | MGMPGDNRPQDVQVSSVMEE | 136 |
| 2511 | 2511 | transmembrane | | | | ERLSVDVGAAGGTCVETGQR | |
| | | protein | | | | KRRWQVFMSRAPALVGLVLL | |
| | | | | | | VAAVVVIWRELQHLSLHDIT | |
| | | | | | | ASLSAIPDSALLAGGAATVL | |
| | | | | | | SYFILSFYDRLACLHVRAKV | |
| | | | | | | SYQRSAFAAFCSYVLSHNLG | |
| | | | | | | CAAISGAAVRFRLYRSWGVA | |
| | | | | | | PGAIAQIIAFCSATYLLGTM | |
| | | | | | | ALIGGILIIEPHAVPVLSHL | |
| | | | | | | PEFLLRLAGLALWGVLLAYV | |
| | | | | | | LVARVRRHVRIWKYEIELPG | |
| | | | | | | PGIAIAQIAVSAADMAATAL | |
| | | | | | | IAYCVLPPLPPEAHFGFGTF | |
| | | | | | | LAIYLASYTAGLMASVPGGL | |
| | | | | | | GVEDGAMLLALQAYLPASQI | |
| | | | | | | MGAILVFRLFYYIIPLLLAG | |
| | | | | | | LMFAGHELFLRGEQALVSAG | |
| | | | | | | QTPRRVRPSQVIRESEADFS | |
| | | | | | | VAVATSVQAVVGILLVLYAL | |
| | | | | | | VADLPPLQTSLGAAVSQIAD | |
| | | | | | | LLLTVAGVGLVALSWGLSQR | |
| | | | | | | VALAWKFSLGMLGSAALLLM | |
| | | | | | | LRHAPWEGPVVIVLVMLLLL | |
| | | | | | | PFRNCYYRRAHLLAAPLTPS | |
| | | | | | | MLAPLSLWGLGLLGVGWVAV | |
| | | | | | | QRHLGPIWWRSMIYDAHTAV | |
| | | | | | | GRWFLGFSALCGFYVLWRGM | |
| | | | | | | RRTRIRFEAWTAENAHRYHS | |
| | | | | | | LAHALPQLGARRPTGLLLDE | |
| | | | | | | AGRAAIPFLRTGQFIIGLGD | |
| | | | | | | PAGSERNCVAAIWRLRDLAL | |
| | | | | | | QEGCKLAFIQVGQSLMAVYN | |
| | | | | | | DLGLTVCPDRTAGTVCCFSE | |
| | | | | | | DYRMLRAFLKGEERLARKRQ | |
| | | | | | | PQTASGLTGS | |
| GO_ | GO_ | Glycerol-3- | H | | Up | MTTRPHIAVIGAGAWGTALA | 137 |
| 2544 | 2544 | phosphate | | | | CATAATGADVTLWMRNPVPP | |
| | | dehydrogenase | | | | GTRTLPRLPDITLPDNVTIT | |
| | | [NAD(P)+](EC | | | | GDFPRTADIALLVTPVQTAR | |
| | | 1.1.1.94) | | | | DVSTRLQTVLDPAVPVVTCC | |
| | | | | | | KGLEQATSLLPLDVLAETMP | |
| | | | | | | GRPTGVLSGPNFAIEVAKGL | |
| | | | | | | PAAATLACTDLALAQKLTAL | |
| | | | | | | LNTSSFRLYASDDAAGVQLA | |
| | | | | | | GAAKNVIAIGAGITIGAGLG | |
| | | | | | | ENARAALITRAVAEIGRLAE | |
| | | | | | | ATGGRASTLAGLAGMGDLIL | |
| | | | | | | TCTGRGSRNYSVGLELGEGR | |
| | | | | | | PLTDILASRTTVAEGVLTAP | |
| | | | | | | AMLALARQHNVRVPIIETVT | |
| | | | | | | RLLNDGVSIEEARHLLLDRP | |
| | | | | | | PTRE | |
| GO_ | GO_ | Heat shock | H | | Up | MSGRLFTSPMFLGFDHLEQM | 138 |
| 2589 | 2589 | protein, | | | | LERAAKSTSDGYPPYNIEQL | |
| | | Hsp20 family | | | | SSTALRITLAVAGFVMDDLQ | |
| | | | | | | ITQEDNQLVIRGRQADDSQG | |
| | | | | | | RIFLHRGIAARQFHKAFVLA | |
| | | | | | | EGIEIGGAWLDNGLLHIDLL | |
| | | | | | | RPEPEVRVKRIAISQGRRST | |
| | | | | | | APGPAVHHEVVPPVVKARRT | |
| | | | | | | TRPARDIDDE | |
| GO_ | GO_ | tRNA | H | | Up | MSDTQAAEPIAEPAIEPTGL | 139 |
| 2592 | 2592 | pseudouridine | | | | NRWALRIEYDGTGYLGWQKQ | |
| | | (38-40) | | | | NDGTSIQGLIEAAASKLVRN | |
| | | synthase | | | | RPVPSITAGRTDAGVHAAGM | |
| | | (EC | | | | VIHLDFPDDAPIDARQIRDG | |
| | | 5.4.99.12) | | | | MGYHLKPHRVVVLETAKVGP | |
| | | | | | | EWNARFSATWRSYRYTILNR | |
| | | | | | | PARPGLMENRVWHIKRPLDV | |
| | | | | | | DLMQQAANHLLGPHDFTSFR | |
| | | | | | | AVACQARSPIRTLDVLNIHR | |
| | | | | | | DGELVVIDTKARSFLHHQVR | |
| | | | | | | NMAGTLMMIGSRQWPVEKII | |
| | | | | | | EILEAKDRCAAGQTAPPEGL | |
| | | | | | | CLMDVGYPDDPFNRF | |
| GO_ | GO_ | UDP-3-O-[3- | H | | Up | MSDMTASESRPGDSRFFQRS | 140 |
| 2607 | 2607 | hydroxymyristoyl] | | | | GPFGLERLAEVSGSEIIPAA | |
| | | glucosamineN- | | | | SGKGLSEFRGVAPLHVAGPD | |
| | | acyltransferase | | | | EISFLDNRRYLPLLAETKAG | |
| | | (EC 2.3.1.191) | | | | AVILSPAFTDKLPPDTAGLA | |
| | | | | | | CKAPYLAWARVATLFHPAPA | |
| | | | | | | STGVRHPSAWIAEDAEIGEN | |
| | | | | | | VEIGPFAVIGSGVRIGRDSI | |
| | | | | | | VASHVSIGQSVEIGERCRIG | |
| | | | | | | AHAAISHARIGDRVTLYPGV | |
| | | | | | | RIGQDGFGFAVGPEGFETVP | |
| | | | | | | QLGLVVLEDGVEVGANSTID | |
| | | | | | | RGSMRDTLIGAGTRIDNLVQ | |
| | | | | | | IGHNARLGRCCIVVSQAGIS | |
| | | | | | | GSTELGDFVTIAAQAGLIGH | |
| | | | | | | IKIGSKARIGAQCGVMSDVD | |
| | | | | | | AGADVIGSPAMPFREFFRNV | |
| | | | | | | ATLRKLSRKSGD | |
| GO_ | GO_ | Periplasmic | H | | Up | MTLNSLMRTVSAGALLAATI | 141 |
| 2608 | 2608 | chaperone | | | | LVSAPGAHAQASGGNGGWFV | |
| | | of outer | | | | PKAAHPDAPPPRPVQRRVPE | |
| | | membrane | | | | AAPDEEEDSAPAEQQQAPPI | |
| | | proteins | | | | LPLPPIPAPPSIAKASPPPA | |
| | | Skp @ Outer | | | | AVIGVINVQAVMQISSAWQE | |
| | | membrane | | | | IQQVLGARRDRLAQAVQREE | |
| | | protein H | | | | AAWRGEQQKLQAQARSLTSD | |
| | | precursor | | | | QIQLRERHLQERRAKDQHDF | |
| | | | | | | GNQARIIQEAAQVAMHQIER | |
| | | | | | | ELEEPNGIIAAVAAAHNMNL | |
| | | | | | | ILHAEQVVLHVGGQDITEEV | |
| | | | | | | GTQLNKTLPHVFIPDDGVDP | |
| | | | | | | EQLARSGKMPTTADEQRQAQ | |
| | | | | | | GPQAPGQQQAPASAPSESVL | |
| | | | | | | RQHH | |
| GO_ | GO_ | hypothetical | H | | Up | MLLRQNERTALFIDGASLHH | 142 |
| 2622 | 2622 | protein | | | | AARNLGFEVDFRSLRNLFES | |
| | | | | | | QCLFQRAFYYAAMPETDDYS | |
| | | | | | | PLRPLTDWLAYNGYHLVLKN | |
| | | | | | | AREFTDHSGRRRIKGNMDVE | |
| | | | | | | LTVDLLEQAGRLDHAVIVSG | |
| | | | | | | DSDLRRAVEAVQARGVRVTV | |
| | | | | | | ISSMRSTPPMIGDDLRRQAD | |
| | | | | | | LFVELADIAPSFTRRQAEPR | |
| | | | | | | NPSRQGPVRHPADINSDETS | |
| | | | | | | DS | |
| GO_ | GO_ | Glutathione | H | | Up | MRILHHLPLSPQCRLVRLAL | 143 |
| 2643 | 2643 | S-transferase | | | | SEKRLPFEPVIERVWEQREE | |
| | | family | | | | FLHLNPAGEVPVLVEENGLA | |
| | | protein | | | | VPGGRVICEYLEDAYPDTPL | |
| | | | | | | LGRTFADRVETRRLVDWFDT | |
| | | | | | | RFAQEVTRNLLGEKVDKRQF | |
| | | | | | | GRGHPDGNALRAGYANMRFH | |
| | | | | | | LDYIGWLAETRSWLAGPALS | |
| | | | | | | LADFAAAAHLSALDFIGDVN | |
| | | | | | | WSKAPAAKDWYARVKSRPCF | |
| | | | | | | RGLLSDKVSGITPPAHYANL | |
| | | | | | | DF“ | |
| GO_ | czcA/cus | Cobalt-zinc- | H | | Up | MNAIVVTALKRPYTFVVLSI | 144 |
| 2649 | A | cadmium | | | | MILIFGVRAIVSTPTDVFPS | |
| | | resistance | | | | IKIPIVAVIWSYTGLMPDDM | |
| | | protein | | | | SGRIVYYYERALTATVSNIE | |
| | | CzcA; | | | | HIESSSYYGRGIVKIFFQPG | |
| | | Cation efflux | | | | TNTAVAQTQITSVSQTVIKQ | |
| | | system protein | | | | LPNGATPPLILAMDASSVPV | |
| | | CusA | | | | LTLQVNNPTMSGSEIYNMAS | |
| | | | | | | NLIRPELISVPGAAIPNPYG | |
| | | | | | | GLAPDIMVDIDPIKLLAHRL | |
| | | | | | | SPEDVAAALNLQNIVLPAGD | |
| | | | | | | QRIGQLDWMVKTNSTPLDLA | |
| | | | | | | VFNKMPVKQVGNSVIYLRDV | |
| | | | | | | AWVHRGGPPQINAVLVKGQQ | |
| | | | | | | AVLIVILKSGDASTLSVVSG | |
| | | | | | | IKKLLPQVQATLPAGTTVSI | |
| | | | | | | LTDASSFVKESVVDVVREMI | |
| | | | | | | TAAILTSLTVMLFLGSWRST | |
| | | | | | | VIVATSIPLAMLCSIIGLSI | |
| | | | | | | AGQSINVMTLGGLALAVGIL | |
| | | | | | | VDDATVMIENIDAHLETGKE | |
| | | | | | | LEDAIIDAANQIVIPTFVST | |
| | | | | | | TCICIVWLPLFELTGISGWL | |
| | | | | | | FMPMAEAIIFAMIASFILSR | |
| | | | | | | TLVPTMANWMLAAQVRMHRD | |
| | | | | | | PEWHNRKLSIFGRFQRGFEA | |
| | | | | | | RFTSFREHYKTILETLISIR | |
| | | | | | | GRFVTLFLLAAVSSMALLLF | |
| | | | | | | IGQDFFPEIKSGTLQMHMRA | |
| | | | | | | PIGSRLEETGKIAGLAERRI | |
| | | | | | | RSLLPGQVVNVVNNCGLPFS | |
| | | | | | | QLNQAMIPSPTVGSQDCDIT | |
| | | | | | | IQLRNSESPIAEYRRTLRKG | |
| | | | | | | LTNDFPGTIFTFQPGDLTAK | |
| | | | | | | ILNFGLPSPIDVQVVGRDLS | |
| | | | | | | DNFRFATQLAKKLRHIPGIT | |
| | | | | | | DVSIQEPMTQPTIMVNNRRS | |
| | | | | | | FALGTGITERDVALNALVTL | |
| | | | | | | SGSGQVGQTYYLNAQGTSQL | |
| | | | | | | IDVQAPANYLQTMNDLEILP | |
| | | | | | | IDKGDGNPTNQTPQLLGGLS | |
| | | | | | | ALVQTGTPSEIAHYNIMPVF | |
| | | | | | | DIYAAPEDMDLGTVSRAVNR | |
| | | | | | | IVNHERKLLPHGSSMVVRGQ | |
| | | | | | | AVTMNDAYVQLIGGLALSIV | |
| | | | | | | LVYLIIVVNFQSWLDPFVII | |
| | | | | | | TALPGALGGISWSLFLTHTA | |
| | | | | | | MSVPALTGAIMCMGTATANA | |
| | | | | | | ILVVSFARERMDHHGDAITA | |
| | | | | | | ALEAGYERIRPVLMTALAMM | |
| | | | | | | IGMIPMSVSNSDNAPLGKAV | |
| | | | | | | IGGLLVATVATLLFVPCVFA | |
| | | | | | | LIHYKRPAGPEGDRA | |
| GO_ | GO_ | Membrane- | H | | Up | MSHSADQGQVPPTNHPARTG | 145 |
| 2650 | 2650 | fusion | | | | SGTRGKLVLLVVILLAIALA | |
| | | protein | | | | AWGIVQRGAHYHSLTGATED | |
| | | | | | | AAIPPVTLIAPQPGPKTRQV | |
| | | | | | | DLPANLAAWYEAPIYAQVSG | |
| | | | | | | YVKMWYKDYGAHVKRGDVLA | |
| | | | | | | EISTPSIDAQFEAAKAHYNV | |
| | | | | | | ILARYNLALITTKRWTALKG | |
| | | | | | | TQAVSRQEVDVQAANAAAQK | |
| | | | | | | AELEAARHDVDRFQALEDFK | |
| | | | | | | KIVAPFDGIVTSRLVNVGDY | |
| | | | | | | VNAGGGNLNSRGTASELFSV | |
| | | | | | | ADVHRMRVFVSVPQDFASVI | |
| | | | | | | SPKIEAELTVPQYPGSHFRA | |
| | | | | | | TFLATANAFNAATRTVTTEL | |
| | | | | | | TLDNSDNLLWPNSYATAHIS | |
| | | | | | | APGNPNILILPEGAIIFRAE | |
| | | | | | | GTQVAKVINNHAHLVNVTVG | |
| | | | | | | INFGTTVQVLSGITKDDRVV | |
| | | | | | | ANPTADLLEGDEVKIVPTTP | |
| | | | | | | GYNTPSKAQQDADQQPVRQH | |
| | | | | | | DANPEEAGSR | |
| GO_ | GO_ | Efflux | H | | Up | MKPECSQSSPLQSSPSATAS | 146 |
| 2651 | 2651 | transport | | | | SRRNRSRWRITTALAGVFSI | |
| | | system, | | | | GLASCDLSPEYHPQKFLYPE | |
| | | outer | | | | GWEGKGLMVNAQPADGVVRS | |
| | | membrane | | | | DWWTMFNDPILDGLEKRMLA | |
| | | factor | | | | VNPDLQAAAEAFTQARDVAR | |
| | | (OMF) | | | | ETESRLYPQVTGAAHMSDNK | |
| | | lipoprotein | | | | GSIGRLYNNPATSSSLVYES | |
| | | | | | | NQAYSGAATWEPDFWNSIRN | |
| | | | | | | TTRMQKNLAQASAGQYALAR | |
| | | | | | | LSLEAELASDYIALRGLDAQ | |
| | | | | | | NAVYDDSIRYFRAAVEITEL | |
| | | | | | | RQAGSIGAGLDVSRAETQLY | |
| | | | | | | SAQAGKSNLIARRNVMEHAI | |
| | | | | | | AVLLNTAPAGFHIAPVKDVK | |
| | | | | | | MHFGVVKINAGLPASLLERR | |
| | | | | | | PDIAIAERQMAASARAIGVS | |
| | | | | | | RAAFYPHITFSAEGGFEDGG | |
| | | | | | | FDLASISRAFWKIAVQAVEP | |
| | | | | | | AFTGGLRRAALQRSWSQYRS | |
| | | | | | | MVDNYRSVVLSAFQDVEDGL | |
| | | | | | | TQTRQFKIAQDQQQKAVDAA | |
| | | | | | | LRTQSMTMALYTGGLSNYLD | |
| | | | | | | ALVAQQDALQARLAEVEVQT | |
| | | | | | | AQVQSSVRLVRALGGGWSAS | |
| | | | | | | DLPGIKQIDPFGPLQYKDLR | |
| | | | | | | TPKPVNGIDSHASPLDNDLR | |
| | | | | | | GDRVSENVP | |
| GO_ | GO_ | DNA-directed | H | | Up | MNELMKILGQTGQSVTFDQI | 147 |
| 2657 | 2657 | RNA | | | | KIQLASSEQVRSWSYGEIKK | |
| | | polymerase | | | | PETINYRTFKPERDGLFCAR | |
| | | beta′ | | | | IFGPIKDYECLCGKYKRMKF | |
| | | subunit (EC | | | | RGIVCEKCGVEVTLAKVRRE | |
| | | 2.7.7.6) | | | | RMGHIELASPVAHIWFLKSL | |
| | | | | | | PSRIATMLDLPLKDVEPVLY | |
| | | | | | | FEKFLVLDKGVCESDQIDSY | |
| | | | | | | KNGKKRDQYLLDEIRCEDLL | |
| | | | | | | DEYPDAGIDVGIGAEAIKRA | |
| | | | | | | LSSYDWGIPNDQERDLSLAA | |
| | | | | | | KEKGLPDPFDYDADVMEGDS | |
| | | | | | | EKTMMRKKLRKATSEAARKK | |
| | | | | | | LVKRLKLVEAFVESGSRPDW | |
| | | | | | | MIMDIVPVIPPELRPLVPLD | |
| | | | | | | GGRFATSDLNDLYRRVINRN | |
| | | | | | | NRLKRLIELRAPDIIVRNEK | |
| | | | | | | RMLQEAVDALFDNGRRGRAI | |
| | | | | | | TGANKRPLKSLSDMLKGKQG | |
| | | | | | | RFRQNLLGKRVDYSGRSVIV | |
| | | | | | | VGPELKLHQCGLPKKMALEL | |
| | | | | | | FKPFIYSKLEKYGHATTIKA | |
| | | | | | | AKRMVEKERPEVWDILEEVI | |
| | | | | | | REHPVMLNRAPTLHRLGIQA | |
| | | | | | | FEPTLIEGKAIQLHPLVCTA | |
| | | | | | | FNADFDGDQMAVHVPLSLEA | |
| | | | | | | QLEARVLMMSTNNILSPANG | |
| | | | | | | KPIIVPSQDIVLGLYYLSLE | |
| | | | | | | VPEYRETPDEAVIKDGKIVT | |
| | | | | | | AAPPAYSDVAEIESAMLSGS | |
| | | | | | | LKLHDKIRLRLPTIDADGKS | |
| | | | | | | VRQTIVTTPGRALIAQILPK | |
| | | | | | | HQAIPFSLINKQLTKKNVSD | |
| | | | | | | VIDTVYRHCGQKEAVIFCDR | |
| | | | | | | LMGLGFRHAARAGISFGKDD | |
| | | | | | | MIIPEAKATLVGKTSEEVKE | |
| | | | | | | FEQQYQDGLITAGERYNKVV | |
| | | | | | | DAWSRCTDEVQAAMLKEISK | |
| | | | | | | QVIGKPTNSVWMMSHSGARG | |
| | | | | | | SPAQMKQLAGMRGLMVKPSG | |
| | | | | | | EIIEQPIIANFKEGLSVLDY | |
| | | | | | | FTSSHGARKGLADTALKTAN | |
| | | | | | | SGYLTRRLVDVAQDCIIVEP | |
| | | | | | | DCGTERGLTVRAVMDSGEVV | |
| | | | | | | ASLSERILGRTLSKDVIHPV | |
| | | | | | | TQDVILPRNTLIEEAEAELI | |
| | | | | | | EKAGVESVDIRSVLTCDSRV | |
| | | | | | | GICAHCYGRDLARGTPVNIG | |
| | | | | | | EAVGVIAAQSIGEPGTQLTM | |
| | | | | | | RTFHIGGAATRGAEQSMVEA | |
| | | | | | | SRDGIVTIKNRNVVENSQKV | |
| | | | | | | LVVMSRNCEILLTDENGVER | |
| | | | | | | ARYRVPYGARLMVSEGEAVT | |
| | | | | | | RTQKMAEWDPYTLPIITEQA | |
| | | | | | | GTVEYLDLIDSITLVERMDE | |
| | | | | | | VTGLSSKVVVDYKQAAKGVD | |
| | | | | | | LRPRLQLKDASGNVVKLANG | |
| | | | | | | NDARYFLSPDSILSVENGAE | |
| | | | | | | VNAGDVLARIPREGSKTRDI | |
| | | | | | | TGGLPRVAELFEARRPKDHA | |
| | | | | | | IIAEGEGRIEFGKDYKSKRC | |
| | | | | | | VIVKNDDTGEETQYLIPKGK | |
| | | | | | | HVSVQEGDFVQKGDPLVDGP | |
| | | | | | | RVPHDILKVMGVEALSDYLV | |
| | | | | | | NEIQDVYRLQGVKINDKHIE | |
| | | | | | | VIVRQMLQKVEILEPGDSTY | |
| | | | | | | LIGETVDRIEYEGENQRLME | |
| | | | | | | NGDTPAKAMPVLQGITKASL | |
| | | | | | | QTQSFISAASFQETTRVLTD | |
| | | | | | | AATSGKVDTLNGLKENVIVG | |
| | | | | | | RLIPAGTGSVMNRLRGIAAS | |
| | | | | | | QDRQRVGGTSPKAVEDAAE | |
| GO_ | GO_ | hypothetical | H | | Up | MDADLEQYLEAGVGRLQDDA | 148 |
| 2689 | 2689 | protein | | | | STCSRMKGLEKKVSSAARET | |
| | | | | | | EALVSDLMVDQPLFWLLTSF | |
| | | | | | | FIGILLGKGLFRKS | |
| GO_ | GO_ | FIG139612: | H | | Up | MKSPSDTLRSLFRRNRDAAS | 149 |
| 2702 | 2702 | Possible | | | | SAPAVQAESLALPALVLEAE | |
| | | conserved | | | | KIAASLQSGVHGRRRSGAGE | |
| | | membrane | | | | DFWQFRPYHAGEPATSIDWR | |
| | | protein | | | | QSARSPVEDTFWVREREREN | |
| | | | | | | AQSLMLWCDPSPSMQWRSSD | |
| | | | | | | VLPTKAERAQLCTLALASAS | |
| | | | | | | LRGGEHAGLLTGIEAGRALA | |
| | | | | | | GRQVLPRLAASLLRPDADEP | |
| | | | | | | EFPRMALVPARSDLVVISDF | |
| | | | | | | LWDEDRIDTFLKTCASRPVR | |
| | | | | | | THLLCVLDPAERELKRSGRI | |
| | | | | | | RFEGLEGGVLTLPAMESLGP | |
| | | | | | | AYEQAMNAHLAALKQSAASL | |
| | | | | | | HADCIMHDTSQNPLPALLAL | |
| | | | | | | HMALGGGR | |
| GO_ | GO_ | DNA polymerase | H | | Up | MDVGFYHLTRTPLEEALPAL | 150 |
| 2752 | 2752 | III | | | | LGRTLDAGERALVRCPDAAA | |
| | | chi subunit (EC | | | | VMALDAALWACRDPVWLPHG | |
| | | 2.7.7.7) | | | | TAKSGHADRQPIWLTEGEDV | |
| | | | | | | PNGARFLFRVDGAGSDEFAP | |
| | | | | | | FTRIFDLFDGGNPQSVQRAR | |
| | | | | | | QRWVAMKSSGHNLVYWKQEE | |
| | | | | | | RGWKKAG | |
| GO_ | GO_ | hypothetical | H | | Up | MRSVSTSCPDRALHILASLF | 151 |
| 2755 | 2755 | protein | | | | HVDPVSLCLALLVIPACVQA | |
| | | | | | | MQPGRPGRAVVLCLATLAAV | |
| | | | | | | LGLSPAVQAVALGVIAAQDR | |
| | | | | | | EACPAVWSVPALLLSALFPA | |
| | | | | | | QTFVVLAVLPVLFWSALVRR | |
| | | | | | | SDGEQEGGPFPAVMGILGVS | |
| | | | | | | LVWHAPAAVSAELVAGLGAA | |
| | | | | | | VIGLLGRSVLGACRPGDLGL | |
| | | | | | | LRPVLLMVLVVAAQAEGLAV | |
| | | | | | | CARIALEAILLDLSLLVLTA | |
| | | | | | | ALGRVFPVLSVLRLPFPPLP | |
| | | | | | | GLVVLWLGIHAALGMAAGIE | |
| | | | | | | GWSVLGVAVALLLGLLGLSD | |
| | | | | | | ILVVGRVFSAWQGRVSGVSV | |
| | | | | | | LLVGAGSLLLPALVFGVVSP | |
| | | | | | | VLHFMGGEWVWPVWRMGGGD | |
| | | | | | | GASLRLPAFVLTGAVLWCVL | |
| | | | | | | VRPWRVAGGIVQAASTLLPA | |
| | | | | | | LGNLLSVGDGLFEEAPSLGW | |
| | | | | | | KIRCVIVAGRRRFMAVRGVK | |
| | | | | | | APVLPDLRQGAVGLWLVLLG | |
| | | | | | | LVLAVLGVMA | |
| GO_ | GO_ | Hydrogenase-4 | H | | Up | MIGMGRMIRSGERVALSHYH | 152 |
| 2759 | 2759 | component G | | | | LDAEQWSAMLSAPGTTLPLI | |
| | | | | | | SCWADDARAYVLLLEGDRPL | |
| | | | | | | VASTAVEERRYLAPSSRFAG | |
| | | | | | | AEWGERVAYDLYGVEAMDAR | |
| | | | | | | GNGAPALDEGGWTSTWPLSS | |
| | | | | | | RPGPAAGGLRPLAGRHLLRP | |
| | | | | | | EQTGLSGPLELSFEVLKGKV | |
| | | | | | | RSVEVCAGGAHRGVMSRLLG | |
| | | | | | | RTPEEAMPLVSRMTAGGFVA | |
| | | | | | | HPLAFARAVAQARGLVPGPG | |
| | | | | | | IRDVWMLLLEIERMSLHLFD | |
| | | | | | | MARTARGVDAELFATHCDHA | |
| | | | | | | REAIARACAEQGVSRRLMDM | |
| | | | | | | VACDGFREGLEIVPLAQAVH | |
| | | | | | | AAMQPRLAALEELHRVFAPR | |
| | | | | | | LDGFAVLDVRLAERFAVGGV | |
| | | | | | | TGRASGRSMDMRRREAGMRL | |
| | | | | | | EALRATGSSQGDARAREGLR | |
| | | | | | | LAEIRDSLKLLERILGSIGL | |
| | | | | | | EDDEPAPDRTDEGIGVAEGA | |
| | | | | | | RGDVWYWVRLKDGRIESLHV | |
| | | | | | | RDPGVSLLPVLGAMLRGYPV | |
| | | | | | | SRVPAALGSIGISPAGIAL | |
| GO_ | GO_ | SAM-dependent | H | | Up | MLTSLARIRSDDAATFYESR | 153 |
| 2764 | 2764 | methyltransferase 2, | | | | QGQRTALLLNGRLQSIMPPM | |
| | | in cluster | | | | RGRRILGIGYTAPYLPESAE | |
| | | with | | | | FAVSGRLLHPQTQRTTRPTR | |
| | | Hydroxyacyl | | | | SQTRNISWADCIVSSGRLPF | |
| | | glutathione | | | | DDLSMNAVLVVHGLEFTRFA | |
| | | hydrolase | | | | PDFLRAIWRTLSDDGILTLV | |
| | | | | | | VPNRSGYWAHTDATPFGHGI | |
| | | | | | | PYSSGQLTRLLDQALFRIEH | |
| | | | | | | HSTALMTPPAALSVTHGRLM | |
| | | | | | | ERAGRTLRLPCGGVHVVTAR | |
| | | | | | | KNVYAGTPLIDEKLCVPLPR | |
| | | | | | | QVAEPA | |
| GO_ | GO_ | Hydroxyacyl | H | | Up | MPLDIKPIPVLSDNYAWLLT | 154 |
| 2765 | 2765 | glutathione | | | | AMEGQRAVVDPGEAGPIMDE | |
| | | hydrolase (EC | | | | IGEGRLDMILLTHHHADHTA | |
| | | 3.1.2.6) | | | | GTDALRERYGAKVYGPRQKR | |
| | | | | | | EWLPRLDHDVEDGDSFSLGS | |
| | | | | | | AQIRVLSTPGHAVGHVSYVV | |
| | | | | | | PGVPALFCGDVLFSLGCGRL | |
| | | | | | | LEGTAQELFDSLHRYDSLPD | |
| | | | | | | RTLVCAGHEYTRSNLAFALH | |
| | | | | | | VDPDNEALKARAAEVEQLLE | |
| | | | | | | AGRPTLPVSLGVERKTNPFL | |
| | | | | | | LAPDVATFARLRREKDTF | |
| GO_ | ftsX | Cell-division- | H | | Up | MSAPVSPGLRSGSLPLLVAL | 155 |
| 2772 | | associated, | | | | MTLLAGLSLAGLTGVQTLAE | |
| | | ABC- | | | | GWAGAARNATTIEIPSDTPQ | |
| | | transporter- | | | | LEDRTRALIQTLHKTPDITT | |
| | | like | | | | VRELSPQQVQTLLAPWLGQV | |
| | | signaling | | | | SDSGHLPGLSLPVVLIVAHT | |
| | | protein | | | | GTPDLGRVVHEALPEAVVEE | |
| | | FtsX | | | | DRRWGERLNGLGSSLVACAW | |
| | | | | | | LAVSLIAAIAVLSVGMTVRR | |
| | | | | | | SVMAQRKAVEIVHFLGAGDV | |
| | | | | | | TISSRIAGRAALLSLAGGLA | |
| | | | | | | GLFSLSPVITMLARKLAPFS | |
| | | | | | | HDAGSAALPATTWQTMLASW | |
| | | | | | | WNTLHVLPRLLLEELGALPL | |
| | | | | | | IAACLGWLTAQTVVLVWLRR | |
| | | | | | | LP | |
| GO_ | GO_ | hypothetical | H | | Up | MRIECPHCHAVFEVPEALAK | 156 |
| 2774 | 2774 | protein | | | | GVKRLRCANCGDSWELGAAA | |
| | | | | | | VSKSEEAPDGAVAAPEVFEV | |
| | | | | | | PEEGVAAAPAVADSPVSGTE | |
| | | | | | | GTFRATGAMASRSNARRSAV | |
| | | | | | | LHSRSAGDVQVPTDTAGPSM | |
| | | | | | | IGSTGAWIAAWGASLVLAGG | |
| | | | | | | GVAALWYCKGAGVFGFLPGA | |
| | | | | | | GHFS | |
| GO_ | GO_ | Translation | H | | Up | MTVLSADPGRIAERLPLLER | 157 |
| 2813 | 2813 | elongation | | | | RTQMVRSVRTFFEERGYLEV | |
| | | factor | | | | ETPFAVPVPGEEVHLRCFRT | |
| | | PLys34--(R)- | | | | ELERPDGSREARFLHTSPEF | |
| | | beta- | | | | AMKRIVAATGRPVFQMARVW | |
| | | lysine ligase | | | | RNGEASNTHAPEFTMLEWYR | |
| | | | | | | PGADLSSLMDETEAFLRALL | |
| | | | | | | PPVVHRGMDVIDLSLPFERL | |
| | | | | | | TMQAAFARYVGADLLGTAGN | |
| | | | | | | AEALAAQAHVGLRNGENWED | |
| | | | | | | LFFRLLLERIEPVIGRERPT | |
| | | | | | | FLTHWPAEQAALARRDPEDG | |
| | | | | | | RAALRFELYVGGLELANAFE | |
| | | | | | | ELTDPVEQRERFATDRKRRV | |
| | | | | | | ELSPDQDWGLDEDFLAALPD | |
| | | | | | | LPPCSGIALGFDRLVMLATG | |
| | | | | | | APRISDILWLA | |
| GO_ | GO_ | hypothetical | H | | Up | MGQVSIRLNGYVYNVGCQDG | 158 |
| 2819 | 2819 | protein | | | | EEAHLYDMARHVEGWLQRAR | |
| | | | | | | TLGGAASESKTLMMAALLMA | |
| | | | | | | DEIFELKRRQISPQAETQIQ | |
| | | | | | | QAEQLLRLEGARQERLARLA | |
| | | | | | | GQAELLAAELERAS | |
| GO_ | GO_ | hypothetical | H | | Up | MKPWRIGRLFRTSLPADHHI | 159 |
| 2828 | 2828 | protein | | | | RQGNAHNSDRNWAQAARAYQ | |
| | | | | | | AALAVDPGLAHIWIQLGHAL | |
| | | | | | | KEQGDLSQAEHAYRQATLLS | |
| | | | | | | PHDPDGWLQLGHLLSLAGNV | |
| | | | | | | RDSITALEEGQRISGDPLFA | |
| | | | | | | AQDIAALRERQKQPQRSWRQ | |
| | | | | | | PEWVTPDITGLWTSEGFCPA | |
| | | | | | | GMELFDPWAYWQINPEVRQM | |
| | | | | | | FDIPVALDLVQHFCTFGVSI | |
| | | | | | | CLPFSLIESFDPDFYRRFCL | |
| | | | | | | NGIAFTDAGAYRHWLTTGIA | |
| | | | | | | QHVPANEKRWIQSLLGDRIT | |
| | | | | | | KLEDVDPLLSSAFRDENAAT | |
| | | | | | | HTQRTLVEGFISDLLTDPQR | |
| | | | | | | PVTPTPRNAPLLHAIACRGE | |
| | | | | | | KGTELHQEGARRLREKIYLH | |
| | | | | | | VPTYRENTRALTRTMTTQGL | |
| | | | | | | DIAAHPLLKDLARHPDEPAE | |
| | | | | | | TLIALANCENRLGSLDAAVT | |
| | | | | | | TLRTATGKKPGRPDLRLCHD | |
| | | | | | | LQEDRNYHHAWNAALALART | |
| | | | | | | GQLEAGQRHLRAYLDSLPFM | |
| | | | | | | LPDHRPLRPRSGAVAIVGKL | |
| | | | | | | NLLQCRLYRVEQRRDHLLAA | |
| | | | | | | GYTVEIFDVDTDLDVFTAKI | |
| | | | | | | AAFESVIFYRLPAWPAVIRA | |
| | | | | | | IHLARSLGLTTFYDIDDPLF | |
| | | | | | | DADLYPEPFETYGGTISRET | |
| | | | | | | YYGLALGVPLFAKALSLCEY | |
| | | | | | | AIASTEPLAEQMRHHHIGEV | |
| | | | | | | FVQPNGLGEAHAIAMRRHGS | |
| | | | | | | QSPSPDQPVTIFYGSNTKAN | |
| | | | | | | RSEIVSVLEPALMRILKKHG | |
| | | | | | | QRVRLCIVGDLPEDSVLRNL | |
| | | | | | | RENITLLPPLPDVQAYWSLL | |
| | | | | | | SSADINLAILGQSPTTDTKS | |
| | | | | | | GIKWLEAAMFGIPSVLSDTA | |
| | | | | | | GYRDVARENETALFATDTDS | |
| | | | | | | WVRALDQLVTDPALRTRIGK | |
| | | | | | | AAYTHALTTYSATPLASHAK | |
| | | | | | | AFMERTAPPDTTARHRILGV | |
| | | | | | | NVFYPPQAIGGATRVFHDNL | |
| | | | | | | SDLSTPEDRRFLFEVFTSQV | |
| | | | | | | EPDSKKLRVYAQDGILVTSI | |
| | | | | | | APLDVDDKDRIAEDPNMVAQ | |
| | | | | | | FRKVVERFRPHLVHFHCIQR | |
| | | | | | | LTAGIIDVLLELDIPYCITL | |
| | | | | | | HDAWWISDRQFVIDELGQPR | |
| | | | | | | LYNYSSPLETLERCGAKAVA | |
| | | | | | | RMESLRDRLFGAKAVLAVSE | |
| | | | | | | AFAELYRTAGVHQVQTIENG | |
| | | | | | | VSVLTPRPRLREESGRIRLG | |
| | | | | | | FIGGLARHKGWDLIQIALRA | |
| | | | | | | GSFHNLELLAIDHAMSPGDE | |
| | | | | | | RTDVFGQTPVVFRGKMSQNE | |
| | | | | | | VADLYASIDVLLAPSIWPES | |
| | | | | | | FGLVTREATLCGCWVVASDR | |
| | | | | | | GAIGDTISDDINGFRIDVSS | |
| | | | | | | AADLRRVLTLIDESPARFRQ | |
| | | | | | | PAPELKISRTARDQAQDLGV | |
| | | | | | | LYEKLLQPGET | |
| GO_ | GO_ | hypothetical | H | | Up | MIFPFKSAPRLRDLARNADT | 160 |
| 2830 | 2830 | protein | | | | ARDAKQWPEAALAYRNLLTR | |
| | | | | | | YPDRVDMWIQYGHALKESGY | |
| | | | | | | LVDAELAYRQAIQRSPTQAE | |
| | | | | | | GYIQLGHALKLQNRREEAAA | |
| | | | | | | AYREALRHDPDATVATVELA | |
| | | | | | | ALGF | |
| GO_ | GO_ | L-lactate | H | | Up | MNHHGIAALHRRADRVLPRI | 161 |
| 2845 | 2845 | dehydrogenase | | | | FRDYVNGGSHSERTVRANRR | |
| | | | | | | AFDRWAVVPKCLVDVSECDL | |
| | | | | | | SGSFLGATHRLPFMFAPLGF | |
| | | | | | | GGLMYPDGEIRAARVAAASG | |
| | | | | | | LPMAVSTFAIQSLETLSRVP | |
| | | | | | | GVTLAAQIYVFRDRGITRDM | |
| | | | | | | LRRAESCGIRNIILTVDTPI | |
| | | | | | | TPLRLRDVRNGFRNLTRPSL | |
| | | | | | | RHVLSMAAHPRWTAGMLRNG | |
| | | | | | | MPKIGNLAPYGMGDNLMEQA | |
| | | | | | | RNAASQIDPTLTWKDLDWLR | |
| | | | | | | SVWPGQLAIKGIMDAGDALA | |
| | | | | | | CQKAGAQTVIVSNHGGRQMD | |
| | | | | | | PAPSSLSVLPDIVEALKGET | |
| | | | | | | DVILDGGVRWGGDVVTALAL | |
| | | | | | | GAKAVGIGRPWAWALAAGGE | |
| | | | | | | RGVRSLVDGLGGEIRDVLRL | |
| | | | | | | GGMVDLASLRAQGAAALRPV | |
| | | | | | | S | |
| GO_ | GO_ | hypothetical | H | | Up | MMMPRFFRCLPLALLVVLTA | 162 |
| 2905 | 2905 | protein | | | | CGNRYEYSRASFNGPLQCAP | |
| | | | | | | YARERTGLKLSGSAASWWGQ | |
| | | | | | | SVGRYAHTHTPRPGEVLVFR | |
| | | | | | | ATSRVPSGHVSIVRRQVSDR | |
| | | | | | | TILVDHANWEPGRIDRAVPV | |
| | | | | | | TDVSARNDWTLVRVWWAPVH | |
| | | | | | | SLGKRAYPTYGFISSHDSDD | |
| | | | | | | SS | |
| GO_ | GO_ | Paraquat- | H | | Up | MSDSRNNRGEPTVSPKEAPV | 163 |
| 2970 | 2970 | inducible | | | | RRTRFSLILLIPVVAILIAG | |
| | | protein B | | | | WLAWEHFATRGPVITITFET | |
| | | | | | | ADGLTPGQTQVKNKAVTLGT | |
| | | | | | | VQDITLSDDMKHVDVTVQMN | |
| | | | | | | ANSAHILTDHTRFWVVRPRI | |
| | | | | | | NGASITGLDTLFSGAYIALD | |
| | | | | | | PGSDDGHYQKFFKGLESPPG | |
| | | | | | | VRSDQPGETFWLVSPSLGSL | |
| | | | | | | GPGSPVFFRDLQVGEVLGYT | |
| | | | | | | MPPGGKGPIVIQAFVKEPYD | |
| | | | | | | HYLRTDSRFWNVSGVQVGLG | |
| | | | | | | AGGLKVQLKSLQALFSGGIA | |
| | | | | | | FGLPERRRNIDLPDAPANSV | |
| | | | | | | FKLYASEADADNARYHKRLR | |
| | | | | | | VVTYINSSVKGLINGSQVTM | |
| | | | | | | FGLQIGTVTDVRLLLEGPTK | |
| | | | | | | LPRVRVDMELEPERMLSNWD | |
| | | | | | | DRIENSKEPPVEKYLQAFVA | |
| | | | | | | DGMRASVQSASFLTGESMIA | |
| | | | | | | LQFVKNAPVTTLTYEGDVAV | |
| | | | | | | LPSQAGGMDGIMESVSTITD | |
| | | | | | | KIAAMPLTEIGGHVNDLLAH | |
| | | | | | | ADGRLNSPEVTQSLAALRDS | |
| | | | | | | LQNLSRLTKTANQNLPALMK | |
| | | | | | | GLQGTLANAQSVLGAYGGDT | |
| | | | | | | DFHRSLQNMITQLTQMSRSL | |
| | | | | | | RFLTDYLDHHPSALITGRRN | |
| GO_ | GO_ | Outer membrane | H | | Up | MIPGFLQSPYGPPSFGAPYG | 164 |
| 300 | 300 | low | | | | TTHALGDWWGAQPWLQKHGL | |
| | | permeability | | | | YVAIDDYESLSGNPIGGKRQ | |
| | | porin, | | | | SETDTGQTAVTLDVDFQRLL | |
| | | OprBfamily | | | | EMGTWSKNFWLHMLVLNGHG | |
| | | | | | | RNLSQIFGDNGNQVQQIYGA | |
| | | | | | | RGNVVAHLVWAYFEKSWLQN | |
| | | | | | | RIDWSVGWIPTGTFFNNSPW | |
| | | | | | | VCSFMNVWMCGNITPTKYLT | |
| | | | | | | GGRDWPSGNIGTVLRLMPTS | |
| | | | | | | HFYIMGGLFAVSPHSYNGGI | |
| | | | | | | SGWAWGQDGLGKLSTEAELG | |
| | | | | | | WIPEFGKDHLIGHYKVGAMY | |
| | | | | | | DNSKYDDLYDDRYGHAWIVS | |
| | | | | | | GLAPRKQSGQISAWVLADQM | |
| | | | | | | LLRHGEGATNGLILAGAYSY | |
| | | | | | | AQGQTSAMNHHLIAALMDTG | |
| | | | | | | HMWGRSLDSIGIAFQWANFS | |
| | | | | | | RSATLAQEAALTAGQPFQSS | |
| | | | | | | NFGTPYGIQGHENIYEFFYT | |
| | | | | | | YHVMTGMTLQPDFQYINHIG | |
| | | | | | | GTTVFKDAVVFSLAFNVSL | |
| GO_ | GO_ | hypothetical | H | | Up | MRALLFLAVLTGSGLALTDV | 165 |
| 3148 | 3148 | protein | | | | SAGAQELRRDGLSAHAVLTD | |
| | | | | | | EVSQSGVDHRMLECRTDPRL | |
| | | | | | | LHTVWDGRPSPHPEPHVCTG | |
| | | | | | | GANRLGAGYVRYLATSRMVK | |
| | | | | | | AHKPLRG | |
| GO_ | GO_ | hypothetical | H | | Up | MPNHPYFRATLMAALAVESA | 166 |
| 3172 | 3172 | protein | | | | TGLSACGPMGPGKLRNDQLE | |
| | | | | | | YSRALGDTQKHEMLLNIVRL | |
| | | | | | | RYADPPTFLDTTQVIAGYSV | |
| | | | | | | SKSISGGFYAYPATAVGNYL | |
| | | | | | | FGTGTMSLGESPTFTYQPVT | |
| | | | | | | GQQYAENVVRPISPTVIMPL | |
| | | | | | | SLGGLPIDTLLRLTAQSIDG | |
| | | | | | | LSNVRGLGAGPSGGGSVRFY | |
| | | | | | | LLLHDLRQLQIQGAMTIRIT | |
| | | | | | | SETPPPPDSKKNGGKSDSNG | |
| | | | | | | NGGSSTGTERSYLVLTSTSD | |
| | | | | | | SNLLAIQAEVRRLLHLDPGA | |
| | | | | | | EEAEIVYGPYPKHPGKQIAI | |
| | | | | | | LTRSMLAMLTQLAYEVEVPE | |
| | | | | | | DDIKSGRTPPTIGQVGIENR | |
| | | | | | | PEVVIHSSREEPDSRYAAVS | |
| | | | | | | YNNTWFWISDRDFQSKLAFT | |
| | | | | | | MVQVLAALAATNHTAGAVVT | |
| | | | | | | IPAG | |
| GO_ | | hypothetical | H | | Up | MKIAFIYIAEPYQCYHTASV | 167 |
| 3238GO_ | | protein | | | | ASALAAIPGHDVVEYYSFPE | |
| 3238 | | | | | | TVEHLSRIRQALDVPALPLK | |
| | | | | | | AFPKSLKARLLKRARRLDQE | |
| | | | | | | RLVVLRENIAELNRYDAVVA | |
| | | | | | | TEYTAGVLKEMGLSSPKLIL | |
| | | | | | | LMHGAGDRYVNDEHLVREFD | |
| | | | | | | LTLVPGPKVEGYFQDRGLLR | |
| | | | | | | PETTRVVGYPKFDVFEAVQR | |
| | | | | | | EKTISFANGRPFALYNPHYQ | |
| | | | | | | RKLTSASGWMMPLIRGFKAQ | |
| | | | | | | SDYNLVVAPHIKTFHRGFGI | |
| | | | | | | RERQLKRQRSPEVMVDTGSS | |
| | | | | | | AMLDMTYTSQAALYIGDVSS | |
| | | | | | | QVYEFLGIPRPCVFLNPRKL | |
| | | | | | | PWQDDPYFLHWTLGEVVEDL | |
| | | | | | | DDLMPAIARAQERHALYRPA | |
| | | | | | | QEKLFRETFGEPLLGASQRA | |
| | | | | | | ADAIAQFMSVA | |
| GO_ | GO_ | Putative | H | | Up | MTTKPAGTPLSVPDDATVAS | 168 |
| 3262 | 3262 | hemolysin | | | | VSTPASTPAPHTLASSEETL | |
| | | | | | | RQMETLSTLSLNREGGFQEL | |
| | | | | | | RGGTLGVRIAETAEERDAAQ | |
| | | | | | | ALRYRVFFEELGARPDERAF | |
| | | | | | | RTKRDVDEFDEAADHLLVID | |
| | | | | | | HAKGPGAAGVVGTYRLLRSD | |
| | | | | | | AAEKIGRFYTSSEYDISTLT | |
| | | | | | | EFPGRLLEVGRSCVAKEYRG | |
| | | | | | | RSAMQLLWRGIASYIFLHRI | |
| | | | | | | DVLFGCGSLPGTDPDALADQ | |
| | | | | | | LTYMHHNHLAPPALRIRALP | |
| | | | | | | DRYVEMQRTDPHVLDYRACL | |
| | | | | | | NKLPPLIKGYLRLGGYVGDG | |
| | | | | | | AVVDEQFNTTDVAVLVKSEL | |
| | | | | | | LADKYYRHYERRLRDALD | |
| GO_ | GO_ | Acetyltransferase | H | | Up | MLGREIRTARLVLTPVNWPD | 169 |
| 3278 | 3278 | | | | | LEDMVALKGDAGAFARMLGG | |
| | | | | | | VRNRTTTEEEMAEDVSFWAR | |
| | | | | | | RGVGIFAIRENGRFVGITGV | |
| | | | | | | HERPDGRGLGLRFALFPWAA | |
| | | | | | | GRGIAREAAAAALRYVLDCG | |
| | | | | | | EKRIVAVAREDNLASRTVLG | |
| | | | | | | SLGLHHTQTFDRNGDTMLLY | |
| | | | | | | EITAD | |
| GO_ | GO_ | Biotin carboxyl | H | | Up | MIPDLKILDALMARMQALGI | 170 |
| 350 | 350 | carrier | | | | TELDYSRDGEHIRLVRDAQD | |
| | | protein of | | | | SSPQPTSPAPAAAASTLPVF | |
| | | acetyl-CoA | | | | ETASTPPKAETTIDAPMHGQ | |
| | | carboxylase | | | | FYASPTPDAPPFVKPGDIVA | |
| | | | | | | EGQPLYILEVMKTLSRIEAE | |
| | | | | | | FPCRIVAVLAANADAVSPGT | |
| | | | | | | PLFTVEPLDA | |
| GO_ | GO_ | DNA polymerase I | H | | Up | MPPEKPDLGKADLEKPHLVL | 171 |
| 373 | 373 | (EC 2.7.7.7) | | | | IDGSGFIFRAFHALPPMSSP | |
| | | | | | | QGVPVNAVYGFTNMLARLLR | |
| | | | | | | DHVGTHLAVIFDAGRTTFRN | |
| | | | | | | EIYPQYKAHRPEAPEDLRPQ | |
| | | | | | | FGLIRDATAAFNVPSIELAG | |
| | | | | | | WEADDLLAAYAKAAVEAGGC | |
| | | | | | | CTIISSDKDLMQLVRPGVEL | |
| | | | | | | MDPMKQKPIREAEVEAKFGV | |
| | | | | | | RPDQVVDVQALMGDSTDNVP | |
| | | | | | | GVPGIGPKGAAQLVNEYGTL | |
| | | | | | | EQILEAAPGMKASKRRDNLI | |
| | | | | | | EHADAARMSRRLVLLDDNAP | |
| | | | | | | MPQPISELGCREPVRETLRD | |
| | | | | | | WLEEMGFHSTIQRMGLGLAA | |
| | | | | | | KPRPFTRQIVRPEDKAAARD | |
| | | | | | | EVAAVTIPDAPYGPYETVTD | |
| | | | | | | MDALDRWIADARTAGVVAVD | |
| | | | | | | TETDSLNARQANMVGLSLSV | |
| | | | | | | APGKACYVPFLHETIRDLLE | |
| | | | | | | EDSTGEAFVRQLDRTEALER | |
| | | | | | | LKPLLQDASVLKVFQNAKYD | |
| | | | | | | LTVFRGAGIPEISPIDDTML | |
| | | | | | | ISYAQSAGEHGQGMDELSEL | |
| | | | | | | HLGHTPVTYDSVTGTGRKRI | |
| | | | | | | PFAQVAIDTATAYAAEDADV | |
| | | | | | | TLRLWQVLRPQLRTRHALAL | |
| | | | | | | YEEIERPLIQILTDMEEVGI | |
| | | | | | | KVDATELRRMSADFAERMAT | |
| | | | | | | IEQEIHEQVGRSFNVGSPKQ | |
| | | | | | | LGEILFDEMGLPGGKRTKSG | |
| | | | | | | SWGTDSGVLESLAEQGHELP | |
| | | | | | | QKILSWRQLAKLKSTYADAL | |
| | | | | | | VQQMDQDTQRVHTSFQMAIT | |
| | | | | | | TTGRLSSNEPNLQNIPIRTE | |
| | | | | | | EGARIRKAFVAAPGCVLLSA | |
| | | | | | | DYSQIELRLLAHVAKIEPLL | |
| | | | | | | EAFRLGQDIHARTASEVFGI | |
| | | | | | | PLEGMDPLTRRRAKAINFGI | |
| | | | | | | IYGISAFGLAQQLQISPGEA | |
| | | | | | | RSYIDAYFARYPGIRAYMER | |
| | | | | | | TKEEAKRHGYVTTPFGRRCY | |
| | | | | | | VPGITEKNGARRAYAERQAI | |
| | | | | | | NAPLQGGAADIIKRAMVHLA | |
| | | | | | | RRLPAMGLKAKMVLQVHDEL | |
| | | | | | | LFEVEEQDAKALADFVRTEM | |
| | | | | | | EGAARLDVALEVETGIGPSW | |
| | | | | | | ADAH | |
| GO_ | GO_ | ABC transporter, | H | | Up | MKRGAALLAASLLLGAGAFP | 172 |
| 375 | 375 | substrate-binding | | | | ALAQEQTPPDWNGITLGSPH | |
| | | protein | | | | RGGTLHLTADGPGGTLDPQI | |
| | | (cluster 5, | | | | NYGTQYMQVFVNMYDPLLTF | |
| | | nickel/ | | | | RLARGKAGLEVVPDLADAMP | |
| | | peptides/opines) | | | | QISPDGLTWRLHLRSGLHFS | |
| | | | | | | DGAPVRVEDVVASFRRIYRA | |
| | | | | | | GSPTAASFYGGIAGAGECLE | |
| | | | | | | TPDRCTLSGVEGHPETGEIV | |
| | | | | | | FHLTKPDGEFLYKLAFPHAV | |
| | | | | | | ILPASTPVHDMGGTTVPGTG | |
| | | | | | | PYRITQYDPGHGMVLERNPY | |
| | | | | | | FHVWNPQAQTDGFVDRIEYD | |
| | | | | | | FGLSDEAQVTAVEQGRYDWM | |
| | | | | | | LDAKPADRLGELGANYTSQV | |
| | | | | | | HIEPLLGLYYLAMNTHEKPF | |
| | | | | | | TDVRVRRAVSMAVNRHAMTI | |
| | | | | | | LFGGSAISEPLCQMVPHGIP | |
| | | | | | | GADLGLDCPQDMEGARRLIH | |
| | | | | | | EADADGASVTLVVPNRAMEL | |
| | | | | | | GMGTYLRNALQSAGLNVQLR | |
| | | | | | | PITAGLAESYEQNTANHVQI | |
| | | | | | | ALSYWFADYPSASTFLDDLF | |
| | | | | | | GCDNYHPNSAVSPNYTGFCD | |
| | | | | | | QHVQSLFDQAKAQTDPAKAA | |
| | | | | | | PIWQEAGHVIMEQMPGAPMI | |
| | | | | | | QMRTVDFVSKRLGNYYATLL | |
| | | | | | | SHMLLSHVWVQ | |
| GO_ | GO_ | N-acetylmuramoy1- | H | | Up | MIAPSFTAGHWGQGMLTRRT | 173 |
| 382 | 382 | L-alanine | | | | LVSGLSGLPLLSPVAAFGRH | |
| | | amidase | | | | PLHKKLPAPAVHRGAVPPKP | |
| | | (EC 3.5.1.28) | | | | LVMLDPGHGGKDPGAIGISG | |
| | | | | | | TYEKHIAEAAASELSRQLLA | |
| | | | | | | SGQYRVAMTRSEDRFIPLEG | |
| | | | | | | RVELAQRHQAHLFISMHADA | |
| | | | | | | LHDRDVRGASVYTLSSGASD | |
| | | | | | | AQTASLARTENSADRFAGPA | |
| | | | | | | FHGMSPDVQQILASLVSEET | |
| | | | | | | RRGSAHMAASVVNSFRNRIG | |
| | | | | | | LLTHPSRHAAFVVLKSSEIP | |
| | | | | | | SVLVEMGFMSNRLDEAALRQ | |
| | | | | | | AVHRTQVASAMKTAIDRYFA | |
| | | | | | | THAGVMTG | |
| GO_ | GO_ | Outer membrane | H | | Up | MRLRTALLAMTSMVAAPSLA | 174 |
| 441 | 441 | protein | | | | MASTITGPYVNIGGGYNLVQ | |
| | | | | | | QQHGSFSPTTQADGTSSNAA | |
| | | | | | | SSSRYRHHDGFTGFGAFGWG | |
| | | | | | | FGNGLRVEVEGLYNYSQINH | |
| | | | | | | RRPTAVNGMTHGSDQAYGGL | |
| | | | | | | VNVLYDIDLANFGLNVPVTP | |
| | | | | | | FVGVGAGYLWQHYNPTTTNY | |
| | | | | | | VNGAVDRMGGTQGSFAYQGI | |
| | | | | | | VGAAYDIPNVPGLAVTTEYR | |
| | | | | | | FIGQDFVNGPYRSTAYNASG | |
| | | | | | | VHKGNVNFDQRFSHQFILGL | |
| | | | | | | RYAFDTAPPPPPPAPVVVPP | |
| | | | | | | APTPARTYLVFFDWDKSDLT | |
| | | | | | | GRAREIVAQAAQASTHVQTT | |
| | | | | | | RIEVNGYTDNSAAHPGPRGE | |
| | | | | | | KYNLGLSNRRASSVKAELIR | |
| | | | | | | DGVPAAAIDIHGYGESKPLV | |
| | | | | | | PTGPNTREPQNRRVEIILK | |
| GO_ | GO_ | ABC transporter, | H | | Up | MKTVFILLTVLAVGFPASHL | 175 |
| 541 | 541 | substrate-binding | | | | PGTARAADARDTLSIGLAIE | |
| | | protein | | | | PPGLDPTRGSSEAIGMVTYG | |
| | | (cluster 5, | | | | NIYEGLMRLDEQGALTPLLA | |
| | | nickel/ | | | | QDWHISPDGLTYDFTLHDGV | |
| | | peptides/opines) | | | | RFHDGTPLTCDSVKFSLLRA | |
| | | | | | | GAADSTNPHRAIFSQITNIS | |
| | | | | | | CPDSQHVSIRLQHPYGSFLY | |
| | | | | | | QLAWNDAAILSPASVDQNIS | |
| | | | | | | HPVGTGPFMFGEWRRGDHLT | |
| | | | | | | LTRNPDYWGASPALRSVTFR | |
| | | | | | | FMPDPLSASNALLAGELDAY | |
| | | | | | | PSFPSREILSRFTGNTQLQV | |
| | | | | | | VRGSFPFKAILALNNARRPF | |
| | | | | | | SDIRVRQAIAQAIDRKALIQ | |
| | | | | | | AVADGDGTLLQSHIAPDDPD | |
| | | | | | | YVPLPDRYPYDPEHARALLK | |
| | | | | | | EAGVAPGMHLTLTFPPIGYA | |
| | | | | | | RDSSELIAAYLEQVGLIVTL | |
| | | | | | | QPVEWPTWLGQVYGQGQFDM | |
| | | | | | | TVIAHTEPHDIAIYDRTPVY | |
| | | | | | | FHYHSPVFHGLAEQYEATAD | |
| | | | | | | TVRRHQLSVQMQEQLAQDEP | |
| | | | | | | NVFLFAIPRETVMNRRLKGL | |
| | | | | | | WTNQPIAGCPVAGVSWAP | |
| GO_ | GO_ | ABC transporter, | H | | Up | MSSLLHVRNLGVQSPDRPIL | 176 |
| 544 | 544 | ATP-binding | | | | HDVSFTLEPGQILAVTGESG | |
| | | protein | | | | SGKSTLALSLMGLLPPGFQA | |
| | | (cluster 5, | | | | HGSIRLEDTELSTLSEQDWC | |
| | | nickel/ | | | | RIRGKRLGMVFQEPMTALNP | |
| | | peptides/opines) | | | | TRRIGTQIGDTFRLHTTLSR | |
| | | | | | | HEIDTEMRMLLEQVGLSEAG | |
| | | | | | | VSERAYPHQLSGGQRQRVLL | |
| | | | | | | AMALACQPELLIADEFTTPL | |
| | | | | | | DARTQATMMALVTRRCETQG | |
| | | | | | | MAVLMISHDLGLVRHHADHV | |
| | | | | | | LVMKDGACVEQGATASVFDA | |
| | | | | | | PRHPYTQALLAMSLHGAART | |
| | | | | | | PLTPLPIFTAPS | |
| GO_ | GO_ | 5- | H | | Up | MKTLLPTSTAGSLPKPSWLA | 177 |
| 584 | 584 | methyltetra | | | | KPETLWSPWKLQGEELVEGK | |
| | | hydropteroyl | | | | QDALRLTLDDQDRAGIDIVS | |
| | | triglutamate-- | | | | DGEQTRQHFVTTFIEHLGGV | |
| | | homocysteine | | | | DFEKRQTVKIRNRYDASVPS | |
| | | methyltransferase | | | | VVGAVTREKPVFVEDAKYLR | |
| | | (EC 2.1.1.14) | | | | TLTKKPIKWALPGPMTMIDT | |
| | | | | | | LYDGHYKSREKLAWEFAKIL | |
| | | | | | | NQEARELEAAGVDIIQFDEP | |
| | | | | | | AFNVFFDEVNDWGIATLEKA | |
| | | | | | | VEGLKCETAVHICYGYGIKA | |
| | | | | | | NIDWKNALGAEWRQYEEIFP | |
| | | | | | | KLQKSSIDLISLECONSRVP | |
| | | | | | | MDLIELVRGKKVMVGAIDVA | |
| | | | | | | TNTIETPEDVASTLRKALKF | |
| | | | | | | VDADKLYPSTNCGMAPLSRR | |
| | | | | | | VARGKLNALGAGAEIVRKEL | |
| | | | | | | SA | |
| GO_ | GO_ | hypothetical | H | | Up | MSVEFTFNIKKIPFNEDYTP | 178 |
| 585 | 585 | protein | | | | SDSTRLTTNFANLARGEHRQ | |
| | | | | | | ENLRNVLCMIDNRFNSLAHW | |
| | | | | | | DNPNGDRYSVGVDIISAEMT | |
| | | | | | | IRSEGKNESFPLIEVLNTSI | |
| | | | | | | FDKKDDKRIPGIVGNNFSSY | |
| | | | | | | VRDYDFSVLLLKHNKDQSEF | |
| | | | | | | RTPDRFGDLHGNLFKCFINS | |
| | | | | | | ECYKTHFRKMPVICLSVSSN | |
| | | | | | | RTYYRTRNHHPVLGVEYEQR | |
| | | | | | | EFSLTDQYFGKMGMKVRYFM | |
| | | | | | | PENASAPLAFYFFGDLLFDY | |
| | | | | | | SNMELISTISTMESFQKIYR | |
| | | | | | | PEIYNSNSPAADCYQPSLKH | |
| | | | | | | QDHSVTRIIYDREERSQLAI | |
| | | | | | | AQGRFTEESFIKPYQDVLEQ | |
| | | | | | | WSAHYSV | |
| GO_ | GO_ | probable | H | | Up | MPRPVFPFRTTVRTLSLIRA | 179 |
| 634 | 634 | transglycosylase | | | | GAAFGLVGLLAACAGNNADM | |
| | | | | | | GGGSELPVSQEAANYRAHAK | |
| | | | | | | SYYAPPGPPSDPWGPYIQEA | |
| | | | | | | SNRFDVPEAWIRAVMQQESG | |
| | | | | | | GHLFDHNGNFITSVPGAMGL | |
| | | | | | | LQLMPPTYDDMRQQYGLGED | |
| | | | | | | PYDPHDNILAGTAYLRQMYD | |
| | | | | | | IYGSPGFLAAYNDGPGSLDR | |
| | | | | | | YLRRGRALPRETRRYVAAIG | |
| | | | | | | PHIAGITPHNRSAADLLVAQ | |
| | | | | | | HDPNSQVMLAQNTPSADIAP | |
| | | | | | | VTSASERQAVSAAWNHRETA | |
| | | | | | | DTSSDDSDSDTPAATPQTAP | |
| | | | | | | VQVASAAGSEAPASISAAWA | |
| | | | | | | ARGFTPTPARPAVRQASVPD | |
| | | | | | | DEVADNRVTAQEIPIGHHLQ | |
| | | | | | | PQPIMAVMPASRVQPRISLP | |
| | | | | | | AISTSSRAATGNWAIQVGAF | |
| | | | | | | ANAKQASIATSAAHSKGGVV | |
| | | | | | | VASARSQVESVKGGRSHLYR | |
| | | | | | | ARLTGLTHENAVAACRRLSH | |
| | | | | | | GSPCVVVPPGAY | |
| GO_ | GO_ | Queuine tRNA- | H | | Up | MSDHKTDHATKMTWTPEATC | 180 |
| 657 | 657 | ribosyltransferase | | | | GMARAGHLHTRHGTVPTPTF | |
| | | (EC 2.4.2.29) | | | | MPVGTVGTVKGMTMDAVRST | |
| | | | | | | GAGIVLGNTYHLMLRPTADR | |
| | | | | | | VQKLGGLHRMMDWPGPILTD | |
| | | | | | | SGGFQVMSLGALRKLDEDGV | |
| | | | | | | TFRSHIDGSKHRLTPEISTD | |
| | | | | | | IQFKLDATITMAFDECPALP | |
| | | | | | | ATPEVLRKSMEMSMRWAARS | |
| | | | | | | REAFVAREGYGQFGIVQGGT | |
| | | | | | | ERDLREYSIKALTDIGFEGY | |
| | | | | | | AIGGLAVGEGQELMFSTLDF | |
| | | | | | | TTPMLPQDKARYLMGVGTPD | |
| | | | | | | DLLGAVMRGVDMFDCVMPSR | |
| | | | | | | AGRTARAYTERGTINLRNAR | |
| | | | | | | FADDTRPLTPHDDTPVAGRY | |
| | | | | | | SRAYLHHLFRANEMLGPMLL | |
| | | | | | | TWHNLAYYQRLMRQIRAAIV | |
| | | | | | | DGTLDALAVKLRADWAKGDW | |
| | | | | | | TEDEWPMPDLTL | |
| GO_ | GO_ | Heparinase II/III | H | | Up | MTGSRWLQGLRLSLARLPFG | 181 |
| 68 | 68 | family protein | | | | GPASEGPVHAFRDPWKGDPD | |
| | | | | | | QGARLIGGHFRFDRQDYPLP | |
| | | | | | | NGNWERGPWPEPVREWLHGF | |
| | | | | | | SWLRDLRTLGSDRARLTARG | |
| | | | | | | LVSDWLAHPPTDPLVRDACV | |
| | | | | | | TGSRLAAWLSNHEFCLASAD | |
| | | | | | | PRLQQRLMERMLVEGRTIAA | |
| | | | | | | LLPLPPQGVRGLMAFRGLLA | |
| | | | | | | AAMAMPEQTGFMSRFLRYLP | |
| | | | | | | GELERLVLADGTVAERSPEA | |
| | | | | | | QFLVTRELAEMSVMFRTAHA | |
| | | | | | | SVPPFIDRALDRVCPVLRAM | |
| | | | | | | RHGDGGLAVFNGTNERHSAA | |
| | | | | | | VEDVLAQGSRQKLIAPAMPQ | |
| | | | | | | GKFTRLALGKSLLLVDSGPP | |
| | | | | | | APAGFDSMAHAGTLSFEFSH | |
| | | | | | | QRHRLFVNCGSAVVGAWRDA | |
| | | | | | | MRCSAAHTVLVADGLSSADF | |
| | | | | | | GPDGGMTRRPVTVSCDHQTD | |
| | | | | | | GAAHWLDLSHDGYHAPLGAK | |
| | | | | | | WTRRLYFGSDGEDLRGQEII | |
| | | | | | | DGERNIDFAIRFHIHPDVKV | |
| | | | | | | TQDDEDIILQVGGTIWRFRQ | |
| | | | | | | RDGVVRLENDVYLGRGKREI | |
| | | | | | | CQQIIITPRALPDAAPQEGE | |
| | | | | | | AAPADEKTDAGKAVRRTHQS | |
| | | | | | | VTWLLERIPE | |
| GO_ | GO_ | None (90 bp | H | | Up | MKHIRADKSNLSDIVTVAAG | 182 |
| 721 | 721 | upstream | | | | HPFRGKIEPIEGAETAVVQM | |
| | | from hypothetical | | | | RDTSPSGMDWTSCVRTEVAG | |
| | | protein GO_ | | | | RREPDWLRPGDILFPARGNV | |
| | | 721) | | | | SLAVLINESIGSLQAVAAPH | |
| | | | | | | FFLLRVSRSDVLPAYMAWWL | |
| | | | | | | NQEPAQRHLEQQAQSSTLVR | |
| | | | | | | NIARPVLEDTPVILPPLPRQ | |
| | | | | | | RQIVELANAMQREEDLLRRL | |
| | | | | | | RQTNQQIMTGLARDLLAG | |
| GO_ | GO_ | Two-component | H | | Up | MAADSLSRHLADRKTPWPHF | 183 |
| 78 | 78 | system sensor | | | | NQYLVAYLSYLLFYPVPWLL | |
| | | histidine | | | | GYHRPTLAGVLFSTAAMLVF | |
| | | kinase | | | | LLVYFAPYRKDRYYGYGEII | |
| | | | | | | LTDLIGYACAWTHGDWEVYC | |
| | | | | | | IFAAGMCARLPGKRQSVTMV | |
| | | | | | | ILLQVVLIISGHFRHKTTLE | |
| | | | | | | VIPGAFFSIITYMGTLVQWQ | |
| | | | | | | LGIRNFELREAQNEIRTLAT | |
| | | | | | | TAERERIARDLHDLLGHSLT | |
| | | | | | | VISVKAELAERLFTSDTSRA | |
| | | | | | | RHEVTEISQIARTSLREVRE | |
| | | | | | | AVSGMNGASLQRELDRARKA | |
| | | | | | | LNTAGITLVLNGPGPSTDQP | |
| | | | | | | QNSVLALALREAVTNVIRHS | |
| | | | | | | DARTCTLTFQHTAEGHIHTF | |
| | | | | | | TLEDDGPLQTTQSAPAIIEG | |
| | | | | | | NGLRGMRARLAASGGTLTVS | |
| | | | | | | PRPQGFKLTATTLP | |
| GO_ | hfq | RNA-binding | H | | Up | MTSEPSSSAGSRAVQEVFLD | 184 |
| 175 | | protein | | | | HLRRTEASVTVFLVNGVKLQ | |
| | | Hfq | | | | GIVAQSDAHTLLLRRDGHVQ | |
| | | | | | | LVYKHAVSTIMPVAAMSHFV | |
| | | | | | | EEEL | |
| GO_ | hisH | Imidazole | H | | Up | MRVVVIDYNGGNLASAAQAA | 185 |
| 159 | | glycerol | | | | RKAAIRKGIEADVVISRDAS | |
| | | phosphate | | | | DILNADRLILPGQGAFADCA | |
| | | synthaseamido | | | | QGLGPELRNMLETATANGTP | |
| | | transferase | | | | FLGICVGMQLMCEYGLEHGR | |
| | | subunit (EC | | | | TEGLGWISGNIRRMDEAANA | |
| | | 2.4.2. ) | | | | GLRLPHMGWNTLDFTPGAHL | |
| | | | | | | LTDGLTPGNHGYFVHSYALH | |
| | | | | | | DGTDSDLVATAQYGTQVPAI | |
| | | | | | | VARGNRCGTQFHVEKSQDVG | |
| | | | | | | LTILGNFLRWTS | |
| GO_ | hpnA | Hopanoid- | H | | Up | MNDVTLVTGATGFVGSAVAR | 186 |
| 2110 | | associated | | | | VLEERGHRLRLLVRPTSDRS | |
| | | sugar epimerase | | | | NIAELNAELAVGDLSDPDTL | |
| | | HpnA | | | | APALKGVKILFHVAADYRLW | |
| | | | | | | VPDPETMMKANVEGTRNLML | |
| | | | | | | AALEAGVEKIIYCSSVAALG | |
| | | | | | | LRSDGVPADETTPVSESQVI | |
| | | | | | | GIYKLSKYRAEQEVLRLVRE | |
| | | | | | | KNLPAIIVNPSTPVGPRDIK | |
| | | | | | | PTPTGQMILDCASGNMPAYV | |
| | | | | | | ETGLNIVHVDDVAEGHALAL | |
| | | | | | | ERGKIGEKYILGGENIMLGD | |
| | | | | | | LFRMVSQIAGVKPPSVKLKQ | |
| | | | | | | SWLYPVALVSEWLARGFGIE | |
| | | | | | | PRVTRETLAMSKKLMFFSSD | |
| | | | | | | KAKKELGYAPRPARDAVTDA | |
| | | | | | | IVWFRQHGRMK | |
| GO_ | hpnN | Hopanoid- | H | | Up | MSGWGRETLPVLLRVGPMFS | 187 |
| 2346 | | associated | | | | DLTGRLNAVCARRAPLVLAL | |
| | | RND transporter, | | | | FALLCAGCVALSVTRISVTT | |
| | | HpnN | | | | DTSKMFANTLPWKKRSMELT | |
| | | | | | | RLFPQDSNQLVAIIDSRIPE | |
| | | | | | | QGRMAARQLAATLSQDHTHF | |
| | | | | | | KTVSLPGDNAFYNSHGFLFL | |
| | | | | | | DTKDLEPLLDSIVSAQPFLG | |
| | | | | | | TLAADPSARGLFGALGLIGE | |
| | | | | | | GIKAGQGIPSGFDGALDGFA | |
| | | | | | | NSLTAAANGHPQDMSWQNLL | |
| | | | | | | IGKLAELGSQYEFVVTQPKL | |
| | | | | | | DYSSFQPGEGATTAMRQAIN | |
| | | | | | | NLEFVKTKQASGIITGEVKL | |
| | | | | | | SDEEFSTVAHGMVLGLVISL | |
| | | | | | | VLVAVWLILAVHSPRVIIPI | |
| | | | | | | LLTLISGLLLTTGFAALAVG | |
| | | | | | | ELNLISVAFAILFVGIAVDF | |
| | | | | | | AIQYSVRLRGQRNPDGSHPS | |
| | | | | | | LHDAIILTGQESGAQILVAA | |
| | | | | | | LATAAGFLAFYPTSFIGVAQ | |
| | | | | | | LGLIAGFGMLIAFFCTMTLL | |
| | | | | | | PALLSIFHAKLGNGTPGFAF | |
| | | | | | | MAPADAFLRHKRHKVVGVFA | |
| | | | | | | LLGIVGLALMPLLKFDADPL | |
| | | | | | | HTKDPNTEGMRALHLLEANP | |
| | | | | | | LTTPYSAQVLTPNLTEAARQ | |
| | | | | | | AAAFSKLPSVHDVLWLGALV | |
| | | | | | | PDDQKTKLAMIEDTASILLP | |
| | | | | | | TITIANPAPAPDAAAIRAAA | |
| | | | | | | VTAAQKLDAVKDQLPAPLEK | |
| | | | | | | IRQALHTLSTAQDSTLLQAS | |
| | | | | | | TSLTRFLPDELSMLRTALQP | |
| | | | | | | TPVTMESIPQDVRQDYVLPD | |
| | | | | | | GRARLTIHPKGQMSETPVLH | |
| | | | | | | RFVRELRSVNPDVAGPAMEI | |
| | | | | | | TESANTIVHAFTVAAICALI | |
| | | | | | | MIAIILLVVLRRLLDAALVM | |
| | | | | | | APLLMSALLTVILVVTVPET | |
| | | | | | | LNYANIIALPLLLGVGVSFN | |
| | | | | | | IYFVMNWREGVKGPLTSPTA | |
| | | | | | | RAVLFSALTTATAFGSLARS | |
| | | | | | | GHPGTASMGRLLLMSLGCTL | |
| | | | | | | LCTMVFVPALLPKRPIDEA | |
| GO_ | lexA | SOS-response | H | | Up | MAFILHLVFRPASAEAHGSA | 188 |
| 2087 | | repressor and | | | | CMLTRKQHOLLLYIDDHLRR | |
| | | protease LexA | | | | TGYSPSFDEMKDALELRSKS | |
| | | (EC 3.4.21.88) | | | | GIHRLISALEERGFLRRHHH | |
| | | | | | | RARALEVLRLPHMGTEAPVA | |
| | | | | | | TGTGTAFVPAVLNQGQTGLQ | |
| | | | | | | GAFSEDSVANDRQTVSIPLY | |
| | | | | | | GRIAAGLPIEAMQDDSDRID | |
| | | | | | | VPVSLLGTGEHYALTVAGDS | |
| | | | | | | MIEAGILDGDIAIIRRRETA | |
| | | | | | | ENGQIIVALIDEQEVTLKKL | |
| | | | | | | RRRGSMIALEAANRDYETRI | |
| | | | | | | FPAERVHIQGRLVALFRQY | |
| GO_ | 1ptG | Lipopolysaccharide | H | | Up | MNDSTHAMPRHVLIKALNRA | 189 |
| 1655 | | export | | | | LLGRFLLCGAVLVSLLEILA | |
| | | system | | | | LLEKTTPILNRHLGVRGILT | |
| | | permease | | | | FAFLHLPALSIDILPLAFMV | |
| | | proteinLptG | | | | GALFLLTQMTLSSEISALRA | |
| | | | | | | AGLSTPALYRLLLPAVLIAG | |
| | | | | | | IGGTCAQYWLVPACENALTT | |
| | | | | | | WWNRTDPLAGQDGQPEDNIL | |
| | | | | | | WFRAGPTLVRIGQIAQGGTF | |
| | | | | | | LRDVTIYHRDSTGLLTGTEH | |
| | | | | | | TNVLTYRDGQWHPDGAQDLS | |
| | | | | | | LSDDKSYVNVTKGDSTFVIP | |
| | | | | | | ATPSVIMTLSQNGATVTPGQ | |
| | | | | | | VSAILHKGAPASLPRATYRM | |
| | | | | | | ALFSGMILPVEIAVMLLLTL | |
| | | | | | | PVIYIPPRAGLRNPLPVYVL | |
| | | | | | | AAGMGFVILQGMISALGNAG | |
| | | | | | | TLPAPLAVSVGPLLGTLLGL | |
| | | | | | | TWLLRMEER | |
| GO_ | LuxR | Two-component | H | | Up | MRILLVEDDPTVRAFVLKGL | 190 |
| 3200 | | transcriptional | | | | REAGHVVDEADNGKDGLFLA | |
| | | response | | | | VSENYDVVILDRMLPGGIDG | |
| | | regulator, LuxR | | | | LRILETLRGQKNATPVLLLS | |
| | | family | | | | ALADVDERVAGLKAGGDDYM | |
| | | | | | | TKPFAFSELLARVEALGRRG | |
| | | | | | | RPESAPQTRLVVGDLEMDLL | |
| | | | | | | SRTVKRGGEKIDLQPREFRL | |
| | | | | | | LEFLIRHAGQVVTRTMLLER | |
| | | | | | | VWDYHFDPQTNVIDVHVSRL | |
| | | | | | | RQKVDKPFDKPMIHTIRNAG | |
| | | | | | | YILRAD | |
| GO_ | metR | Transcriptional | H | | Up | MSVLQRNHLMIIQEVAREGS | 191 |
| 581 | | activator MetR | | | | LTAASARLNLTQPALSHAVR | |
| | | | | | | KIEMQLGVKIWRREGRSLIL | |
| | | | | | | TQAGQWLLSLANRLLPQFSL | |
| | | | | | | AEERLEQFANGERGTLRIGM | |
| | | | | | | ECHPCYQWLLNVVSPYLERW | |
| | | | | | | PKVDVDVRQKFQFGGIGAIL | |
| | | | | | | SNEIDILVTPDPLYKPGLNF | |
| | | | | | | TPVFDYELVLVVGSGHRLYG | |
| | | | | | | VDRVTPEQLADETLITYPVP | |
| | | | | | | SERLDIYTQFLQPAGIAPRQ | |
| | | | | | | QKQIETTDIMLVMVAHGRGV | |
| | | | | | | AALPRWLVDEYASRFDLHSV | |
| | | | | | | RLGENGIAKQIFLGRRETDA | |
| | | | | | | DVQYLTDFIAFAADPKD | |
| GO_ | metX | Homoserine O- | H | | Up | MRVDQRLIAEMIAPRARVLD | 192 |
| 1750 | | acetyltransferase | | | | VGSGDGTLIDYLYRTRSCDA | |
| | | (EC | | | | RGIEIDMQNVTQSVAHGLPV | |
| | | 2.3.1.31) | | | | MHGDADHDLADYPDDTFDYV | |
| | | | | | | VLQRTLQAVERPREVLRQML | |
| | | | | | | RIGRHAIVSFPNFGHWRLRL | |
| | | | | | | QLLTTGRMPMTPVWNTPWYS | |
| | | | | | | TPNIHPCTIRDFLLLCEEEG | |
| | | | | | | YVIQQWLAIDEDGARAPWRR | |
| | | | | | | SIRLANLFGEQAMFLLKRAG | |
| | | | | | | H | |
| GO_ | minC | Septum site- | H | | Up | MPDPVSATTSNTPMRIRARG | 193 |
| 2642 | | determining | | | | RSFLALVLSPEAPLPLWLEG | |
| | | protein | | | | LDYQIARSGGLFTGKPVILD | |
| | | MinC | | | | LGLLSETTPGLATLLDDIRR | |
| | | | | | | RGVRIIGIEGGSRHWSAVAH | |
| | | | | | | WDWPETLDGGRPAGEVEIPE | |
| | | | | | | DPSASAAGPVSGGGTLIIEQ | |
| | | | | | | TVRSGQSIQHMQGDVIILGS | |
| | | | | | | VSSGAEIVAAGSIHVYGTLR | |
| | | | | | | GRAIAGVGGQSQSRIFASRM | |
| | | | | | | LAELLALDGYYAVTEDIDPA | |
| | | | | | | ILGQAAQATLDEDRVRVLPL | |
| | | | | | | TT | |
| GO_ | minD | Septum site- | H | | Up | MAKVLVVTSGKGGVGKTTST | 194 |
| 2641 | | determining | | | | AALGAALAQSGQNVVVVDFD | |
| | | protein | | | | VGLRNLDLVMGAERRVVFDL | |
| | | MinD | | | | INVVQGDARLSQALIRDKRC | |
| | | | | | | ETLSILPASQTRDKDALTSE | |
| | | | | | | GVARVMDELREKFDWVICDS | |
| | | | | | | PAGIERGAQLAMYHADMAVI | |
| | | | | | | VTNPEVSSVRDSDRIIGLLD | |
| | | | | | | SKTKKAEQGEKVDKHLLLTR | |
| | | | | | | YDPARAARKEMLSVEDVLEI | |
| | | | | | | LSIPLLGIVPESEDVLKSSN | |
| | | | | | | VGAPVTLAAPTSLPARAYFE | |
| | | | | | | AARRLSGEKLEVSVPVEKRG | |
| | | | | | | FFDWLFKRDA | |
| GO_ | mlaD | Phospholipid ABC | H | | Up | MQSTLSGRGGAILASGLVLA | 195 |
| 1943 | | transporter | | | | IAGTFLVYGNALRKGPDFQG | |
| | | substrate- | | | | EILHAAFNSANGLHTGANVD | |
| | | bindingprotein | | | | LAGVPVGRVVSITLDPRTQM | |
| | | MlaD | | | | ADVAFTVDQRLHLPVDTAVG | |
| | | | | | | IGAPTMTADNALQIQPGHSR | |
| | | | | | | TTLSGEGKITDTRDQLSLEQ | |
| | | | | | | QVSNYIFGGGKLGQ | |
| GO_ | mlaE | Phospholipid ABC | H | | Up | MNAVLDPIAALGRAALGLIK | 196 |
| 2354 | | transporter | | | | QAGALALFALEALSHLVRPP | |
| | | permease | | | | FYWRIFFSSLIETGFFSLPV | |
| | | proteinMlaE | | | | VALTALFSGAVIALQSYVGF | |
| | | | | | | GQYHVQSAIAGIVVLAVTRE | |
| | | | | | | LGPVLAGLMVAGRVGAAMSA | |
| | | | | | | QIGTMRVTDQIDALTTLSTN | |
| | | | | | | PMKYLVTPRLLAGTLALPCL | |
| | | | | | | VLVADILGVMGGFTVSVAKL | |
| | | | | | | GFSPSTYITATLDSLKTMDV | |
| | | | | | | VVGLVKAAVFGFLIALLGCY | |
| | | | | | | NGYNSRGGAEGVGSATTAAV | |
| | | | | | | VGASILLLLFDYLLTDLFFS | |
| | | | | | | Q | |
| GO_ | mnmE | tRNA-5- | H | | Up | MTRSSLPDAPDNTPQVIFAL | 197 |
| 2236 | | carboxymethylamino | | | | ATGPSRAAIAIMRASGSGSD | |
| | | methy1-2- | | | | TILKALCNGRLPEPRRVSLR | |
| | | thiouridine(34) | | | | TLRHDGEVLDHAVALWLPGP | |
| | | synthesis protein | | | | NSYTGEDGFELHLHAGPAII | |
| | | MnmE | | | | ARVADALTDLGARPAEPGEF | |
| | | | | | | TRRAVQKGRLDLLQAEAIAD | |
| | | | | | | LVDAETESQRKQALRQADGA | |
| | | | | | | LSRLYDDWAQRLRLVLAHQE | |
| | | | | | | ALIDFPDEELPQDVEDGLVA | |
| | | | | | | ELSKLQIEMSAHLQDNRGEL | |
| | | | | | | MRQGLTVVIAGAPNVGKSSL | |
| | | | | | | LNALSGTDAAIVTHRAGTTR | |
| | | | | | | DAIALDWVLDGVRLRLIDTA | |
| | | | | | | GLRETEDEIEAEGIRRALFH | |
| | | | | | | VKQADVVLHLIGPNESLESL | |
| | | | | | | SGQEIPVRTKIDIAPTPPGM | |
| | | | | | | LGISTQSGEGLAALRQVLSE | |
| | | | | | | RVAELMAGSAAPPLTRARHR | |
| | | | | | | AGIQEAATHLERARTATWPE | |
| | | | | | | LRGEELRLSMLALGRLTGRV | |
| | | | | | | DVESLLDAIFGQFCIGK | |
| GO_ | mnmG | tRNA-5- | H | | Up | MVIGGGHAGCEAAAAAARFG | 198 |
| 2237 | | carboxymethylamino | | | | ARTLLLTHRLETIGAMSCNP | |
| | | methyl-2- | | | | AIGGIGKGHLVREIDALDGL | |
| | | thiouridine(34) | | | | MGKAADRAGIHFKLLNRSKG | |
| | | synthesis | | | | PAVHGPRAQADRSLYRAAIQ | |
| | | protein | | | | DLLAATLNLTILEGAAGDLI | |
| | | MnmG | | | | EENGRITGVICEDGREFRCG | |
| | | | | | | AVVLTTGTFLRGVIHVGHTQ | |
| | | | | | | TEAGRIGEAPAKRLGERLYA | |
| | | | | | | LGLQMGRLKTGTPPRLAKNS | |
| | | | | | | IDWENLPADPGDAEPEAFSP | |
| | | | | | | MTAAITNPQVVCRISHTTAE | |
| | | | | | | THRIINENLHRSAMYGGAIA | |
| | | | | | | GRGPRYCPSIEDKVVRFAER | |
| | | | | | | TSHQVFLEPEALPGNPGGDL | |
| | | | | | | VYPNGISTSLPADVQAAMIA | |
| | | | | | | TMPGLEKARIVTAGYAVEYD | |
| | | | | | | YVDPRELLPSLQLRRLPGLY | |
| | | | | | | LAGQINGTTGYEEAGAQGLL | |
| | | | | | | AGLNAARATAGNEALTLDRS | |
| | | | | | | DAYLGVMIDDLTLHGISEPY | |
| | | | | | | RMFTSRAEYRLTLRADNADL | |
| | | | | | | RLTPKGIAAGCVLSERAAAF | |
| | | | | | | TAQKAELDTAMTRAAETTFL | |
| | | | | | | PQTLRDVGFEVSLDGRRRTV | |
| | | | | | | LDVLASNGDHTKLDTLAPWF | |
| | | | | | | AELPLRVRRHVETEARYGGY | |
| | | | | | | LHRQDREIRQLASESAIALP | |
| | | | | | | ADLDYSAIGGLSSEMRERFS | |
| | | | | | | QARPTSFAAAQRVRGVTPAA | |
| | | | | | | LVALLAHVRTLS | |
| GO_ | mntR | Mn-dependent | H | | Up | MERKRRLRDIAPKETLPDVE | 199 |
| 1674 | | transcriptional | | | | THSEGFRANREARRNVLVED | |
| | | regulator MntR | | | | YVELISDLLSEGQEARQVDI | |
| | | | | | | AGRLGVSQPTVAKMLARLAT | |
| | | | | | | EGYVTQKPYRGVFLTPAGQD | |
| | | | | | | MADRARHRHRIVEAFLLALG | |
| | | | | | | VSEENARVDAEGVEHYVGSE | |
| | | | | | | TLTLFEKALRGNLKQFMQAL | |
| | | | | | | PDA″ | |
| GO_ | mrdA | Peptidoglycan | H | | Up | MRSGRGVFTRRALLVMAVQA | 200 |
| 390 | | D,D- | | | | GVLGVLGRRLYTLQVVDGGH | |
| | | transpeptidase | | | | LRQLAERNRTSKRLLAPARG | |
| | | MrdA | | | | TIHDRFGVALADNKVSWRAL | |
| | | (EC 3.4.16.4) | | | | LMPEETTDIPAVIERFSQIV | |
| | | | | | | PLDEHDRARIERDLRHVHKY | |
| | | | | | | VPVTLHEFLSWDDMARIELN | |
| | | | | | | APSLPGVLVDVGSTRLYPFR | |
| | | | | | | DLTAHIVGYVAPPNEEDVAK | |
| | | | | | | DTTLSLPGMRVGRAGIEQTQ | |
| | | | | | | EAVLRGEPGSVEMEVNAVGR | |
| | | | | | | VIGEIDRVEGQQGEDVRLTL | |
| | | | | | | DSVLQQQVLNRLEDRVASAV | |
| | | | | | | VMDCRNGEVMAMVSTPSFDP | |
| | | | | | | SLFDSGVSHAQWNEWANDPR | |
| | | | | | | TPLVDKAVSGLYPPGSTFKP | |
| | | | | | | AVALAALKSGSVTAQDRFNC | |
| | | | | | | PGYYDLGGVRFHCWNRWGHG | |
| | | | | | | LINMREGLKYSCDVYFYEVA | |
| | | | | | | RRCGMDPIQAVGNAMGLGVK | |
| | | | | | | LGIELPHVRSGVIPTPEWRQ | |
| | | | | | | KHGFHWNGGDTVNAGIGQGF | |
| | | | | | | VQVTPLALATYVSRIASGRD | |
| | | | | | | VQPHLLRATNDQMSAMASVD | |
| | | | | | | DVAKVDLPPEYLDVVRGGMF | |
| | | | | | | AVVNDPHGTAPKARLDLPGI | |
| | | | | | | QMAGKTGSAQVRRVSRALRE | |
| | | | | | | SGHFNSMNLPWEYRPHALFI | |
| | | | | | | CFAPYDNPKYAVSVVVEHGN | |
| | | | | | | AGADEAAPLAKLIMTDTLLR | |
| | | | | | | DPASDVRPPAPSVAQTPSPV | |
| | | | | | | ADGTAPAPTVPVLPDPAPAA | |
| | | | | | | PSEQTDPGAAQ | |
| GO_ | mreB | Rod shape- | H | | Up | MFSRLLGLMSADMAIDLGTA | 201 |
| 387 | | determining | | | | NTLVYVKGRGIVLDEPSVVA | |
| | | protein | | | | IAEVRGKKQVLAVGNEAKQM | |
| | | MreB | | | | VGRTPGNITAIRPLRDGVIA | |
| | | | | | | DFEVAEEMIKHFIRKVHNRR | |
| | | | | | | AFASPQIIICVPSGATAVER | |
| | | | | | | RAIQESAESAGARKVMLIEE | |
| | | | | | | PMAAAIGAGLPVTEPSGSMI | |
| | | | | | | VDIGGGTTEVAVISLGGIVY | |
| | | | | | | ARSARVGGDKMDEAIISYIR | |
| | | | | | | KTYNLLVGESSAERIKIELG | |
| | | | | | | SAMMPDDPANPDGPLTEIKG | |
| | | | | | | RDLINGVPREVIVSQAQIAE | |
| | | | | | | SLAEPVMQIVDAVTTALENT | |
| | | | | | | PPELAADIVDKGIVLSGGGA | |
| | | | | | | LLYRLDEVLRLYTGLPVTVA | |
| | | | | | | ENALSCVALGTGRALEEMRR | |
| | | | | | | LRSVLSSMY | |
| GO_ | ntrY | Nitrogen | H | | Up | MRFPALRRLFARLTSANGAV | 202 |
| 173 | | regulation | | | | LLTVMALVLAFATFVVLSGG | |
| | | protein NtrY | | | | MSLAHRPQVQAIVFLLDFIM | |
| | | (EC 2.7.3. ) | | | | LMLIAAAAVVQIGRMLAERR | |
| | | | | | | LGLAGARLHVRLITLFGIVA | |
| | | | | | | VAPTIVVGAVATLFFHYGVE | |
| | | | | | | IWFSNRVNDALNEARSVAVG | |
| | | | | | | YLQEHNDNARTEAFALANTL | |
| | | | | | | ITVQNDELFSRGTDLFHDPA | |
| | | | | | | RLQEFLDDEVTERGLTDAEV | |
| | | | | | | FDPLTYKVLAVGGLLGSDAD | |
| | | | | | | MTTAPPLPPKSVVELARHGE | |
| | | | | | | AVILDRPDQRRVRAVVALGG | |
| | | | | | | NSGLMLVITRPVDPDVVEHM | |
| | | | | | | RRTDTLVADYQRLITNRGKT | |
| | | | | | | QVLFAVIFALMGLLVLAVGM | |
| | | | | | | LTGLALANRIANPLGLLILA | |
| | | | | | | AQRISKGDLGVRVPVPDDAG | |
| | | | | | | QDKDDEVTGLSRAFNSMTDQ | |
| | | | | | | LESQRSELMQAYDQINERRR | |
| | | | | | | FTETVLAGVSAGVIGLDRMQ | |
| | | | | | | VIELPNRAASSVLQRDLQPA | |
| | | | | | | IGMKLTDRVPEFSGLLEAAR | |
| | | | | | | MAPERVHTAEIQVDTEGAQR | |
| | | | | | | PGGPGEGAGAGAGRTLLVRV | |
| | | | | | | VAELRGQEVAGYVVTFDDIT | |
| | | | | | | DLQSAQRKAAWADVARRVAH | |
| | | | | | | EIKNPLTPIQLAAERLKRRF | |
| | | | | | | LKEIHSDPETYTQLVDTIVR | |
| | | | | | | QVGDIGRMVDEFSAFARMPQ | |
| | | | | | | PVMQPEDFSRLCREALVLQR | |
| | | | | | | NAHPEIVFETTGLPPSGPIV | |
| | | | | | | RCDRRLIGQALTNLLQNAAD | |
| | | | | | | AISMAGRGKPGENASGQPVQ | |
| | | | | | | PIGHIVVGLHIRSGHVLLDI | |
| | | | | | | EDDGIGLPTVERHRLTEPYV | |
| | | | | | | THKAKGTGLGLAIVKKIMED | |
| | | | | | | HSGMLQLTDRAPDQSRGASP | |
| | | | | | | GQRGTRVTLSLPLWEQESHV | |
| | | | | | | PGGTDLQTMRTADGT | |
| GO_ | oprB | Outer membrane | H | | Up | MPVLASFARTSSRIRSEHLR | 203 |
| 630 | | low | | | | SVLMGLFSVSMLSAAVDSAQ | |
| | | permeability | | | | AQSDPDPNSARHRVSRAAAL | |
| | | porin, | | | | SSPAPQNSETPANGGSNTPI | |
| | | OprBfamily | | | | MQNTVSGFAQTEGDPGPYFS | |
| | | | | | | APMGSQHFLGDWGGVQPWLL | |
| | | | | | | KRGIHLMAAINEEFAGNFTG | |
| | | | | | | GKERAYSDAGQFGIELDIDW | |
| | | | | | | DKLAGVRNFWTHTLIVNGHG | |
| | | | | | | QSVSRNFGDSIAGVQQIYGA | |
| | | | | | | RGNVVAHMVAMYGEMSFVHN | |
| | | | | | | RIDVSAGWIPVGSFFAASPL | |
| | | | | | | FCDFMNVAMCGNPAPNKYTE | |
| | | | | | | GNRDWPSGNLGAVVRAMPTM | |
| | | | | | | DTYIMAGLFAVSPHSYNGGI | |
| | | | | | | SGWSWAQSGLGKFSTPVEIG | |
| | | | | | | WTPRFGHNQLQGHYVIGYSY | |
| | | | | | | DNSRYLNLYEDIHGNSWQLT | |
| | | | | | | GQPRRYEAGRNSAWLILDQM | |
| | | | | | | LVRNGPGNTNGLIAMAGAMY | |
| | | | | | | SDGKTVAMRDHEWAGLVDTG | |
| | | | | | | SAWGRPLDTVGAMYQHFDMS | |
| | | | | | | HTAALQQESSLALGLPYQDN | |
| | | | | | | QWGAVYGPQSHENVYELFYS | |
| | | | | | | AHIARAMALQPDFQYIQRPS | |
| | | | | | | ATTTFHDAAVLGVQFTVVL | |
| GO_ | pa2737 | Transcriptional | H | | Up | MTDNSTSQAVMVSPNDDFAS | 204 |
| 1855 | | regulator | | | | APEKVRKAPEAYRTISEVAE | |
| | | PA2737, | | | | ELHIPQHRLRSWETIYPGVK | |
| | | MerR family | | | | PFRGESGRRYYSPEHIETLR | |
| | | | | | | LISDLLYVQGYKGQGVLRVL | |
| | | | | | | RERRAEKARAAQKAVPETAP | |
| | | | | | | ETVPEVSAVSAEAVHVPLVM | |
| | | | | | | VEATEENPAPVVDATPVTEP | |
| | | | | | | VENEDSPVTLTEASVDDIVF | |
| | | | | | | PEVPAAAHEVVVTETVDSTA | |
| | | | | | | ENESPEAEAEAEAEAEAEAE | |
| | | | | | | AEAEAEAEAEAEAEAPDSAL | |
| | | | | | | LVTLRDENELLENTLEKTEA | |
| | | | | | | ENSLLRSELREILEELRNLR | |
| | | | | | | NLLPV | |
| GO_ | pemT | Phosphatidyl | H | | Up | MSSTLSPRSALDAEAVKTAY | 205 |
| 2563 | | ethanolamine N- | | | | RRWARVYDTVFGGISGYGRK | |
| | | methyltransferase | | | | RAVAAVNALPGERVLEVGVG | |
| | | (EC 2.1.1.17) | | | | TGLALPSYSRDKRITGIDLS | |
| | | | | | | EDMLERARIRVLQDHLTNVD | |
| | | | | | | DLLEMDAEATTFEDDSFDIA | |
| | | | | | | VAMFVASVVPHPDRLLAELK | |
| | | | | | | RVVKPGGHILFVNHFLATGG | |
| | | | | | | LRLSVERGMARASRSLGWHP | |
| | | | | | | DFAIESLLPPEDLRRATLTP | |
| | | | | | | VPPAGLFTLVTLPQCESKSD | |
| | | | | | | AAALVA | |
| GO_ | plsY | Acyl- | H | | Up | MSGFQAQLILLSLISYVIGS | 206 |
| 2931 | | phosphate: | | | | IPFGLLLTALGGGGDIRKIG | |
| | | glycerol-3- | | | | SGNIGATNVLRTGRRGLAAA | |
| | | phosphateO- | | | | TLLLDALKGAMAVLIARFFF | |
| | | acyltransferase | | | | PGASETTMAVAAVAVVIGHC | |
| | | PlsY | | | | FPVWLGFRGGKGVATGLGTI | |
| | | (EC 2.3.1.n3) | | | | WVLSWPVGLACCVVWLLVAR | |
| | | | | | | LSRISSAGALAAFFIAPVLM | |
| | | | | | | VLLSGRTLHSPIPVATLLVS | |
| | | | | | | LLIWVRHSSNIARLLTGREP | |
| | | | | | | RVNVDPASRR | |
| GO_ | pqqB | None (82 bp | H | | Up | MIDVIVLGAAAGGGFPQWNS | 207 |
| 2302 | | upstream from | | | | AAPGCVAARTRQGAKARTQA | |
| | | Coenzyme PQQ | | | | SIAVSADGKRWFILNASPDL | |
| | | synthesis | | | | RQQIIDTPALHHQGSLRGTP | |
| | | protein B) | | | | IQGVVLTCGEIDAVTGLLTL | |
| | | | | | | REREPFTLMGSDSTLQQLAD | |
| | | | | | | NPIFGALDPEIVPRVPLLLD | |
| | | | | | | EATSLMNKDGIPSGLLLTAF | |
| | | | | | | AVPGKAPLYAEAAGSRPDET | |
| | | | | | | LGLSITDGCKTMLFIPGCAQ | |
| | | | | | | ITAEIVERVAAADLVFFDGT | |
| | | | | | | LWRDDEMIRAGLSPKSGQRM | |
| | | | | | | GHVSVNDAGGPVECFTTCEK | |
| | | | | | | PRKVLIHINNSNPILFEDSP | |
| | | | | | | ERKDVERAGWTVAEDGMTFR | |
| | | | | | | LDTP | |
| GO_ | priA | Helicase PriA | H | | Up | MASNSLSKPAPWRKPSSAGT | 208 |
| 1198 | | essential for | | | | RVSVLVPLPFPGPLDYLAPM | |
| | | oriC/DnaA- | | | | PLEPGELVTVPLGRRETVGC | |
| | | independentDNA | | | | VWETDRTLPADFALPVGREV | |
| | | replication | | | | PLARLRPVAGKLDVRPLPQS | |
| | | | | | | LRRFIDWVAAYTLTPPGLVL | |
| | | | | | | AMATRIHLKDAPRPTLGWVR | |
| | | | | | | TEMPEGDLRITPARRSVLNF | |
| | | | | | | ASSTPMTTAELGSRSGASAA | |
| | | | | | | VIRGLATAGLLREAVIAVAA | |
| | | | | | | PFATPDPSHGAPKLSEEQAE | |
| | | | | | | VAQELCGPVEAGRFQVTLLE | |
| | | | | | | GVTGSGKTEVYFEAVAACLA | |
| | | | | | | KGKQVLVLLPEIALSAQWTD | |
| | | | | | | RFIRRFGAEPAVWHSDLGAK | |
| | | | | | | RRRETWRAVAEGSARVVVGA | |
| | | | | | | RSALFLPFSDLGLVVVDEEH | |
| | | | | | | ESAFKQEEGVMYHGRDMAVV | |
| | | | | | | RARLADAPAILVSATPSLET | |
| | | | | | | LANVESGRYRHELLTARHGG | |
| | | | | | | ATLPDVSLVDMRADPPERGL | |
| | | | | | | FLSPKLCTAIDETLEKGEQA | |
| | | | | | | MLFLNRRGYAPLTLCRACGH | |
| | | | | | | RMQCPNCSAWLVEHRARGIL | |
| | | | | | | TCHHCGHTERIPKDCPECHA | |
| | | | | | | ENSLVPIGPGIERITEEAKL | |
| | | | | | | RFPDAKLLVMSSDTLGSPAA | |
| | | | | | | TAEAVRKISDGEVNLIIGTQ | |
| | | | | | | IVAKGWHFPKLTLVGVVDAD | |
| | | | | | | LGLGGGDLRAAERTVQLLHQ | |
| | | | | | | VAGRAGRAEHRGRVLLQSYV | |
| | | | | | | GEHPVMTALVSNDFQTFMEQ | |
| | | | | | | EAEQRRPSFWPPYGRLAALI | |
| | | | | | | VSAPSAEAADALAREIALSA | |
| | | | | | | PEREGVQVLGPAPAPLAVLR | |
| | | | | | | GRHRRRLLLRTMRGIAVQPI | |
| | | | | | | LRQWLGDIKPTGGAKVDIDI | |
| | | | | | | DPISFL | |
| GO_ | proX | Glycine | H | | Up | MTQMTIGHLYTSLHAGCASA | 209 |
| 1638 | | betaine/L- | | | | VARVLEAYEVEVEYVDLDPD | |
| | | proline | | | | DIEDALEGAEVDLLVSAWFP | |
| | | transportsubstrate- | | | | RDEKFAGPGRRVLGDLYQPV | |
| | | binding | | | | VSFAALLPLAAVGTLTSADV | |
| | | protein ProX | | | | DRIIVSDDSRQALEDALKQL | |
| | | (TC 3.A.1.12.1) | | | | PALSVLPIESIGEGALIERL | |
| | | | | | | EKARDAGEKPLVVATQPHAV | |
| | | | | | | FHTDLLTVIEDPAHLLGGEM | |
| | | | | | | SARMIMREDVARQADSDLLD | |
| | | | | | | ELSEMMLGNRVMSALDYVIS | |
| | | | | | | VEGQDPEGAAEAWQRGRLIG | |
| | | | | | | R | |
| GO_ | putR | Predicted | H | | Up | MSEAEPIFTPVDTKSGSASL | 210 |
| 548 | | regulator | | | | EIAEALRRAITNGILTDGQP | |
| | | PutR for | | | | MRQSELARNFGVSTIPVREA | |
| | | prolineutilization, | | | | LKQLEAEGLIAFLPHRGAVV | |
| | | GntR family | | | | TGLSEADILEYSDIRASLES | |
| | | | | | | MAAGLAMTSLTRVDLARIED | |
| | | | | | | AYEAFVSGTGGTHGMEQSGR | |
| | | | | | | LNWEFHGAIYAAAQRPRLYG | |
| | | | | | | MIHDLHSRLDRYIRAHLELP | |
| | | | | | | GRKTATDAEHFQILQACRAR | |
| | | | | | | DGEELGRLTKQHILEAASLS | |
| | | | | | | LDVIRNRTTP | |
| GO_ | rarA | Replication- | H | | Up | MTDMTDLFGGAPEANRAPGH | 211 |
| 1388 | | associated | | | | ASARGPSRAPESMRQQRPIE | |
| | | recombination | | | | APQVQRRPPSRTQPLADRLR | |
| | | proteinRarA | | | | PRRLEDVRGQDHLLGPEGTL | |
| | | | | | | TRMLERGTLSSLILWGGPGC | |
| | | | | | | GKTTIARLLAGRAGLFYSQI | |
| | | | | | | SAVFSGVADLRRAFEEADQK | |
| | | | | | | QAATGKGTLLFVDEIHRFNR | |
| | | | | | | AQQDGFLPYVERGTVVLVGA | |
| | | | | | | TTENPSFALNAALLSRCQVL | |
| | | | | | | VLNRLDDASLESLLLHAEEE | |
| | | | | | | VGRPLPLDPEARASLRAMAD | |
| | | | | | | GDGRYLLNMVEQLVALDPSK | |
| | | | | | | VLTPRDLAAILSRRAILYDR | |
| | | | | | | DREEHYNLISALHKSLRGSD | |
| | | | | | | PDAALYWFARMLEGGEDPRY | |
| | | | | | | IARRLTRFAAEDVGQADPSA | |
| | | | | | | LPMAVAGWQTYERLGSPEGE | |
| | | | | | | LALAQVVVHLATAPKSNAVY | |
| | | | | | | TAYKAARALARDTGSLMPPS | |
| | | | | | | HILNAPTSLMKDIGYGKGYE | |
| | | | | | | YDHDSEDAFSGQNYFPDGMP | |
| | | | | | | RASLYHPTDRGHEGRIRKLL | |
| | | | | | | DMRAAKRASREP | |
| GO_ | rbfA | Ribosome- | H | | Up | MKGPAGVSAHGPSTRQLRVA | 212 |
| 1471 | | binding | | | | EEVRRVLAELFARTEFRDPE | |
| | | factor A | | | | LLDVRITVTEVRISPDFRHA | |
| | | | | | | TAFVTRLGRSDVEVLLPALK | |
| | | | | | | RVAPFLRTGLSKALNLRTVP | |
| | | | | | | EIHFQPDTALDNAMELDEIM | |
| | | | | | | RSPEVQRDLNSKPE | |
| GO_ | reck | Regulatory | H | | Up | MEDRPAPLPVPDAASLREAA | 213 |
| 2904 | | protein | | | | LAHLARFATTEQGLRQVLDR | |
| | | RecX | | | | RLRRWGVCASRAGLPNEDIE | |
| | | | | | | STIAAVSPAIDGIVASMTDL | |
| | | | | | | GAVDDAGYARNRAVSLTRTG | |
| | | | | | | RSRRAVEAHLANKGVDQNTT | |
| | | | | | | REALNDSLGERSDSSAQEAE | |
| | | | | | | LAAALVLARKRRLGPFQRPD | |
| | | | | | | REEEDPLKALGVFARNGFSR | |
| | | | | | | DVAQSVLDMDSDEAEDRIIA | |
| | | | | | | FRSL | |
| GO_ | rfbD | O-antigen | H | | Up | MSVSSPASDVARNDAPHRQV | 214 |
| 635 | | export | | | | SRDALRGSPAARAWADWKET | |
| | | system permease | | | | RRLWRLGVRLGWLDIRLRYR | |
| | | protein RfbD | | | | GSALGPFWLTITSALMVASM | |
| | | | | | | GVLYSKLFHMQLASYLPFLS | |
| | | | | | | LSLTLWSVGFSSLIQESCTC | |
| | | | | | | FLDAEDMVRSVRLPFLLYAV | |
| | | | | | | RVVVRNAIVFAHNIVVPLGV | |
| | | | | | | FALYHLWPGMDALLAIPALL | |
| | | | | | | LWGLDGFAACMLFGSLCARF | |
| | | | | | | RDVAPIIGALLQIVFYVTPV | |
| | | | | | | IWMPQQLGPRAAYLLYNPFY | |
| | | | | | | PLLEIVREPLLGHVPSLQIW | |
| | | | | | | GIALATSAVFWLIAVRSFIR | |
| | | | | | | VRSRLVFWI | |
| GO_ | rneE | Ribonuclease | H | | Up | MRSDCTVPSTPHNLTFRLAK | 215 |
| 380 | | E (EC 3.1.26.12) | | | | AAIVERRGVQFSMTKRMLID | |
| | | | | | | TTHAEETRVVVMDGDRVEDY | |
| | | | | | | DVETSTKKQLKGNIYLAKVI | |
| | | | | | | RVEPSLQAAFVEYGGNRHGF | |
| | | | | | | LAFSEIHPDYFQIPVADREK | |
| | | | | | | LIALQEEEIAERGDAADDGE | |
| | | | | | | TETVSEEATDSEDGENQDRR | |
| | | | | | | APETVGGEHDTGEESASSRR | |
| | | | | | | TARFLRNYKIQEVIRRRQVL | |
| | | | | | | LVQVVKEERGNKGAALTTYI | |
| | | | | | | SLAGRYCVLMPNALRGGGVS | |
| | | | | | | RKITSDSDRRRLRDVIAELD | |
| | | | | | | LPKSMAMIVRTAGAGRPAQE | |
| | | | | | | VTRDCEYLLQLWDDIRSHAL | |
| | | | | | | SSVAPTLVYEEASLIKRVIR | |
| | | | | | | DLYSKDIEDILVDGEAAWKS | |
| | | | | | | AREFMRLLMPSNANKVKLWQ | |
| | | | | | | NRGQSLFARYNVEGHLDAMF | |
| | | | | | | SPTARLPSGGYLVINQTEAL | |
| | | | | | | VAIDVNSGKSTSQRNIEETA | |
| | | | | | | LRTNLEAAEEVGRQLRLRDL | |
| | | | | | | AGLVVIDFIDMESRRHNAQV | |
| | | | | | | EKRLKEALRSDRARIQVGHI | |
| | | | | | | SHFGLLEMSRQRLRPSIAES | |
| | | | | | | VLTPCPHCQGTGFIRGTESS | |
| | | | | | | ALHVLRAIDEEGARQRSSEI | |
| | | | | | | EVHVGSDIALYILNHKRSWL | |
| | | | | | | ADIERHHRMQVIFRTEENLA | |
| | | | | | | AADMRIERLQAQTPAPAQVR | |
| | | | | | | APERAPEHVRTIEIIEEEAP | |
| | | | | | | VVVRTETPVVDAEIIEDVAT | |
| | | | | | | SESEEESNGGRRRRRRRRRG | |
| | | | | | | GRREQNGDVPAAENSQEQDA | |
| | | | | | | PKAETREIAAPEAADENGII | |
| | | | | | | PGRRRTRFKRVVKDTEGSED | |
| | | | | | | TAAQPVSEVRDVAPTRPAAT | |
| | | | | | | TRSSAPAPTRTPRRREDREE | |
| | | | | | | RPAPRRYTGPTPADPFGGSF | |
| | | | | | | DIFDVIEQAELEGTTEALQT | |
| | | | | | | GISTPTEIVIEHVPAAETTI | |
| | | | | | | VVEEPVAEEAPKPRRGRGRR | |
| | | | | | | PARAKAVEAAPAETVAEEAP | |
| | | | | | | AAEAAPAPEEPVAEEPPKPR | |
| | | | | | | RTRTRRTPKAQAVEAEAPAE | |
| | | | | | | PTAEVAASPAETVSEEAVKP | |
| | | | | | | KRTRARRTPKAKPVEAQTTE | |
| | | | | | | EGNILQPVNVDEVAPTKRRA | |
| | | | | | | GWWKR | |
| GO_ | rodA | Rod shape- | H | | Up | MRRNGMNTFGFLKSDRRLLR | 216 |
| 391 | | determining | | | | AEPDFRPMARLLQVNWLYVL | |
| | | protein | | | | LVCLLAGVGYIALYSAGGGS | |
| | | RodA | | | | AKPFAGPQMVRFGFGLVMMI | |
| | | | | | | AVSLVSPRILRMASVPIYLL | |
| | | | | | | SVTLLALVLRMGHVGKGAER | |
| | | | | | | WINLAGMQFQPSEFAKIGLV | |
| | | | | | | LMLATWFHRIGNERMGNPLR | |
| | | | | | | LIPPALLTLLPVLLVLKEPN | |
| | | | | | | LGTATIIGVIGATMFFAAGM | |
| | | | | | | RLWQILLLVAPLPFMGKLIY | |
| | | | | | | SHLHDYQKARIDTFLHPEHD | |
| | | | | | | PLGAGYNIIQSKIALGSGGM | |
| | | | | | | WGEGYLHGSQGQLNFLPEKQ | |
| | | | | | | TDFIFTMIGEEWGFVGGIAV | |
| | | | | | | ITLLGTLVMGGMLIAIRSRN | |
| | | | | | | QFGRLLGLGIAMDFFLYCAV | |
| | | | | | | NLSMVMGAIPVGGVPLPLIS | |
| | | | | | | YGGSAMLTMMFGFGLLMSAW | |
| | | | | | | VHRNERDPGEDEDEDD | |
| GO_ | slp | SSU ribosomal | H | | Up | MDIRSAKGTGIREYSIYMAS | 217 |
| 1056 | | protein Slp | | | | ATQTPTANHGTEDFAALLEE | |
| | | | | | | TLGRDTGFDGSVVTGRVVRL | |
| | | | | | | TDEFAIVDVGLKSEGRVSLK | |
| | | | | | | EFGPAGVAPDVKPGDVVELY | |
| | | | | | | VERYEDRDGSIVLSREKARR | |
| | | | | | | EEAWTALERAFANNQRVNGT | |
| | | | | | | IYGRVKGGFTVDLGGAMAFL | |
| | | | | | | PGSQVDIRPVRDVGPLMGQP | |
| | | | | | | QPFQILKMDRARGNIVVSRR | |
| | | | | | | AVLEETRAEQRSELIQGLKE | |
| | | | | | | GMILDGVVKNITDYGAFVDL | |
| | | | | | | GGVDGLLHVTDIAWKRINHP | |
| | | | | | | SEALQIGQPVRVQVIRFNPD | |
| | | | | | | TQRISLGMKQLEADPWENVA | |
| | | | | | | IKYPAGARFTGRVTNITDYG | |
| | | | | | | AFVELEPGVEGLVHVSEMSW | |
| | | | | | | TKKNVHPGKIVATSQEVDVM | |
| | | | | | | VLDVDSAKRRISLGLKQVQR | |
| | | | | | | NPWEQFEEEHKVGSIIEGEI | |
| | | | | | | RNITEFGLFVGLSADIDGMV | |
| | | | | | | HMSDLSWDEPGEAAMAHYEK | |
| | | | | | | GQVVKAKVLDVDPEKERISL | |
| | | | | | | GIKQLQEDPAADTLSRVQKG | |
| | | | | | | AVVTCVVTAVQSNGIEVKVD | |
| | | | | | | DVLTGFIRRAELARDKAEQR | |
| | | | | | | PERFAVGEKVDAKVVSVDRA | |
| | | | | | | SRKLALTIRGREVEEDKQAI | |
| | | | | | | NEYGSSDSGASLGDILGAAI | |
| | | | | | | RRRNTDA | |
| GO_ | s21p | SSU ribosomal | H | | Up | MQVLVRDNNVDQALKALKKK | 218 |
| 1844 | | protein S21p | | | | MQREGVFREMKLRRHFEKPS | |
| | | | | | | EKRAREAAEAVRRARKMERK | |
| | | | | | | RLEREGF | |
| GO_ | s9p | SSU ribosomal | H | | Up | MSETQERTGTLQDLASVAPA | 219 |
| 188 | | protein S9p | | | | VTSNAAPVHEVKRDAQGRSY | |
| | | (S16e) | | | | ATGRRKDAVARVWIKPGKGD | |
| | | | | | | IIVNGRPVTTYFARPVLRML | |
| | | | | | | LTQPFLIADRYNQFDVYCTV | |
| | | | | | | TGGGLSGQAGAVRHGISRAL | |
| | | | | | | TYYEPSLRGILKAAGFLTRD | |
| | | | | | | SRIVERKKYGRAKARRSFQF | |
| | | | | | | SKR“ | |
| GO_ | sldB | Broad- | H | | Up | MPNTYGSRTLTEWLTLLLGG | 220 |
| 3173 | | specificity | | | | VIILVGLYFVIAGGDLAMLG | |
| | | glycerol | | | | GSVYYVICGILLVAGGVFMV | |
| | | dehydrogenase | | | | MGRTLGAYLYLGALAYTWVW | |
| | | (EC 1.1.99.22), | | | | SLWEVGFSPVDLLPRAFGPT | |
| | | subunit SldB | | | | LLGILVALTIPVLRRMETRR | |
| | | Gluconate 5- | | | | TLRGAV | |
| | | dehydrogenase, | | | | | |
| | | smallsubunit | | | | | |
| GO_ | tolQ | Tol-Pal system | H | | Up | MDHEVSSSALGAVGAAGLSP | 221 |
| 1367 | | protein TolQ | | | | LDLFLHASIVVKLVMLGLLL | |
| | | | | | | CSAGVWAIIAEKIILIRRVN | |
| | | | | | | REATEFEDRFWSGGSLDDLY | |
| | | | | | | ESDGARPTHPMAAVFGAAMG | |
| | | | | | | EWRRSARIGGIDLSRGGVRE | |
| | | | | | | RVDRAIDITIMRENDRLTRR | |
| | | | | | | LIFLATIGPVAPFVGLFGTV | |
| | | | | | | WGIMHSFASIAQMHNTNLSV | |
| | | | | | | VAPGISEALFATAIGLVTAI | |
| | | | | | | PAYIAYNGLSNSFEKFADRM | |
| | | | | | | EAFGTEFAAILSRQSEERAD | |
| | | | | | | DTTGGKA | |
| GO_ | tolR | Tol biopolymer | H | | Up | MGMSAGGRAGGGGRRKRRPA | 222 |
| 1366 | | transport system, | | | | SEINVTPLVDVMLVLLIIFM | |
| | | TolR protein | | | | VTAPMLTSGVNVDLPKTDAS | |
| | | | | | | PVNSDTKPITVSLRTDGSLY | |
| | | | | | | LGDQQVTSDQLIDQLKAQSQ | |
| | | | | | | NDPTHRIFVRADAHIDYGQV | |
| | | | | | | MQVMGQITSGGFTHVALLAQ | |
| | | | | | | QPQSGQ | |
| GO_ | trxa | Thioredoxin | H | | Up | MSANTVAVSDSSFEADVLKS | 223 |
| 1087 | | | | | | EGPVLVDFWAEWCGPCKMIA | |
| | | | | | | PALEEIGAEYQGRLKVAKVN | |
| | | | | | | IDSNPEAPTKYGVRSIPTLI | |
| | | | | | | VFKDGKPVAQQMGALPKSQL | |
| | | | | | | KAWIDQSL | |
| GO_ | uvrD/ | ATP-dependent DNA | H | | Up | MPQDLPSPTPEYLSRLNPEQ | 224 |
| 2012 | pcrA | helicase UvrD/PcrA | | | | RRAIETTEGPLLILAGAGTG | |
| | | (EC 3.6.4.12) | | | | KTRVLTTRFAHILLTGRAYP | |
| | | | | | | SQILAVTFTNKAAREMRERV | |
| | | | | | | SAILGEPAEGLWLGTFHAIC | |
| | | | | | | ARMLRRHAEYVGLTSSFNIL | |
| | | | | | | DTDDQIRLLKQVMEPWKIDT | |
| | | | | | | KRWPPNQLMGIIQRWKDRGL | |
| | | | | | | TPDRVTPVEDSDFANGHALA | |
| | | | | | | IYRAYQERLIALNTCDFGDL | |
| | | | | | | MLHMTEILRNQPNVLAQYHR | |
| | | | | | | IFRYILVDEYQDTNTVQYLW | |
| | | | | | | LRLLAKREHGPSNIACVGDD | |
| | | | | | | DQSIYSWRGAEVENILRFEK | |
| | | | | | | DFPGAEVVRLERNYRSTAQI | |
| | | | | | | LAAAAGLIAHNEGRLGKTLR | |
| | | | | | | PGRDDAQGEKVQIIGVRDSD | |
| | | | | | | EEARIVGGAIERLRGDGHPL | |
| | | | | | | SEIAILMRAGFQTRPFEERL | |
| | | | | | | MMIGIPYRVVGGLRFYERSE | |
| | | | | | | IRDCLAYLRVLSQPADDLAF | |
| | | | | | | ERIINVPKRGVGAVAVQKLH | |
| | | | | | | AQARALPGPLTAAVIWQLQE | |
| | | | | | | GLLKGKSKEALDGLMAAFQR | |
| | | | | | | AKATLETEGHVVAAEQLLED | |
| | | | | | | SGYLQMWRDDRSVEAPGRLD | |
| | | | | | | NIRELLRALGEFGTLQGFLE | |
| | | | | | | HVALVMDTESETSDDKVSLM | |
| | | | | | | TLHGAKGLEFDTVFLPGWEE | |
| | | | | | | GVFPSQRSLDEGGNRALEEE | |
| | | | | | | RRLAYVGLTRARRRAIVLHA | |
| | | | | | | ASRRIYANWQSSMPSRFIEE | |
| | | | | | | IPSEYVQLTGQAFETRRQAA | |
| | | | | | | AAPSMFASGPLTSTRPSGFR | |
| | | | | | | APRPKIHDVKPEPTVAIGAT | |
| | | | | | | VFHHRFGEGTVIGADGPQLH | |
| | | | | | | INFENGGVKRVMANFVEIRS | |
| GO_ | ybbN | FIG000875: | H | | Up | MSSTFDEHLIGQSSGKAPAG | 225 |
| 3271 | | Thioredoxin | | | | AAATIIDADQTTFMADVVEA | |
| | | domain- | | | | SREIPVLVDFWAPWCGPCKQ | |
| | | containing | | | | FTPVLEKVVHAAGGRIRLVK | |
| | | proteinEC- | | | | VDVEANQALAAQLGQLGLPL | |
| | | YbbN | | | | QSIPLVVAFWKGQVLDLFQG | |
| | | | | | | AQPESEVRRFVESLLKLAGD | |
| | | | | | | VMPATEILAAARQALADGHA | |
| | | | | | | DQAAGLFSQLLEAEPENPEA | |
| | | | | | | WGGMIRALIALNEPEAAQDA | |
| | | | | | | SAQVPAKLDSHPEITGARAA | |
| | | | | | | LALHAEGAKAASELETLRQQ | |
| | | | | | | SAAAPDDFDLRVRLAAALNG | |
| | | | | | | AGERAEAANTLLDILRKDRN | |
| | | | | | | WNEGAAKTELLRFFEAWGHT | |
| | | | | | | DPDTLAARRKLSSLLFS | |
| GO_ | ycaR | FIG002473: | H | | Up | MTTELDPRLLSLLVCPVTKG | 226 |
| 3269 | | Protein | | | | PLTYDRETQELISPRAKLAF | |
| | | YcaR in KDO2- | | | | PIRDGIPIMLPEEARQIDA | |
| | | Lipid | | | | | |
| | | Abiosynthesis | | | | | |
| | | cluster | | | | | |
| GO_ | ftsJ | 23S rRNA | H | | Up | MKPPRSRSGSSKDTGPKRIP | 227 |
| 2100 | | (uridine(2552)- | | | | GKALKSASNPGENDATLDSA | |
| | | 2′-O)- | | | | TARTARNKTVSLRTARGRTT | |
| | | methyltransferase | | | | AQQRWLNRQLNDPYVAAARK | |
| | | (EC | | | | QGWRSRAAFKLIEIDDRFKL | |
| | | 2.1.1.166) | | | | IGEGTRIIDLGAAPGGWTQV | |
| | | | | | | AVKRGAQHVVGLDLLPVDPV | |
| | | | | | | AGAEIIEGDFTDPEMPDRLK | |
| | | | | | | DMLGGPADLVMSDMAPNTTG | |
| | | | | | | HAATDHMRIMGLAEGALDFA | |
| | | | | | | FQVLAEGGSFIAKVFQGGSE | |
| | | | | | | KDMLALMKTAFSSVKHVKPP | |
| | | | | | | ASRKESSELYVIATGFRPER | |
| | | | | | | LPEGGKGA | |
| GO_ | GO_ | Alkyl | H | | Up | MVRINSPLKPFSTDAFRNGE | 228 |
| 47 | 47 | hydroperoxide | | | | FLTVTDSDVRGKWSVFFFYP | |
| | | reductase | | | | ADFTFVCPTELEDLADNQAA | |
| | | protein C | | | | FDKLGVEIYSVSTDKHFTHK | |
| | | (EC 1.11.1.15) | | | | AWHDTSPAIGKIKYTMLGDP | |
| | | | | | | TGAIARNFDVLIEEAGLADR | |
| | | | | | | GTFLIDPEGKIQYIEITAGG | |
| | | | | | | VGRSATELLEKIQAAQYVAT | |
| | | | | | | HPGEVCPAKWKEGGETLKPS | |
| | | | | | | LDLVGKI | |
| GO_ | g6pd | Glucose-6- | H | | Up | MEHFQQVEPFDYVIFGATGD | 229 |
| 1805 | | phosphate | | | | LTMRKLLPALYNRLRMGQIP | |
| | | 1-dehydrogenase | | | | DDACIIGAARTELDREAYVA | |
| | | (EC 1.1.1.49) | | | | RARDALERFLPSDILSPGLV | |
| | | | | | | ERFLARLDYVTLDSSREGPQ | |
| | | | | | | WDALKSLLAKAQPDRVRVYY | |
| | | | | | | FATAPQLYGSICENLNHYGL | |
| | | | | | | ITPTSRVVLEKPIGTNMATA | |
| | | | | | | TAINDGVGQYFPEKQIYRID | |
| | | | | | | HYLGKETVQNVLALRFANPL | |
| | | | | | | MNAAWSGEHIESVQITAVET | |
| | | | | | | VGVESRAAYYDTSGALRDMI | |
| | | | | | | QNHLLQVLCLVAMEAPDSLE | |
| | | | | | | ADAVRNAKLAVLNALRPITD | |
| | | | | | | ATAATETVRAQYTAGVVDGE | |
| | | | | | | NVPGYLEELGKPSTTETYAA | |
| | | | | | | IRAWVDTPRWKNVPFYIRTA | |
| | | | | | | KRSGKKVSEIVITFRPAATT | |
| | | | | | | MFGASPASNRLVLRIQPNEG | |
| | | | | | | VDLRLNVKNPALDVFNLRPA | |
| | | | | | | DLDTSIRMEGGLPFPDSYER | |
| | | | | | | LLLDAVRGDPVLFIRRDEVE | |
| | | | | | | AAWRWAEPILDAWKNDKAPM | |
| | | | | | | QTYSAGSYGPEQATQLLASH | |
| | | | | | | GDTWHEASE | |
| GO_ | GO_ | Membrane | H | | Up | MTDLHDTLLTLDSHIDIPWP | 230 |
| 2091 | 2091 | dipeptidase | | | | DRQDAWVEETPRQVNVPKTR | |
| | | (EC: 3.4.13.19) | | | | KGGLRAVCLAAYIPQGPRDE | |
| | | | | | | AGHAEGWERVQGMLDVIAGL | |
| | | | | | | EGRQGDQGARVCRTAADVRA | |
| | | | | | | AYKAGELAVIPAIENGHGLG | |
| | | | | | | GRPERIAELARRYGVRYMTL | |
| | | | | | | THNGHNALADAAIPRKDLAD | |
| | | | | | | NETLHGGLSDLGRETIAKMN | |
| | | | | | | RSGVLVDVSHAAKSTMMQAT | |
| | | | | | | EVSITPVFASHSCARALCDH | |
| | | | | | | PRNLDDEQLDRLKETGGLIQ | |
| | | | | | | VTAMGSFLKKGGGGTVEDLV | |
| | | | | | | RHVSYIADRIGVEHVGISSD | |
| | | | | | | FDGGGGIPGWKDATETANVT | |
| | | | | | | QALQAAGFSDTDISSIWGGN | |
| | | | | | | MLRLLETAERAAEKV | |
| GO_ | GO_ | Multimodular | H | | Up | MTRRSFRSRNPQGSGNPQGP | 231 |
| 2951 | 2951 | transpeptidase- | | | | GAPPPRPPRFRRYRQSLAII | |
| | | transglycosylase | | | | AGVGLVGIAGAGVLGWTTYA | |
| | | (EC 2.4.1.129) (EC | | | | KLVADLPSVDSLRAYQPPTV | |
| | | 3.4.—.—) | | | | SRIYASDDRLMAELANERRI | |
| | | | | | | FVPINAIPERVKNAFIATED | |
| | | | | | | HNFYTHGGVDFMAIGRAGLT | |
| | | | | | | DIFARHGRRPLGASTITQQV | |
| | | | | | | AKVMLLNSNVLSFDRKIKEA | |
| | | | | | | LLAMKMEQVLSKDKILEIYL | |
| | | | | | | NGIYLGNGAYGVAAAAQSYF | |
| | | | | | | NKPLDQLDDAEAASLAALPK | |
| | | | | | | SPTNYNPFLHPQAAMARRNL | |
| | | | | | | VLDLMVEAGVLTRQQADQEK | |
| | | | | | | QEPLVPQQKQRFGPLPDSEW | |
| | | | | | | FGEEVRRQLIAQYGQERAAQ | |
| | | | | | | GGLEVHTSLDQSLQVTETRL | |
| | | | | | | LHEGLMNYDRVHSGWRGPLR | |
| | | | | | | NLPDIQDDGWESALDHITPP | |
| | | | | | | GGMLREWRLAVVLPGATHVG | |
| | | | | | | WIEEGTARKGALLATDMAWA | |
| | | | | | | RRMRPLRAGDVIMIEPQEGG | |
| | | | | | | SAALRQIPQVEGAAVTLDVH | |
| | | | | | | TGRVLAMVGGWSFHESQFNR | |
| | | | | | | VTQALRQPGSSFKPFVYLAA | |
| | | | | | | MEKGISPSERFDDSPVSYGD | |
| | | | | | | WHPQNYEHDNWGPTTLHDAL | |
| | | | | | | RESRNLVTIRVAAHLGMKAV | |
| | | | | | | ADTAIRAGLVAQMPHVLPAA | |
| | | | | | | LGAVETTVMREAAAYATIAN | |
| | | | | | | GGHIVTPTLVDDIQDRAGTV | |
| | | | | | | LWQAGGLTLGTAMQAPPAEQ | |
| | | | | | | PVPADGTTTSAAPATAPVAP | |
| | | | | | | SATPTTPPPGSVAVPALTDG | |
| | | | | | | RPVLASEQSTFQIVKMMQDV | |
| | | | | | | IARGTGRMAGVGIDRPIAGK | |
| | | | | | | TGTSQDFHDAWFAGFSPDIV | |
| | | | | | | TVVWVGFDTPQTLGRNSDGG | |
| | | | | | | RVAGPIWNRIMKVALANRPK | |
| | | | | | | LDFRVPDGITLASYDTGRIS | |
| | | | | | | AVDGFKTDQVPGASVELHGF | |
| | | | | | | GAGTEALTAADTGADSVISD | |
| | | | | | | SESDMAQTPGQGGGMAGAAP | |
| | | | | | | GSGTAAAPGQAQKPAPSDGD | |
| | | | | | | IGVGGLY | |
| | GO_ | Prephenate | H | | Up | MTPLFRSLAVIGPGLIGSSV | 232 |
| | 2112GO_ | dehydrogenase | | | | LRRARETGAIAETLIAADSN | |
| | 2112 | (EC | | | | PGVLERVRELGIADITTSDP | |
| | | 1.3.1.12) | | | | AVAAQADCVMICVPVGAVEP | |
| | | | | | | VARQVLPFMKPGSILTDVAS | |
| | | | | | | VRGQLGPTLAAILPENVAYV | |
| | | | | | | PGHPMAGTEHSGPDAGFSTL | |
| | | | | | | FEDRWALLVPPEGADPKAVT | |
| | | | | | | TIGQLWTLCGARTKILSDEK | |
| | | | | | | HDRICAMVSHLPHLLAFTIC | |
| | | | | | | DTADNLSDEIRAAVLDYAAS | |
| | | | | | | GFRDFTRIAASDPVMWRDIF | |
| | | | | | | LANKEALLGTLDKFVADTQA | |
| | | | | | | MADAIRTGDEATITSKIERG | |
| | | | | | | RAIRRTLIENRQA | |
| | GO_ | Pyrroline-5- | H | | Up | MPSVPSVLLAGCGKMGGALL | 233 |
| | 2168GO_ | carboxylate | | | | DGWLASPTPPRLVILDRHRT | |
| | 2168 | reductase | | | | GTDDAITVVRTAAEIPAGFK | |
| | | (EC 1.5.1.2) | | | | PDVIVLAVKPATAEIMIDAI | |
| | | | | | | GKALGPRMSNAAILSVMAGR | |
| | | | | | | TCAALSEAAQLAGADMPVLR | |
| | | | | | | AMPNTPSAVGAGISGLYASP | |
| | | | | | | SATPEQKSICHDLLFAVGDV | |
| | | | | | | VPVEKEADLSIVTAISGSGP | |
| | | | | | | AYVFLLAELLEKAGQQHGLS | |
| | | | | | | PTIARRLARGTIYGAGRMLD | |
| | | | | | | DLPDSAEDLRKAVTSPNGTT | |
| | | | | | | AAALAVLMKSDAWPETVPKA | |
| | | | | | | IDAATKRADELAG | |
| GO_ | GO_ | Ribonuclease | H | | Up | MKLSHTEALASMQAALGHDF | 234 |
| 2617 | 2617 | III (EC | | | | KKPELLSEALTHRSAVSGRD | |
| | | 3.1.26.3) | | | | PRRARRNQRPKGSGSNERLE | |
| | | | | | | FIGDRVLGLLMAEWLLERYP | |
| | | | | | | DEQEGALGPRHAHLVSRTVL | |
| | | | | | | AEIADQVGLSASLQISPHEE | |
| | | | | | | DAGIRRLSSIRADAMEALLG | |
| | | | | | | ALYLDGGLEPARRVVRQYWQ | |
| | | | | | | SRIESADRPHKEPKTLLQEY | |
| | | | | | | MLSQGLPLPHYELLSSDGPS | |
| | | | | | | HAPVFRVSVTTMGHTGTGEA | |
| | | | | | | GSKRLAESAAATALLKHLGO | |
| | | | | | | NVAS | |
| GO_ | GO_ | Sugar phosphate | H | | Up | MRDFTHDHSALALNTATLGH | 235 |
| 493 | 493 | isomerases/ | | | | NMLGAGAGWSAERTIDACAE | |
| | | epimerase | | | | RGIGGIVFWRPELQGRASAI | |
| | | | | | | GQHARQVGVEVVGLCRSPFL | |
| | | | | | | VGPLAPHGRQAVVDDFRRSI | |
| | | | | | | DETADLGGKILTIVVGGVEP | |
| | | | | | | GTKGVRESLDIVTDRLSEVL | |
| | | | | | | DYASERDVKLALEPLHPMYG | |
| | | | | | | GDRSCLPTVRDALDICDALN | |
| | | | | | | SDTLGVAVDVYHVWWDTDLP | |
| | | | | | | RQLERAGTRIMGFHLCDWLV | |
| | | | | | | PTTDFLLDRGMMGDGVADLK | |
| | | | | | | GLRAGVERAGYDGFCEVEIF | |
| | | | | | | SANNWWKRDPAEVLDVIIQR | |
| | | | | | | FRDIC | |
| GO_ | GO_ | TonB-dependent | H | | Up | MTTSVQHSLKRRTPAPRTVI | 236 |
| 2343 | 2343 | outer membrane | | | | RMFLMAGISYSVLPSFGAHA | |
| | | receptor | | | | QTTDATKHAAVHKTAAKKKT | |
| | | | | | | AAKQQVMPAAKNTPATTPVA | |
| | | | | | | AAAPAAKPATTTSVQTSNRT | |
| | | | | | | TLTTDPTPVAETVTVTGTRL | |
| | | | | | | SQTRLTNVMAGTTVDAEQLR | |
| | | | | | | ARGYTDLGLALLRENTAFTV | |
| | | | | | | GDNSPIGSQGSFGAGQSFIA | |
| | | | | | | LLGLGSQRTLTLIDGMRMVG | |
| | | | | | | GSTASNYGAGSGSQVDVSTI | |
| | | | | | | PTSLIKKIDTKLGGAGAAYG | |
| | | | | | | ADAVAGVVNYQLDDHFKGVD | |
| | | | | | | FNAQGNWTQKLDAPQEKITF | |
| | | | | | | KAGTSFDHDKGGVVFDVEYR | |
| | | | | | | NSGGMVANDRRYLTGDQATT | |
| | | | | | | YARAPVGSTSPYSYVLTPAV | |
| | | | | | | RFLQNSVTGVPMTSAAYGSL | |
| | | | | | | PLTYGQASQYGIANASGAGL | |
| | | | | | | MFSQDGKSLVPITTNAALKD | |
| | | | | | | GLRGSGGNGLALTDYNQLYA | |
| | | | | | | PSSKLNLTTLGHYDFTDHLH | |
| | | | | | | ATWQGWYARGTASSLTAQGT | |
| | | | | | | WNTPQFDDPLSAESYQQDTV | |
| | | | | | | VNGAYTLSTSNPYLTSAERT | |
| | | | | | | AIKSALAAAGQSTDTFYLSR | |
| | | | | | | LNQDLDAGNYTTTVQMFRFQ | |
| | | | | | | GGLNGDFDAVGRHFDWSVKG | |
| | | | | | | EYSKYMSDTWEPMIDTQNLV | |
| | | | | | | NALNATTDASGNIICTPGYT | |
| | | | | | | NSTAKTRSSTCSPLDPFGYD | |
| | | | | | | QMTLGAKNYITADAHSKESN | |
| | | | | | | VQRDIQAEIHSTVFRLPAGD | |
| | | | | | | IRWDLGYEHRREGYDFNPGS | |
| | | | | | | FMEGEEQADGTYKQYGNFTS | |
| | | | | | | IPYTGGAYHTHEVFGELDVP | |
| | | | | | | FVSPSMHIPGIYNLSATANG | |
| | | | | | | RYINNSVTGSYWTYMFGGAW | |
| | | | | | | WPTQDFGLSGNYAQSVRNPS | |
| | | | | | | VTELYSPTSTSYETANDPCS | |
| | | | | | | VEYISSGPNPATRAANCAKG | |
| | | | | | | GMAGGFESNINYYTLPGTTG | |
| | | | | | | GNRNLKNEVSKSFTGNLEIR | |
| | | | | | | PRFMKGFDFTGSFVDVKVNN | |
| | | | | | | EITSLDASDLMAACYDSTSY | |
| | | | | | | PSNAYCNAFTRDPSTHQVTS | |
| | | | | | | FTDGYFNIANQHMQVLQAKL | |
| | | | | | | DYYIPLRRFGLPSSAGNLEL | |
| | | | | | | QGNYTHYVKNQQTYLGSTYL | |
| | | | | | | LTGSTTSPNNLFTLDLNYTR | |
| | | | | | | GPLFVQWQTVYYGKSKYALQ | |
| | | | | | | VSDYTYQHNNRPDFAYFNTT | |
| | | | | | | IGYKITKNFDVNFMMNNITN | |
| | | | | | | ALPKYPGTVSLTRYYEAIMG | |
| | | | | | | RSFQMNLGAHF | |
| GO_ | GO_ | Tryptophan | H | | Up | MSRIQTRFAALKKEGRGALI | 237 |
| 2864 | 2864 | synthase | | | | PYLQACDPDYDTSLELLRAM | |
| | | alpha chain (EC | | | | PAAGADLIEIGVPFSDPSAD | |
| | | 4.2.1.20) | | | | GPTIQAAALRGLKAGATLGC | |
| | | | | | | VLEMVRAFRETDNETPIVLM | |
| | | | | | | GYLNPIDSYGPAEFCFDAAQ | |
| | | | | | | AGVDGIIVVDMPTEEADLLD | |
| | | | | | | AHAREAGLDIISLVAPTTSN | |
| | | | | | | SRLFHVLRDASGFVYYVSIT | |
| | | | | | | GITGTNSASAADLEKALPRI | |
| | | | | | | REATSLPVAVGFGISTPEQA | |
| | | | | | | RTASRIGDAAVVASALIKTM | |
| | | | | | | AGTLDDGRATERTVPAVLKQ | |
| | | | | | | LEGLAAAVRA | |
| GO_ | GO_ | Two-component | H | | Up | MVPNLSDPDLPARILVVEDD | 238 |
| 3253 | 3253 | transcriptional | | | | AGMRTLILRALQGGGFRARG | |
| | | responseregulator, | | | | VACGDEMWAALEVAPVDLII | |
| | | LuxR family | | | | MDIMLPGKSGIELCRALRSG | |
| | | | | | | QGATHENETPPAQVPIIMVS | |
| | | | | | | ARGEERDRVNGLESGADDYV | |
| | | | | | | PKPFGQRELLARVRAVLRRG | |
| | | | | | | IATGAPQERVRRETLRFAGW | |
| | | | | | | TLDLRRRELTDPSGATVDIS | |
| | | | | | | GAEHDLLTSFLDNPQRVIAR | |
| | | | | | | DRLLELSRTRLGDVSDRSID | |
| | | | | | | VLVSRLRRKLGSDADQLIRT | |
| | | | | | | VRGLGYIFVAEVERV | |
| GO_ | GO_ | Fructose- | L | F | Down | MSTAETMMSQMAEKGGFIAA | 239 |
| 1509 | 1509 | bisphosphate | | | | LDQSGGSTPGALKQYGIPES | |
| | | aldolase | | | | DYSGESEMFARMHEMRVRII | |
| | | class I | | | | TAPSFTGDKVIGAILFERTM | |
| | | (EC 4.1.2.13) | | | | DGDVKGEPVPAYLWRERGVV | |
| | | | | | | PFVKIDKGLEAEADGVQLMK | |
| | | | | | | PMPGLDDLLERAVKLGVYGT | |
| | | | | | | KERSTIRLPSESGIRAIVQQ | |
| | | | | | | QFAVAEQVRRHGLVPILEPE | |
| | | | | | | VLIKSPDKKACEQLLHDYVL | |
| | | | | | | EELQKLPEDARIMLKVTIPE | |
| | | | | | | VPDLYDDITRDPRMVRVVAL | |
| | | | | | | SGGYPLDQACEKLRHNHRMI | |
| | | | | | | ASFSRALIGDLRHQMDEAQF | |
| | | | | | | DATLGKTIDEIYDASVHKV | |
| GO_ | GO_ | Transport- | L | F | Down | MSGSTISSGQPTPDNTTTRP | 240 |
| 1829 | 1829 | related | | | | APLRPRARPFLSWLYAPSPS | |
| | | membrane protein | | | | SVTFALRNTIAACLAVGIAF | |
| | | | | | | WMELDDPAWAAMTVWAVAQT | |
| | | | | | | SRGESQSKAKWRIVGTISGA | |
| | | | | | | IAAITIMAAAPQAPWMFFPM | |
| | | | | | | IALWIGLCSGFATFVSNFRS | |
| | | | | | | YALVLAGYTCSIICMGAASN | |
| | | | | | | PDNVFMVAMSRGTYIVLGVL | |
| | | | | | | CEAFMGLIFATSQERHARAQ | |
| | | | | | | LRQKLESALVLVTTTLCSLL | |
| | | | | | | GEERGALNAARRQFGTILTI | |
| | | | | | | NDQIEFAEVEMGPHGHEGDH | |
| | | | | | | ARAALAAVSALLSRGFGMAM | |
| | | | | | | RLQVLNHNHPAFTKTADEIL | |
| | | | | | | AFLKQFPQRLPDQNAVPALL | |
| | | | | | | ADLQHFRDICRLYAAPHRES | |
| | | | | | | DIRPDLEPIPGLEPFDQTDE | |
| | | | | | | GQGMRADRLDERILFVSLGE | |
| | | | | | | LLGDLEQAIKEYEASTHRIK | |
| | | | | | | GDHFHFQLETHRDTREAVHN | |
| | | | | | | GLRGACAVLITAYIYEVTAW | |
| | | | | | | PNGLGFIAITTLVCGLFATK | |
| | | | | | | ENPVLGTTEFLKGAVAAYFM | |
| | | | | | | AWILVFVLMPKVTTFETLAL | |
| | | | | | | FLGPAMFLGGLAKGNPATAG | |
| | | | | | | GSAAYGLLLPAMLGLENHHV | |
| | | | | | | MNEIAFYNGNMATVLAVAVS | |
| | | | | | | VIVFRSVLPFSSDAERFRLR | |
| | | | | | | RIMLGELQRLAHPGFTPRIS | |
| | | | | | | VWIGRNTDRFARLIRHAGPT | |
| | | | | | | PAPLIEACILGTLATLTLGL | |
| | | | | | | NVIRLRTLLDREYLPESARR | |
| | | | | | | PILLVLHYVEQSTKRHDKAA | |
| | | | | | | RIAEAAVRRLRVLDAQETDL | |
| | | | | | | VTRLELTRAITYLVVIAYTM | |
| | | | | | | RTNEDFLDASKPFRGERNSR | |
| | | | | | | LLNSGQG | |
| GO_ | LepA | None (6 bp | L | F | Down | MTDTPLSLIRNFSIIAHIDH | 241 |
| 2805 | | upstream | | | | GKSTLADRLIQACGALTARE | |
| | | from Translation | | | | MKNQVLDSMELEQERGITIK | |
| | | elongation factor | | | | AQTVRLTYPAKDGKVYTLNL | |
| | | LepA) | | | | MDTPGHVDFAYEVSRSLAAC | |
| | | | | | | EGSLLVVDASQGVEAQTLAN | |
| | | | | | | VYQALDANHEIVPVLNKIDL | |
| | | | | | | PAAEPERVRAQIEDVVGIPA | |
| | | | | | | DDAVEISAKTGINIEGVLEA | |
| | | | | | | LVQRLPAPTGDAEAPLQALL | |
| | | | | | | VDSWYDAYLGVIILVRIKDG | |
| | | | | | | RLKRGDRIRMMQTGATYHVD | |
| | | | | | | QVGVFLPKMQSVESLGPGEM | |
| | | | | | | GYINAAIKTVADCNVGDTVT | |
| | | | | | | LDKRPAEKALPGFKPSIPVV | |
| | | | | | | WCGLFPIDADDFEKLRDSLG | |
| | | | | | | KLRLNDASFHFEAETSAALG | |
| | | | | | | FGFRCGFLGLLHLEIIQERL | |
| | | | | | | SREFNLDLIATAPSVVYKMH | |
| | | | | | | MTDGTSEDLHNPADMPELSK | |
| | | | | | | IETIEEPWIKATIMVQDEYL | |
| | | | | | | GPVLTLCSERRGIQVDLTYV | |
| | | | | | | GNRAMAVYRLPLNEVVFDFY | |
| | | | | | | DRLKSISRGYASFDYQMDGY | |
| | | | | | | EESDLVRISILVNHEPVDAL | |
| | | | | | | SFISHRTVAEQRGRSICAKL | |
| | | | | | | KDLIPKQLFKIAIQAAIGSK | |
| | | | | | | VIARETIGALSKDVTAKCYG | |
| | | | | | | GDISRKRKLLDKQKEGKKRM | |
| | | | | | | RQFGKVEIPQSAFLAALKMD | |
| GO_ | GO_ | Fructose- | L | F | Down | MSRVSPGVVSGASYTALIRA | 242 |
| 3252 | 3252 | bisphosphate | | | | CHEGGYALPAINVVGTDSVN | |
| | | aldolase | | | | AVLEAAARNGSDVIIQVSNG | |
| | | class II | | | | GARFYAGEGLPDAHRARVLG | |
| | | (EC 4.1.2.13) | | | | AASMARHVHLLAKEYGIAVI | |
| | | | | | | LHTDHADRKLIPWLDDLITM | |
| | | | | | | SEDEFKATGKPLFTSHMLDL | |
| | | | | | | STEPLEENLATSASMLKRLA | |
| | | | | | | PLGMGLEIELGVTGGEEDGI | |
| | | | | | | GHDLEEGADNAHLYTQPEDV | |
| | | | | | | LKAWELLSPIGTVSIAASFG | |
| | | | | | | NVHGVYKPGNVQLRPEILKN | |
| | | | | | | SQEAVQKATQSGPKPLPLVF | |
| | | | | | | HGGSGSTIAEIDAAVSYGVF | |
| | | | | | | KMNLDTDIQFAFAHGVGSYV | |
| | | | | | | LEHPVAFQHQIDPGTDKPMK | |
| | | | | | | SLYDPRKWLRVGEKSIVERL | |
| | | | | | | DEAFEILGSKGRSVARKS | |
| GO_ | GO_ | Transcriptional | L | F | Down | MSQTIVPHTLPLKNVHVPAR | 243 |
| 1025 | acrR | regulator, AcrR | | | | DRTATRAPGRPVNLRLKDNI | |
| | | family | | | | LAAIAQVLVEEGYQGLTISR | |
| | | | | | | VAKAAGVSTATVYRRWPTKQ | |
| | | | | | | AMFFDAMRLWRDDLTPQIDT | |
| | | | | | | GSFAGDVDELIAARIRFLVT | |
| | | | | | | PLGRTYGTLLGEAVHDPEFG | |
| | | | | | | QVLWEVSVVPARAQMTLFLE | |
| | | | | | | RASRRGEKVFCQNPDTVLDM | |
| | | | | | | LLSTIHFRAMNLLGREPMDA | |
| | | | | | | DSTLKEIRSLARSLIAG | |
| GO_ | GO_ | MBL-fold | L | S | Both | MHDAAVQAATGQIRRTEEGT | 244 |
| 1035 | 1035 | metallo- | | | | APAPVVCTFFDEATNTASHV | |
| | | hydrolase | | | | VHCPVTKRAAVIDSVLDYDA | |
| | | superfamily | | | | AAGRTSHGSAQAIVDYVERE | |
| | | | | | | GLTVDWQLETHAHADHLSAA | |
| | | | | | | PWLQEKIGGKLGIGADITRV | |
| | | | | | | QAVFGKIFNAGTRFARDGSQ | |
| | | | | | | FDHLFTDGETFRIGDLPVTV | |
| | | | | | | LHVPGHTPADMAFVIGNAAF | |
| | | | | | | VGDTIFMPDFGTARADFPGG | |
| | | | | | | DPRQLFRSIRRLLSLPVETR | |
| | | | | | | LFLCHDYKAPGRDEYAWLTT | |
| | | | | | | VGHERDYNIHVHDGVSEEEF | |
| | | | | | | VKMRTERDATLAMPRLLMPS | |
| | | | | | | VQVNMRGGHLPEPEADGVSY | |
| | | | | | | IKIPVNRV | |
| GO_ | lipB | Octanoate-[acy1- | L | S | Both | MPKTRAMTRNEIYEEILWKS | 245 |
| 1921 | | carrier-protein]- | | | | SPGLTAYPEALTFMEERARA | |
| | | protein-N- | | | | IHQGTAAPLVWLVEHPPVFT | |
| | | octanoy | | | | AGTSARDTDLYNPHGYPTYS | |
| | | ltransferase | | | | AGRGGQWTYHGPGQRLGYVM | |
| | | EC 2.3.1.181) | | | | MDLTKPNGTVPPRDLRAFVA | |
| | | | | | | GLEGWMTGALAQLGVTAFTR | |
| | | | | | | EGRIGVWTIDPLTGLEAKIG | |
| | | | | | | ALGIRVSRWVSWHGVSINVS | |
| | | | | | | PDLTDFDGIVPCGIREFGVT | |
| | | | | | | SLQRFDSSLTMADLDDALAA | |
| | | | | | | AWPGRFGSIPRAA | |
| GO_ | elf-2B | Nucleoside- | L | | Down | MIVIPMAGLSSRFTKAGYTK | 246 |
| 1731 | | diphosphate-sugar | | | | PKYMLPLAGKSVFAHSIESF | |
| | | pyrophosphorylase | | | | SAYFGLIPFVFIARPVADTE | |
| | | involved in | | | | AFIRKETAKLGIKDVRIIIL | |
| | | lipopolysaccharide | | | | DHETAGQAETVELGVRKAGL | |
| | | biosynthesis | | | | GLETPLTIFNIDTFRKNFRF | |
| | | /translation | | | | PDEPWFKKSDGYLEVFKGEG | |
| | | initiation | | | | ANWSYVGPEENSNEPLVART | |
| | | factor | | | | TEKKPISDLCCDGLYHFAHT | |
| | | 2B, gamma/epsilon | | | | RDFLDALTQERLTPSASELY | |
| | | subunits(eIF- | | | | IAPLYNYLIKAGRKIHYNLL | |
| | | 2Bgamma/eIF- | | | | PADMITFCGVPAEYTALLNA | |
| | | 2Bepsilon) | | | | IED | |
| GO_ | GO_ | Transcriptional | L | | Down | MTTDAEQDGPLRQRKKDRTH | 247 |
| 1683 | 1683 | regulator, AcrR | | | | AALVREAMRLFSTHGYEETS | |
| | | family | | | | VDEIAEAAQISRRTLFRYFP | |
| | | | | | | GKADIILAWTGSVTTILTEA | |
| | | | | | | IRDVPLDVPLQDAVLAGLVP | |
| | | | | | | VVACYSSDRMDAYAVVRLVE | |
| | | | | | | RTPALRDMALRRYSEWEETL | |
| | | | | | | ASALIARLPATEMPSLVARL | |
| | | | | | | LARTAIATFRTALDEWMKTE | |
| | | | | | | GKSDLEAILRQTFTLQPLLW | |
| | | | | | | QDDFPLSSG | |
| GO_ | bamE | Outer membrane | L | | Down | MNNASSPTPQSRTRLLRRFA | 248 |
| 1849 | | beta- | | | | QAGVACVPLLLGGCSFFSPI | |
| | | barrel assembly | | | | PEPRGSLIEKTDYAQLVPGT | |
| | | protein BamE | | | | STRTDVLDLLGSPTAHATFD | |
| | | | | | | DNTWIYVSMITSPTPLTFPS | |
| | | | | | | VKKQQVVVLNFDNGGVLRKM | |
| | | | | | | DTLNKKNAMYVGMVGAKTPT | |
| | | | | | | PGTSSSILQELLGNVGRYNP | |
| | | | | | | MSGMSSQFGGSTGPMGGQGT | |
| | | | | | | GNGGAGNTLP | |
| GO_ | envZ | Osmolarity | L | | Down | MRERDDAWQKLDRPLRRILP | 249 |
| 1848 | | sensor | | | | RSLMGRSMLIVLIPLLVTQA | |
| | | protein EnvZ (EC | | | | IALGLFYGTYLNVVSRRLSD | |
| | | 2.7.3. ) | | | | GVTGEVSLVIAMIEHTSSEA | |
| | | | | | | ERTLVLQDASSRTQLGFSFQ | |
| | | | | | | PGETLSRYGTNHVLGPMDDD | |
| | | | | | | FARSIRQNLGRPYDVDWSES | |
| | | | | | | PQTVRASIQLPQGVMVVMVP | |
| | | | | | | VKRLNIGPIWLFVVWAVSSS | |
| | | | | | | IVLFLIAGLFMRNQVRAIRR | |
| | | | | | | LGHAAELFGMGRDQGPIRPQ | |
| | | | | | | GAREVRQAAIAFNRMQARVN | |
| | | | | | | RFVAQRTAVLAGVSHDLRTP | |
| | | | | | | LTRLRLTVAMFPTFGPIRAE | |
| | | | | | | TLKPDLADMVADIEDMERLI | |
| | | | | | | GSYLSFARGEGAEEPVLTDL | |
| | | | | | | RGMLDDVAAATVRAGGQVLG | |
| | | | | | | VEGREGVEATVRPDALRRVL | |
| | | | | | | TNLAENARRHGGAMRFSLRE | |
| | | | | | | GERNVEITVEDNGPGLSASR | |
| | | | | | | RAAISELNGTTASQDGNSGL | |
| | | | | | | GLTIVRDIIHAHGGSIRLVE | |
| | | | | | | SPLGGLGVVLSLPK | |
| GO_ | ggt1 | Gamma- | L | | Down | MAHRQHQISTPDATQARRVS | 250 |
| 1517 | | glutamyl | | | | SRLPASLMASVASGLLLGAC | |
| | | transpeptidase (EC | | | | SWIPGSHLVGISETSAPAGG | |
| | | 2.3.2.2) | | | | SIGTVVADEPQAALVGHDVL | |
| | | Glutathione | | | | ARGGNAADAATATALALGVT | |
| | | hydrolase (EC | | | | LPSRASFGGGGACLVSRPGE | |
| | | 3.4.19.13) | | | | VAQSIAFQPRAGTSKGADRP | |
| | | | | | | AATPADFRGLYLMQLHYGTV | |
| | | | | | | DFNDLIAPALNLASTGITVS | |
| | | | | | | RTLAADLAAVRPALLADDSA | |
| | | | | | | RAIFGRGDASTLVAGDSLAQ | |
| | | | | | | PRLAGFLERIRVAGVGDLYN | |
| | | | | | | GALADVYVTAAQKAGGGLND | |
| | | | | | | DDMRQTLPLQTEALITRQGG | |
| | | | | | | VDASFLAPPADGGLGAAVRY | |
| | | | | | | TTGASAQGTVAAWRASGNTS | |
| | | | | | | LAAAQAALNSGRSGGGSLPA | |
| | | | | | | LPASTSFVVTDHSGMAVACV | |
| | | | | | | LSDNNLFGTGRIAGSTGVVL | |
| | | | | | | GASPAHTPQPLLSAAVLRDQ | |
| | | | | | | RNLRAVIAASGQNEAADAVG | |
| | | | | | | DAARAAAHETPIPHEGAGRI | |
| | | | | | | NAILCHGNDSCRGSTDPRAA | |
| | | | | | | GLAAGTTNDSRN | |
| GO_ | GO_ | Putative outer | L | | Down | MLNDNLPHFDGRHYRNPEAW | 251 |
| 1348 | 1348 | membrane | | | | RPDETRTARTTSQRIGNIFR | |
| | | protein | | | | WQMGLRPRWPDALPPQKSWP | |
| | | | | | | QVTPAPGECCVTFIGHATVL | |
| | | | | | | LQFGRTGRAPLRIITDPVFS | |
| | | | | | | ERCSPFRSFGPRRVRPPGIP | |
| | | | | | | LDALPRIDIILVSHCHYDHL | |
| | | | | | | DLPSLRALAERDDPLCLSLP | |
| | | | | | | GNRRHLEKADLPRIAELDWW | |
| | | | | | | ESTTDDGARITATPALHGSA | |
| | | | | | | RTPFDSNRALWGGFTIEADG | |
| | | | | | | HTVFFAGDTAWGKHFDAIHR | |
| | | | | | | RWPDINLAILPIGAYDPRDL | |
| | | | | | | MRRVHMSPEEALMAFDTLRA | |
| | | | | | | KQGLPIHFGTFQLTDEAILE | |
| | | | | | | PPTRLEFASRPGSRPFAALE | |
| | | | | | | NGESLTLPPADTKS | |
| GO_ | GO_ | hypothetical | L | | Down | MGIAGASCVLDVMINDRSAL | 252 |
| 1415 | 1415 | protein | | | | VRDSAAFIVLLERIWKARDV | |
| | | | | | | EASLVWSELDERIRLADELR | |
| | | | | | | ASGIRPYKGGRFRSTKLP | |
| GO_ | GO_ | Glycosyl | L | | Down | MQHPVLTILPPRERYEEGHA | 253 |
| 1438 | 1438 | transferase, | | | | GAISLLVSREAQFSDVVAGM | |
| | | group 1 | | | | GRIGTPLPGGQYRPLILPRV | |
| | | | | | | PLLRNWRLRLACVAMMRFCR | |
| | | | | | | PALTEIHNRPDLARFLARFG | |
| | | | | | | PVRLILHNDPCTMRGARSPR | |
| | | | | | | QREDLARHVLVCGVSEWVTG | |
| | | | | | | RFCEGCGPIRTEVQPNCIDL | |
| | | | | | | SVLPTVGLRQKIVLFAGRVV | |
| | | | | | | ADKGVDAFVRAWGAIRAQYP | |
| | | | | | | GWRAVIMGADRFGPDSPETP | |
| | | | | | | FLEKLRPQAAAVGIMMTGYR | |
| | | | | | | PHDDVLEAMAGAAVVVVPSR | |
| | | | | | | WEEPFGMTALEAMGCGAAVV | |
| | | | | | | ASPVGALPDLVGDAALLAAP | |
| | | | | | | DEAGALEQALSRLMGDEGLR | |
| | | | | | | TRLGERGRERAALFDVAAAR | |
| | | | | | | VRQEELRRAAIAFARRPPRL | |
| GO_ | GO_ | Two-component | L | | Down | MHIVPRSLVARTSLLLIVGL | 254 |
| 1455 | 1455 | system sensor | | | | AVVEAAGLGIHALDRFDLEE | |
| | | histidine | | | | RSQVHEEQVQVFSIYRTVAE | |
| | | kinase | | | | AKPADRHDAIDDLHVPSNVT | |
| | | | | | | VLLLKEPDPLIEGHEIPFPA | |
| | | | | | | MTSPSFLHRLHEDPQGHEGF | |
| | | | | | | LPPGPMDPGGPHPGPDMGGP | |
| | | | | | | GFPGAGPGPMGPMGGGPGGP | |
| | | | | | | EFHFPADHADGERADAEHLN | |
| | | | | | | GDPRFPGAIRRRDMPPFMRW | |
| | | | | | | ALLPSSLYPRKVLIGQKMRT | |
| | | | | | | HSTSILLPDRDCWLVVRFVT | |
| | | | | | | PLPNPFGSPTFMIAFLVMSI | |
| | | | | | | AGSAMIVWATQRLIAPVTTL | |
| | | | | | | ANAAEALGRDVHTAALPENG | |
| | | | | | | PSEIRRAAIAFNTMAMRIRR | |
| | | | | | | FVTDRTLMLTAIGHDLRTPI | |
| | | | | | | TRLKLRAEFIDDEELRNKVL | |
| | | | | | | ADLDEMESMVSATLAFGRDS | |
| | | | | | | ASAEPIVSLDLRALLQTIMD | |
| | | | | | | EAAESVPDKADDLFYEPPNV | |
| | | | | | | PVRIKARSVALKRALNNLIL | |
| | | | | | | NALKYGGSAHVTLIPATNPG | |
| | | | | | | EKDNVVKILIEDNGPGLPES | |
| | | | | | | ELDRVFEPFVRIESSRNRET | |
| | | | | | | GGSGLGLSIARTILHGQGGN | |
| | | | | | | VRLENRPSGGLRVIVTLAP | |
| GO_ | GO_ | hypothetical | L | | Down | MPPRDRRTRPTVDRRRALVG | 255 |
| 1728 | 1728 | protein | | | | LGLAAPFLASRSIAAEKSVP | |
| | | | | | | CLRIDEVELRRFGAALDGIT | |
| | | | | | | DDLHVLQACIDSSGVNTPDV | |
| | | | | | | PCPSILFDFPAVTVCLSGPI | |
| | | | | | | VTGGRNITLRGAGQDLTVLI | |
| | | | | | | MKTGASGILTHGTSEYPAEG | |
| | | | | | | YLQLQDIGFDDGNIKGSGCT | |
| | | | | | | GISVHFAPGAPQAAMTWQGV | |
| | | | | | | ALRKWSQAATITNCPRNWHC | |
| | | | | | | ENVTVFGPDFTMQDDAGFRI | |
| | | | | | | ISEPHFAQGCFTYVFINVLV | |
| | | | | | | ANYSWGWDYSISAPLEGQRF | |
| | | | | | | YSCTCYNGWGMVRSRVHATP | |
| | | | | | | DQQSGIDETYRSVIWYFMEC | |
| | | | | | | DWQGFGYALDLVHCRNIIVR | |
| | | | | | | GGFYIANRNVDHLPIPEGRV | |
| | | | | | | RRRYMSFVDCGDILLDGVKF | |
| | | | | | | DVFTGSEPDLALIYVDGRSD | |
| | | | | | | NFRARDTNILSYAPIYCAFE | |
| | | | | | | FADPSHPNLKRNTLSEIDTL | |
| | | | | | | WASWSGGEKVRDAGGNQITQ | |
| | | | | | | TSVRDLGGDMTSGGRISFQG | |
| | | | | | | HVTLSRGKSMIAFPHRQNGN | |
| | | | | | | SWFSKTPIVFLQTEGTEDVP | |
| | | | | | | KLLNVTSDGISIEVTSNSAA | |
| | | | | | | IMWQATGT | |
| GO_ | GO_ | Transcription- | L | | Down | MEQNETMTREMSTASVGRSF | 256 |
| 1912 | 1912 | repair | | | | GPTVWGVPDGSVAFLLRQRL | |
| | | coupling | | | | AEHDGPLLHVARDDAAVAAL | |
| | | factor | | | | ADMLAWLMPEVEVLRLPAWD | |
| | | | | | | CLPYDRVSPNPVLIAERAGT | |
| | | | | | | LCRLLEPTKARRIVLTTVHS | |
| | | | | | | LIQRVPPRSAFRGQSISVKT | |
| | | | | | | GESLDQAMLIELLIANGYTR | |
| | | | | | | TDTVMEAGEFATRGGIFDLF | |
| | | | | | | PAGESDPIRLDLFGDEVENI | |
| | | | | | | RAFDVGTQRSTETLKRFELR | |
| | | | | | | PVSEFSLGPDSISRFRTGWR | |
| | | | | | | DSFGPAATSDTIYENVSDGR | |
| | | | | | | RYPGLEHYLPLFHDGDGHQM | |
| | | | | | | ETLLDYLPGGAVSFDHHAPE | |
| | | | | | | ILKARLDMIADHYQARRVPT | |
| | | | | | | REGEIPYRPLPPHRLYLDAH | |
| | | | | | | GWESMLADVPSVILNAFAMP | |
| | | | | | | DTAQGVDAGYRPGPLFARAK | |
| | | | | | | DGSRAGMFEAFGQQVKTWAE | |
| | | | | | | AGRRTYVTAWSRGSRERISH | |
| | | | | | | LLAEHGVTATSYEDWPDAAG | |
| | | | | | | MPKGTTGLLTLGLERGFVSD | |
| | | | | | | RLCFVSEQDLLGERISRPPR | |
| | | | | | | RRKRGEQFISQVGEIAEGDL | |
| | | | | | | VVHSDYGIGRYVGLETVVEG | |
| | | | | | | RVAHDYLSILYDGEQRLLLP | |
| | | | | | | VENIELLSRFGSEQAGVQLD | |
| | | | | | | KLGGTAWQARKAKMKSRIRV | |
| | | | | | | MAGELIKTAAARALKDAPTL | |
| | | | | | | APAEGLWDEFCARFPFVETE | |
| | | | | | | DQSRAIADVLEDMSAGRPMD | |
| | | | | | | RLVCGDVGFGKTEVALRAAF | |
| | | | | | | VAALSGMQVAVVVPTTLLAR | |
| | | | | | | QHFRSFSTRFEGFPVNVAQL | |
| | | | | | | SRLITPKEATKVREGMADGT | |
| | | | | | | VDIVVGTHALLAKTVSFERL | |
| | | | | | | GLLIIDEEQHFGVAHKERLK | |
| | | | | | | ALREDVHVLTLSATPLPRTL | |
| | | | | | | QLSLSGVREMSLIATPPTDR | |
| | | | | | | LAVRTFITPFDSVMIREAIQ | |
| | | | | | | RERFRGGQIFCVVPRLADMD | |
| | | | | | | RMAERLTEIVPDAKTVQAHG | |
| | | | | | | RLTPTELERVMTEFADGKYD | |
| | | | | | | ILLSTNIVESGLDMPSVNTI | |
| | | | | | | IIHRADMFGLGQLYQLRGRV | |
| | | | | | | GRGKQRGYAYLTWPQTRPLS | |
| | | | | | | PSSEKRLEVMQTLDSLGAGF | |
| | | | | | | TLASHDLDLRGAGNLLGDEQ | |
| | | | | | | SGHIKEVGIELYQQMLEDAV | |
| | | | | | | IDMRRERGKRQDDEDSWTPT | |
| | | | | | | IILGLPVLIPEAYVPDLPVR | |
| | | | | | | LGLYRRIASLTNEAEVEAMR | |
| | | | | | | AELVDRFGSLPPEVGNLLDV | |
| | | | | | | VLIKRLCREAGVERLETGPK | |
| | | | | | | GMVIQFRRNRFANPAALIDW | |
| | | | | | | VARKKESGVKIRPDHKLAVI | |
| | | | | | | REMTNATRIGYAKKVTTVLC | |
| | | | | | | KMIRKLEAAKSGD | |
| GO_ | GO_ | Xanthine | L | | Down | MTAAAQFSPLPQWPLYGLTD | 257 |
| 192 | 192 | and CO | | | | DLFPTLERWSAEGKRAALAT | |
| | | dehydrogenases | | | | LVSITGSSPRPLGSEMAICE | |
| | | maturation | | | | DGEVQGYVSGGCVEAAVRAE | |
| | | factor, | | | | ALESLKDGQPRMLDYGAGSP | |
| | | XdhC/CoxF | | | | VLDIQLTCGGRIGIFVRALP | |
| | | family | | | | DLTAHVATLKAARENRRPIT | |
| | | | | | | LLTDLDTGAMQFCPEARATG | |
| | | | | | | LEGRTFARQYLPPLRLILSG | |
| | | | | | | GDPITLALLSLSALMGLETT | |
| | | | | | | LLRPYGPPGPPPGLSPTRYI | |
| | | | | | | RGSLDAALPTLSLDRWTAVY | |
| | | | | | | SLTHDAFADLTLTAHALKSE | |
| | | | | | | AFCVSILGSRRKIPGRLEAL | |
| | | | | | | RTAGVPEEAFSRLHLPAGLE | |
| | | | | | | IGARTPMEIALSILTQMITT | |
| | | | | | | RPR | |
| GO_ | GO_ | hypothetical | L | | Down | MKKEKSAIVLFVKDEAHDIM | 258 |
| 2170 | 2170 | protein | | | | AWLSWHISLGFDKIFVYDDH | |
| | | | | | | STDGTYEIAKSCEGIYNVEV | |
| | | | | | | CRTSMLEGNFYYRQRDSYFD | |
| | | | | | | AIKKSIDHYEWIAMLDGDEY | |
| | | | | | | VSIDGTQDINGFLDKFRKDD | |
| | | | | | | TGIALSWCIYGSSSRVLKDK | |
| | | | | | | VPTYQVFNHRSTPELNDNTL | |
| | | | | | | VKSFVRPEAVSFVYENPHKF | |
| | | | | | | NLSYGNYADAAGQPVEWRPG | |
| | | | | | | ATKNILWEGARINHYICRSM | |
| | | | | | | EHFIDCIKRRLGSDLSNSTV | |
| | | | | | | YWSHFDRNDVYDPQDQARIN | |
| | | | | | | AANTVYNNIKKSVFQYSMKN | |
| | | | | | | FLEKGNALENTENSLTPHRK | |
| | | | | | | VDLFHLKSIHGEYLSLNNID | |
| | | | | | | GHLFQGEGFERILAAIPYDS | |
| | | | | | | NKIWLFRNPFVYSSNVRFHI | |
| | | | | | | SHSAQSNYCYEFAFDSNESD | |
| | | | | | | GSIFIQSPKTQKYLTCIPVG | |
| | | | | | | HGGSVEFSREEASDWEKFYF | |
| | | | | | | GEKVSELTFVGDNEGPASDL | |
| | | | | | | IYYMLNSAGSFPYEEFLLKS | |
| | | | | | | STLESNDRMNLKSLLGPQIM | |
| | | | | | | SII | |
| GO_ | GO_ | Glycosyl | L | | Down | MRDDPRETLTESYRSAARLN | 259 |
| 2173 | 2173 | transferase | | | | GLRAEHLQKENQKLLRQLFL | |
| | | | | | | IQTSLSWRVTLPLRAVRALT | |
| | | | | | | FGRLLSGRPVSELPGRFLRL | |
| | | | | | | WGREGLAGIRKTVIHRVRRL | |
| | | | | | | KRIHGDRQKVSTASTAETGG | |
| | | | | | | LHPYRATPECGAARLKPQVL | |
| | | | | | | IIAELSLRQCAKYRVWQKRD | |
| | | | | | | FLQTLGWSVQVVDWRDLAEA | |
| | | | | | | QSALQLCTHVIFYRVPGFDD | |
| | | | | | | VMKLVQEAHRLGLAPRWEVD | |
| | | | | | | DLIFDEGEYRQNGNIDTLPA | |
| | | | | | | AERDLLLSGVALFRRCMLAC | |
| | | | | | | GRGIASTAALAGAMREAGLT | |
| | | | | | | DVAVLENALDAQTLGIAEVL | |
| | | | | | | PLPVPSGRIWVSYGSGTNTH | |
| | | | | | | DADFRQAEAGLLAAMDEEPR | |
| | | | | | | LCLRVIGQLQLSSAFSRFGD | |
| | | | | | | RVERLTELTYPDYLKALSQT | |
| | | | | | | DIAIAPLEKTLFNDCKSNIK | |
| | | | | | | FLEAAIVRVAAICSPCAAFL | |
| | | | | | | TVLRDGKNGLLAADTAAWRN | |
| | | | | | | GFLALARDGDYRKRLAEAAY | |
| | | | | | | KDVMARYAPKAMAQTQARAL | |
| | | | | | | FGLPPSREAEGLRILMANVY | |
| | | | | | | FAPRSFGGATIVAEEMGRRL | |
| | | | | | | VRKGVQVSVMTSRPPAVDIP | |
| | | | | | | DGDVRYDVDGMMVFASVLPD | |
| | | | | | | GLDGVGHLDNPAMASRFADM | |
| | | | | | | LDACQPDVVHVHAAQGLGTG | |
| | | | | | | ILRICQERGIPYVLTLHDAW | |
| | | | | | | WLCERQFMVKGDGRYCFQTT | |
| | | | | | | IDPAVCQACVPGVRHLADRT | |
| | | | | | | VVMRQALAGAALLISPSHAH | |
| | | | | | | RELYLANGIAPERIVVNRNG | |
| | | | | | | FCWPKRARRPRSPGTPLRFG | |
| | | | | | | FVGGTEAVKGYGLLKEAMQS | |
| | | | | | | LSRSDWELVVVDNKLNLGFQ | |
| | | | | | | SIFPEDWTVRGKLRVVPAYT | |
| | | | | | | QASLDDFYDQIDVLLFPSQW | |
| | | | | | | KESYGLTVREALARDVWVVT | |
| | | | | | | TSPGGQSEDVVDGVNGTWLP | |
| | | | | | | LDGKPQTLVQAVSALLEAPE | |
| | | | | | | RFEGYVNPYKEQLATYDMQA | |
| | | | | | | DELYGFLKRAAGQPSGRGAG | |
| | | | | | | L | |
| GO_ | GO_ | hypothetical | L | | Down | MDLLRWTIAFLILALCAAVL | 260 |
| 2178 | 2178 | protein | | | | GFGGISADFAYIGKILFFIF | |
| | | | | | | LVLLIISLIFGRGRGTRL | |
| GO_ | GO_ | Uricase | L | | Down | MTQDSYPRDMIGYGRTPPDP | 261 |
| 2386 | 2386 | (urate | | | | KWPNGARIAVQFVINYEEGA | |
| | | oxidase) (EC | | | | ENSVLHGDAGSEAFLSEMVG | |
| | | 1.7.3.3) | | | | TKSIIGARCAQMESLYEYGS | |
| | | | | | | RAGFWRLRRLFDEAGMPVTV | |
| | | | | | | FGVAKALARNPDAVAAMKES | |
| | | | | | | GWEIASHGLRWIDYQDFPED | |
| | | | | | | LERAHIRKAIALHTEVTGER | |
| | | | | | | PLGWYQGRTSPNTARLVAEE | |
| | | | | | | GGFVYDADSYADDLPYYDRS | |
| | | | | | | NGRAQLIVPYTLDANDMKFA | |
| | | | | | | ALNGFTEGEQFFIYLRDAFD | |
| | | | | | | MLYREGGRMMSVGLHCRLAG | |
| | | | | | | KPARAMGLLKFLEHIRKHED | |
| | | | | | | VWVATRLDIARHWLSVHPA | |
| GO_ | GO_ | Uncharacterized | L | | Down | MMSRIQLTVLRPLHRPTTRL | 262 |
| 2634 | 2634 | protein | | | | ALGFLALGALAACGSGRDPS | |
| | | CC_3748 | | | | TLTAPRNHLLGVDRGAEGGA | |
| | | | | | | DELRGGVNAYLWRGAIDTLS | |
| | | | | | | FMPLASADAVGGVILTDWYQ | |
| | | | | | | PSASQNERFKIAAYVLDRRL | |
| | | | | | | RSDALRVSVFRQVLQDGQWE | |
| | | | | | | DTPVSATTTSDITTRILTRA | |
| | | | | | | RQLRAENGERDN | |
| GO_ | GO_ | Type II | L | | Down | MSGEFPVDQILRGECIETMK | 263 |
| 2698 | 2698 | restriction | | | | TLPDGSVDCIFADPPYNLQL | |
| | | adenine- | | | | RGELRRPDETVVDGVDDDWD | |
| | | specific | | | | KFADYATYDNFTREWLSEAR | |
| | | methylase | | | | RILHKDGTIWVIGSYHNVFR | |
| | | (EC 2.1.1.72) | | | | LGAIMQDLGFWILNDIVWRK | |
| | | | | | | SNPMPNFRGRRFTNAHETLI | |
| | | | | | | WAARGPQSKYRFNYQAMKAL | |
| | | | | | | NDDLQMRSDWYLPLCTGNER | |
| | | | | | | LKNEHGLKLHPTQKPESLLH | |
| | | | | | | RVLVASTNANDVVLDPFCGS | |
| | | | | | | GTTPAMAKRLGRHYIAIERH | |
| | | | | | | PDYVKAARERVAREERLTSE | |
| | | | | | | QLATTPAKREMPRIPFGSFV | |
| | | | | | | ETGVLPAGTLLYDRQKRLKA | |
| | | | | | | TVTPDGTLVSGNQRGSIHKL | |
| | | | | | | GAMLTNAPSCNGWTFWYFER | |
| | | | | | | DGQYVQIDVLRQESQALRNV | |
| | | | | | | G | |
| GO_ | GO_ | Two-component | L | | Down | MPLVTPNMPRVVLVEPDCDH | 264 |
| 2776 | 2776 | system sensor | | | | ARQIVQVLVKEGFALTCATS | |
| | | histidine | | | | GEEALGTIEETMPDLVVACT | |
| | | kinase | | | | ELPGMSGGQLARRLRLDALT | |
| | | | | | | RNIPILMLTEDASPGVEREG | |
| | | | | | | LESGADAYISKSAHPDLMVL | |
| | | | | | | RMRALLREGPELLQVDEASR | |
| | | | | | | LRRARIVIVNSPREDEDEEE | |
| | | | | | | VVEDVPETTLGELLWRDGHT | |
| | | | | | | VTSIERSDDLIEGGWLRGAD | |
| | | | | | | SPDCLVLELGSGDEDLKFCR | |
| | | | | | | LLDARRQAVLEAGGIPFRTL | |
| | | | | | | GIVEASRFRRQSSGEFFEAG | |
| | | | | | | IDDLVPSDIALEALAMRIRT | |
| | | | | | | LAQRRMAQDEFRQQEIERQQ | |
| | | | | | | NALTLEAARAKAEMAEALAQ | |
| | | | | | | ANMELARTNERLLQVQSKLV | |
| | | | | | | QTAKMASLGELVAGIAHEIN | |
| | | | | | | NPLAFTIAHADTVTRTLKRL | |
| | | | | | | QGVNASDEAMSLTKKGMSRL | |
| | | | | | | ESMKLGLQRIQNLVLSLRRF | |
| | | | | | | SRLDESSFQKVDVPAALETA | |
| | | | | | | LALLAHKLGPGIIVQKDLQA | |
| | | | | | | PAELVCQPAFLNQVVMNIIS | |
| | | | | | | NAADALADMSTDGDIVRGRI | |
| | | | | | | VIASRLENGRYEMRVSDDGP | |
| | | | | | | GLPPDLRTRIFDPFFTTKPV | |
| | | | | | | GTGTGLGLAIAYSVMEAHDG | |
| | | | | | | VIEVTDANLPDGRGIGACFR | |
| | | | | | | MSLPVRMTEEGPVATGRAA | |
| GO_ | GO_ | Oxidoreductase | L | | Down | MKLFELGERTAQVHDVLVTV | 265 |
| 2861 | 2861 | probably | | | | AELAERDDARAVLLESADDP | |
| | | involved in | | | | ALLKPYLNRLDLVVLRFPVF | |
| | | sulfitereduction | | | | RDGRGFTQARELREYLRFSG | |
| | | | | | | EIRAEGHILPDQAAFLRRCG | |
| | | | | | | VDSVVLPKDGNGDPALWEKQ | |
| | | | | | | LRQFPVAYQRSVLPERSVGP | |
| | | | | | | GLRVEEAS | |
| GO_ | GO_ | hypothetical | L | | Down | MTRIILQHGQNAPLTLDIEE | 266 |
| 3031 | 3031 | protein | | | | GSTTPIHLHFSQALPVAAPQ | |
| | | | | | | PEIAPVGPQAAPASKIRRFL | |
| | | | | | | PVAATAAVCAGLLFTFGGPR | |
| | | | | | | ASAPAMPESAPLPPLPSSGP | |
| | | | | | | LETQPQGPAPTAPQQILKAL | |
| | | | | | | HQPAHVEMPPSAPAQAGSPF | |
| | | | | | | GLEN | |
| GO_ | GO_ | hypothetical | L | | Down | MTQGVAEEMANSESQTPKAL | 267 |
| 967 | 967 | protein | | | | LAAVILMGVLIIAAVFGLIG | |
| | | | | | | VIAYRFLHPRPSVTATVPLA | |
| | | | | | | EGGFSRLALPLGAGEHITAV | |
| | | | | | | TSRPDGLMAVTLSGAGSDRV | |
| | | | | | | LLWNPEAGKIAAELDFGTPA | |
| | | | | | | QSTP | |
| GO_ | hII | Ribonuclease HII | L | | Down | MPDYALEAAHGGLVVGIDEV | 268 |
| 2697 | | (EC 3.1.26.4) | | | | GRGPLAGPVVASAVAFTAPP | |
| | | | | | | SETLSSLLDDSKKLTARRRM | |
| | | | | | | LAYEALMADEQALIGVGAAS | |
| | | | | | | VAEIERINIAQACYLAMRRA | |
| | | | | | | LSRLGCTPDLALVDGKHAPK | |
| | | | | | | LPCPIKMVIGGDGISLSIAA | |
| | | | | | | ASIIAKVTRDRLMARLAVRH | |
| | | | | | | DAYGWERNAGYGTAAHLQGL | |
| | | | | | | KLRGVTPHHRRGFAPIRNMI | |
| | | | | | | EAEAHAA | |
| GO_ | htrB | Lipid A | L | | Down | MTSFLYRAETLLVRALLAAI | 269 |
| 2440 | | biosynthesis | | | | RTLPPAASSSLGGFVARTVG | |
| | | lauroy1 | | | | PLLPVSKVADRNLQLALPEY | |
| | | acyltransferase | | | | DASARRRIVRDCWENLGSTV | |
| | | (EC 2.3.1.241) | | | | GEFPHISRLKQNTPSGAGWS | |
| | | | | | | VEGAEHLEAARASGRPVIFF | |
| | | | | | | SGHIGNWELMPPVVARYGMP | |
| | | | | | | FASFYRAASNPGVDRLIHRL | |
| | | | | | | RQDAMGQDVPMFPKGAKGAR | |
| | | | | | | AALKYLSKGGNLGVLGDQKM | |
| | | | | | | NDGIEARLFGRPAMTASAAA | |
| | | | | | | VFALRHDALIVTGHVRRDGP | |
| | | | | | | ARLVLVVDAPFMPTKTDDRA | |
| | | | | | | KDVLVLTQLFNDRLESWIRD | |
| | | | | | | IPGSWLWLHRRWDKSLYRNM | |
| | | | | | | TTQC | |
| GO_ | moeA | Molybdopterin | L | | Down | MSELLSVTRAMELVLDYAGS | 270 |
| 193 | | molybdenum | | | | FGTETVDLTMAAGRILRQTV | |
| | | transferase | | | | RAERAQPPYDRVMMDGIAFR | |
| | | (EC 2.10.1.1) | | | | HGSGPTLVSHGIQRAGASGQ | |
| | | | | | | VLPEGHVCLEVMTGAVLPDG | |
| | | | | | | ADTVVPVERLVREGRLIRFE | |
| | | | | | | EGYEPRKGQFIHRRGSDCAA | |
| | | | | | | GTELLAPGQRLDGPALAVLA | |
| | | | | | | GNGHAQVSVSRIPSIGIVAT | |
| | | | | | | GDELVDVSAPVRDWEIRRSN | |
| | | | | | | EYALTGAMLSRGFNCIERSV | |
| | | | | | | VPDDLAETVVALREQLSRHD | |
| | | | | | | VLILSGGVSMGAFDHVPKAL | |
| | | | | | | SKIGVERVFHKVAQRPGKPL | |
| | | | | | | WFGVGPEGQRVFGLPGNPVS | |
| | | | | | | ATTCGVRYVMPMLLAGQGLC | |
| | | | | | | RPEPYTVMLDAEADLIPTLT | |
| | | | | | | RFLPVKLRHDATGQALATPC | |
| | | | | | | PMPTSGDFSFLASTDGFMEL | |
| | | | | | | PRGEGVAPRGTAAMFHGW | |
| GO_ | nifS | Cysteine | L | | Down | MIYLDYLSTTPCDPAVVKAM | 271 |
| 85 | | desulfurase | | | | LPWFGEDFGNPHSPHGPGRK | |
| | | (EC 2.8.1.7) | | | | AAEAVERAREIVARLLGVEA | |
| | | | | | | REIVFTSGATEANNLAIKGA | |
| | | | | | | VRHLARVGDPRRRIVTLATE | |
| | | | | | | HKCVLESVRDLESEGFEAVV | |
| | | | | | | LGVDSEGRVDPEALRDALKV | |
| | | | | | | PTLLVSIMAANNETGVLQDI | |
| | | | | | | PQLAQIVKERDALMHVDLAQ | |
| | | | | | | MAGKMPVSLRNVDLASVSAH | |
| | | | | | | KMYGPKGIGALYVRRKPRVR | |
| | | | | | | LEPLFSGGGQERGLRSGTLA | |
| | | | | | | TPLVVGFGEAADLAAETMGD | |
| | | | | | | EAARQVFLRDDLWAQLREGL | |
| | | | | | | PGIVLNGAGAPRLAGALNVC | |
| | | | | | | LPEGCRALDVLEACPDVAAS | |
| | | | | | | TGSACTAAEIAPSYVLTAMG | |
| | | | | | | LSAEKASRCLRLSVGRFTSK | |
| | | | | | | ADIDRAASFLIAAARACHPN | |
| GO_ | oprB | Carbohydrate- | L | | Down | MGKFGQDWTKALLTACAMTA | 272 |
| 628 | | selective | | | | VLPGITAVGHAQTTPAGVVD | |
| | | porin OprB | | | | GHQKAAARTSTATMGTPQIR | |
| | | | | | | TKSISPVPLLVKPAPTKTGV | |
| | | | | | | ETAAKTSDTTESRFFPASFH | |
| | | | | | | DWLTQSTMTGDWGGWRTWLT | |
| | | | | | | DKGINIGGHYLEDSAGNPMG | |
| | | | | | | GKTKAVRYADEFGINVDFNL | |
| | | | | | | KKLTGLNLGMFHTLITARQG | |
| | | | | | | LGIGATLPALDSPQQIFGSG | |
| | | | | | | ETVRLTRLSWEMPWNKYVTT | |
| | | | | | | EVGEINTENDFEQSSVYWGM | |
| | | | | | | SQYCQFESNAICGMPQSIAM | |
| | | | | | | NSGYGWYPTAHPGAWVKFYP | |
| | | | | | | AGNDHYLVQFGAYSVDPVIS | |
| | | | | | | NTHNGWKLNLHDATGTYLPF | |
| | | | | | | QLGWHQGGKDDYSGPLQTNI | |
| | | | | | | KIGGYWDTSEVSDVYSHLGT | |
| | | | | | | FGVPAQYLISAPSEKVRGRF | |
| | | | | | | GGWFQFDRMLQRDEADPNRG | |
| | | | | | | TTLFTSFTWGDPRTSVAPYF | |
| | | | | | | ITWGVTRKGTFRSRPNDTIS | |
| | | | | | | IGMKMLWVNPKLTNWVRQIQ | |
| | | | | | | AAGGTDIYKPSGEHALELNY | |
| | | | | | | GWRPTPWLVIRPGAQYIWST | |
| | | | | | | GGTNRYKNPLLLDFETGITF | |
| GO_ | pdh1 | Pyruvate | L | | Down | MASLILMPALSPTMTEGTLA | 273 |
| 2072 | | dehydrogenase E1 | | | | RWVRKAGDTVAAGDVIAEIE | |
| | | component beta | | | | TDKATMEVEAVDEGVIGKTL | |
| | | subunit | | | | VDEGTQNIAVNTPIAVLLAE | |
| | | (EC 1.2.4.1) | | | | GEDASAADNVVRSSDPAVGA | |
| | | | | | | PVAIETPSDPAITEAPAVAQ | |
| | | | | | | AEDDRDWGETSEITVRQALR | |
| | | | | | | DAMAAELRRDEDVFLIGEEV | |
| | | | | | | AQYQGAYKISQGLLEEFGEK | |
| | | | | | | RVIDTPITEHGFTGMAVGAA | |
| | | | | | | LTGLKPIVEFMTMNFSLQAI | |
| | | | | | | DHIINSAAKTLYMSGGQMGC | |
| | | | | | | PIVFRGPNGAAARVGAQHSQ | |
| | | | | | | CFASWYAHIPGLKVVAPWSA | |
| | | | | | | ADAKGLLRAAIRDPNPVIVL | |
| | | | | | | ENEILYGQKFPCPVDEDFIL | |
| | | | | | | PIGRAKIEREGTDVTLVAFS | |
| | | | | | | IMVGVALEAAAILADEGISA | |
| | | | | | | EVINLRSIRPLDTETIVRSV | |
| | | | | | | KKTNRIVSVEEGWPVAGIGA | |
| | | | | | | EICTVAVEQAFDWLDAPPAR | |
| | | | | | | VCGLDLPMPYAANLEKLALP | |
| | | | | | | KPEWVVDAVRKQLRD | |
| GO_ | petP | HTH-type | L | | Down | MDVSSSATAHLYLREDRIRQ | 274 |
| 1846 | | transcriptional | | | | SYEAMMLAWRTLNADCEALL | |
| | | regulator PetP | | | | REKGLGPAHHRILFLTAAHP | |
| | | | | | | GITPGVLLNSLGITKQSLGR | |
| | | | | | | ALGDLRERKLLIQEEDRHDR | |
| | | | | | | RKRPLRLTASGEALERELFL | |
| | | | | | | MIREVMTRAYREAGMTAVEG | |
| | | | | | | FRRVLAPLQVPAESRAR | |
| GO_ | petR | DNA-binding | L | | Down | MSDEHILVVDDDPRLLRLLQ | 275 |
| 1847 | | response | | | | RYLSENGYRVSTALDAQTAR | |
| | | regulator | | | | DVLQRIQPDALVLDVTMPGE | |
| | | PetR | | | | DGLSLTNSLRRDGLSLPILL | |
| | | | | | | LTARGEPADRIGGLEAGADD | |
| | | | | | | YLGKPFEPRELLLRLRAHLR | |
| | | | | | | RMVPSLPAVDEVPDVLRLGE | |
| | | | | | | LEFDVKRGLLSGPQGAVHLT | |
| | | | | | | GGESALLGVLTRQPGTVLSR | |
| | | | | | | EAIARALEMDEIGERAVDVQ | |
| | | | | | | VTRLRRRIEADPKEPRFLHT | |
| | | | | | | VRGKGYVLKPGR | |
| GO_ | phoR | Phosphate | L | | Down | MVLVCVALAGWVLAVWLLLR | 276 |
| 1161 | | regulon | | | | QPRVPTSPPDDYLTPVSPAD | |
| | | sensor protein | | | | LPVDPLPACAVVLDGLGAIV | |
| | | PhoR | | | | QVNEEAASQFGETVGAILRH | |
| | | (SphS) | | | | PAARAALSAALRAPVPASPS | |
| | | (EC 2.7.13.3) | | | | GSSNRGDLPPVCSTTFTLDV | |
| | | | | | | PVPRTLHLMLRRLPGGKGQD | |
| | | | | | | RRVLVVLTDRSEAQAADRMR | |
| | | | | | | MDFVAHASHELRTPLASLSG | |
| | | | | | | FIDALQGAAGENPVMRQQFL | |
| | | | | | | DIMRQQSERLKRLIDRLLYL | |
| | | | | | | SRVQAHEHQRPRDVVDVADL | |
| | | | | | | MAVVLGEVAPRFEQEGRTLK | |
| | | | | | | LEIEDDLLVRADEDEMVQVL | |
| | | | | | | LNLIENALRYGAQEGDPLTI | |
| | | | | | | TLSARRAASPDDRWPADGGV | |
| | | | | | | ILGIEDNGCGMEAHHLPRLT | |
| | | | | | | ERFYRVGAPTDGAGQGTGLG | |
| | | | | | | LSIVRHILDRHGGRLRIASA | |
| | | | | | | PGKGTTCLVWLPPAGATLAV | |
| | | | | | | SMVE | |
| GO_ | pstA | Phosphate | L | | Down | MSETVVSTTGWKPNPRAARR | 277 |
| 1165 | | transport | | | | RRADHLATAFGMVMAGILVL | |
| | | system permease | | | | VLASILWTLLSRGLAGLSAA | |
| | | protein PstA(TC | | | | AIMKPMGPPGSSSGLANAIV | |
| | | 3.A.1.7.1) | | | | GSLIQTFMALLMATPLGLGC | |
| | | | | | | GIYLSEYGTETNKFASCVRF | |
| | | | | | | VSDVLMSVPSILVGLFVYQV | |
| | | | | | | LVAPFGHFSALAGSVALAIL | |
| | | | | | | AVPIIVRTTEDMLRLVPTSM | |
| | | | | | | REAGAALGATRWRVTLSLCL | |
| | | | | | | RSAKTGVLTGILLALARVSG | |
| | | | | | | ETAPLLFTSLGNQNWSFSLN | |
| | | | | | | RPMASLPVTIYQYAGASYED | |
| | | | | | | WVQLAWAGALLVTMGVLAIN | |
| | | | | | | IAVRVSARRG | |
| GO_ | pstB | Phosphate | L | | Down | MIQDSMMTETDPQVAKSPEV | 278 |
| 1164 | | transport | | | | ALAVRNLNFYYGENHALHDI | |
| | | ATP-binding | | | | SIDFPARRVTAMIGPSGCGK | |
| | | protein | | | | STLLRVFNRMYDLYPGQRAT | |
| | | PstB (TC | | | | GEVIFDGRNVLERDLDLNIL | |
| | | 3.A.1.7.1) | | | | RARVGMVFQKPTPFPMSIYD | |
| | | | | | | NIAFGVRLHEKLNKADMDAR | |
| | | | | | | VQDVLTRVALWNEVRDRLNA | |
| | | | | | | PASGLSGGQQQRLCIARSIA | |
| | | | | | | TRPEVLLLDEPTSALDPIST | |
| | | | | | | ARIEELLDELKEEFTIAIVT | |
| | | | | | | HNMQQAARCADQVAFFYMGR | |
| | | | | | | LIEVDSADRMFTNPKQQQTQ | |
| | | | | | | DYITGRFG | |
| GO_ | pstC | Phosphate | L | | Down | MTLATATQPEEARDKAGVSH | 279 |
| 1166 | | transport | | | | SGSRRSGPDTAFHLLVAASA | |
| | | system permease | | | | LLVLVVLGGLVVLMGVGGSQ | |
| | | protein PstC(TC | | | | AFRTFGLGFAFHDVWNPVAD | |
| | | 3.A.1.7.1) | | | | QYGAWAPLFGTIVSTLIGVA | |
| | | | | | | IALPLAFGTAFWLTAMAPPR | |
| | | | | | | IAAIVGTAVQLLAAVPSIIF | |
| | | | | | | GMWGFFTIVPFMARTVQPFL | |
| | | | | | | THHFRHVPGIRFIIHGAPFG | |
| | | | | | | TGLMTAGLVLAVMIAPFMTA | |
| | | | | | | VMRDVFAAMPAMLRESAYGL | |
| | | | | | | GATRWDVMWKVVVPWSRTGM | |
| | | | | | | IGAIVLGMGRALGETMAVTF | |
| | | | | | | VIGNVTAVGWSLFAPRSTVA | |
| | | | | | | SLIALQFPESPAGSLRLSAL | |
| | | | | | | LALGFILMLLSFASLALARM | |
| | | | | | | LRGDTK | |
| GO_ | pstS | Phosphate ABC | L | | Down | MIKSRAPLFGLLVATALGTA | 280 |
| 2369 | | transporter, | | | | AMTPFVSSAKAADITGAGSS | |
| | | periplasmicphosphate- | | | | FAAPIYGAWGTAAKSQAGIA | |
| | | binding protein PstS | | | | VNYQSVGSSAGQDQVIARTV | |
| | | (TC 3.A.1.7.1) | | | | DFGASDKPMSGDRLAKEKLY | |
| | | | | | | QFPTVMSGIVVVANVPGIAP | |
| | | | | | | GQLRLDGPTLAGLYDGEITT | |
| | | | | | | WDDDRIKALNPGLKLPDTDV | |
| | | | | | | APIHRADGSGTSYVFTSYLS | |
| | | | | | | QVSPTWKQKLGAGTSIAWPG | |
| | | | | | | GSGARGNDGIAAMVRQTEGG | |
| | | | | | | VGYVEYSYAAQNHLNIAQMK | |
| | | | | | | NHSGAFVAPTLASFAEAAKA | |
| | | | | | | ADWVHADHYAVNLLDTDGAS | |
| | | | | | | SWPIVTATFVLVPVDAAQKE | |
| | | | | | | SGKAVRNFFAWGFQHGDADN | |
| | | | | | | ARLDYVGLPQNVKTDILANW | |
| | | | | | | PK | |
| GO_ | speC | Pyridoxal 5- | L | | Down | MTPKITRFLAEQQPATPCLV | 281 |
| 201 | | phosphate (PLP)- | | | | VDLDVVGAHYRALHDALPEA | |
| | | dependent ornithine | | | | KIYYAIKANPAPAILDRLVA | |
| | | decarboxylase (EC | | | | LGSSFDVASPAEIRMCLDAG | |
| | | 4.1.1.17) | | | | AAPDRISYGNTLKKAEWIRE | |
| | | | | | | AHDLGISLFVFDSIEELEKL | |
| | | | | | | AKHAPGARVFCRLAVENEGA | |
| | | | | | | DWPLSRKFGTTLSNARALML | |
| | | | | | | RARELGLKPYGLSFHVGSQQ | |
| | | | | | | TGVAAYDHAIAKAAGLYHDL | |
| | | | | | | RAQGVDLQMLNLGGGFPTHY | |
| | | | | | | RENVPSVQDFAHTIHTSLKT | |
| | | | | | | HFPDGAPEILLEPGRYMVGQ | |
| | | | | | | SGVVSSEVILVSRRGGALTD | |
| | | | | | | PRWVYLDIGRFGGLAETEGE | |
| | | | | | | AIRYTFRTSRDSEDAARSPC | |
| | | | | | | VVAGPSCDGVDIMYEKNRIP | |
| | | | | | | LPDSLECGDRVEILATGAYV | |
| | | | | | | STYCSIGFNGFPPLTEYYI | |
| GO_ | surA | Periplasmic | L | | Down | MSKTHCIASTALAALLAFSA | 282 |
| 2743 | | chaperone and | | | | ALPATAAPHHKADPKAASKT | |
| | | peptidyl-prolylcis- | | | | AATKQEAPPAKPPEDQILAV | |
| | | trans isomerase of | | | | INGQVLTQRDVDNRAKLFVL | |
| | | outer membrane | | | | STGLPISPEIMNRMRGQIIH | |
| | | proteins SurA | | | | QLIDERLKTQEILKLHINVE | |
| | | (EC | | | | PDQIAGAISNIEQRNGMPKN | |
| | | 5.2.1.8) | | | | ALRDRLASDGVSLTTLIDQI | |
| | | | | | | RVQIGWMQVLREKLGEEGRI | |
| | | | | | | TATQISQREQALQAEQGRAQ | |
| | | | | | | YFMSEIFVPVADPRHDENEL | |
| | | | | | | AFTKTIISQLREGAPFPIVA | |
| | | | | | | AQFSQAQSALDGGSMGWVQE | |
| | | | | | | DNLDPQVVNIVRQMPIGAIS | |
| | | | | | | NPIQVAGGFVIATVQSKRVV | |
| | | | | | | GKQMGTLLDLRQAFFPFDAP | |
| | | | | | | LNPQNPTEQQRAALQKATTA | |
| | | | | | | VQTVHSCDAMEALNKSLGEK | |
| | | | | | | RPSNPGSQILERLMPQMKAV | |
| | | | | | | LEALPPNRVSRPLVSMDGIA | |
| | | | | | | LLMVCNRQQKNLAQQSPSEI | |
| | | | | | | ADQLMNERVEQASRQLQRDL | |
| | | | | | | QRRAIIEMRPAAKTAFN | |
| GO_ | tonB | TPR domain protein, | L | | Down | MSLSLYRRLSARNLLLAGVF | 283 |
| 2957 | | putative component | | | | GIAALAGSAHADDVLGQAVG | |
| | | of TonBsystem | | | | KDLQQAQSALAAKNYAKAMD | |
| | | | | | | AVDAADAVKGKTDYEAYTTA | |
| | | | | | | QMRAAIAAQSGNTDAAIKAY | |
| | | | | | | DVLINSSRTPKATKGQMLMA | |
| | | | | | | QATMAYSAKQYARAIPATER | |
| | | | | | | YLKEYGADPRMQTMLIQCYY | |
| | | | | | | LQQDWKGTAKAAQEQVDATI | |
| | | | | | | KAGKVPAENQLQMLATAYTN | |
| | | | | | | LKDADAKTHAYVLLAKYYSK | |
| | | | | | | PDYWSMLIHDLVANPNLSPP | |
| | | | | | | LVFYVERLRLATGVLKDPSD | |
| | | | | | | YQDMGERAVOMGLPQLALNL | |
| | | | | | | LNQGYANHSLGNGPTAAADA | |
| | | | | | | KFHAFVAQQAATNRSQLASA | |
| | | | | | | VTQAASAPNAGPALTAGYNQ | |
| | | | | | | VLNGQVDAGLALMKTGLGKN | |
| | | | | | | PRYPDLAQVEYGMAQMDGGQ | |
| | | | | | | KAEAIKTFASVQGNGPAKDV | |
| | | | | | | AELWSLLLSRPTK | |
| GO_ | top1 | DNA topoisomerase | L | | Down | MTDVVVVESPAKAKTINKYL | 284 |
| 2929 | | I (EC 5.99.1.2) | | | | GSGYTVLASFGHVRDLPPKD | |
| | | | | | | GSVRPDENFAMSWQTDERGA | |
| | | | | | | KQISAITKALKGAKNLYLAT | |
| | | | | | | DPDREGEAISWHVRSVLEEK | |
| | | | | | | KLLKNVDVHRVTFNEITKSA | |
| | | | | | | VTAAMAAPRELDRPLIDAYL | |
| | | | | | | ARRALDYLVGFTLSPVLWRK | |
| | | | | | | LPGSRSAGRVQSVALRLICE | |
| | | | | | | REAEIEVFRPKEYWTVTGGF | |
| | | | | | | TTPGKAAFQARLTHLKGEKL | |
| | | | | | | DQFDLNNEQLAFGARDTVLG | |
| | | | | | | GQFTVRSVERKRTKRNPPPP | |
| | | | | | | FTTSTLQQEASRKLGMSAQT | |
| | | | | | | TMRTAQQLYEGVDLRGETTG | |
| | | | | | | LITYMRTDGVTMAKEAVGAI | |
| | | | | | | RGHIGKAFGDEYVPDYPRSY | |
| | | | | | | STKAKNAQEAHEAIRPTDVF | |
| | | | | | | LTPQQVAHALTPEQKKLYEL | |
| | | | | | | IWKRSVASQMQSAELDQVAV | |
| | | | | | | TLADASGQTLLRATGSTIAF | |
| | | | | | | DGFLKLYIEGRDDTKAEDED | |
| | | | | | | GKLLPPMKEGDRLTTGTVDA | |
| | | | | | | EQHFTQPPPRYSEASLVKKM | |
| | | | | | | EEIGIGRPSTYASILGVLRD | |
| | | | | | | RNYVRLDARRFVPEDRGRLV | |
| | | | | | | TAFLTSFFERYVDTGFTASL | |
| | | | | | | EEQLDDISGGRADWHDVMAA | |
| | | | | | | FWHDFSAAVAQTKDLKISDV | |
| | | | | | | IDALDEDLGPHFFPPRPDGA | |
| | | | | | | DPRVCTSCGTGRLGLRLGKF | |
| | | | | | | GAFIGCSNYPTCQFTRRLVA | |
| | | | | | | EEGDSEGLNDGPKVLGQDPE | |
| | | | | | | TGEDISIRRGPYGLYIQRGE | |
| | | | | | | PNPEDKKAKPKRTTIPKGID | |
| | | | | | | GNTMTMEQALGLLSLPRLIG | |
| | | | | | | LHPETGEKIEAGLGRFGPYV | |
| | | | | | | KMGAIYGTLDKDDDVLTVGL | |
| | | | | | | NRAVDALAKKLASIRNLGPH | |
| | | | | | | PKDGEPVMIRKGRFGPYAQH | |
| | | | | | | GQLITNLPKGQDMDEVTLDE | |
| | | | | | | AVALLAEKGKPLKGGAKKTS | |
| | | | | | | AKKAAPKAAKAKKAVAAAEG | |
| | | | | | | DEAAPKKVTKRSPAKSATKT | |
| | | | | | | KTTTPRKRKTVSDTSSEG | |
| GO_ | ykoH | Two-component | L | | Down | MSRADLRFSELFRADLFRTA | 285 |
| 2648 | | system sensor | | | | TFRLTLAFVVAIIAGMALQF | |
| | | histidine kinase | | | | GLVYGQMSGYEQQRSTDLLQ | |
| | | | | | | REAALLVHETPAELEYEVRE | |
| | | | | | | RSKTDLRVILNGAALFDMSR | |
| | | | | | | NRIAGDIKKWPVGLEVSPKT | |
| | | | | | | QRLWDAPPGDTPYEMRYLAV | |
| | | | | | | EVEGTNGNRDRILVLARSLH | |
| | | | | | | MANELRYITKRAALMSVVPV | |
| | | | | | | VAFALMAGIFLSHRALGRIK | |
| | | | | | | DMHEAIDRIMDGDLHERLPT | |
| | | | | | | GRERDDIERLAVSVNRMLDR | |
| | | | | | | LEHLLDEIRDVGNDIAHDLR | |
| | | | | | | TPLARVKARLERVSAITNDP | |
| | | | | | | AALQGAIERAALDLEQCFSV | |
| | | | | | | ITALLRIGEIENGRRRAGFA | |
| | | | | | | MLDLRELLAGVVDLYEPIAE | |
| | | | | | | TEGVMLEVVDSDKPVPLFGD | |
| | | | | | | KDLLNEVLANLVDNAIKFTP | |
| | | | | | | EPGTVRLSAGQGPDGATWLQ | |
| | | | | | | VADTGIGIAEDERKAVMGRF | |
| | | | | | | YRSDKSRHVPGSGLGLSLVS | |
| | | | | | | AILRLHGASVDIVSAHPGQA | |
| | | | | | | LPGAVFTIHFAPPASV | |
| GO_ | GO_ | Amidohydrolase | | F | Down | MTIRSSFAALLLATPAALSV | 286 |
| 2942 | 2942 | precursor | | | | GSAMAEPVAFEHARLIDGTG | |
| | | | | | | ALAQPDATVVIDNGTIISVG | |
| | | | | | | IPAPAQVRHVDLTGKTLMPA | |
| | | | | | | LISDHVHVGLVKGTGASRDN | |
| | | | | | | YTRANILAALKQYSDYGVLT | |
| | | | | | | VTALGLNRSPLFDTLRQEQH | |
| | | | | | | DGRNPGADLYGVDQGIGAPD | |
| | | | | | | GVPPAAMVKGVGPDQVFRPT | |
| | | | | | | TPEEARKDVDQMIAEHTDLV | |
| | | | | | | KLWVDDFRNDVPDGKTYPML | |
| | | | | | | PPAIYQAVIDEAHQHGTRVA | |
| | | | | | | VHIHDLAVAKAIVASKADIL | |
| | | | | | | AHGVRDQPVDHDLIAAMLRQ | |
| | | | | | | GTWYIATLDLDEANYLYAEQ | |
| | | | | | | PELLSNPFVLAGVNPALRRQ | |
| | | | | | | FTDPKWRAETLAKPLTKASH | |
| | | | | | | YALSVNQKNLAVLYRAGVKV | |
| | | | | | | GFGTDSGAAPTRIPGFAEHR | |
| | | | | | | ELYLTVQAGLSPVQAISLAT | |
| | | | | | | GNAAALLHLSDRGVIAPGRR | |
| | | | | | | ADLLVVNGNADENIGAVDQI | |
| | | | | | | DQVWQRGMLVSHGPVRSKN | |
| GO_ | GO_ | Uncharacterized | | F | Down | MTLSVIHDKTTTLTGAPVAA | 287 |
| 313 | 0313 | SAM-dependentO- | | | | LLERLFAEAETATNPAIADI | |
| | | methyltransferase | | | | PREEFQRLAGSRTEYRKFYG | |
| | | | | | | LAKHLWLPVSRETGTLLYML | |
| | | | | | | ARATWAKNIVEFGTSFGIST | |
| | | | | | | IHLAAALRDNGGGKVITTEF | |
| | | | | | | EPSKVARARAHLEEAGLADL | |
| | | | | | | VEFREGDALQTLAAGLPESI | |
| | | | | | | DIVLLDGAKPLYPDILDLLE | |
| | | | | | | DRLQAGALIVADNADHSPEY | |
| | | | | | | LARVRSPAAGYLSLPFAEDV | |
| | | | | | | ELSVRLH | |
| GO_ | GO_ | Glutamate N- | | F | Down | MAKPLPVSPLARPLPDLATI | 288 |
| 1376 | 1376 | acetyltransferase | | | | AGVRLSAVAAGIRYQGRTDL | |
| | | (EC | | | | MLAEFVPGTVAAGVYTKNAC | |
| | | 2.3.1.35) | | | | PGAPVLWCREALTTPYARAL | |
| | | N- | | | | LVNAGNANVFTGRAGMQACE | |
| | | acetylglutamate | | | | DCADATAQLLDCPPQDVFLA | |
| | | synthase (EC | | | | STGVIGEKLPQDRIIAALPA | |
| | | 2.3.1.1) | | | | ARAGLEENGWADAARAIMTT | |
| | | | | | | DTFPKAARRDVKINGTPVRI | |
| | | | | | | QGIAKGSGMVAPDMATMLAY | |
| | | | | | | VATDARLPQNVLQSLLASGC | |
| | | | | | | AQSFNSITVDSDTSTSDMLM | |
| | | | | | | IFATGLADNPEVDDVNDPAL | |
| | | | | | | AEFTLALNDLLLELALMVVR | |
| | | | | | | DGEGATKLVRIAVTGADSNL | |
| | | | | | | SAHRIALCIANSPLVKTAIA | |
| | | | | | | GEDANWGRVVMAVGKSGEPA | |
| | | | | | | DRDRLSVAIGGTWIAKDGGV | |
| | | | | | | VENYDEAPVVAHMKGQEIEI | |
| | | | | | | AVDLGLGDGQARVWTCDLTH | |
| | | | | | | GYIDINGSYRS | |
| GO_ | GO_ | hypothetical | | F | Down | MIDFSSWHSLLATLIGLALF | 289 |
| 178 | 178 | protein | | | | TLIGVGIRLLTMLTIQQRRE | |
| | | | | | | RMNRQINERLRVLMAAYRTL | |
| | | | | | | GGSFTGTLLVDPTHKRDLEP | |
| | | | | | | DQLSGSDRNRRIRDAVEAAL | |
| | | | | | | SDIILLGTEEQVRMAGRAAA | |
| | | | | | | ELVAGRPVPTHDLVVSLRNF | |
| | | | | | | IRKALNLESLPSDLVLPEQG | |
| | | | | | | PARPSSSGGNKGDGKEGGKG | |
| | | | | | | GGGGGDGGGGGGMGMQGGMD | |
| | | | | | | PALHHSETDSHTL | |
| GO_ | GO_ | Phage shock | | F | Down | MSPDNLALLIPIVAIIAWSV | 290 |
| 2357 | 2357 | protein | | | | TSMVKHTTRQADPPGQPDPM | |
| | | B | | | | LQAALTQAEAHATRLEERID | |
| | | | | | | OLERILDEDIPGWRARNAR | |
| GO_ | GO_ | Threonine | | F | Down | MRYRSTRGELTADAPNFSDI | 291 |
| 2556 | 2556 | synthase | | | | LLAGLAGDGGLYMPESWPRI | |
| | | (EC 4.2.3.1) | | | | SPQTLREWRTLSYPDLAAEV | |
| | | | | | | IALFTEGAIDVDTLRDMTRD | |
| | | | | | | AYADFDHAAVVPLVEVEQDL | |
| | | | | | | YSLELFHGPTLAFKDMAMQM | |
| | | | | | | LGRLFDHVLTQRNRHVTIVG | |
| | | | | | | ATSGDTGSAAIEACRGRERL | |
| | | | | | | SVVILHPKGRTSDVQRRQMT | |
| | | | | | | TVQDDNILNIAVEGDFDTCQ | |
| | | | | | | DLVKAMFADHAFRDEVSLSA | |
| | | | | | | VNSINWARIAAQIPYYVRAA | |
| | | | | | | LALGAPDREVSFSVPTGNFG | |
| | | | | | | NVLAAWAAKQMGLPIKKLCI | |
| | | | | | | GSNRNDILTRFVIDNDMSVR | |
| | | | | | | TVEPSLSPSMDIQVSSNFER | |
| | | | | | | LLFELLDRNSTRCAAIMREF | |
| | | | | | | RETGRMAVPHDAWTRMKEVF | |
| | | | | | | EGMTLTDEQTSEAMRLFYNE | |
| | | | | | | SLYLADPHSAIGLAVGKRFQ | |
| | | | | | | EPGIPMVAAATAHPAKFPDA | |
| | | | | | | VIAATGIHPKLPPHLSDLFE | |
| | | | | | | RTERYECMDSQIAGLQDAVR | |
| | | | | | | AHIRRG | |
| GO_ | GO_ | hypothetical | | F | Down | MIASQGPSMRRNRLSVARLS | 292 |
| 2916 | 2916 | protein | | | | VQMGAMASVGLLLSGCSGAD | |
| | | | | | | VSRAIGLERAMPDEYTVTTR | |
| | | | | | | APLSMPPSEQMQLPGAADAH | |
| | | | | | | RPDESPRMQALETLSPDTAL | |
| | | | | | | HPDAGQGSSGQTALVGQVDK | |
| | | | | | | SASAPNNAELGAADAGFVDN | |
| | | | | | | LMFWKGGNAGSVVDGDAENR | |
| | | | | | | RIRENSALGRNPATGATPTV | |
| | | | | | | RKKKAFLGVL | |
| GO_ | GO_ | ATP synthase | | F | Down | MPLRIEIVSPEKRLVEREVD | 293 |
| 2979 | 2979 | epsilon chain | | | | MAVVPGMEGDIAAMPDRAPL | |
| | | (EC | | | | MLQLRGGVVALYQGDKIVDR | |
| | | 3.6.3.14) | | | | YFVTGGFADMGADHCTILAD | |
| | | | | | | SAQLMSELSVDEAKSRLRDL | |
| | | | | | | ESRWAEIGPNDVDMHDQISR | |
| | | | | | | ELQSVRAELEAVQEHGPA | |
| GO_ | idnk | Gluconokinase | | F | Down | MTENETQLGLKPHFLVVMGV | 294 |
| 1341 | | (EC | | | | SGTGKTTVASGLATRLGWHF | |
| | | 2.7.1.12) | | | | QEGDALHPPANVEKMSTGQP | |
| | | | | | | LTDADRAPWLALCHEWLRKQ | |
| | | | | | | VEAGHGAVLTCSALKRSYRE | |
| | | | | | | QLRGEDLPIEFVHIDTSVGE | |
| | | | | | | LADRLQRREGHFMPASLLPS | |
| | | | | | | QLATLEVPGDDEPVIRVSGE | |
| | | | | | | KHPDVVLEELIRHFQAED | |
| GO_ | mobA | Molybdenum | | F | Down | MTPLYGLILAGGASKRMGTD | 295 |
| 196 | | cofactor | | | | KAALDYHGKPQLQVAFEVLS | |
| | | guanylyl | | | | PLVEKCFVSVRPDQTADPLR | |
| | | transferase | | | | SSFPQIVDTVDVDGPAAGLL | |
| | | (EC 2.7.7.77)/ | | | | SAHRAYPDVAWLVLACDLPM | |
| | | Molybdopterin | | | | LDRGTLDTLIAARDAGHVAV | |
| | | synthase sulfur | | | | SYRSEHDGLPEPLCAIWEPE | |
| | | carrier subunit | | | | ALDRLEKQVAGGRICPRKLL | |
| | | | | | | INSPTKLLEPHRRGALDNIN | |
| | | | | | | TPEERDDAARRLKDLPGGPM | |
| | | | | | | IRLTLEYFAQLRELAGTREQ | |
| | | | | | | SLETAFVTVGPLYEELREKY | |
| | | | | | | AFPFEASKLRVAINGDFAPW | |
| | | | | | | TQALKDGDHIVFIPPVTGG | |
| GO_ | nifS | Cysteine | | F | Down | MTALKTVSGTTYLDANATEP | 296 |
| 84 | | desulfurase | | | | LRPCAKEAAVEGMMLSGNPS | |
| | | (EC 2.8.1.7) | | | | SVHAEGREARRFLEDARSRV | |
| | | | | | | AAGFGRISGTCVFTSGATEA | |
| | | | | | | DAMAVHAFGQERRIFVGSTE | |
| | | | | | | HDAILRAAPEAEILPVNRDG | |
| | | | | | | ILDVEHLRSRLQDTGPALVC | |
| | | | | | | VMSANNETGVLSPLEDVLAV | |
| | | | | | | CRDSGAHLHVDAVQSAGRLP | |
| | | | | | | FALGGCSVAVSGHKMGGPKG | |
| | | | | | | AGALLLAEDEPMDALVAGGG | |
| | | | | | | QERGRRGGTQALPAILGMAA | |
| | | | | | | AFDAARAQDWAPVQRLRDRV | |
| | | | | | | EAAAKSVGARVAGEAVDRLP | |
| | | | | | | NTSCLILDGVAAQVQLMALD | |
| | | | | | | LAGFCVSAGSACSSGKVSSS | |
| | | | | | | HVLRAMGETEGASQAIRVSL | |
| | | | | | | PWNVREAQVEAFCEAYEAMA | |
| | | | | | | RRLRK | |
| GO_ | phgdH | D-3- | | F | Down | MSSKPDILTIDPLVPVMKER | 297 |
| 2626 | | phosphoglycerate | | | | LEKSFTLHPYTSLENLKSIA | |
| | | dehydrogenase | | | | PAIRGITTGGGSGVPSEIMN | |
| | | (EC | | | | ALPNLEVISVNGVGTDRINL | |
| | | 1.1.1.95) | | | | DEARRRNIGVATTQNTLTDD | |
| | | | | | | VADMAVALMMTVMRGIVTND | |
| | | | | | | AFVRAGKWPSATPPLGRSLT | |
| | | | | | | RKKVGIAGFGHIGQAIAKRV | |
| | | | | | | SAFGMEVAYFNSHARPESTC | |
| | | | | | | HFEPDLKALATWCDVLILAV | |
| | | | | | | SGGPRSANMIDRDILNALGK | |
| | | | | | | DGFLVNIARGTVVDEAALLS | |
| | | | | | | ALQEKRIAGAGLDVFQNEPN | |
| | | | | | | INPVFLSLPNTVLQAHQASA | |
| | | | | | | TVETRTAMAHLVVDNLIAYF | |
| | | | | | | TDKTLLTPVI | |
| GO_ | lychF | GTP-binding and | | F | Down | MGFNCGIVGLPNVGKSTLFN | 298 |
| 2153 | | nucleic | | | | ALTETAAAQAANYPFCTIEP | |
| | | acid-binding | | | | NTGRVAVPDPRLDELARIGK | |
| | | proteinYchF | | | | SIRKVPTSLEFVDIAGLVRG | |
| | | | | | | ASKGEGLGNQFLANIREVDA | |
| | | | | | | IVHVLRCFEDDDITHVEGGV | |
| | | | | | | DPVRDADIIETELMLADLES | |
| | | | | | | LEKRQVGLQKRARGNDREAQ | |
| | | | | | | AQLELMEPLLAALRDGKPAR | |
| | | | | | | TAVSKGQEAEASRLQLLTTK | |
| | | | | | | PVLYVCNVEEASAATGNAFS | |
| | | | | | | EAVRKRAEAEGAGVVVVSAA | |
| | | | | | | IEAEVSQLPQEDRTEFLEGL | |
| | | | | | | GLTDSGLDRVIAAGYKLLGL | |
| | | | | | | RTYFTVGPKESRAWTITAGT | |
| | | | | | | KAPQAAAVIHNDFERGFIAC | |
| | | | | | | ETVAFDDYVACNGEAGAKES | |
| | | | | | | GKLRIEGKEYVVQDGDVLLF | |
| | | | | | | RFNV | |
| GO_ | GO_ | Transcriptional | | F | Down | MSDDPPFLRDADQTRKNILE | 299 |
| 29 | 29 | regulator, | | | | VALKEFAEYGLAGARVDRIA | |
| | | AcrR | | | | RGTRTTKGMIYYHFGDKDGL | |
| | | family | | | | YKAVLEKVYPSLRSDEEHLD | |
| | | | | | | VRNTDPVEALERIIDFTLDY | |
| | | | | | | HEKHEDFVRIVMIENINKGE | |
| | | | | | | HLRKTGIDSTVSYRIMMVIA | |
| | | | | | | DILNRGMALGLFKREITPVD | |
| | | | | | | LHIFYTSFCFYRVGNHHTVS | |
| | | | | | | SVLGINMLSAQSCARHRRMV | |
| | | | | | | KDAVIAYLASSD | |
| GO_ | GO_ | Pyruvate | | F | Down | MTYTVGHYLAERLTQIGLKH | 300 |
| 2220 | 2220 | decarboxylase | | | | HFAVAGDYNLVLLDQLIEQG | |
| | | (EC | | | | GTKQIYDCNELNCSFAAEGY | |
| | | 4.1.1.1) | | | | ARANGAAAAVITFSVGAISA | |
| | | | | | | MNGLGGAYAENLPILVISGA | |
| | | | | | | PNSNDHGSGHILHHTIGTTE | |
| | | | | | | YSYQMEMAKHVTCAAESITS | |
| | | | | | | AEAAPAKIDHVIRTMLREKK | |
| | | | | | | PAYLEIACNISAAPCVRPGP | |
| | | | | | | VSSLHAHPRPDEASLKAALD | |
| | | | | | | ESLSFLNKANKVAILVGTKL | |
| | | | | | | RAAEALKETVELADKLGCPV | |
| | | | | | | TVMAAAKSYFPETHPGFRGV | |
| | | | | | | YWGDVSSPGAQEIIEGADAV | |
| | | | | | | ICLAPVWNDYSSGGWKSIVR | |
| | | | | | | GEKVLEVDPSRVTVNGKTFD | |
| | | | | | | GFRLKEFVKALTEKAPKKSA | |
| | | | | | | ALTGEYKPVMLPKADPSKPL | |
| | | | | | | SNDEMTRQINELVDGNTTLF | |
| | | | | | | AETGDSWFNAVRMHLPEGAK | |
| | | | | | | VETEMQWGHIGWSVPSMFGN | |
| | | | | | | ATASPERKHVLMVGDGSFQL | |
| | | | | | | TAQEVAQMVRYELPVIIFLV | |
| | | | | | | NNHGYVIEIAIHDGPYNYIQ | |
| | | | | | | NWDYAALMQCFNQGVPGEES | |
| | | | | | | GKYGLGLHATTGAELAEAIA | |
| | | | | | | KAKKNTRGPTLIECKLDRTD | |
| | | | | | | CTKTLVEWGKAVAAANSRKP | |
| | | | | | | QSV | |
| GO_ | asns | Asparagine | | S | Up | MCGIAGLSCLPGHHPDQDAL | 301 |
| 2458 | | synthetase | | | | ERMSQAIFHRGPDGEGRLDL | |
| | | [glutamine- | | | | GGAALRHRRLSIVDIAGGAQ | |
| | | hydrolyzing] (EC | | | | PFRLGAAALIANGEIYNDPA | |
| | | 6.3.5.4) | | | | IRRRFPKTCFQTYSDCEPPL | |
| | | | | | | HLWLHDGAGYTHELRGMYAI | |
| | | | | | | AIVENEHGRHEMVLSRDPFG | |
| | | | | | | IKPLYIAAYEGGIAFASEPQ | |
| | | | | | | ALLAGGFGKRSIRDSARDEL | |
| | | | | | | MQLQFTTGQDIIFDGIRRLL | |
| | | | | | | PGETLRIVDGRIVESRRRHV | |
| | | | | | | LHEARDTVPARLSDEQALER | |
| | | | | | | LDTALLDSVSAHLRADVPLG | |
| | | | | | | LFLSGGIDSSVILAAAHRLG | |
| | | | | | | LPHPRTWTARFDAGKADESA | |
| | | | | | | DAAALAASVGAEHHVLTVTE | |
| | | | | | | DMVWRELPSIVACMDDPAAD | |
| | | | | | | YATIPTWFLAREARKDVTVI | |
| | | | | | | LSGEGGDELFAGYGRYRRVM | |
| | | | | | | KPWWKGGRAPYRSGTFGRRF | |
| | | | | | | AEHGRQWRRGIAMTELALGV | |
| | | | | | | SGLEGAQALDIAEWLPNDLL | |
| | | | | | | LKLDRCLMAHSVEGRTPLLD | |
| | | | | | | PVVAKAIWPLPEHFKVRDGY | |
| | | | | | | GKWLLRRWLQDALPQARPFA | |
| | | | | | | PKQGFTVPVGPWIEKQAHRL | |
| | | | | | | GPLVARQPCIRAMMPAVDVE | |
| | | | | | | RLFARASQRGVARQAWTLLF | |
| | | | | | | FALWHRHHIEGVPVEGDTFE | |
| | | | | | | TLARAS | |
| GO_ | czcD | None (Cobalt- | | S | Up | MTPDPHHDCACDHAHHGTTV | 302 |
| 1427 | | zinc-cadmium | | | | PHEHHAHDHADHDHDDHHHD | |
| | | resistance | | | | HDHCDGHSHGFGFGHQHVHA | |
| | | protein | | | | PASFGMAFAVGITLNTAYVA | |
| | | CzcD) | | | | GEALWGVWAHSLSLLADAGH | |
| | | | | | | NLSDVLGLAGAWLAQVLATR | |
| | | | | | | PSSARFTYGLRRSTILSALA | |
| | | | | | | NAMILLLVTGGIVWESVLRL | |
| | | | | | | FSHQNVQGEVISWVALVGIA | |
| | | | | | | VNAVTALLFMKGASSDLNVR | |
| | | | | | | GAFLHMAADAVMAFSVVIAG | |
| | | | | | | LLIAFTGYTIIDPIMSLIVS | |
| | | | | | | VSIVIGTWSLLRSSLDLALD | |
| | | | | | | AVPAGIDPDAVQAALLSLDG | |
| | | | | | | VSGLHHLHIWAMSTTETALT | |
| | | | | | | VHLVCDPTKPVSTDLVIARA | |
| | | | | | | AELVRTRFDIAHPTFQLETQ | |
| | | | | | | PSVCDTHQPCC | |
| GO_ | GO_ | hypothetical | | S | Up | MLASGIFQFLPAGVVTEKGV | 303 |
| 1177 | 1177 | protein | | | | FSIHEKMNWRSCLWCRPLVR | |
| | | | | | | LGATTA | |
| GO_ | GO_ | Putative | | S | Up | MSHPVSRRDFAVGLVAGVAA | 304 |
| 1928 | 1928 | hemagglutinin- | | | | AGTGVAGAADPEKAVPTPPP | |
| | | related | | | | PPKPVAKTICFVGGYTKHGP | |
| | | protein | | | | PGGGTGNGQGISVFDMDRDT | |
| | | | | | | GVLTPITTFTDIASPSFLAI | |
| | | | | | | SKDQRFLYALSEIDDFNKDG | |
| | | | | | | DGSVTAFAIDPKTGSLRKLN | |
| | | | | | | VVSSKGAVPAHLSIHHSGRY | |
| | | | | | | VLVANYVGGCVAVLPIRSDG | |
| | | | | | | SLGEASDVVHNTGPRQPERA | |
| | | | | | | SDNPQGNFAVSDHSGSHPHM | |
| | | | | | | IHSDPSGKFVLADDAGLDRV | |
| | | | | | | YVWTLNIDTGKLIPAKTPYY | |
| | | | | | | DMEPGSAPRHFQFNHSGRIL | |
| | | | | | | YNLCEQDSKVVVSNFDPATG | |
| | | | | | | AINDIQTVSTVTSHFRGSTL | |
| | | | | | | AAEILISASGKFVYVSNRLG | |
| | | | | | | DSLAVFAIGADGTLTLQDEV | |
| | | | | | | WMHADYGRALMFDPSGAFLF | |
| | | | | | | CANQRSDAVTSFKVDKKTGE | |
| | | | | | | IAFTNNFTPVGSPTTFAFMN | |
| | | | | | | TQV | |
| GO_ | GO_ | hypothetical | | S | Up | MGSYCGIHFDKLSICGSKSE | 305 |
| 783 | 783 | protein | | | | VPGDWAALFQERDRRETRPT | |
| | | | | | | QEVGADPDLCVEYAASRDVI | |
| | | | | | | LRRLSILGATDEAVQRAFET | |
| | | | | | | WLTEEQEQWRDNTEGWSDQE | |
| | | | | | | VISEHAAKMLKGLNGLTYQE | |
| | | | | | | WCRFASGALRIRYDFANYDR | |
| | | | | | | IQSDLASDPFRNQFHEPDDG | |
| | | | | | | YLWFAGYGSHLGLRALLDAV | |
| | | | | | | PDIKEVRLDISDLLGEYVDE | |
| | | | | | | HEPICSRARENAPCQLQMLA | |
| | | | | | | PTMVMAEGSSDLTALRLGLG | |
| | | | | | | AMHPDLMDYFSFFNHAELSV | |
| | | | | | | DGGAHYLVKFLKAIAAARST | |
| | | | | | | SRILAIFDNDTVGIQAYEQA | |
| | | | | | | RALKLPFNIIVTRYPDSDVA | |
| | | | | | | KAYPTVGRSGPAILDVNGQA | |
| | | | | | | AGIELYMGREALLSNGELRP | |
| | | | | | | VRWASYVASAGKYQGEVDGK | |
| | | | | | | RQVLEAFRKNIATVEGPEAA | |
| | | | | | | RASFPDLERVWQHNFDLVQE | |
| | | | | | | NSGLVYLRTGKRLA | |
| GO_ | hlyD | HlyD family | | S | Up | MSSSDMIPNEGQPSGQSDED | 306 |
| 1175 | | secretion | | | | FNQHVPRTATDPFAPNDMPL | |
| | | protein | | | | ALLEFHSPTAGLINLPATPA | |
| | | | | | | ARYIILLIGGLFLACLAVMA | |
| | | | | | | LFPINRVVSTPGRLISTQPT | |
| | | | | | | IVVQPLETSIIRSIDVHVGD | |
| | | | | | | FVKKGDVLAHLDPTITEADI | |
| | | | | | | TNMHLQRDAYQAEYDRLKAE | |
| | | | | | | AAGQDYHVNLNDPASVEQGA | |
| | | | | | | AFLRRKTEYQAHVENYAQQI | |
| | | | | | | ASLESDIQGYRANAAMYGSK | |
| | | | | | | MRVASEVLQMRQREQADQVG | |
| | | | | | | SRLSTLGAQTELMEAERAEI | |
| | | | | | | AAQQSANSAEKKLAAMKAER | |
| | | | | | | DGYIGNWQAKIYSDLTEAGH | |
| | | | | | | HLAEYRSSYEKARLRQDLVL | |
| | | | | | | LRAPEDGIVLTIAQGSVGSV | |
| | | | | | | LQSAGQFITLVPTGYGLEME | |
| | | | | | | AVLRSQDVGFVQVGDHALLK | |
| | | | | | | FATFPYDQYGGAEATVRVIS | |
| | | | | | | ADAFTPSSQNAGGGSNGNTP | |
| | | | | | | SDDATASGVYRVRLRIDRYT | |
| | | | | | | LHGQPSFFHPMPGMSLTADI | |
| | | | | | | DVGKRTVLQYLFNKITPALT | |
| | | | | | | NGMREP | |
| GO_ | pal | Tol-Pal system | | S | Up | MKFKVFGALGLALVLAACSN | 307 |
| 1363 | | peptidoglycan- | | | | GNTNKGDSTGAGAVAQEAGP | |
| | | associated | | | | TPGSEADLVANVGDRVFYEL | |
| | | lipoprotein | | | | NQSQLSEEARATLDKQVAWL | |
| | | PAL | | | | AKYPQVSIQVAGNCDDRGTE | |
| | | | | | | EYNIALGORRANAARDYLVA | |
| | | | | | | KGVSASRITTISYGKDRPTA | |
| | | | | | | DGDDEQSWAQNRNAITSVR | |
| GO_ | PhoB | Phosphate regulon | | | Up | MKGPSLARAKGLVLLVEDDP | 308 |
| 1162 | | transcriptional | | | | ALLLMTCYNLEQRGYRVETA | |
| | | regulatory | | | | EDGEAALLALETARPDAVVL | |
| | | protein | | | | DWMLPGLSGLDVCRRIRANP | |
| | | PhoB (SphR) | | | | ALRDVPVLLLTARSAEQDAI | |
| | | | | | | RGLDTGADDYLMKPCSIDTL | |
| | | | | | | DARLRALLRRHQSSYDRLSF | |
| | | | | | | ADITLDPETHRVERAGRMLS | |
| | | | | | | LGPTEYRLLDLLIRNPRKVF | |
| | | | | | | SREDLLRRIWGQNIHVEIRT | |
| | | | | | | IDVHIRRLRKAINGPGEVDL | |
| | | | | | | VRTVRAAGYALDDGPTTDGA | |

In embodiments, the modified bacteria have at least one engineered genetic change that is correlated with improved bioleaching of REEs, relative to REE bioleaching by unmodified bacteria of the same species as the modified bacteria. The disclosure includes the proviso that the set of modified genes may exclude a disruption of membrane bound glucose dehydrogenase (mgdh) gene as the only modification of the described bacteria. However, this gene may also be disrupted, provided it is in the context of at least one other gene modification that is described herein. In non-limiting examples, at least one genetic change increases acidification of a medium in which the modified bacteria are present. In a non-limiting example, the at least one genetic change is in a gene that is part of a phosphate transport system. In embodiments, the bacteria are modified such that they comprise a mutated gene that comprises or consists of at least one of: GO_1415, pstA, pstB, pstC, pstS, ggtl, surA, petP, ykoH, speC, and tonB. In embodiments, the modification comprises a disruption of at least GO_1415, or pstC, or a combination thereof.

The disclosure includes compositions comprising one or more REEs and modified bacteria of the disclosure. The disclosure includes a biolixiviant produced by the modified bacteria and one or more REEs. In embodiments, the disclosure relates to separating combinations of REEs. In embodiments, the disclosure relates to separating any one or combination of lanthanum, cerium, praseodymium, neodymium, promethium, samarium, europium, gadolinium, terbium, dysprosium, holmium, erbium, thulium, ytterbium, lutetium, scandium, and yttrium, from a composition comprising one or more of the REEs. The composition comprising the REEs may be any composition of matter, including but not limited to solids, semi-solids, and liquids. In embodiments, the REEs are present in a feedstock. In non-limiting embodiments, the REEs are present in coal fly ash, virgin ore, electronic waste, fluid cracking catalysts, and the like.

The disclosure includes a method comprising contacting a composition comprising one or more types of REEs with a biolixiviant produced by modified bacteria of this disclosure. In an embodiment, the method further comprising separating and optionally purifying one or more types of REEs from the composition comprising the REEs and the biolixiviant.

The disclosure comprises isolated modified bacteria, cell cultures comprising the modified bacteria, and kits comprising the modified bacteria. In an embodiment, a kit comprises one or more sealed containers comprising the modified bacteria, which can be used in REE bioleaching approaches.

The disclosure includes media in which the bacteria are cultured, and bacterial secretions. In an embodiment, the disclosure provides a biolixiviant produced by the described bacteria. In embodiments, the kit contains a sealable or sealed container that contains a biolixiviant produced by the described bacteria. The disclosure also includes modifying bacteria so that they comprise at least one of the described gene modifications.

The disclosure includes all modified microorganisms described herein. The described approaches may be used to engineer any type of bacteria. In embodiments, the bacteria are Gram-negative bacteria. In embodiments, the bacteria are obligate aerobes. In embodiments, the bacteria modified as described herein comprise any member of the bacteria family Acetobacteraceae. In an embodiment, the bacteria is a type of Gluconobacter. In an embodiment, the modified bacteria are Gluconobacter oxydans. In this regard, G. oxydans secretes a biolixiviant rich in gluconic acid. This is produced by periplasmic glucose oxidation by the pyrroloquinoline quinone (PQQ)-dependent membrane-bound glucose dehydrogenase (mGDH). The final pH of the biolixiviant is a major factor in REE bioleaching. But, gluconic acid alone fails to explain bioleaching by G. oxydans: pure gluconic acid is far less effective at bioleaching than the biolixiviant produced by G. oxydans. This means that even the most previous successful efforts to up-regulate mGDH activity and gluconic acid production are unlikely to take full advantage of G. oxydans' biolixiviant production capabilities. Thus, the present disclosure reveals a curated set of genes that can be modified to improve REE extraction, as demonstrated in the following Examples.

### EXAMPLES

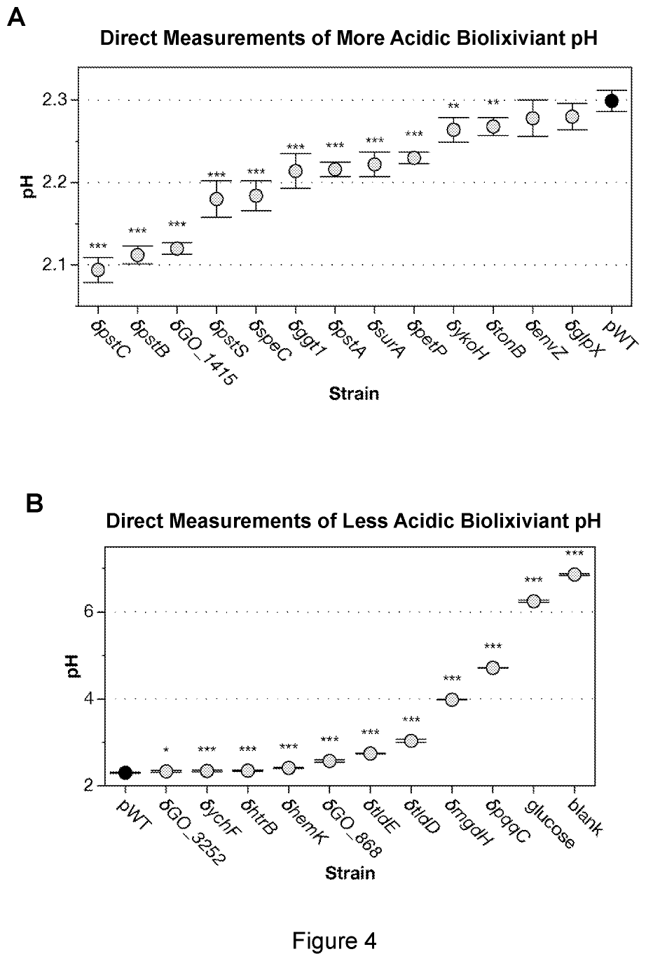

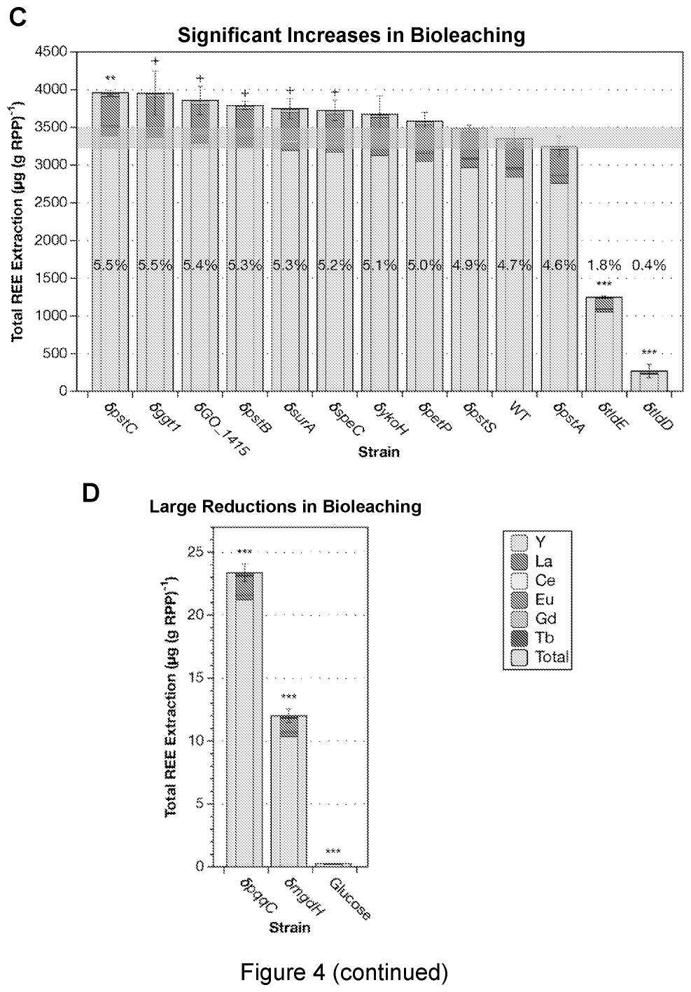

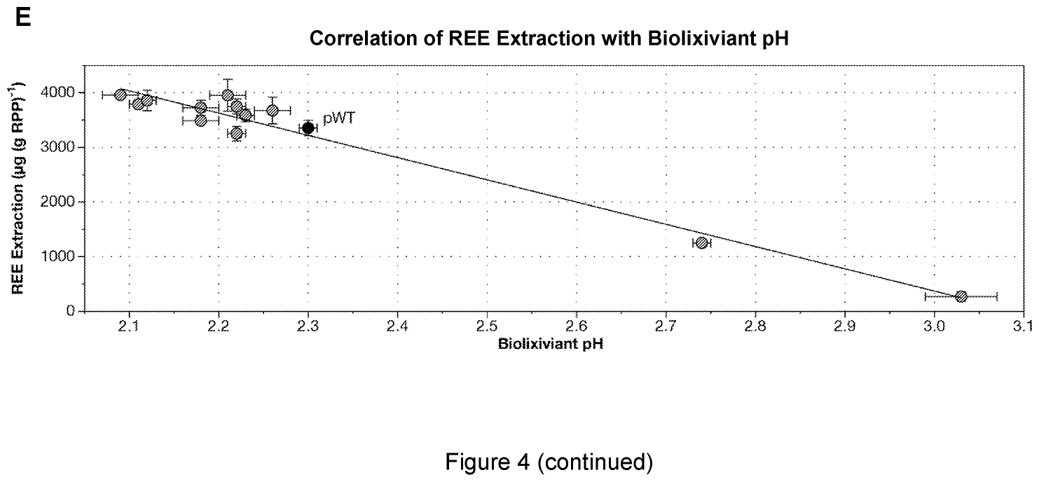

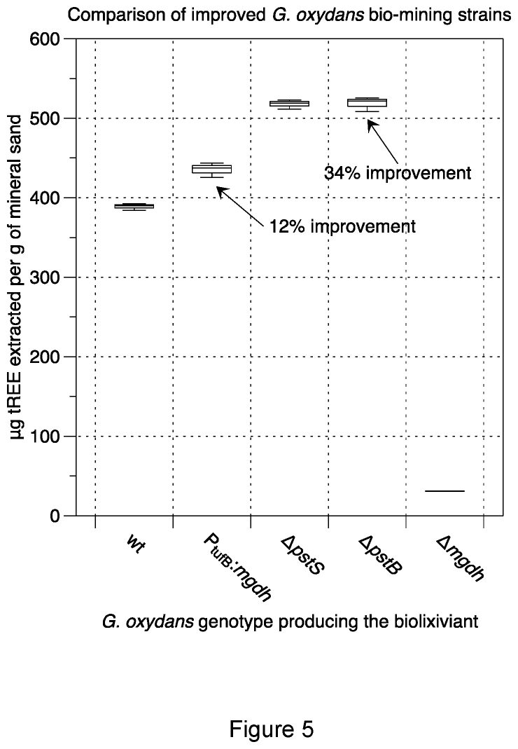

To characterize the genome of G. oxydans and identify a comprehensive set of genes underlying its bioleaching capabilities, we built a carefully curated whole-genome knockout collection of single-gene transposon disruption mutants using Knockout Sudoku (FIG. 1). Final pH of the biolixiviant is a good predictor for bioleaching efficiency, thus we used acidification as a proxy for bioleaching potential and have thoroughly screened the collection to identify mutants that differ in their ability to produce acidic biolixiviant (FIGS. 2 and 3). In one non-limiting example, we demonstrate that a single gene disruption—only one of several potential enhancement strategies—can significantly improve G. oxydans bioleaching capabilities (FIG. 4). In one limiting example, we demonstrate that increasing expression of mgdh or cleanly deleting pstb or psts can significantly improve G. oxydans bioleaching capabilities (FIG. 5).

Development of a Knockout Collection for G. oxydans Covering 2,733 Genes

We built a saturating coverage transposon insertion mutant collection for G. oxydans B58 and catalogued and condensed it with the Knockout Sudoku combinatorial pooling method (FIG. 1). We sequenced the G. oxydans B58 genome and identified 3,283 open reading frames (FIG. 1A). Following the recommendation of Monte Carlo simulations, we constructed a saturating coverage transposon insertion collection (the progenitor collection; PC) containing 49,256 mutants (FIG. 1B).

The progenitor collection catalog indicates that we were able to generate at least one disruption mutant for almost every non-essential gene in the G. oxydans genome. In total, we identified disruption strains for 2,733 genes out of the 3,283 genes in the G. oxydans B58 genome. Since every predicted gene contains at least seven AT or TA transposon insertion sites, the remaining 550 non-disrupted genes are likely to be essential. A Fisher's Exact Test for gene ontology (GO) enrichment representing 268 of the non-disrupted genes demonstrated significant enrichment in several essential ontologies, with the greatest enrichment in those relating to the ribosome and translation (FIG. 1C).

The progenitor collection catalog was used to create a condensed G. oxydans disruption collection with at least one representative per non-essential gene. 47 progenitor strains were verified by Sanger sequencing prior to condensing, of which 43 (92%) were confirmed to have the predicted transposon coordinate. We selected one mutant for all 2,733 disrupted genes, a second mutant for 2,354 genes, and a third mutant for 50 genes where mutant location information was poor. All mutants were struck out for single colonies, and 2-10 colonies per mutant were picked, depending on the predicted number of cross-contaminating disruption strains in the originating well. This condensed collection contains 17,706 mutants in 185 96-well plates.

The condensed collection catalog was validated by a second round of combinatorial pooling and sequencing. Of the 17,706 wells in the condensed collection, we were able to confirm the identity for 15,257. We confirmed 25 of these wells by Sanger sequencing, and 100% have the predicted transposon coordinate. Among these wells, we were able to verify the identity and location of 4,419 independent transposon insertion sites, representing a disruption mutant for 2,556 unique genes (FIG. 1D). 1,587 genes are represented by more than one disruption, and 3,317 of all disruptions occur in the first half of the gene.

### Genome-Wide Screening Discovers 165 Genes Significantly Linked to Acid Production

We screened the new G. oxydans B58 whole genome knockout collection for disruption mutants with differential acidification capability (FIG. 2). We used the colorimetric pH sensitive dyes Thymol Blue (TB) to screen for changes in final biolixiviant pH (FIG. 2A), and Bromophenol Blue (BPB) to screen for changes in rate of acidification (FIG. 2B).

In total, we observed 304 genes that apparently controlled acidification (FIG. 2C). The TB screen discovered 282 genes whose disruption leads to a differential change in biolixiviant acidity (FIG. 2C). 47 mutants produced a more acidic biolixiviant, while 235 produced a less acidic one (FIG. 2C). The BPB screen identified 82 gene disruptions with differential rate of acidification: 49 with a faster rate, and 33 with a slower rate. 60 mutants were identified by both screens (FIG. 2C). Overall, we identified 165 genes that statistically significantly changed the final biolixiviant pH, rate of acidification, or both, but did not change the growth rate (FIG. 2C). We re-arrayed disruption strains with differential acidification into new 96-well plates alongside proxy wild-type (pWT) strains that have a transposon insertion in an intergenic region and show non-differential growth or biolixiviant production. The new collection was re-assayed with the TB and BPB assays and the strength and significance of each result was determined by comparison with pWT through a Bonferroni-corrected t-test. Mutants that cause the 25 largest reductions, and 50 largest increases in endpoint acidity yet do not affect growth rate are shown in FIG. 2D. 31 mutants that cause significant changes in acidification rate without changing growth rate are shown in FIG. 2E. However, 14 of the faster strains, including δGO_868, a disruption of a LacI type transcriptional repressor which was the fastest strain, produced a less acidic biolixiviant than the wild-type, indicating that targeting these genes for engineering a faster acidifier would likely be at the expense of a more acidic biolixiviant. None of the strains with a faster rate of acidification also created a more acidic biolixiviant. These results indicated that multiple genetic engineering interventions are needed to construct a strain of G. oxydans that simultaneously produces a more acidic biolixiviant than the wild-type at a faster initial rate.

### Phosphate Transport and PQQ Synthesis are the Biggest Controllers of Acidification

We used gene ontology enrichment to determine which biological processes, metabolic functions, and cellular components the most significant gene disruption mutants are involved in (FIG. 3). Among the disrupted genes that led to a stronger acidity (FIG. 3A), the most significant enrichment for all three GO categories involves the phosphate-specific transport system, represented by pstA, pstB, pstC, pstS, and phoR. Other enriched ontologies include those related to phosphate signaling and binding.

Among the disrupted genes that led to a weaker acidity, several enrichment groups are related to the synthesis or use of the redox cofactor, PQQ, represented by pqqB, pqqC, pqqE, tldD, and mgdh (FIG. 3B). Other enriched ontologies include those related to carbohydrate metabolism.

Acidification rate is controlled by carbohydrate metabolism and respiration. Disruptions in the pentose phosphate pathway increase acidification rate (FIG. 3C). Meanwhile, disruptions of the electron transport pathway components are the most significantly enriched group of mutants that decrease acidification rate (FIG. 3D).

### Single Gene Knockout Mutants can Significantly Change REE Bioleaching

### Validation of Dye Assays by Direct pH Measurements

We selected 14 strains with some of the most significantly increased or decreased acidification for further testing (FIGS. 2D and E). Dye pH measurements were validated by direct pH measurements. 11 of the 13 strains that produced significantly lower pH biolixiviant in TB assays did the same in direct pH measurements. The most acidic biolixiviant was produced by a disruption in the phosphate transport gene, δpstC at pH 2.09 (FIG. 4A). 4 of the 11 mutant strains that produced a more acidic biolixiviant were disrupted in genes involved in the pst phosphate-specific transport system (δpstA, δpstB, δpstC, and δpstS).

Additional disruptions that led to a more acidic biolixiviant included those in a hypothetical protein with no similarity to anything previously characterized (δGO_1415S); a gamma-glutamyltranspeptidase (δggtl); a periplasmic chaperone (δsurA); an HTH-type transcriptional regulator (δpetP); a two-component system sensor histidine kinase (δykoH); a Pyridoxal 5-phosphate (PLP)-dependent ornithine decarboxylase (δspeC); and a TPR domain protein that is a putative component of the TonB iron uptake system (δtonB).

9 of the tested strains produced biolixiviant significantly higher in pH than pWT (FIG. 4B). The most alkaline biolixiviant was produced by a disruption in the PQQ synthesis system, δpqqC. at pH 4.71. While δpqqC produced very little acid, this is below the pH of glucose in media alone, indicating that some bacterial acidification still occurred. 3 of the 9 mutants that produce reduced acidity biolixiviant either synthesize PQQ (δpqqC and δtldD), or use it as a cofactor (δmgdh). Additional disruptions that led to a more alkaline biolixiviant than pWT include a Fructose-bisphosphate aldolase class II (δGO_3252); a GTP and nucleic acid binding protein (δchF); a lipid A biosynthesis protein (δhtrB); a peptide chain release factor (δhemK); the LacI type transcriptional repressor that increases initial acidification rate (δGO_868); components of a proteolytic complex (δtldD and δtldE); and the glucose dehydrogenase (δmgdh).

### Disrupting the Phosphate Transport System Significantly Increases Bioleaching

We tested if 10 of the mutants that produced a more acidic biolixiviant could bioleach REE from retorted phosphor powder (RPP) from spent fluorescent lightbulbs more efficiently than pWT (FIG. 4C). For each mutant, the elemental composition of REE leachate was similar to previous reported values. Six of these mutants significantly increased bioleaching. Two of the better bioleaching mutants disrupted the pst phosphate transport system (δpstC and δpstB). Overall, we found that bioleaching efficiency correlates with biolixiviant pH, as expected (FIG. 4E).

The δpstC mutant produced the most acidic biolixivant, and extracted the most REE from RPP: 5.5% total extraction efficiency as compared with pWT's 4.7%. Stated differently, δpstC removed 18% more REE from RPP than pWT. This increase in REE extraction remains significant even under a Bonferonni correction, the most stringent statistical test for significance. Without the adjustment, six of the better acidifiers were also better bioleachers than pWT (FIG. 4C). The remaining better bioleachers increased REE extraction by between 11% (δspeC) and 18% (δggtl) (FIG. 4C).

Without intending to be bound by any particular theory, it is considered that disrupting the phosphate transport system de-represses acid production in G. oxydans. Six of the disruption strains that resulted in a lower biolixiviant pH (δpstC, δpstB, δggtl, δpstA, δpstS, and δykoH), including three that increased bioleaching (δpstC, δpstB, δggtl), are involved in phosphate transport, sensing and signaling.

In its natural environment, G. oxydans produces biolixiviants to liberate phosphate from minerals, not metals. Under phosphate-limiting conditions, the PstSCAB phosphate transporter will activate the histidine kinase, PhoR, which in turn phosphorylates the transcription factor PhoB, and activates the pho regulon, enabling phosphate assimilation and uptake. Under sufficient phosphate conditions, PhoB is deactivated by PhoR, which in turn inhibits expression of these genes. Without intending to be constrained by any particular view, it is considered that disrupting any of these genes prevents G. oxydans from sensing when it has released adequate phosphate and when to stop producing biolixiviants.

### Disrupting Mgdh and PQQ Synthesis Genes Significantly Decreases Bioleaching

We also tested REE extraction by 4 mutants that produce a less acidic biolixiviant than pWT. Even under the most stringent statistical test, the Bonferonni correction, they were all worse bioleachers than pWT (FIGS. 4C and D).

The δmgdh mutant was the worst bioleacher of all tested, considering its lack of gluconic-acid production. δmgdh reduced bioleaching by 97%. Disruption mutants that knocked out synthesis of mGDH's essential redox cofactor, PQQ, also produced significant reductions in biolixiviant acidity. δpqqC reduced bioleaching by ≈94%. While bioleaching by δmgdh and δpqqC was negligible compared to pWT, they were able to bioleach a statistically significant amount of REE compared to glucose alone. This indicates, that a bioleaching mechanism independent of mGDH exists in G. oxydans (FIG. 4D).

Disruption mutants in tldD and tldE were also much worse at bioleaching than pWT. δtldD reduces bioleaching by 92%, while δtldE reduces it by 63% (FIG. 4C). It is considered that TldD and TldE may contribute to the supply of the PQQ cofactor to mGDH. δtldD strongly attenuates acid production (FIG. 4B), and the gene has already been implicated in PQQ synthesis in G. oxydans 621H. In E. coli, TldD and TldE form a two-component protease for the final cleavage step in the processing of the peptide antibiotic, Microcin B17. In a similar manner, PqqF and PqqG from Methylorubrum extorquens form a protease that releases PQQ in the final step of its synthesis. It is considered that TldD in G. oxydans may play the same role as PqqF from M. extorquens, while TldE plays the same role as PqqG. Deletion of pqqF in M. extorquens completely inhibits final cleavage of PQQ, while we find that disruption of tldD in G. oxydans reduces REE bioleaching by 92%. Moreover, deletion of pqqG in M. extorquens only reduces PQQ cleavage to 50%, while disruption of tldE only reduces REE bioleaching by 63%. These parallels strongly indicate a novel role for TldE in the biosynthesis of PQQ in G. oxydans.

It will be recognized from the foregoing description that bioleaching has the potential to revolutionize the environmental impact of REE production, and dramatically increase access to these critical ingredients for sustainable energy technology. The present disclosure related to this potential by providing for improved bioleaching by genetic engineering.

By constructing a whole genome knockout collection for G. oxydans, we are able to characterize the genetics of this process with high sensitivity and high completeness. In total we identified 165 gene disruption mutants that significantly change the acidity of its biolixiviant, rate of production, or both.

REE bioleaching by G. oxydans is predominantly controlled by two well-characterized systems: phosphate signaling and glucose oxidation that is supported by production of the redox cofactor PQQ. Interrupting phosphate signaling control of biolixiviant production by disrupting a single gene (pstC) can increase REE extraction by 18%. Disrupting the supply of the PQQ cofactor to the membrane bound glucose dehydrogenase reduces REE extraction by up to 92%.

Comprehensive screening of the G. oxydans genome also discovers completely new targets that contribute as much to REE bioleaching as previously characterized ones. For example, disrupting GO_1415, which encodes a protein of completely unknown function, increases REE bioleaching by 15%. Additionally, these results highlight the potential for a previously uncharacterized role of TldE in PQQ synthesis.

The discovery of the potential contribution of TldE to PQQ biosynthesis may allow for marked enhancement of the cofactor production through the additional over overexpression of this gene, and a consequent increase in dehydrogenase activity, including production of gluconic acid by mGDH and any useful downstream products. PQQ is an essential cofactor important for several other industrial applications of G. oxydans, including production of L-sorbose. Furthermore, PQQ alone has many applications across many biological processes from plant protection to neuron regeneration.

Without intending to be bound by any particular theory, it is believed that the present disclosure provides the first demonstration of improvement of bioleaching through genetic engineering. Furthermore, the creation of a whole-genome knockout collection in G. oxydans can facilitate its use as a model species for further studies in REE bioleaching and other industrially important applications of similar acetic acid bacteria. The findings of the two major systems contributing to acidification in G. oxydans according to this disclosure show that, for greatly improving bioleaching: reduce inhibition of regulation of acid production by disabling the phosphate-specific transport system, while over-expressing mgdh along with the expanded synthesis pathway for its cofactor PQQ.

### Materials and Methods

Gluconobacter oxydans B58 Genome Sequencing

Gluconobacter oxydans strain NRRL B-58 (GoB58) was obtained from the American Type Culture Collection (ATTC), Manassas, VA. In all experiments, G. oxydans was cultured in yeast peptone mannitol media (YPM; 5 g L⁻¹ yeast extract, 3 g L⁻¹ peptone, 25 g L⁻¹ mannitol), with or without antibiotic, as specified.

Genomic DNA was extracted from saturated culture using a Quick-DNA Miniprep kit from Zymo Research (Part number D3024, Irvine, CA). Genomic DNA library was prepared and sequenced using a TruSeq DNA PCR-Free Library Prep Kit (Illumina, San Diego, CA).

The prepared library was sequenced on a MiSeq Nano (Illumina, San Diego, CA, USA) with a 500 bp kit at the Cornell University Institute of Biotechnology (Ithaca, NY, USA). Resulting paired end reads were trimmed using Trimmomatic and assembled with SPAdes using k-mer sizes 21, 33, 55, 77, 99, and 127, and an auto coverage cutoff. Assembly quality was checked with QUAST and genome completeness was verified with BUSCO using the proteobacteria_odb9 database for comparison. The resulting 62 contigs were annotated online using RAST (rast.nmpdr.org).

### Gene Ontology Enrichment

DIAMOND was used to assign annotated protein models with a closest blast hit using the uniref90 database, an E-value threshold of 10⁻¹⁰, and a block size of 10. InterProScan (version 5.50-84.0) was used to assign family and domain information to protein models.

Output from both of these searches was used to assign gene ontologies with BLAST2GO. Gene set enrichment analysis was done with BioConductor topGO package, using the default weight algorithm, the TopGO Fisher test, with a p-value threshold of 0.05.

### Mating for Transposon Insertional Mutagenesis

The transposon insertion plasmid, pMiniHimarFRT was delivered to GoB58 by conjugation with E. coli WM3064. E. coli WM3064 transformed with pMiniHimarFRT was grown overnight to saturation in 50 mL LB (10 g L⁻¹ tryptone, 5 g L⁻¹ yeast extract, and 10 g L⁻¹ NaCl) supplemented with 50 μg mL⁻¹ kanamycin (kan) and 90 μM diaminopimelic acid (DAP), rinsed once with 50 mL LB, then re-suspended in 20 mL YPM.

We used a Monte Carlo numerical simulation (collectionmc) to approximate how many insertions would need to occur before a mutant is found representing a knockout of each gene in the genome. Our calculations demonstrated that we would be able to identify mutants in at least 99% of all G. oxydans B58 genes if we generated and selected at least 55,000 mutants (FIG. 1B).

GoB58 was grown for approximately 24 hours in YPM, then back-diluted to an optical density (OD) of 0.05 in 750 mL YPM and incubated at 30° C. for two doublings until the OD reached 0.2. GoB58 culture was distributed into 13 50 mL conical tubes, to which rinsed and re-suspended WM3064 was added at a ratio of 1:1 by density (approximately 1 mL WM3064 to 50 mL B58). Bacteria were mixed by inversion then spun down at 1900 g for 5 minutes. Supernatant was poured off, and the mixture was resuspended in the remaining liquid (≈0.5 mL), pipetted onto a YPM plate in 5 spots of 0.1 mL, and allowed to dry on the bench under a flame.

Mating plates were incubated at 30° C. for 24 hours. Mating spots were collected by adding 4 mL YPM to a plate, scraping the spots into the liquid, then suspending by pipetting up and down several times. Suspended cells were collected from each plate, and the suspension was plated onto YPM agar with 100 μg mL⁻¹ kanamycin at 100 μL per plate.

After 3 days of incubation at 30° C., colonies were picked into 96-well microplates using a CP7200 colony picking robot (Norgren Systems, Ronceverte WV, USA). Each well contained 150 μL YPM with 100 μg mL⁻¹ kanamycin. For all experiments, GoB58 was grown in polypropylene microplates sealed with a sterile porous membrane (Aeraseal, Catalog Number BS-25, Excel Scientific) and incubated at 30° C. shaking at 800 rpm. Isolated disruption strains were grown for three days to allow nearly all wells to reach saturation. Wells B2 and E7 of each plate were reserved as no-bacteria controls.

In total 18 matings were required to recover and pick a progenitor collection of 49,256 disruption strains into 525 microplates over the course of about two months. Microplates with saturated wells were maintained at 4° C. for up to 3 weeks and incubated an extra night at 30° C. before pooling.

Combinatorial pooling which was done in three batches. The 525 plates were virtually arranged in a 20 by 27 grid, and combinatorial pooling, cryopreservation, pool amplicon library generation, and sequencing were all done as previously described.

### Curation of a Whole-Genome Knockout Collection

Sequencing data for the progenitor collection was processed into a progenitor collection catalog using the KOSUDOKU suite of algorithms. To create a condensed collection, a disruption strain was chosen for each of the 2,733 disrupted genes available in the progenitor collection, first prioritizing close proximity to the translation start, then the total probability of the proposed progenitor collection address. A second strain was chosen from the remaining strains for each gene that had another available. For 50 genes, both disruption strains selected were ambiguously located, and thus a third strain was selected from the remaining collection.

In total, 5,137 disruption strains were isolated and struck-out for single colonies. Many progenitor wells were predicted to have more than one possible strain per well, so for each strain, the number of colonies isolated was two times the predicted number of strains in the progenitor well, up to ten. The condensed collection, which amounted to 17,706 wells, was pooled, sequenced, and validated as previously described. Unknown disruption strains significantly linked to acidification were identified with Sanger sequencing, also as previously described, with the exception of the transposon-specific primers. For the first and second rounds of nested PCR, the transposon-specific primers were (5′-GTATCGCCGCTCCCG-3′ (SEQ ID NO: 309), and (5′-CATCGCCTTCTATCGCCTTC-3′ (SEQ ID NO: 310)), respectively.

### Thymol Blue Endpoint Acidity Assay

Endpoint acidity was measured using the pH indicator thymol blue (TB, Sigma-Aldrich, St. Louis, MO), which changes from red to yellow below a pH of 2.8 (www.sigmaaldrich.com/US/en/product/sial/114545). The lowest pH of biolixiviant generated by GoB58 was 2.3 (Reed2016a), thus TB allows for distinguishing strains that lower the pH below that of the wild type biolixiviant. To generate biolixiviant, the condensed collection was pin replicated into new growth plates containing 100 μL YPM with 100 μg mL⁻¹ kanamycin per well. After two days of growth, an equal volume of 40% w/v glucose was added to the cultures for a final solution of 20% w/v glucose. The amount of glucose needed to lower the pH below 2.3 via the production of gluconic acid was estimated to be 13% w/v, but the higher concentration was used to account for any use of glucose as a carbon source and still maintain an excess amount. Viability tests demonstrated that the bacteria were still viable after two days of culture in such a solution (data not shown).

Bacteria were incubated with glucose for 48 hours to allow acid production to reach completion. Plates were then centrifuged for 3 minutes at 3200 g (top speed) and 90 μL of the biolixiviant supernatant was removed and add to TB at a final concentration of 40 μg mL⁻¹. After 1 minute of vortexing, absorbance was measured for each well at 435 nm and 545 nm on a Synergy 2 plate reader (Biotek Instruments, Winooski, VT, USA). Because of variation in background absorbance from well to well on each plate, absorbance was measured at these two wavelengths, and their ratio was used as a proxy for pH, which correlates linearly within the range of pH for the majority of biolixiviants produced by the collection.

### Bromophenol Blue Acidification Rate Screen

Acidification rate was measured using the pH indicating dye, Bromophenol Blue (BPB). Knockout collection strains were grown for two days. OD was measured at 590 nm for each well, then 5 μL of culture was transferred to a polystyrene assay plate containing 95 μL of 2% w/v glucose and 20 μg mL⁻¹ BPB in deionized water. The initial pH of the culture is just above 5, and within moments of adding culture to glucose with BPB, the color begins to change rapidly. Assay plates were vortexed for one minute after addition of bacterial culture, then immediately transferred to a plate reader where the change in color was tracked by measuring absorbance at 600 nm every minute for 6 minutes, resulting in 7 reads. Mean rate (V) and R-squared were calculated by the Gen5 microplate reader and imager software (Biotek Instruments). A plot of all V relative to OD demonstrated that the two are correlated, thus V was normalized to OD for each well.

### Hit Identification in Acidification End Point and Rate Screens

Once every well had its assigned data point (A435/A545 for TB, and V/OD for BPB), hits were determined by first identifying outliers for each plate. The interquartile range and upper and lower bounds were calculated in Microsoft Excel considering all wells with cultured disruption strains. Any data point that was more than 1.5 times over or under the upper or lower bound, respectively, was considered an outlier. A disruption strain was considered a hit if over half of the wells for that strain (or 1 of 2) were outliers.

Acidification End Point and Rate Quantification with Colorimetric Dyes

For each assay, knockout strains identified as hits were isolated from the knockout collection into new microplates, along with several blanks per plate, and proxy wild type strains—GoB58 strains with an intergenic transposon insertion that should not affect the acidification phenotype. OD and acidification phenotypes were measured for each proxy WT strain separately to verify that growth and acidification are unaffected in these strains.

Acidification phenotypes for the disruption strains were compared to that of proxy WT with a Student's t-test in Microsoft Excel, two-tailed with equal variance. A Bonferroni correction was used to determine significance to account for the possibility a comparison is significant by chance alone: a phenotype was considered significant if p>0.05/n, where n is the number of comparisons being made (n=120 or n=242 for endpoint acidity comparisons with pWT set A or set B, respectively; n=60 for rate of acidification comparisons with pWT).

### Choice of Proxy Wild-Type Comparison

The biolixiviant end point pH and acidification rate of each G. oxydans mutant were compared against a proxy wild-type set of mutants for each phenotype. To account for the presence of a kanamycin cassette in the genome, the proxy wild-type set for each phenotype was constructed of several mutants with the transposon inserted in an intergenic region, that had no growth defect, and no apparent change in phenotype.

As the efficiency of the E. coli WM3064 to G. oxydans mating was low, we constructed the G. oxydans progenitor collection in 18 mating batches. As a result of this, the possibility existed that there might be slight variations in the wild type background from batch to batch.

For the acidification rate, we found that these variations did not affect the wild-type behavior across the collection, and a single set of proxy wild-type strains could be used as a comparison with notable disruption strains in the quantification assays. For the end point pH measurement, we found two distinct proxy wild-type behaviors in the condensed collection. For plates 1 to 76; 110 to 130; and 160 to 185, we used proxy wild-type set A, and for plates 77 to 109 and 130 to 159 we used proxy wild-type set B.

For both wild-type sets, we compared ODs after two days of growth, and endpoint acidity using the TB absorbance ration (A435/A545). For wild-type set A, which we used for the BPB quantification assay, we also compared acidification rate of individual proxy WT strains of set A. Comparisons were all made using a linear model, one-way ANOVA, and post-hoc Tukey HSD in R.

### Direct Measurement of Biolixiviant pH

Bacteria were grown for 48 hours in tubes containing 4 mL YPM with 100 μg mL⁻¹ kanamycin. One tube was left uninoculated as a no-bacteria control. OD was normalized to 1.9 and diluted in half with 40% glucose for a final 20% solution in 1.5 mL. Five replicates were created for each strain and controls, and all mixtures were randomly distributed across two deep well plates. 750 μL of mixture was transferred from each well to a second set of deep-well plates for bioleaching experiments. All plates were incubated shaking at 900 rpm at room temperature.

After two days, one set of deep-well plates was centrifuged for 10 minutes at 3200 g (top speed), and the pH of the supernatant was measured by insertion of a micro-probe to the same depth in each well.

Four standards were used for meter calibration—pH 1, 2, 4, and 7—and the meter was re-calibrated after every 12 measurements. pH measurements for each disruption strain were compared with those of proxy WT using a Student's t-test in Microsoft Excel, two-tailed, with equal variance. A biolixiviant pH was considered significantly different if p<0.05/n, with n=27.

### Direct Measurement of REE Bioleaching

The second set of deep-well plates was centrifuged for 10 minutes at 3200 g (top speed), and 500 μL of biolixiviant was transferred from each well to a 1.7 mL Eppendorf tube. 20 mg (4% w/v) of retorted phosphor powder was added to each tube for bioleaching. Tubes were shaken horizontally for 36 hours at room temperature, then centrifuged to pellet remaining solids. Supernatant with leached REE was filtered through a 0.45 μm AcroPrep Advance 96-well Filter Plates (Pall Corporation, Show Low, AZ, USA) by centrifuging at 1500×g for 5 minutes.

All samples were diluted 1/200 in 2% trace metal grade nitric acid (Thermo Fisher Scientific) and analyzed by an Agilent 7800 ICP-MS for all REE concentrations using a rare earth element mix standard (Sigma-Aldrich) and a rhodium in-line internal standard (Sigma-Aldrich). Quality control was performed by periodic measurement of standards, blanks, and repeat samples

An additional 1/20 dilution in 2% nitric acid was analyzed for mgdh and pqqc disruption strains, and the no-bacteria control (glucose).

Bioleaching measurements for each disruption strain were compared with those of proxy WT or glucose using a Student's t-test in Microsoft Excel, two-tailed, with equal variance. Total REE extracted was considered significantly different if p<0.05/n, with n=12 for those compared to pWT, and n=2 for those compared to gluco

1. Modified bacteria for use in bioleaching rare earth elements (REEs) from a composition comprising the REEs, the modified bacteria comprising at least one engineered genetic change that is correlated with improved bioleaching of the REEs, relative to REE bioleaching by unmodified bacteria of the same species as the modified bacteria, and wherein the at least one genetic change comprises a change that results in decreased expression, or increased expression, of at least one gene, and wherein the at least one gene optionally encodes a protein that participates phosphate-specific transport system signaling, or encodes a protein that participates in pyrroloquinoline quinone (PQQ) synthesis.

2. The modified bacteria of claim 1, wherein expression of the gene that encodes a protein that participates in the phosphate-specific transport system signaling is suppressed, and wherein said gene is optionally pstS, pstB or pstC.

3. The modified bacteria of claim 1, wherein expression of the gene that encodes a protein that participates in the PQQ synthesis is increased, and wherein said gene is optionally selected from the group consisting of pqqA, pqqB, pqqC, pqqD, pqqE, tldD and tldE.

4. The modified bacteria of claim 1, wherein, in addition to the at least one genetic change, the modified bacteria have been modified to increase expression of mgdh relative to expression of mgdh by unmodified bacteria.

5. The modified bacteria of claim 1, wherein expression of pstS is reduced.

6. The modified bacteria of claim 1, wherein expression of pstB is reduced.

7. The modified bacteria of claim 1, wherein pstS, pstB, pstC, or a combination thereof is reduced, wherein expression of pqqA, pqqB, pgqC, pqqD, pqqE, tldD, tldE, or a combination thereof is increased, and wherein the expression of mgdh is also increased.

8. The modified bacteria of claim 5, wherein the modified bacteria are Gluconobacter oxydans.

9. The modified bacteria of claim 6 wherein the modified bacteria are Gluconobacter oxydans.

10. The modified bacteria of claim 7, wherein the modified bacteria are Gluconobacter oxydans.

11. A method comprising contacting a composition comprising rare earth elements (REEs) with a biolixivant produced by modified bacteria of claim 1.

12. The method of claim 11, further comprising separating REEs from the composition.

13. The method of claim 11, wherein expression of pstS is reduced in the modified bacteria.

14. The method of claim 13, further comprising separating REEs from the composition.

15. The method of claim 11, wherein expression of pstB is reduced in the modified bacteria.

16. The method of claim 15, further comprising separating REEs from the composition.

17. The method of claim 11, wherein pstS, pstB, pstC, or a combination thereof is reduced, wherein expression of pqqA, pqqB, pqqC, pqqD, pqqE, tldD, tldE, or a combination thereof is increased, and wherein the expression of mgdh is also increased in the modified bacteria.

18. The method of claim 17, further comprising separating REEs from the composition.

19. A kit comprising modified bacteria of claim 1 the kit further comprising one more sealable containers in which said modified bacteria are held.

20. The kit of claim 19, wherein the modified bacteria are modified Gluconobacter oxydans.
